(19) United States
(12) Patent Application Publication
(10) Pub. No.: US 2023/0365542 A1
(43) Pub. Date: Nov. 16, 2023

(54) Selective Agonists of 5-HT2A Receptor and Methods of Use

(71) Applicants: Yale University, New Haven, CT (US); The University of North Carolina at Chapel Hill, Chapel Hill, NC (US); THE REGENTS OF THE UNIVERSITY OF CALIFORNIA, Oakland, CA (US)

(72) Inventors: Jonathan Ellman, New Haven, CT (US); Danielle Confair, New Haven, CT (US); Oh Sang Kweon, New Haven, CT (US); Bryan Roth, Durham, NC (US); Kuglae Kim, Chapel Hill, NC (US); Brian Shoichet, Kentfield, CA (US); Anat Levit, Foster City, CA (US); John Irwin, Orinda, CA (US)

(73) Assignee: The Regents of the University of California, Oakland, CA (US)

(21) Appl. No.: 18/247,057

(22) PCT Filed: Sep. 27, 2021
(86) PCT No.: PCT/US2021/052163

§ 371 Date: Mar. 28, 2023

Related U.S. Application Data
(60) Provisional application No. 63/084,143, filed on Sep. 28, 2020.

(51) Int. Cl. C07D 405/14; C07D 401/14; C07D 401/04; C07D 471/04

(52) U.S. Cl. CPC C07D 405/14; C07D 401/14; C07D 401/04; C07D 471/04

(57) ABSTRACT

In one aspect, the present disclosure describes tetrahydropyridine compounds of formula (I), which are 5-HT_{2A} receptor agonists that exhibit selective binding to the 5-HT_{2A} receptor over the 5-HT_{2B} receptor. In certain embodiments, the compound of formula (I) is a compound of formula (II). Also provided herein are methods of treating, ameliorating, and/or preventing neurological diseases and disorders with compounds of formula (II).

### CROSS-REFERENCE TO RELATED APPLICATIONS

This application claims priority under 35 U.S.C. § 119(e) to U.S. Provisional Patent Application No. 63/084,143, filed Sep. 28, 2020, which is incorporated herein by reference in its entirety.

### STATEMENT REGARDING FEDERALLY SPONSORED RESEARCH

This invention was made with government support under GM122481, GM071896, GM122473, and MH112205 awarded by National Institutes of Health and under HR0011-20-2-0029 awarded by the Defense Advanced Research Projects Agency. The government has certain rights in the invention.

### BACKGROUND OF THE DISCLOSURE

Agonists of the 5-hydroxytryptamine 2A receptor (5-HT_{2A}R) are sought after as potential pharmaceuticals for a variety of neuropsychiatric diseases including but not limited to depression, anxiety, substance abuse, migraine headaches, and/or cluster headaches, and various somatic illnesses including but not limited to various inflammatory, cardiovascular, genitourinary, and/or pain disorders. While many 5-HT_{2A}R agonists are known, few if any are selective for this receptor over related subtypes, especially over the 5-HT_{2B} receptor, a toxicology anti-target strongly implicated in serious side effects including drug-induced valvular heart disease.

There is thus an unmet need in the art for novel 5-HT_{2A} receptor agonists. In certain embodiments, these agonists exhibit selective binding to the 5-HT_{2A} receptor over the 5-HT_{2B} receptor. The present disclosure meets this need.

### BRIEF SUMMARY OF THE DISCLOSURE

The disclosure provides certain compounds of formula (I), or a salt, solvate, tautomer, N-oxide, geometric isomer, and/or stereoisomer thereof, wherein the substituents in (I) are defined elsewhere herein:

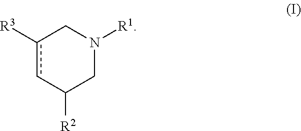

In certain embodiments, the compound of formula (I) is a compound of formula (II), wherein the substituents in (II) are defined elsewhere herein.

The disclosure further provides pharmaceutical compositions comprising at least one compound of the disclosure and at least one pharmaceutically acceptable excipient. In certain embodiments, the pharmaceutical compositions comprise at least one additional therapeutic agent that treats, ameliorates, and/or prevents a neurological disease and/or disorder.

The disclosure further provides a method of treating, ameliorating, and/or preventing a neurological disease and/or disorder, the method comprising administering to a subject in need thereof a therapeutically effective amount of a compound of a compound of formula (II) and/or a pharmaceutical composition thereof. In certain embodiments, the neurological disease or disorder is selected from the group consisting of depression, anxiety, substance abuse, and headaches.

The disclosure further provides a method of selectively agonizing the 5-hydroxytryptamine 2A (5-HT_{2A}) receptor in a subject in need thereof, the method comprising administering to a subject in need thereof a therapeutically effective amount of a compound of formula (II) and/or a pharmaceutical composition thereof.

### BRIEF DESCRIPTION OF THE FIGURES

The drawings illustrate generally, by way of example, but not by way of limitation, various embodiments of the present application.

FIG. 1 illustrates an X-ray crystal structure of compound 19 and picrylsulfonic acid dihydrate, in accordance with various embodiments. Thermal ellipsoids at 50% probability levels are shown. The hydrogen atoms are shown as circles for clarity.

FIG. 2 illustrates an X-ray crystal structure of compound 34 and picrylsulfonic acid dihydrate, in accordance with various embodiments. Thermal ellipsoids at 50% probability levels are shown. The hydrogen atoms are shown as circles for clarity.

FIG. 3 illustrates an X-ray crystal structure of compound 38 and picrylsulfonic acid dihydrate, in accordance with various embodiments. Thermal ellipsoids at 50% probability levels are shown. The hydrogen atoms are shown as circles for clarity.

FIGS. 4A-4C: behavioral response of C57BL/6J mice to compounds 38 and 33. FIG. 4A: head twitch responses (HTRs) from C57BL/6J mice during first 30 min after injection (i.p.) of the vehicle (blue), 1 mg/kg 38 (green), 1 mg/kg 33 (red), or 0.3 mg/kg LSD (yellow). One-way ANOVA: treatment [F(1,35)=72.008, p<0.001] HTRs were stimulated with LSD relative to the vehicle and the 38 and 33 groups (p-values<0.001), that were not different from each other. FIG. 4B: distance travelled in the open field by C57BL/6J mice given the same injections. Left: baseline locomotion (0-30 min); Right: locomotion post injection (31-60 min). RMANOVA: pre-post [F(1,35)=28.926, p<0.001], treatment [F(3,35)=10.390, p<0.001], pre-post by treatment [F(3,35)=39.901, p<0.001]. No treatment effects were found during the pre-injection period (0-30 min). During the post-injection period (31-60 min) locomotor activities were significantly higher in the LSD than the other groups (p-values≤0.016), that were not different from one another. (n=9-10 mice/treatment). FIG. 4C: immobility time during the tail suspension test in wild-type (WT, open bars) and vesicular monoamine transporter 2 (VMAT2) heterozygous (HET, cross-hatched bars) mice 30 min after administration of the vehicle, 20 mg/kg fluoxetine, 0.5 or 1 mg/kg 38, or 0.5 or 1 mg/kg 33 (i.p.). Two-way ANOVA: treatment [F(5,90)=9.593, p<0.001] and genotype by treatment [F(5,90)=9.103, p<0.001]. Vehicle treated HET mice spent more time immobile than WT controls (p<0.001); HET mice treated with 1 mg/kg 33 spent less time immobile than WTs given the same treatment (p=0.051). All treatments reduced immobility time significantly in HET mice compared to its vehicle (p-values<0.001); no effects among WT mice. n=7-9 WT and 8-9 VMAT2-HET mice/treatment). *p<0.05, compared to WT; +p<0.05, compared to vehicle within genotype.

### DETAILED DESCRIPTION OF THE DISCLOSURE

The present disclosure provides in one aspect certain 5-HT_{2A} receptor agonists. In some embodiments, the agonists of the disclosure exhibit selective binding to the 5-HT_{2A} receptor over the 5-HT_{2B} receptor. In certain embodiments, compounds of the disclosure can be used to treat a variety of neuropsychiatric diseases including, but not limited to, depression, anxiety, substance abuse, migraine headaches, and/or cluster headaches.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in various embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any one of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in various embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in various embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In various embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In various embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In various embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “heteroalkyl” as used herein refers to alkyl groups as defined herein in which a which a hydrogen or carbon bond of an alkyl group is replaced with at least one heteroatom such as, but not limited to, N, O, and S.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise.

Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15-28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Preparation of Compounds

In various embodiments, a compound of formula (I), or a salt, solvate, tautomer, N-oxide, geometric isomer, and/or stereoisomer thereof, is provided. In various embodiments, the compound of formula (I) has the structure:

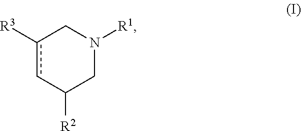

wherein:
[symbol] represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂;
each occurrence of R is independently H, C₁-C₁₂ alkyl, C₃-C₁₂ cycloalkyl, or —(C₁-C₁₂ alkyl)C₃-C₁₂ cycloalkyl.

In various embodiments, the compound has the structure of formula (I-A):

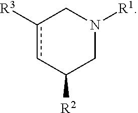

In various embodiments, the compound has the structure of formula (I-B):

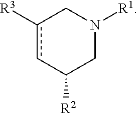

In various embodiments, the compound has the structure of formula (II-A):

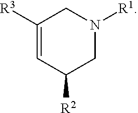

In various embodiments, the compound has the structure of formula (II-B):

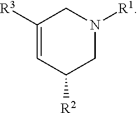

In various embodiments, the compound has the structure of formula (II-C):

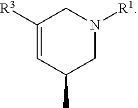

In various embodiments, the compound has the structure of formula (II-D):

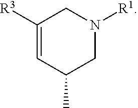

In various embodiments, the compound has the structure of formula (II-E):

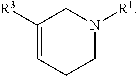

In various embodiments, the compound has the structure of formula (III-A):

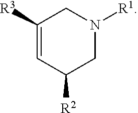

In various embodiments, the compound has the structure of formula (III-B):

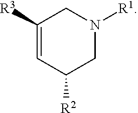

In various embodiments, the compound has the structure of formula (III-C):

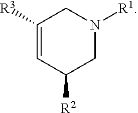

In various embodiments, the compound has the structure of formula (III-D):

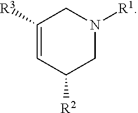

In various embodiments, the compound has the structure of formula (III-E):

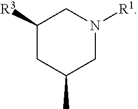

In various embodiments, the compound has the structure of formula (III-F):

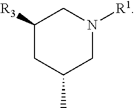

In various embodiments, the compound has the structure of formula (III-G):

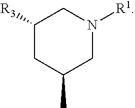

In various embodiments, the compound has the structure of formula (III-H):

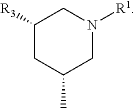

In various embodiments, the compound has the structure of formula (III-I):

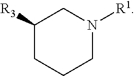

In various embodiments, the compound has the structure of formula (III-J):

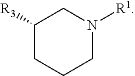

In various embodiments, [symbol] is a double bond. In various embodiments, [symbol] is a single bond.

In various embodiments, R¹ is H. In various embodiments, R¹ is optionally substituted C₁-C₁₂ alkyl. In various embodiments, R¹ is C₁-C₁₂ alkyl. In various embodiments, R¹ is optionally substituted C₁-C₁₂ heteroalkyl. In various embodiments, R¹ is optionally substituted C₃-C₁₂ cycloalkyl. In various embodiments, R¹ is optionally substituted —(C₁-C₁₂ alkyl)C₃-C₁₂ cycloalkyl. In various embodiments, R¹ is —(C₁-C₁₂ alkyl)C₃-C₁₂ cycloalkyl. In various embodiments, R¹ is optionally substituted C₂-C₁₈ heterocyclyl. In various embodiments, R¹ is optionally substituted —(C₁-C₁₂ alkyl)C₂-C₁₈ heterocyclyl. In various embodiments, R¹ is —(C₁-C₁₂ alkyl)C₂-C₁₈ heterocyclyl.

In various embodiments, R¹ is methyl. In various embodiments, R¹ is ethyl. In various embodiments, R¹ is n-propyl. In various embodiments, R¹ is n-butyl. In various embodiments, R¹ is i-pentyl. In various embodiments, R¹ is n-pentyl. In various embodiments, R¹ is —(CH₂)ₙ-cyclopropyl.

In various embodiments, R¹ is selected from the group consisting of —(CH₂)ₙ-cyclobutyl, —(CH₂)ₙ-cyclopentyl,

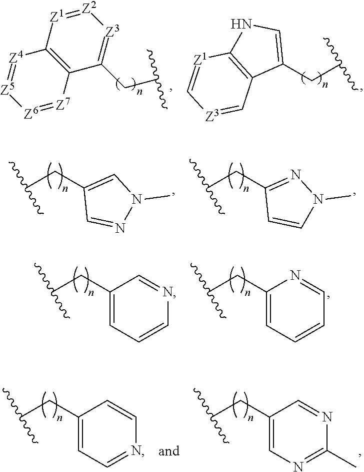

wherein
each Z¹ to Z⁷ is independently CH or N, and
each n is independently an integer from 0 to 6.

In various embodiments, R¹ is

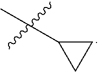 [SMILES: *C1CC1]

In various embodiments, R¹ is

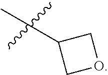 [SMILES: *C1COC1]

In various embodiments, R¹ is

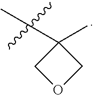 [SMILES: *C1(C)COC1]

In various embodiments, R¹ is

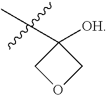 [SMILES: *C1(O)COC1]

In various embodiments, R¹ is

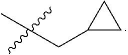 [SMILES: *CC1CC1]

In various embodiments, R¹ is

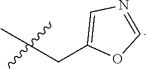 [SMILES: *Cc1cnco1]

In various embodiments, R¹ is

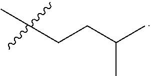 [SMILES: *CCC(C)C]

In various embodiments, R¹ is

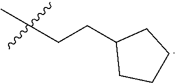 [SMILES: *CCC1CCCC1]

In various embodiments, R¹ is

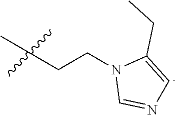 [SMILES: *CCn1cncc1CC]

In various embodiments, R¹ is

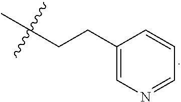 [SMILES: *CCc1cccnc1]

In various embodiments, R¹ is

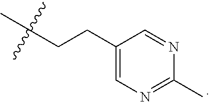 [SMILES: *CCc1cnc(C)nc1]

In various embodiments, R¹ is

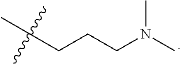 [SMILES: *CCCN(C)C]

In various embodiments, R¹ is

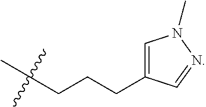 [SMILES: *CCCc1cnn(C)c1]

In various embodiments, R² is C₁-C₁₂ alkyl. In various embodiments, R² is H. In certain embodiments, R² is methyl, ethyl, or propyl in various non-limiting embodiments.

In various embodiments, R³ is an optionally substituted C₂-C₁₀ heterocyclyl. Variable R³ is, in various non-limiting embodiments, a C₂, C₃, C₄, C₅, C₆, C₈, C₉, or C₁₀ heterocyclyl, each of which is optionally substituted. In various embodiments, R³ is an optionally substituted C₂-C₁₀ heteroaryl. Variable R³ is, in various non-limiting embodiments, a C₂, C₃, C₄, C₅, C₆, C₈, C₉, or C₁₀ heteroaryl, each of which is optionally substituted.

In various embodiments, R³ is an optionally substituted —(C₁-C₁₂ alkyl)C₂-C₁₈ heterocyclyl. Variable R³ is, in various non-limiting embodiments, optionally substituted-(C₁-C₁₂ alkyl)-[C₂, C₃, C₄, C₅, C₆, C₈, C₉, or C₁₀ heterocyclyl], each of which is optionally substituted. In various embodiments, R³ is an optionally substituted —(C₁-C₁₂ alkyl)-[C₂-C₁₀ heteroaryl]. Variable R³ is, in various non-limiting embodiments, optionally substituted —(C₁-C₁₂ alkyl)-[C₂, C₃, C₄, C₅, C₆, C₈, C₉, or C₁₀ heteroaryl], each of which is optionally substituted

In various embodiments, R³ is selected from the group consisting of

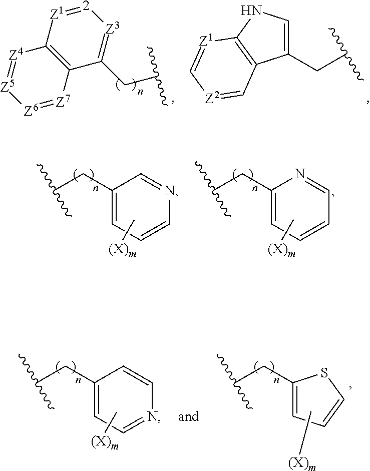

wherein:
each m is independently an integer from 0 to 4,
each n is independently an integer from 0 to 6,
each Z¹ to Z⁷ is independently CH or N, and
each X is independently selected from the group consisting of H, F, Cl, Br, I, OR, CN, NO₂, CF₃, OCF₃, R, N(R)₂, SOR, SO₂R, SO₂N(R)₂, C(O)R, and C(O)N(R)₂.

In various embodiments, in R³, n is 0 and m is 1. In various embodiments, in R³, X is C₁-C₃ alkyl. In various embodiments, in R³, X is methyl. In various embodiments, in R³, X is F. In various embodiments, in R³, X is Cl. In various embodiments, in R³, X is Br. In various embodiments, in R³, X is OH. In various embodiments, in R³, X is C₁-C₃ alkoxy.

In various embodiments, R³ is

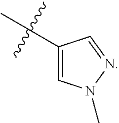 [SMILES: *c1cnn(C)c1]

In various embodiments, R³ is

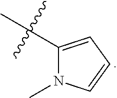 [SMILES: *c1cccn1C]

In various embodiments, R³ is

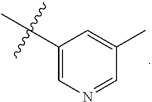 [SMILES: *c1cncc(C)c1]

In various embodiments, R³ is

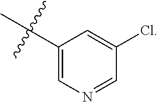 [SMILES: *c1cncc(Cl)c1]

In various embodiments, R³ is

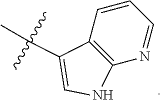 [SMILES: *c1c[nH]c2ncccc12]

In various embodiments, R³ is

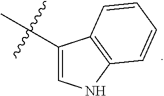 [SMILES: *c1c[nH]c2ccccc12]

In various embodiments, R³ is

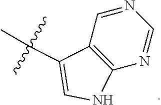 [SMILES: *c1c[nH]c2ncncc12]

In various embodiments, R³ is

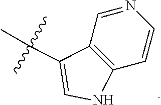 [SMILES: *c1c[nH]c2ccncc12]

In various embodiments, R³ is

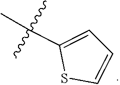 [SMILES: *c1cccs1]

In various embodiments, R³ is

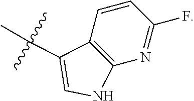 [SMILES: *c1c[nH]c2nc(F)ccc12]

In various embodiments, R³ is

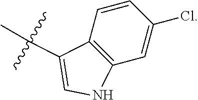 [SMILES: *c1c[nH]c2cc(Cl)ccc12]

In various embodiments, R³ is

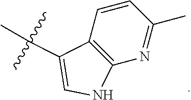 [SMILES: *c1c[nH]c2nc(C)ccc12]

In various embodiments, R³ is

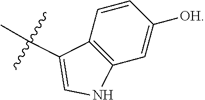 [SMILES: *c1c[nH]c2cc(O)ccc12]

In various embodiments, R³ is

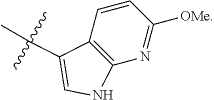 [SMILES: *c1c[nH]c2nc(OC)ccc12]

In various embodiments, R³ is

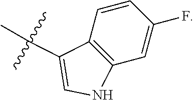 [SMILES: *c1c[nH]c2cc(F)ccc12]

In various embodiments, R³ is

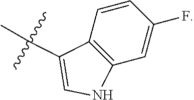 [SMILES: *c1c[nH]c2cc(F)ccc12]

In various embodiments, R³ is

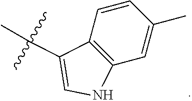 [SMILES: *c1c[nH]c2cc(C)ccc12]

In various embodiments, R³ is

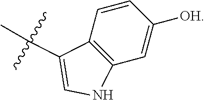 [SMILES: *c1c[nH]c2cc(O)ccc12]

In various embodiments, R³ is

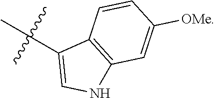 [SMILES: *c1c[nH]c2cc(OC)ccc12]

In certain embodiments, R³ is

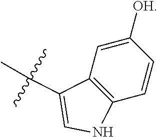 [SMILES: *c1c[nH]c2ccc(O)cc12]

In certain embodiments, R³ is

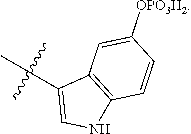

In certain embodiments, R³ is

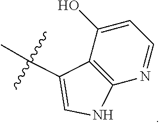 [SMILES: *c1c[nH]c2nccc(O)c12]

In certain embodiments, R³ is

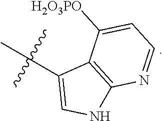

In certain embodiments, R³ is

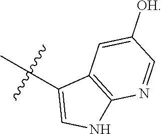 [SMILES: *c1c[nH]c2ncc(O)cc12]

In certain embodiments, R³ is

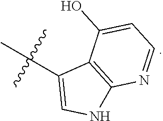 [SMILES: *c1c[nH]c2nccc(O)c12]

In certain embodiments, the compound is

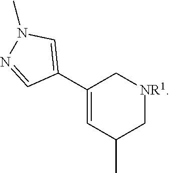

In certain embodiments, the compound is

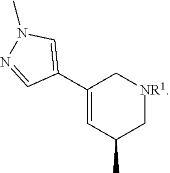

In certain embodiments, the compound is

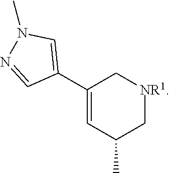

In certain embodiments, the compound is

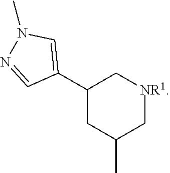

In certain embodiments, the compound is

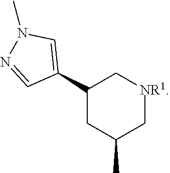

In certain embodiments, the compound is

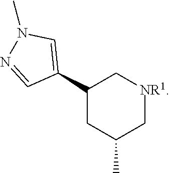

In certain embodiments, the compound is

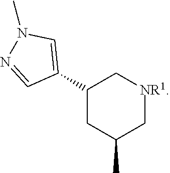

In certain embodiments, the compound is

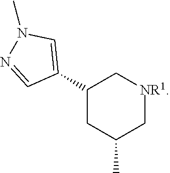

In certain embodiments, the compound is

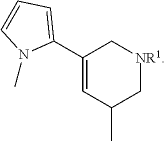

In certain embodiments, the compound is

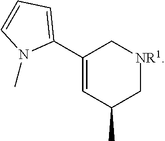

In certain embodiments, the compound is

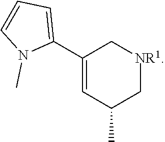

In certain embodiments, the compound is

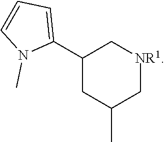

In certain embodiments, the compound is

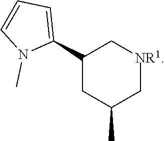

In certain embodiments, the compound is

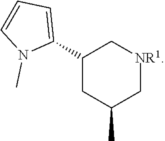

In certain embodiments, the compound is

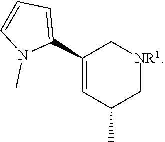

In certain embodiments, the compound is.

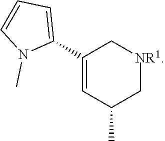

In certain embodiments, the compound is

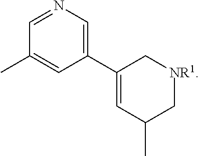

In certain embodiments, the compound is

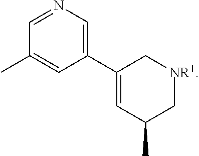

In certain embodiments, the compound is

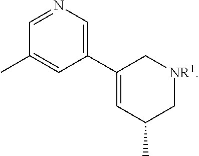

In certain embodiments, the compound is

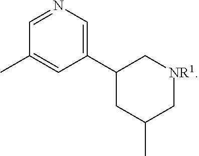

In certain embodiments, the compound is

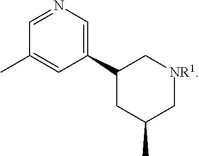

In certain embodiments the compound is

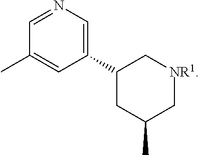

In certain embodiments, the compound is

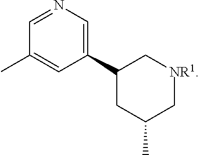

In certain embodiments, the compound is

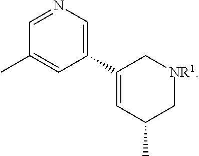

In certain embodiments, the compound is

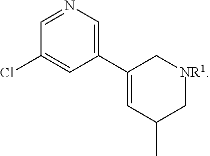

In certain embodiments, the compound is

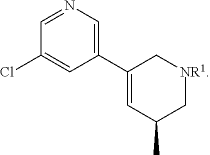

In certain embodiments, the compound

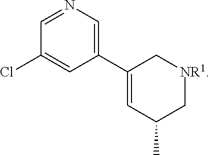

In certain embodiments, the compound is

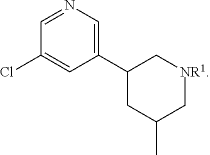

In certain embodiments, the compound is

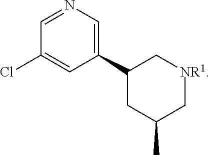

In certain embodiments, the compound is

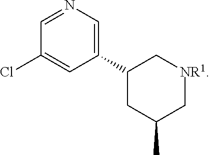

In certain embodiments, the compound is

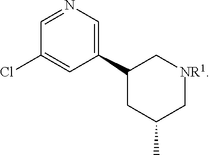

In certain embodiments, the compound is

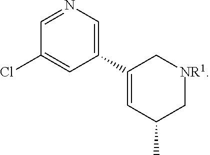

In certain embodiments, the compound is

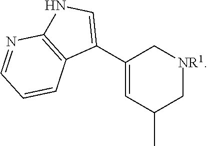

In certain embodiments, the compound is

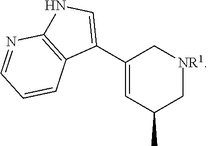

In certain embodiments, the compound

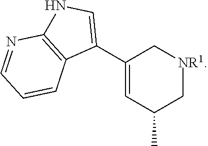

In certain embodiments, the compound is

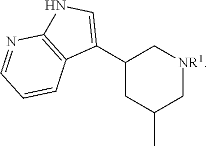

In certain embodiments, the compound is

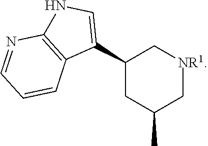

In certain embodiments, the compound is

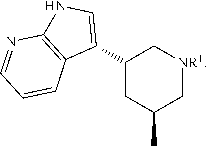

In certain embodiments, the compound is

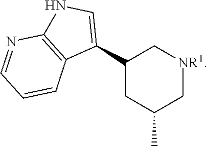

In certain embodiments, the compound is

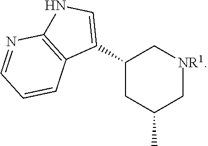

In certain embodiments, the compound is

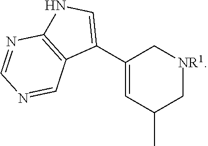

In certain embodiments, the compound is

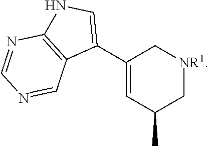

In certain embodiments, the compound

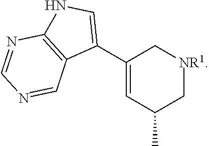

In certain embodiments, the compound is

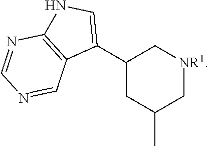

In certain embodiments, the compound is

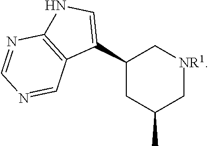

In certain embodiments, the compound is

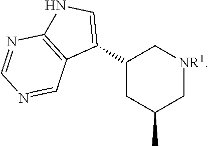

In certain embodiments, the compound is

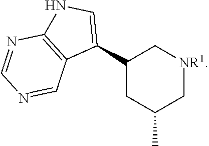

In certain embodiments, the compound is

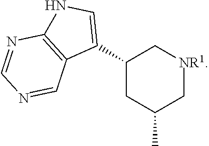

In certain embodiments, the compound is

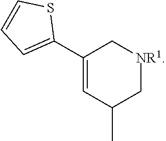

n certain embodiments, the compound is

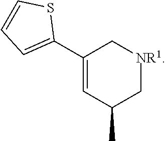

In certain embodiments, the compound is

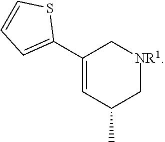

In certain embodiments, the compound is

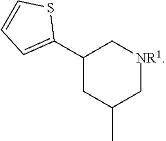

In certain embodiments, the compound is

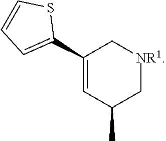

In certain embodiments, the compound is

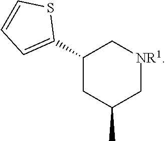

In certain embodiments, the compound is

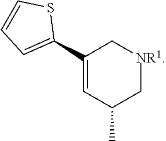

In certain embodiments, the compound is

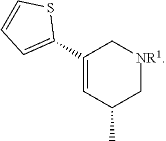

In certain embodiments, the compound is

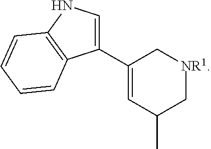

In certain embodiments, the compound is

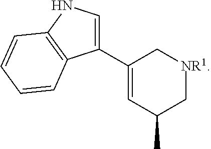

In certain embodiments, the compound is

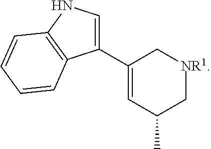

In certain embodiments, the compound is

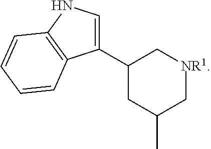

In certain embodiments, the compound is

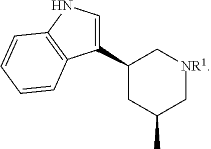

In certain embodiments, the compound is

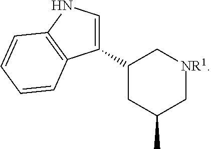

In certain embodiments, the compound is

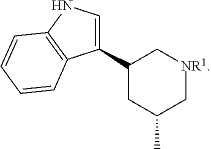

In certain embodiments, the compound is

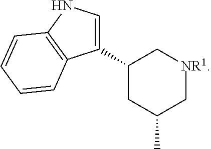

In certain embodiments, the compound is

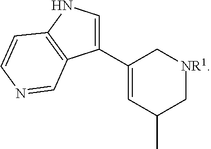

In certain embodiments, the compound is

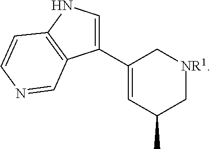

In certain embodiments, the compound is

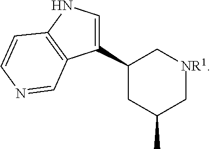

In certain embodiments, the compound is

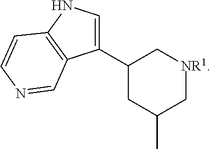

In certain embodiments, the compound is

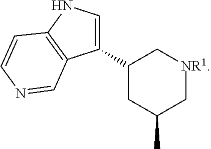

In certain embodiments, the compound is

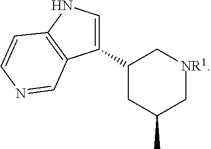

In certain embodiments, the compound is

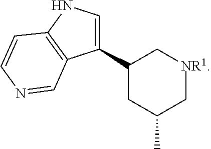

In certain embodiments, the compound is

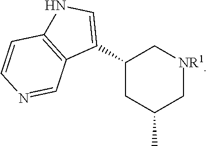

In certain embodiments, the compound is

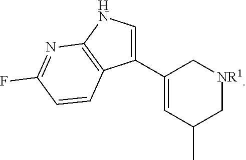

In certain embodiments, the compound is

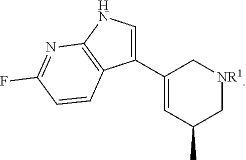

In certain embodiments, the compound is

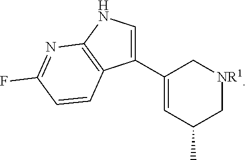

In certain embodiments, the compound is

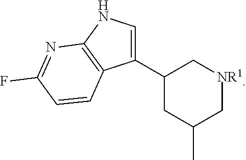

In certain embodiments, the compound is

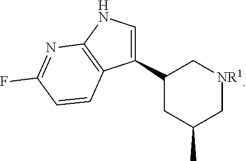

In certain embodiments, the compound is

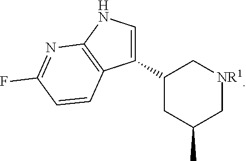

In certain embodiments, the compound is

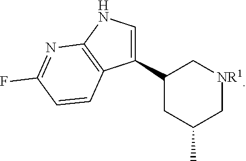

In certain embodiments, the compound is

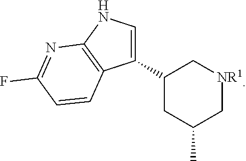

In certain embodiments, the compound is

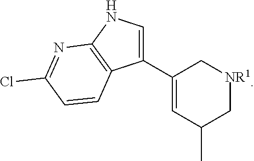

In certain embodiments, the compound is

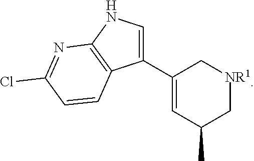

In certain embodiments, the compound is

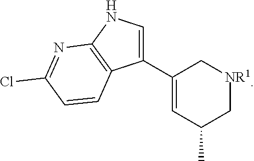

In certain embodiments, the compound is

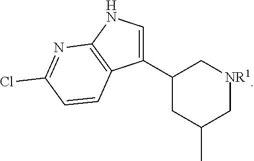

In certain embodiments, the compound is

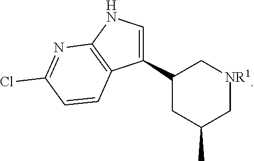

In certain embodiments, the compound is

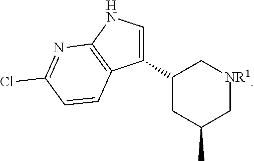

In certain embodiments, the compound is

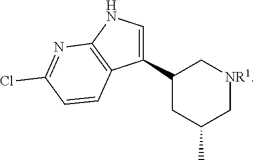

In certain embodiments, the compound is

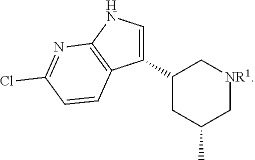

In certain embodiments, the compound is

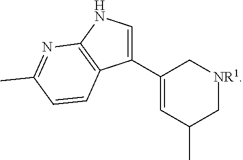

In certain embodiments, the compound is

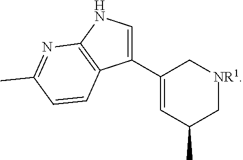

In certain embodiments, the compound is

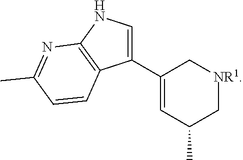

In certain embodiments, the compound is

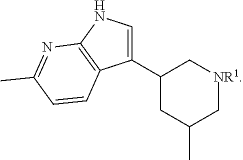

In certain embodiments, the compound is

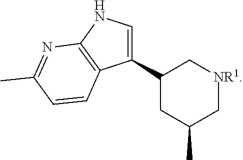

In certain embodiments, the compound is

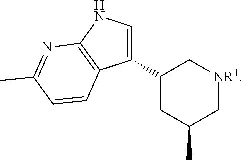

In certain embodiments, the compound is

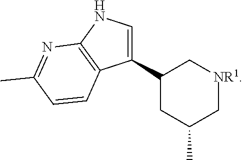

In certain embodiments, the compound is

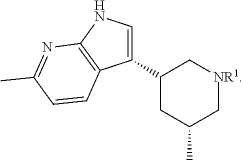

In certain embodiments, the compound is

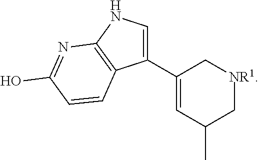

In certain embodiments, the compound is

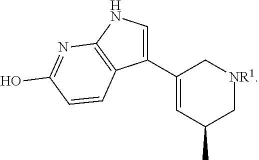

In certain embodiments, the compound is

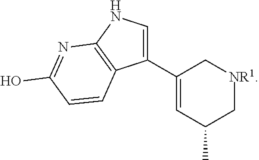

In certain embodiments, the compound is

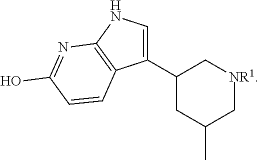

In certain embodiments, the compound is

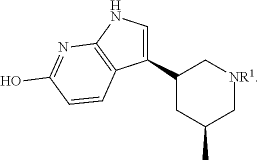

In certain embodiments, the compound is

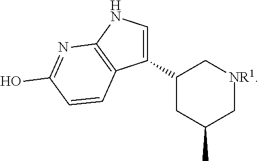

In certain embodiments, the compound is

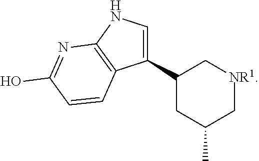

In certain embodiments, the compound is

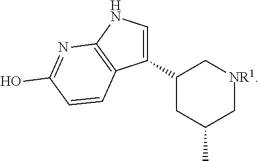

In certain embodiments, the compound is

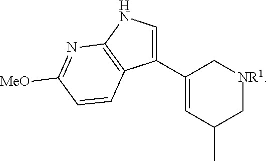

In certain embodiments the compound is

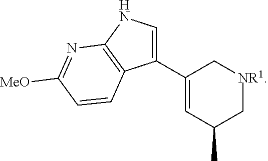

In certain embodiments, the compound is

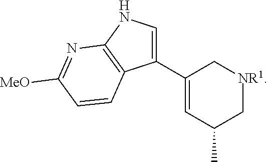

In certain embodiments, the compound is

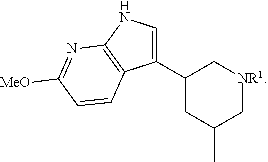

In certain embodiments, the compound is

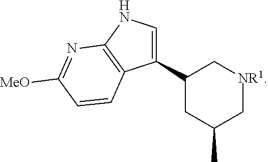

In certain embodiments, the compound is

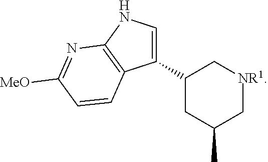

In certain embodiments, the compound is

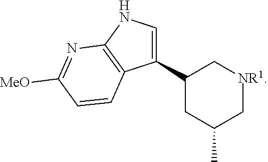

In certain embodiments, the compound is

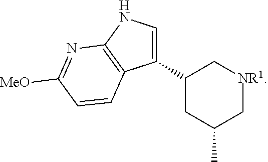

In certain embodiments, the compound is

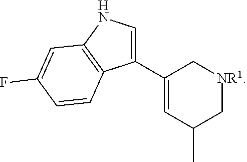

In certain embodiments, the compound is

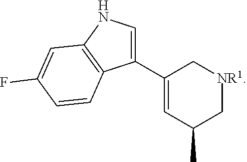

In certain embodiments, the compound is

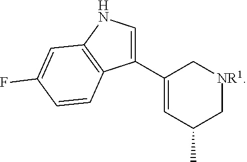

In certain embodiments, the compound is

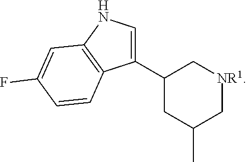

In certain embodiments, the compound is

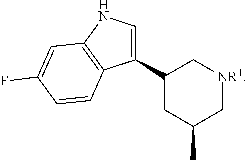

In certain embodiments, the compound is

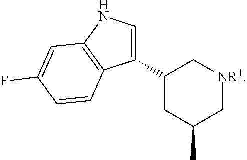

In certain embodiments, the compound is

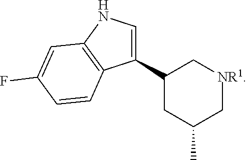

In certain embodiments, the compound is

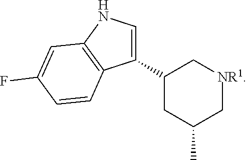

In certain embodiments, the compound is

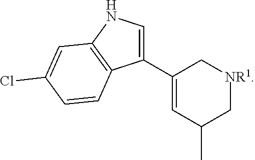

In certain embodiments, the compound is

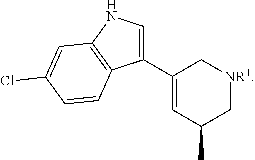

In certain embodiments, the compound is

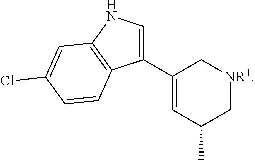

In certain embodiments, the compound is

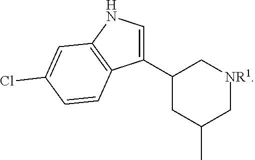

In certain embodiments, the compound is

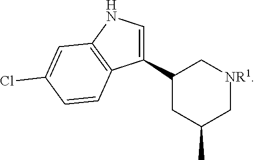

In certain embodiments, the compound is

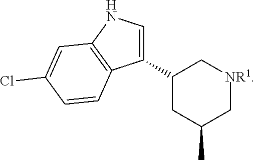

In certain embodiments, the compound is

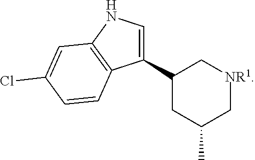

In certain embodiments, the compound is

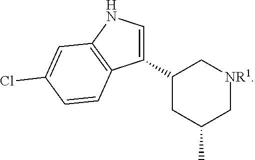

In certain embodiments, the compound is

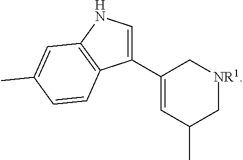

In certain embodiments, the compound is

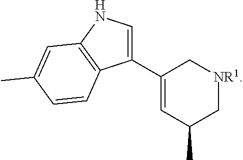

In certain embodiments, the compound is

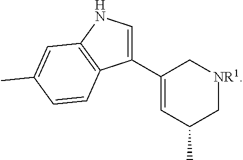

In certain embodiments, the compound is

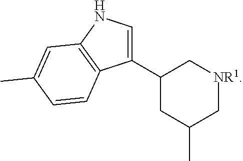

In certain embodiments, the compound is

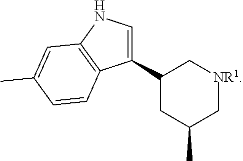

In certain embodiments, the compound is

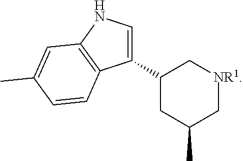

In certain embodiments, the compound is

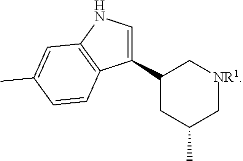

In certain embodiments, the compound is

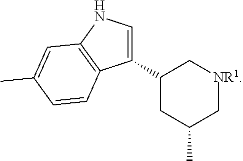

In certain embodiments, the compound is

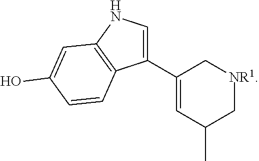

In certain embodiments, the compound is

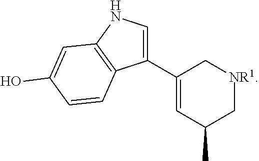

In certain embodiments, the compound is

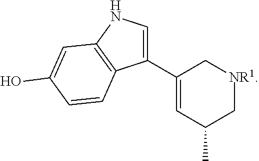

in certain embodiments, the compound is

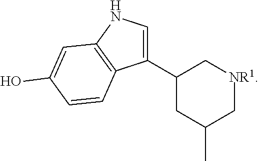

In certain embodiments, the compound is

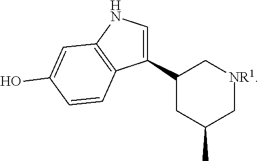

In certain embodiments, the compound is

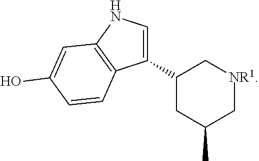

In certain embodiments, the compound is

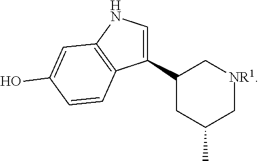

In certain embodiments, the compound is

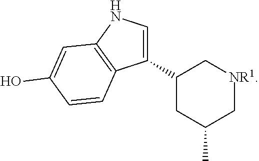

In certain embodiments, the compound is

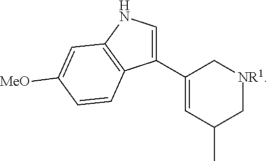

In certain embodiments, the compound is

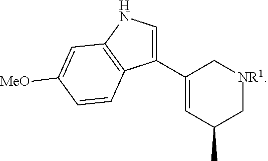

In certain embodiments, the compound is

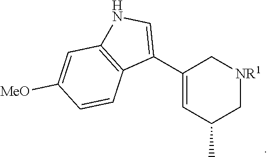

In certain embodiments, the compound is

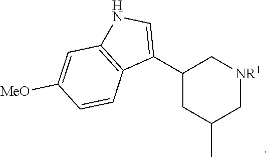

In certain embodiments, the compound is

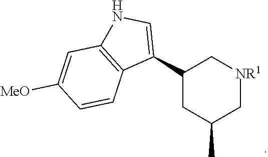

In certain embodiments, the compound is

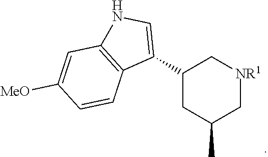

In certain embodiments, the compound is

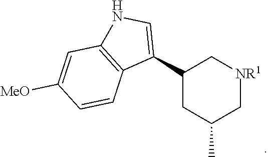

In certain embodiments, the compound is

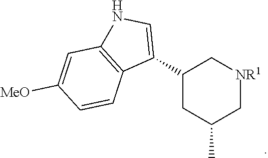

In certain embodiments, the compound is

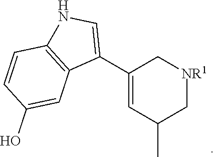

In certain embodiments, the compound is

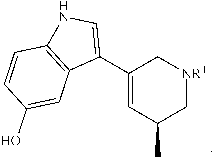

In certain embodiments, the compound is

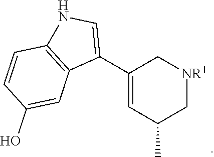

In certain embodiments, the compound is

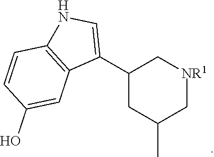

In certain embodiments, the compound is

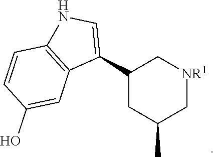

In certain embodiments, the compound is

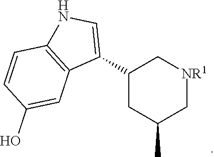

In certain embodiments, the compound is

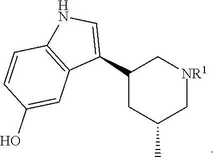

In certain embodiments, the compound is

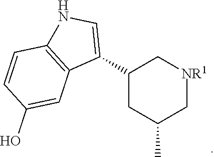

In certain embodiments, the compound is

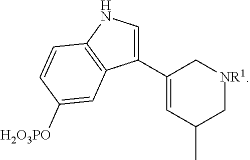

In certain embodiments, the compound is

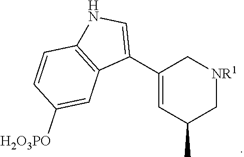

In certain embodiments, the compound is

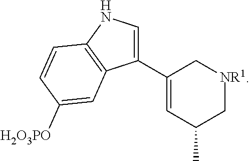

In certain embodiments, the compound is

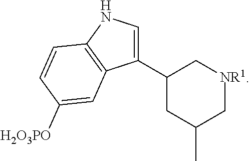

In certain embodiments, the compound is

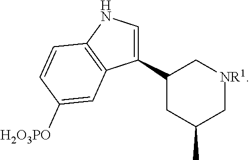

In certain embodiments, the compound is

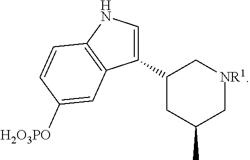

In certain embodiments, the compound is

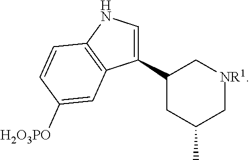

In certain embodiments, the compound is

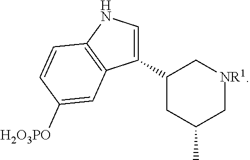

In certain embodiments, the compound is

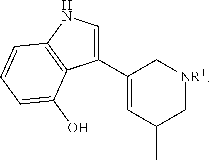

In certain embodiments, the compound is

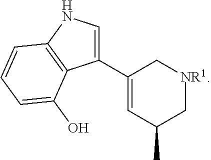

In certain embodiments, the compound is

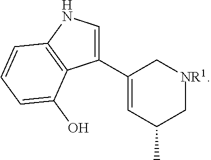

In certain embodiments, the compound is

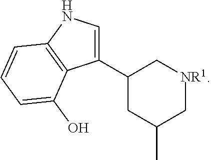

In certain embodiments, the compound is

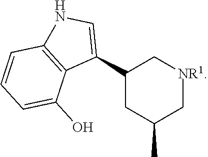

In certain embodiments, the compound is

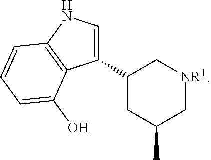

In certain embodiments, the compound is

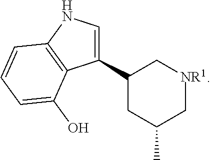

In certain embodiments, the compound is

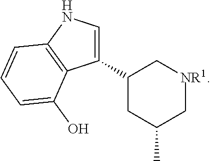

In certain embodiments, the compound is

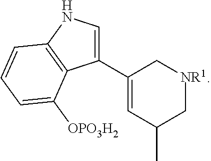

In certain embodiments, the compound is

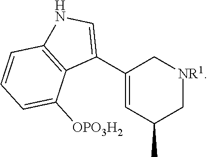

In certain embodiments, the compound is

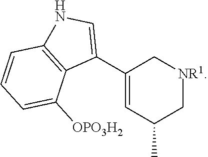

In certain embodiments, the compound is

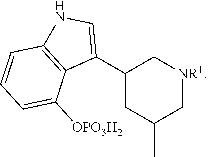

In certain embodiments, the compound is

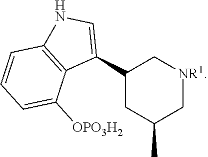

In certain embodiments, the compound is

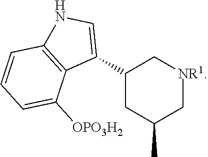

In certain embodiments, the compound is

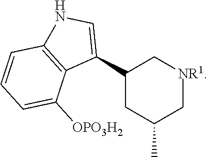

In certain embodiments, the compound is

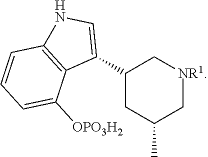

In certain embodiments, the compound is

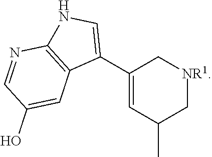

In certain embodiments, the compound is

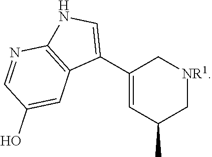

In certain embodiments, the compound is

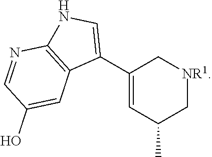

In certain embodiments, the compound is

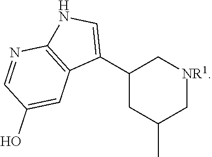

In certain embodiments, the compound is

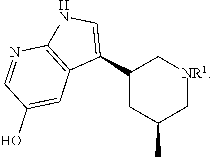

In certain embodiments, the compound is

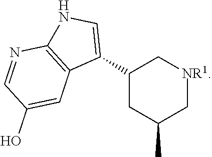

In certain embodiments, the compound is

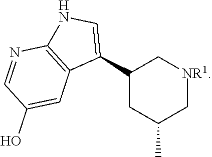

In certain embodiments, the compound is

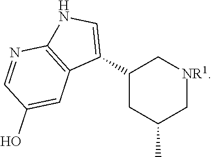

In certain embodiments, the compound is

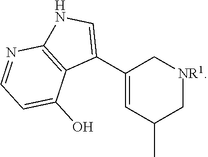

In certain embodiments, the compound is

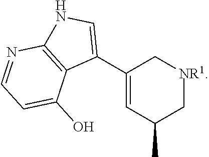

In certain embodiments, the compound is

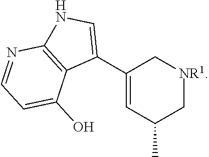

In certain embodiments, the compound is

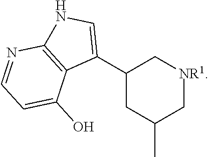

In certain embodiments, the compound is

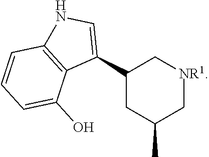

In certain embodiments, the compound is

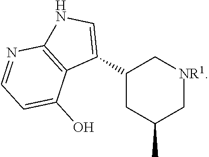

In certain embodiments, the compound is

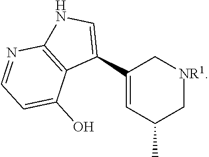

In certain embodiments, the compound is

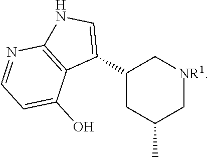

In certain embodiments, the compound is

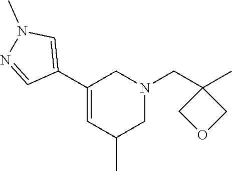 [SMILES: CC1C=C(c2cnn(C)c2)CN(CC2(C)COC2)C1]

In certain embodiments, the compound is

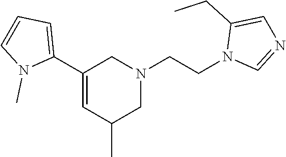 [SMILES: CCc1cncn1CCN1CC(c2cccn2C)=CC(C)C1]

In certain embodiments, the compound is

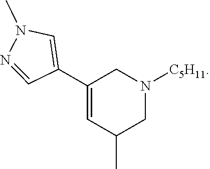

In certain embodiments, the compound is

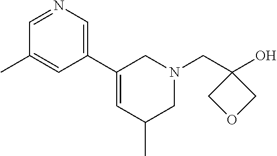 [SMILES: Cc1cncc(C2=CC(C)CN(CC3(O)COC3)C2)c1]

In certain embodiments, the compound is

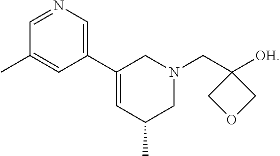 [SMILES: Cc1cncc(C2=C[C@@H](C)CN(CC3(O)COC3)C2)c1]

In certain embodiments, the compound is

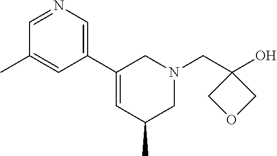 [SMILES: Cc1cncc(C2=C[C@H](C)CN(CC3(O)COC3)C2)c1]

In certain embodiments, the compound is

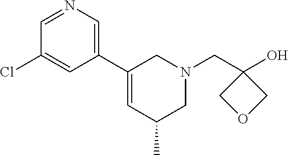 [SMILES: C[C@@H]1C=C(c2cncc(Cl)c2)CN(CC2(O)COC2)C1]

In certain embodiments, the compound is

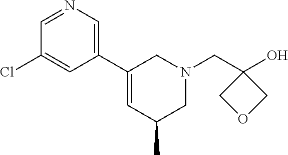 [SMILES: C[C@H]1C=C(c2cncc(Cl)c2)CN(CC2(O)COC2)C1]

In certain embodiments, the compound is

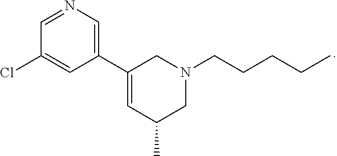 [SMILES: CCCCCN1CC(c2cncc(Cl)c2)=C[C@@H](C)C1]

In certain embodiments, the compound is

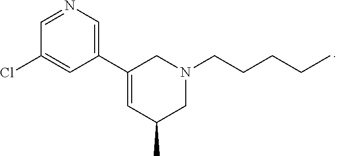 [SMILES: CCCCCN1CC(c2cncc(Cl)c2)=C[C@H](C)C1]

In certain embodiments, the compound is

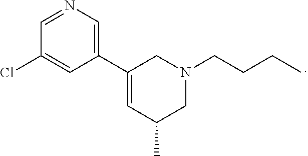 [SMILES: CCCCN1CC(c2cncc(Cl)c2)=C[C@@H](C)C1]

In PS certain embodiments, the compound is

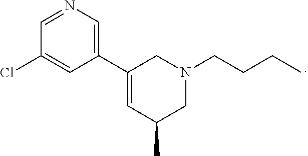 [SMILES: CCCCN1CC(c2cncc(Cl)c2)=C[C@H](C)C1]

In certain embodiments, the compound is

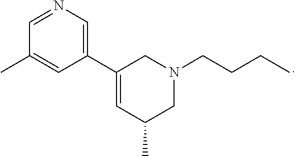 [SMILES: CCCCN1CC(c2cncc(C)c2)=C[C@@H](C)C1]

In certain embodiments, the compound is

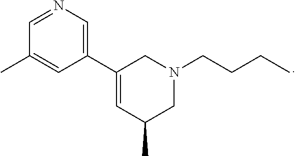 [SMILES: CCCCN1CC(c2cncc(C)c2)=C[C@H](C)C1]

In certain embodiments, the compound is

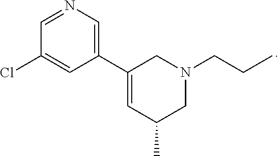 [SMILES: CCCN1CC(c2cncc(Cl)c2)=C[C@@H](C)C1]

In certain embodiments, the compound is

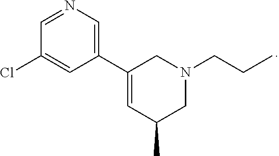 [SMILES: CCCN1CC(c2cncc(Cl)c2)=C[C@H](C)C1]

In certain embodiments, the compound is

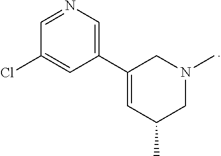

In certain embodiments, the compound is

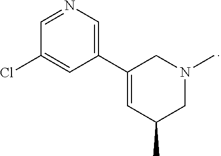

In certain embodiments the compound is

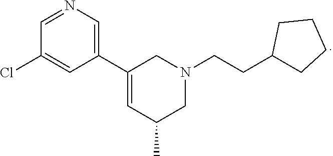 [SMILES: C[C@@H]1C=C(c2cncc(Cl)c2)CN(CCC2CCCC2)C1]

In certain embodiments, the compound is

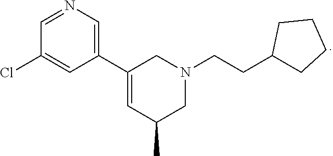 [SMILES: C[C@H]1C=C(c2cncc(Cl)c2)CN(CCC2CCCC2)C1]

In certain embodiments, the compound is

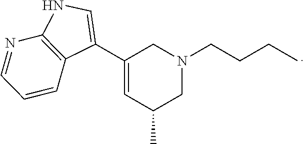 [SMILES: CCCCN1CC(c2c[nH]c3ncccc23)=C[C@@H](C)C1]

In certain embodiments, the compound is

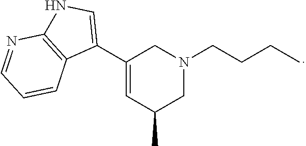 [SMILES: CCCCN1CC(c2c[nH]c3ncccc23)=C[C@H](C)C1]

In certain embodiments, the compound is

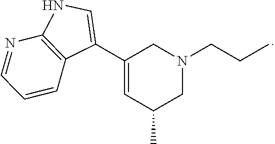 [SMILES: CCCN1CC(c2c[nH]c3ncccc23)=C[C@@H](C)C1]

In certain embodiments, the compound is

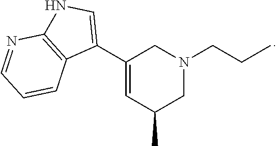 [SMILES: CCCN1CC(c2c[nH]c3ncccc23)=C[C@H](C)C1]

In certain embodiments, the compound is

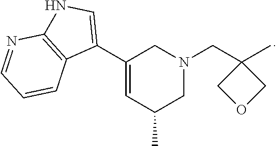 [SMILES: C[C@@H]1C=C(c2c[nH]c3ncccc23)CN(CC2(C)COC2)C1]

In certain embodiments, the compound is

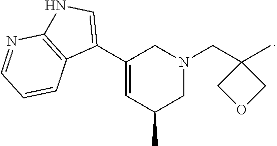 [SMILES: C[C@H]1C=C(c2c[nH]c3ncccc23)CN(CC2(C)COC2)C1]

In certain embodiments, the compound is

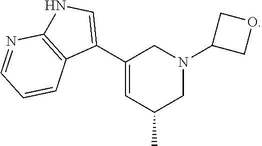 [SMILES: C[C@@H]1C=C(c2c[nH]c3ncccc23)CN(C2COC2)C1]

In certain embodiments, the compound is

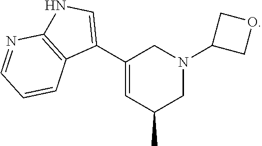 [SMILES: C[C@H]1C=C(c2c[nH]c3ncccc23)CN(C2COC2)C1]

In certain embodiments, the compound is

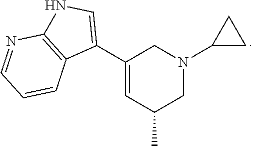 [SMILES: C[C@@H]1C=C(c2c[nH]c3ncccc23)CN(C2CC2)C1]

In certain embodiments, the compound is

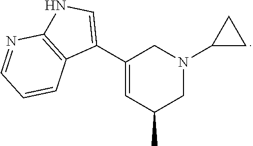 [SMILES: C[C@H]1C=C(c2c[nH]c3ncccc23)CN(C2CC2)C1]

In certain embodiments, the compound is

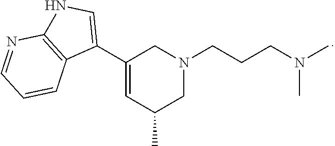 [SMILES: C[C@@H]1C=C(c2c[nH]c3ncccc23)CN(CCCN(C)C)C1]

In certain embodiments, the compound is

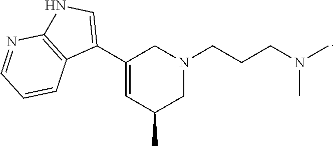 [SMILES: C[C@H]1C=C(c2c[nH]c3ncccc23)CN(CCCN(C)C)C1]

In certain embodiments, the compound is

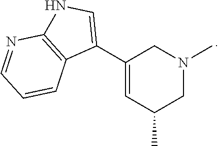 [SMILES: C[C@@H]1C=C(c2c[nH]c3ncccc23)CN(C)C1]

In certain embodiments the compound is

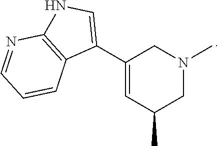 [SMILES: C[C@H]1C=C(c2c[nH]c3ncccc23)CN(C)C1]

In certain embodiments, the compound is

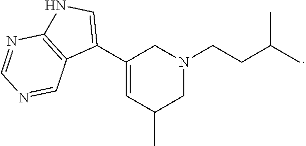 [SMILES: CC(C)CCN1CC(c2c[nH]c3ncncc23)=CC(C)C1]

In certain embodiments, the compound is

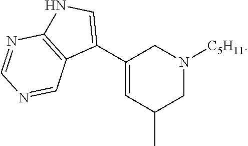

In certain embodiments, the compound is

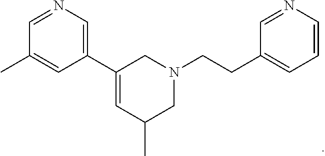 [SMILES: Cc1cncc(C2=CC(C)CN(CCc3cccnc3)C2)c1]

In certain embodiments, the compound is

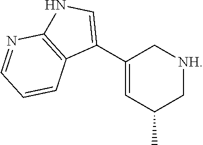 [SMILES: C[C@@H]1C=C(c2c[nH]c3ncccc23)CNC1]

In certain embodiments, the compound is

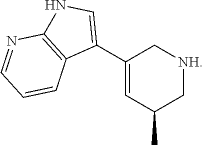 [SMILES: C[C@H]1C=C(c2c[nH]c3ncccc23)CNC1]

In certain embodiments, the compound is

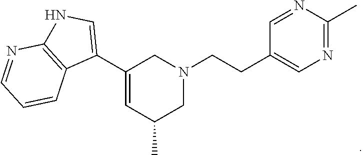 [SMILES: Cc1ncc(CCN2CC(c3c[nH]c4ncccc34)=C[C@@H](C)C2)cn1]

In certain embodiments, the compound is

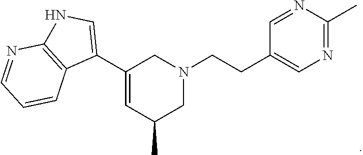 [SMILES: Cc1ncc(CCN2CC(c3c[nH]c4ncccc34)=C[C@H](C)C2)cn1]

In certain embodiments, the compound is

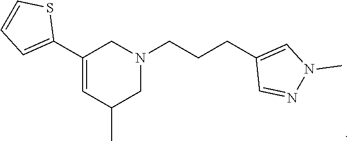 [SMILES: CC1C=C(c2cccs2)CN(CCCc2cnn(C)c2)C1]

In certain embodiments, the compound is

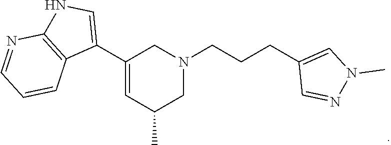 [SMILES: C[C@@H]1C=C(c2c[nH]c3ncccc23)CN(CCCc2cnn(C)c2)C1]

In certain embodiments, the compound is

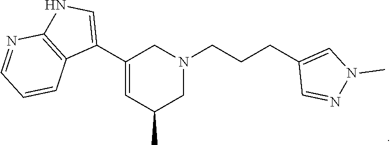 [SMILES: C[C@H]1C=C(c2c[nH]c3ncccc23)CN(CCCc2cnn(C)c2)C1]

In certain embodiments, the compound is

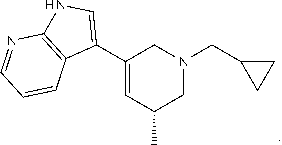 [SMILES: C[C@@H]1C=C(c2c[nH]c3ncccc23)CN(CC2CC2)C1]

In certain embodiments, the compound is

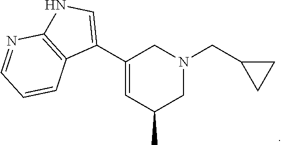 [SMILES: C[C@H]1C=C(c2c[nH]c3ncccc23)CN(CC2CC2)C1]

In certain embodiments, the compound is

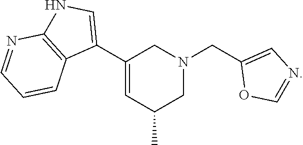 [SMILES: C[C@@H]1C=C(c2c[nH]c3ncccc23)CN(Cc2cnco2)C1]

In certain embodiments, the compound is

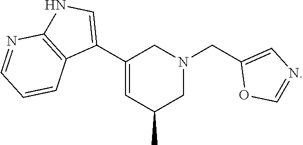 [SMILES: C[C@H]1C=C(c2c[nH]c3ncccc23)CN(Cc2cnco2)C1]

In certain embodiments, the compound is

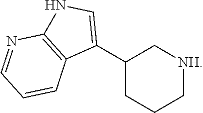 [SMILES: c1cnc2[nH]cc(C3CCCNC3)c2c1]

In certain embodiments, the compound is

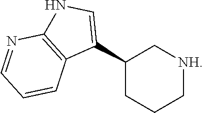 [SMILES: c1cnc2[nH]cc([C@@H]3CCCNC3)c2c1]

In certain embodiments, the compound is

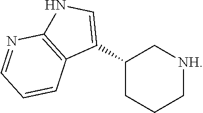 [SMILES: c1cnc2[nH]cc([C@H]3CCCNC3)c2c1]

In various embodiments, the compound of formula (I) has a selectivity ratio for the 5-HT_{2A} receptor over the 5-HT_{2B} receptor of at least, equal to, or greater than about 1.1:1, 1.2:1, 1.4:1, 1.6:1, 1.8:1, 2:1, 3:1, 4:1, 5:1, 6:1, 7:1, 8:1, 9:1, 10:1, 15:1, 20:1, 30:1, 40:1, 50:1, 60:1, 70:1, 80:1, 90:1, 100:1, 200:1, 300:1, 400:1, 500:1, 600:1, 700:1, 800:1, 900:1, 1000:1, 5000:1, 10000:1, 50000:1, 100000:1, or more.

In certain embodiments, the compound of formula (I) is a 5-HT_{2B} receptor agonist. In certain embodiments, the compound of formula (I) is a 5-HT_{2B} receptor antagonist. In certain embodiments, the compound of formula (I) is a 5-HT_{2C} receptor agonist. In certain embodiments, the compound of formula (I) is a 5-HT_{2C} receptor antagonist.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

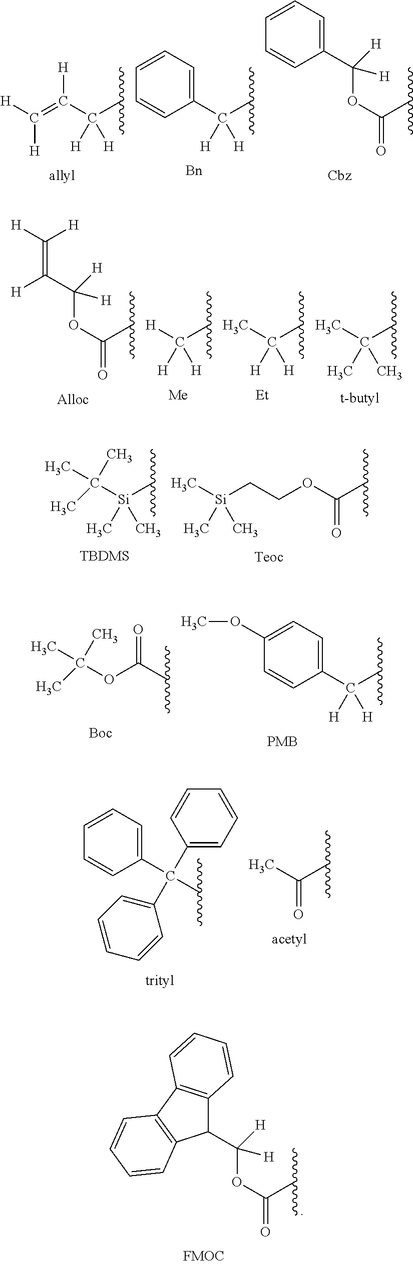

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Pharmacology

In various embodiments, the compound(s) described herein can be administered to a subject in an amount ranging from about 0.01 mg/kg to about 200 mg/kg, or about 0.5 mg/kg to about 190 mg/kg, or about 0.75 mg/kg to about 180 mg/kg, or about 1 mg/kg to about 170 mg/kg, or about 1.5 mg/kg to about 160 mg/kg, or about 2 mg/kg to about 150 mg/kg, or about 2.5 mg/kg to about 140 mg/kg, or about 3 mg/kg to about 130 mg/kg, or about 3.5 mg/kg to about 120 mg/kg, or about 4 mg/kg to about 110 mg/kg, or about 4.5 mg/kg to about 100 mg/kg, or about 5 mg/kg to about 95 mg/kg, or about 5.5 mg/kg to about 90 mg/kg, or about 6 mg/kg to about 85 mg/kg, or about 6.5 mg/kg to about 80 mg/kg, or about 7 mg/kg to about 75 mg/kg, or about 7.5 mg/kg to about 70 mg/kg, or about 8 mg/kg to about 65 mg/kg, or about 8.5 mg/kg to about 60 mg/kg, or about 9 mg/kg to about 55 mg/kg or about 9.5 mg/kg to about 50 mg/kg, or about 10 mg/kg to about 45 mg/kg.

In various embodiments, the compound(s) described herein can be administered to a subject in an amount that is less than, equal to, or greater than about 0.01 mg/kg, 0.05 mg/kg, 0.1 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 100 mg/kg, 105 mg/kg, 110 mg/kg, 115 mg/kg, 120 mg/kg, 125 mg/kg, 130 mg/kg, 140 mg/kg, 145 mg/kg, 150 mg/kg, 155 mg/kg, 160 mg/kg, 170 mg/kg, 175 mg/kg, 180 mg/kg, 185 mg/kg, 190 mg/kg, 195 mg/kg, or 200 mg/kg.

### Compositions

The compositions containing the compound(s) described herein include a pharmaceutical composition comprising at least one compound as described herein and at least one pharmaceutically acceptable carrier. In one embodiment, a pharmaceutical composition includes at least one compound of formula (I) and at least one pharmaceutically acceptable excipient or carrier.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

In certain embodiments, the compound of formula (I) is a compound of formula (II). The disclosure includes a method of treating, ameliorating, and/or preventing a neurological disease or disorder using the compounds of formula (II):

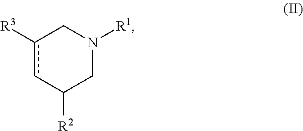

wherein:
[symbol] represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, and optionally substituted C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂; and
each occurrence of R is independently H, C₁-C₁₂ alkyl, C₃-C₁₂ cycloalkyl, or —(C₁-C₁₂ alkyl)C₃-C₁₂ cycloalkyl.

In certain embodiments, the compound of formula (II) is not (R)—N,N-dimethyl-3-(3-methyl-5-(1H-pyrrolo[2,3-b]pyridin-3-yl)-3,6-dihydropyridin-1(2H)-yl)propan-1-amine.

In certain embodiments, the compound of formula (II) is not (S)—N,N-dimethyl-3-(3-methyl-5-(1H-pyrrolo[2,3-b]pyridin-3-yl)-3,6-dihydropyridin-1(2H)-yl)propan-1-amine.

In certain embodiments, the compound of formula (II) is not (S)-3-(1-(cyclopropylmethyl)-5-methyl-1,2,5,6-tetrahydropyridin-3-yl)-1H-pyrrolo[2,3-b]pyridine.

The method includes administering to a subject in need thereof a therapeutically effective amount of a composition containing a compound formula (II), or a pharmaceutically acceptable salt, solvate, enantiomer, or N-oxide thereof.

Non-limiting examples of a neurological disease or disorder include depression, anxiety, substance abuse, and headaches. Headaches that can be treated with the methods herein include, but are not limited to, migraine headaches and cluster headaches.

This disclosure also includes a method of selectively agonizing the 5-hydroxytryptamine 2A (5-HT_{2A}) receptor. The method includes administering to a subject a compound of formula (II), or a pharmaceutically acceptable salt, solvate, enantiomer, or N-oxide thereof, and where the compound of formula (II) selectively binds to the 5-HT_{2A} over the 5-HT_{2B} receptor. The method of selectively agonizing the 5-HT_{2A} receptor can be used to treat, ameliorate, and/or prevent diseases or disorders that are affected by, associated with, or would benefit from selective agonist activity at the 5-HT_{2A} receptor. By selectively binding to and agonizing the 5-HT_{2A} receptor over the 5-HT_{2B} receptor, the method provides, in various embodiments, reduced side-effects such as, but not limited to, drug-induced valvular heart disease associated with binding and agonizing or antagonizing the 5-HT_{2B} receptor. In certain embodiments, the compound of formula (II) is a 5-HT_{2B} receptor agonist. In certain embodiments, the compound of formula (II) is a 5-HT_{2B} receptor antagonist. In certain embodiments, the compound of formula (II) is a 5-HT_{2C} receptor agonist. In certain embodiments, the compound of formula (II) is a 5-HT_{2C} receptor antagonist.

The methods described herein include administering to the subject a therapeutically effective amount of at least one compound of formula (II), as described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats a neurological disease or disorder or that treats a disease or disorder that is affected by, associated with, or would benefit from selective agonist activity at the 5-HT_{2A} receptor.

In certain embodiments, administering the compound(s) described herein to the subject allows for administering a lower dose of the additional therapeutic agent as compared to the dose of the additional therapeutic agent alone that is required to achieve similar results in treating, ameliorating, and/or preventing a neurological disease or disorder or in treating, ameliorating, and/or preventing a disease or disorder that is affected by, associated with, or would benefit from selective agonist activity at the 5-HT_{2A} receptor in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating, ameliorating, and/or preventing a neurological disease or disorder or treating a disease or disorder that is affected by, associated with, or would benefit from selective agonist activity at the 5-HT_{2A} receptor. These additional therapeutic agents may comprise compounds that are commercially available or synthetically accessible to those skilled in the art. These additional therapeutic agents are known to treat or reduce the symptoms, of a neurological disease or disorder or treat a disease or disorder that is affected by, associated with, or would benefit from selective agonist activity at the 5-HT_{2A} receptor.

In certain embodiments, the compounds described herein can be used in combination with radiation therapy. In other embodiments, the combination of administration of the compounds described herein and application of radiation therapy is more effective in treating or preventing a neurological disease or disorder or treating or preventing a disease or disorder that is affected by, associated with, or would benefit from selective agonist activity at the 5-HT_{2A} receptor than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a neurological disease or disorder or a disease or disorder that is affected by, associated with, or would benefit from selective agonist activity at the 5-HT_{2A} receptor. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a neurological disease or disorder or treat a disease or disorder that is affected by, associated with, or would benefit from selective agonist activity at the 5-HT_{2A} receptor in the patient. An effective amount of the therapeutic compound necessary to achieve a therapeutic effect may vary according to factors such as the state of the disease or disorder in the patient; the age, sex, and weight of the patient; and the ability of the therapeutic compound to treat a neurological disease or disorder or treat a disease or disorder that is affected by, associated with, or would benefit from selective agonist activity at the 5-HT_{2A} receptor in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In various embodiments, the dose of a compound described herein is from about 1 mg and about 2,500 mg. In various embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in various embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms in a patient of a neurological disease or disorder or a disease or disorder that is affected by, associated with, or would benefit from selective agonist activity at the 5-HT_{2A} receptor.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any one of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%0, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a neurological disease or disorder or a disease or disorder that is affected by, associated with, or would benefit from selective agonist activity at the 5-HT_{2A} receptor in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### General Information

Air-sensitive experiments were set up inside a Vacuum Atmospheres glovebox under nitrogen atmosphere with oxygen and moisture levels not exceeding 10 ppm or were performed under a nitrogen gas atmosphere in flame-dried glassware cooled down under nitrogen gas. Solvents for air-sensitive reactions were purified by elution through a column of activated alumina under an argon atmosphere and stored over 3 Å molecular sieves in a glovebox. Triethylamine was distilled over calcium hydride. For work-up and purification, ACS reagent grade solvents were used. Benzylamine and methacrolein were distilled prior to use. Zinc acetate was heated at 60° C. under vacuum overnight. All other reagents were purchased from commercial sources and used without further purification.

Flash-column chromatography was performed on SILIAFLASH® P60 silica gel (230-400 mesh), and silica gel-coated glass plates from Analtech (1 mm SiO₂, 20×20 cm) were used for preparative thin-layer chromatography. Reverse-phase column chromatography was performed on pre-packed cartridges of C18 silica gel using an automated purification system. Enantiomerically pure products were obtained using an Agilent 1100 series HPLC equipped with a semi-preparative Chiralpak AD-H column (250×10 mm) and a multi-wavelength detector.

For NMR characterization, the magnetic field strength of the instrument is indicated for each spectrum. NMR chemical shifts (6) are reported in ppm relative to CHCl₃ (δ=7.26 ppm), MeOH (δ=3.31 ppm) or C₆H₆ (δ=7.16 ppm) for ¹H NMR, and CDCl₃ (δ=77.16 ppm), CD₃OD (δ=49.00 ppm), or C₆H₆ (δ=128.06 ppm) for ¹³C-NMR. All ¹³C NMR are proton decoupled. The multiplicity and shape of NMR signals are designated by the following abbreviations: s=singlet, d=doublet, t=triplet, q=quartet, p=pentet, sx=sextet, sept=septet, m=multiplet, br=broad signal, and dd=doublet of doublets. Coupling constants Jare reported in Hertz (Hz). High-resolution mass spectra (IRMS) were recorded on a quadrupole time-of-flight (TOF) mass spectrometer after electrospray ionization (ESI).

### Example 1: Synthesis of Imines

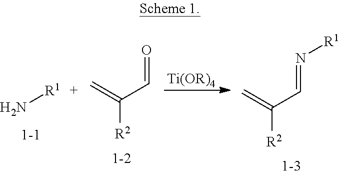

All imines (1-3) were synthesized from commercially available amines (1-1) and enals (1-2), wherein R¹, R², and R³ in Scheme 1 are defined within the scope of the present disclosure.

### General Procedure A (Imine Synthesis)

To a flame-dried round-bottom flask was added enal (1.0 equiv), dry THF (1.2 M), titanium (IV) ethoxide (2.0 equiv) and amine (1.05 equiv). The reaction solution was stirred under N₂ at rt for 1 h. Upon reaction completion, NH₄OH (aq) solution was added, and the reaction mixture was filtered through a pad of celite. The filtrate was then extracted three times with Et₂O, and the combined organic layers were washed with brine, dried over MgSO₄, filtered, and concentrated in vacuo. The crude material was filtered through a plug of basic Al₂O₃ (eluting with pentane) and concentrated to provide the desired imine, which was stored at −20° C. in a nitrogen filled glovebox and taken on to the next step without further purification.

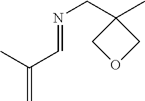

(E)-2-Methyl-N-((3-methyloxetan-3-yl)methyl)prop-2-en-1-imine: General procedure A was followed with minor modifications using (3-methyloxetan-3-yl)methanamine (212 mg, 2.10 mmol, 1.05 equiv), methacrolein (0.17 mL, 2.0 mmol, 1.0 equiv), Ti(OEt)₄ (2.2 mL, 10 mmol, 5.2 equiv), and THE (1.7 mL). (E)-2-Methyl-N-((3-methyloxetan-3-yl)methyl)prop-2-en-1-imine was obtained as a pale-yellow oil (191 mg, 62% yield). ¹H NMR (400 MHz, CDCl₃) δ 7.95 (s, 1H), 5.60 (s, 1H), 5 38 (s, 1H), 4.54 (d, J=5.6 Hz, 2H), 4.37 (d, J=5.7 Hz, 2H), 3.66 (s, 2H), 1.91 (s, 3H), 1.30 (s, 3H).

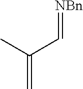

(E)-N-Benzyl-2-methylprop-2-en-1-imine: (E)-N-Benzyl-2-methylprop-2-en-1-imine was synthesized following a modified version of general procedure A with benzylamine (3.6 mL, 33 mmol, 1.05 equiv), methacrolein (2.6 mL, 31 mmol, 1.0 equiv), Ti(OEt)₄ (13 mL, 63 mmol, 2.0 equiv), and THE (26 mL). The reaction mixture was stirred at 55° C. for 2.5 h. To this crude mixture was added N,N,N′,N-tetrakis(2-hydroxyethyl)ethylenediamine (EDTE) (17 mL, 79 mmol, 2.5 equiv), and the reaction mixture was reheated to 55° C. for an additional 25 min. Work-up proceeded in accordance with general procedure A, however, filtration prior to extraction was no longer necessary. (E)-N-Benzyl-2-methylprop-2-en-1-imine was obtained as a clear oil (3.14 g, 63% yield). Spectroscopic data are in agreement with reported values.

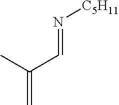

(E)-2-Methyl-N-pentylprop-2-en-1-imine: General procedure A was followed with minor modifications using 1-aminopentane (0.74 mL, 6.4 mmol, 1.05 equiv), methacrolein (0.50 mL, 6.1 mmol, 1.0 equiv), Ti(OEt)₄ (6.6 mL, 31 mmol, 5.2 equiv), and THE (5.1 mL). (E)-2-Methyl-N-pentylprop-2-en-1-imine was obtained as a pale-yellow oil (220 mg, 26% yield). ¹H NMR (400 MHz, CDCl₃) δ 7.88 (s, 1H), 5.56 (s, 1H), 5.34 (s, 1H), 3.48 (t, J=7.1 Hz, 2H), 1.92 (s, 3H), 1.62 (p, J=7.2 Hz, 2H), 1.38-1.22 (m, 4H), 0.90 (t, J=6.6 Hz, 3H).

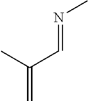

(E)-N,2-Dimethylprop-2-en-1-imine: To an oven-dried scintillation vial was added 3 Å molecular sieves (5 g), methacrolein (0.41 mL, 5.0 mmol, 1.0 equiv), and methylamine (5.0 mL, 10 mmol, 2.0 equiv, 2 M solution in THF). The reaction mixture was stirred under N₂ at rt for 4 h. The reaction mixture was then filtered through a plug of Celite and rinsed with dry THE (3 mL) to create an approximately 0.6 M imine solution. The resulting imine stock solution was used directly for THP synthesis without concentration.

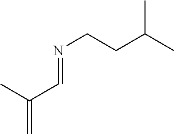

(E)-N-Isopentyl-2-methylprop-2-en-1-imine: General procedure A was followed using 3-methylbutan-1-amine (1.5 mL, 13 mmol, 1.05 equiv), methacrolein (1.0 mL, 12 mmol, 1.0 equiv), Ti(OEt)₄ (5.0 mL, 24 mmol, 2.0 equiv), and THE (10 mL). (E)-N-Isopentyl-2-methylprop-2-en-1-imine (784 mg, 47% yield) was obtained as a yellow oil. ¹H NMR (400 MHz, CDCl₃) δ 7.90 (s, 1H), 5.56 (s, 1H), 5.34 (s, 1H), 3.50 (t, J=7.4 Hz, 2H), 1.92 (s, 3H), 1.66-1.57 (m, 1H), 1.51 (apparent q, J=7.1 Hz, 2H), 0.91 (d, J=6.6 Hz, 6H).

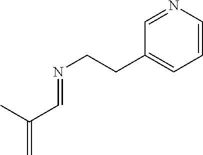

(E)-2-Methyl-N-(2-(pyridin-3-yl)ethyl)prop-2-en-1-imine: General procedure A was followed using 2-(pyridin-3-yl)ethan-1-amine (385 mg, 3.15 mmol, 1.05 equiv), methacrolein (0.25 mL, 3.0 mmol, 1.0 equiv), Ti(OEt)₄ (1.3 mL, 6.0 mmol, 2.0 equiv), and THE (2.5 mL). (E)-2-Methyl-N-(2-(pyridin-3-yl)ethyl)prop-2-en-1-imine (391 mg, 75% yield) was obtained as a yellow oil. ¹H NMR (400 MHz, CDCl₃) δ 8.49-8.42 (m, 2H), 7.76 (s, 1H), 7.50 (d, J=7.8 Hz, 1H), 7.20 (dd, J=7.6, 4.9 Hz, 1H), 5.57 (s, 1H), 5.31 (s, 1H), 3.74 (t, J=7.1 Hz, 2H), 2.96 (t, J=7.2 Hz, 2H), 1.92 (s, 3H).

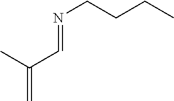

(E)-N-Butyl-2-methylprop-2-en-1-imine: General procedure A was followed using 1-aminobutane (3.15 mL, 31.8 mmol, 1.05 equiv), methacrolein (2.50 mL, 30.3 mmol, 1.0 equiv), Ti(OEt)₄ (13 mL, 61 mmol, 2.0 equiv), and THE (25 mL). (E)-N-Butyl-2-methylprop-2-en-1-imine (2.12 g, 56% yield) was obtained as a pale-yellow oil. ¹H NMR (400 MHz, CDCl₃) δ 7.89 (s, 1H), 5.56 (s, 1H), 5.35 (s, 1H), 3.49 (t, J=7.1 Hz, 2H), 1.93 (s, 3H), 1.61 (p, J=7.6 Hz, 2H), 1.33 (sx, J=7.6 Hz, 2H), 0.92 (t, J=7.4 Hz, 3H).

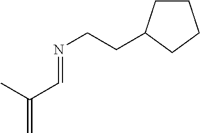

(E)-N-(2-Cyclopentylethyl)-2-methylprop-2-en-1-imine: General procedure A was followed using 2-cyclopentylethan-1-amine (475 mg, 4.20 mmol, 1.05 equiv), methacrolein (0.33 mL, 4.0 mmol, 1.0 equiv), Ti(OEt)₄ (1.7 mL, 8.0 mmol, 2.0 equiv), and THE (3.3 mL). (E)-N-(2-Cyclopentylethyl)-2-methylprop-2-en-1-imine (407 mg, 62% yield) was obtained as a pale-yellow oil. ¹H NMR (400 MHz, CDCl₃) δ 7.90 (s, 1H), 5.56 (s, 1H), 5.34 (s, 1H), 3.50 (t, J=7.4 Hz, 2H), 1.93 (s, 3H), 1.83-1.68 (m, 3H), 1.68-1.58 (m, 4H), 1.58-1.44 (m, 2H), 1.20-1.04 (m, 2H).

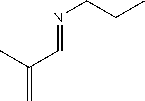

(E)-2-Methyl-N-propylprop-2-en-1-imine: General procedure A was followed using 1-aminopropane (1.7 mL, 21 mmol, 1.05 equiv), methacrolein (1.7 mL, 20 mmol, 1.0 equiv), Ti(OEt)₄ (8.4 mL, 40 mmol, 2.0 equiv), and THE (17 mL). (E)-2-Methyl-N-propylprop-2-en-1-imine (422 mg, 19% yield) was obtained as a pale-yellow oil. ¹H NMR (400 MHz, CDCl₃) δ 7.89 (s, 1H), 5.57 (s, 1H), 5.35 (s, 1H), 3.46 (t, J=7.0 Hz, 2H), 1.93 (s, 3H), 1.65 (sx, J=7.3 Hz, 2H), 0.90 (t, J=7.4 Hz, 3H).

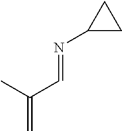

(E)-N-Cyclopropyl-2-methylprop-2-en-1-imine: General procedure A was followed using cyclopropanamine (1.46 mL, 21.0 mmol, 1.05 equiv), methacrolein (1.65 mL, 20.0 mmol, 1.0 equiv), Ti(OEt)₄ (8.4 mL, 40 mmol, 2.0 equiv), and THE (17 mL). (E)-N-Cyclopropyl-2-methylprop-2-en-1-imine (994 mg, 46% yield) was obtained as a pale-yellow oil. ¹H NMR (400 MHz, CDCl₃) δ 8.08 (s, 1H), 5.48 (s, 1H), 5.31 (s, 1H), 2.91-2.84 (m, 1H), 1.88 (s, 3H), 0.92-0.83 (m, 4H).

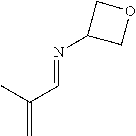

(E)-2-Methyl-N-(oxetan-3-yl)prop-2-en-1-imine: General procedure A was followed using oxetan-3-amine (614 mg, 8.40 mmol, 1.05 equiv), methacrolein (0.66 mL, 8.0 mmol, 1.0 equiv), Ti(OEt)₄ (3.4 mL, 16 mmol, 2.0 equiv), and THE (6.6 mL). (E)-2-Methyl-N-(oxetan-3-yl)prop-2-en-1-imine was obtained as a pale-yellow oil (718 mg, 72% yield). ¹H NMR (500 MHz, CDCl₃) δ 7.81 (s, 1H), 5.64 (s, 1H), 5.40 (s, 1H), 4.90 (apparent t, J=6.6 Hz, 2H), 4.79 (apparent t, J=6.0 Hz, 2H), 4.64 (p, J=6.4 Hz, 1H), 1.96 (s, 3H).

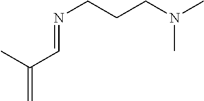

(E)-N,N-Dimethyl-3-((2-methylallylidene)amino)propan-1-amine: General procedure A was followed using N,N-dimethyl-1,3-propanediamine (0.66 mL, 5.3 mmol, 1.05 equiv), methacrolein (0.41 mL, 5.0 mmol, 1.0 equiv), Ti(OEt)₄ (2.1 mL, 10 mmol, 2.0 equiv), and THE (4.2 mL). (E)-N,N-Dimethyl-3-((2-methylallylidene)amino)propan-1-amine was obtained as a pale-yellow oil (429 mg, 56% yield). ¹H NMR (400 MHz, CDCl₃) δ 7.90 (s, 1H), 5.57 (s, 1H), 5.36 (s, 1H), 3.52 (t, J=7.0 Hz, 2H), 2.30 (t, J=7.3 Hz, 2H), 2.23 (s, 6H), 1.92 (s, 3H), 1.86-1.76 (m, 2H).

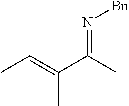

(2E,3E)-N-Benzyl-3-methylpent-3-en-2-imine: General procedure A was followed with minor modifications using benzylamine (0.55 mL, 5.1 mmol, 1.0 equiv), methacrolein (0.56 mL, 5.0 mmol, 1.0 equiv), Ti(OEt)₄ (5.4 mL, 26 mmol, 5.2 equiv), and THE (4.2 mL). (2E,3E)-N-Benzyl-3-methylpent-3-en-2-imine was obtained as a yellow oil (542 mg, 58% yield). ¹H NMR (400 MHz, CDCl₃) δ 7.38 (d, J=7.5 Hz, 2H), 7.33 (t, J=7.5 Hz, 2H), 7.22 (t, J=7.2 Hz, 1H), 6.18 (q, J=7.1 Hz, 1H), 4.63 (s, 2H), 2.04 (s, 3H), 1.94 (s, 3H), 1.82 (d, J=6.8 Hz, 3H).

### Example 2: Synthesis of Alkynes

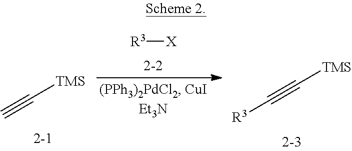

### General Procedure B (Alkyne Synthesis)

To a flame-dried round-bottom flask was added bis(triphenylphosphine)palladium(II) dichloride (0.05 equiv), copper(I) iodide (0.05 equiv), and heteroaryl bromide (1.0 equiv) dissolved in triethylamine (0.4 M). To this solution was then added ethynyltrimethylsilane (2.0 equiv). The reaction mixture was stirred at 70-80° C. After the reaction was complete as monitored by thin-layer chromatography, the mixture was then diluted with water, extracted with ethyl acetate, washed with brine, dried with Na₂SO₄ and filtered. The organic layer was plugged through silica and concentrated in vacuo. The crude residue was purified via silica gel chromatography to afford the desired alkyne.

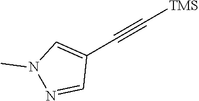

1-Methyl-4-((trimethylsilyl)ethynyl)-1H-pyrazole: 1-Methyl-4-((trimethylsilyl)ethynyl)-1H-pyrazole was synthesized using a modification of the general procedure B. To a schlenk tube was added 4-bromo-1-methyl-1H-pyrazole (4.00 g, 24.8 mmol, 1.0 equiv), Pd cat (1.74 g, 2.48 mmol, 0.10 equiv), CuI (472 mg, 2.48 mmol, 0.10 equiv), Et₃N (62 mL), and TMS-alkyne (14 mL, 99 mmol, 4.0 equiv), and the reaction mixture was stirred at 100° C. for 48 h. The crude product was purified by flash-column chromatography (20% EtOAc/hexanes) and reverse-phase flash chromatography over C18 silica gel (60-70% MeCN/H₂O+0.1% TFA) to afford the desired product (1.67 g, 38% yield) as a white solid. Spectroscopic data are in agreement with reported values.

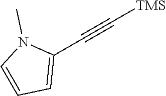

1-Methyl-2-((trimethylsilyl)ethynyl)-1H-pyrrole: 2-Bromo-1-methyl-1H-pyrrole was synthesized according to a previously reported literature procedure. 1-Methyl-2-((trimethylsilyl)ethynyl)-1H-pyrrole was synthesized using a modification of the general procedure B. To a schlenk tube was added 2-bromo-1-methyl-1H-pyrrole (8.30 g, 51.9 mmol, 1.0 equiv), Pd cat (3.64 g, 5.19 mmol, 0.10 equiv), CuI (988 mg, 5.19 mmol, 0.10 equiv), Et₃N (90 mL), and TMS-alkyne (8.6 mL, 62 mmol, 1.2 equiv), and the reaction mixture was stirred at 100° C. for 45 h. The crude product was purified by flash-column chromatography (0.5% Et₂O/pentane) to afford the desired product (2.72 g, 30% yield) as an orange oil. ¹H NMR (500 MHz, CDCl₃) δ 6.63-6.59 (m, 1H), 6.44-6.40 (m, 1H), 6.07-6.02 (m, 1H), 3.66 (s, 3H), 0.24 (s, 9H).

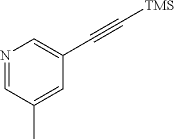

3-Methyl-5-((trimethylsilyl)ethynyl)pyridine: General procedure B was followed using 3-bromo-5-methyl-pyridine (1.50 g, 8.82 mmol, 1.0 equiv), Pd cat (306 mg, 0.436 mmol, 0.05 equiv), CuI (83.0 mg, 0.436 mmol, 0.05 equiv), Et₃N (22 mL), and TMS-alkyne (2.4 mL, 17 mmol, 2.0 equiv). The reaction mixture was stirred for 3 h at 70° C. The crude product was purified by flash-column chromatography (15% Et₂O/pentane) followed by a second flash-column chromatography purification (2% EtOAc/hexanes) to afford 3-Methyl-5-((trimethylsilyl)ethynyl)pyridine (1.33 g, 81% yield) as a yellow oil. ¹H NMR (400 MHz, CDCl₃) δ 8.50 (s, 1H), 8.36 (s, 1H), 7.58 (s, 1H), 2.32 (s, 3H), 0.26 (s, 9H).

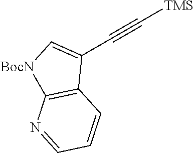 [SMILES: CC(C)(C)OC(=O)n1cc(C#C[Si](C)(C)C)c2cccnc21]

tert-Butyl 3-((trimethylsilyl)ethynyl)-1H-pyrrolo[2,3-b]pyridine-1-carboxylate: 3-Bromo-1H-pyrrolo[2,3-b]pyridine (4.93 g, 25.0 mmol, 1.0 equiv) was protected using Boc₂O (8.18 g, 37.5 mmol, 1.5 equiv), iPr₂NEt (6.4 mL, 38 mmol, 1.5 equiv), and DMAP (916 mg, 7.50 mmol, 0.30 equiv) in CH₂Cl₂ (83 mL). The reaction was performed at rt for 2 h and was then concentrated in vacuo and purified by flash-column chromatography (10% EtOAc/hexanes+1% Et₃N) to afford tert-butyl 3-bromo-1H-pyrrolo[2,3-b]pyridine-1-carboxylate (7.43 g, quantitative yield) as a pale-yellow oil. Spectroscopic data are in agreement with reported values.

Next, general procedure B was followed using tert-butyl 3-bromo-1H-pyrrolo[2,3-b]pyridine-1-carboxylate (7.43 g, 25.0 mmol, 1.0 equiv), Pd cat (877 mg, 1.25 mmol, 0.05 equiv), CuI (238 mg, 1.25 mmol, 0.05 equiv), Et₃N (60 mL), and TMS-alkyne (7.0 mL, 50 mmol, 2.0 equiv). The reaction mixture was stirred for 3 h at 80° C. The crude product was purified by flash-column chromatography (10% EtOAc/hexanes) to afford tert-Butyl 3-((trimethylsilyl)ethynyl)-1H-pyrrolo[2,3-b]pyridine-1-carboxylate (5.49 g, 70% yield) as a cream-colored solid. ¹H NMR (400 MHz, CDCl₃) δ 8.54 (dd, J=4.7, 1.4 Hz, 1H), 7.99 (dd, J=7.8, 1.5 Hz, 1H), 7.83 (s, 1H), 7.29-7.24 (m, 1H), 1.66 (s, 9H), 0.28 (s, 9H).

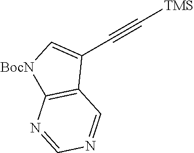 [SMILES: CC(C)(C)OC(=O)n1cc(C#C[Si](C)(C)C)c2cncnc21]

tert-Butyl 5-((trimethylsilyl)ethynyl)-7H-pyrrolo[2,3-d]pyrimidine-7-carboxylate: 5-Bromo-7H-pyrrolo[2,3-d]pyrimidine (825 mg, 4.17 mmol, 1.0 equiv) was protected using Boc₂O (1.36 g, 6.25 mmol, 1.5 equiv), iPr₂NEt (1.1 mL, 6.3 mmol, 1.5 equiv), and DMAP (153 mg, 1.25 mmol, 0.30 equiv) in CH₂Cl₂ (14 mL). The reaction was performed at rt for 1.5. The reaction mixture was then concentrated in vacuo followed by purification by flash-column chromatography (15% EtOAc/hexanes+1% Et₃N) to afford tert-butyl 5-bromopyrrolo[2,3-d]pyrimidine-7-carboxylate (1.19 g, 96% yield) as a white solid. ¹H NMR (400 MHz, CDCl₃) δ 9.14 (s, 1H), 8.95 (s, 1H), 7.72 (s, 1H), 1.68 (s, 9H).

Next, general procedure B was followed using tert-butyl 5-bromopyrrolo[2,3-d]pyrimidine-7-carboxylate (1.19 g, 3.98 mmol, 1.0 equiv), Pd cat (140 mg, 0.199 mmol, 0.05 equiv), CuI (37.9 mg, 0.199 mmol, 0.05 equiv), Et₃N (10 mL), and TMS-alkyne (1.1 mL, 8.0 mmol, 2.0 equiv). The reaction mixture was stirred for 3 h at 80° C. The crude product was purified by flash-column chromatography (12% EtOAc/hexanes) to afford tert-Butyl 5-((trimethylsilyl)ethynyl)-7H-pyrrolo[2,3-d]pyrimidine-7-carboxylate (1.11 g, 89% yield) as a pale-yellow solid. ¹H NMR (400 MHz, CDCl₃) δ 9.13 (s, 1H), 9.06 (s, 1H), 7.82 (s, 1H), 1.68 (s, 9H), 0.29 (s, 9H).

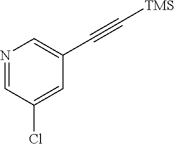 [SMILES: C[Si](C)(C)C#Cc1cncc(Cl)c1]

3-Chloro-5-((trimethylsilyl)ethynyl)pyridine: General procedure B was followed using 3-bromo-5-chloro-pyridine (4.00 g, 20.8 mmol, 1.0 equiv), Pd cat (730 mg, 1.04 mmol, 0.05 equiv), CuI (198 mg, 1.04 mmol, 0.05 equiv), Et₃N (52 mL), and TMS-alkyne (5.8 mL, 42 mmol, 2.0 equiv). The reaction mixture was stirred for 3 h at 70° C. The crude product was purified by flash-column chromatography (5% Et₂O/pentane) to afford 3-Chloro-5-((trimethylsilyl)ethynyl)pyridine (2.23 g, 51% yield) as a cream-colored solid. Spectroscopic data are in agreement with reported values.

### Example 3: Synthesis of THPs (Diels-Alder)

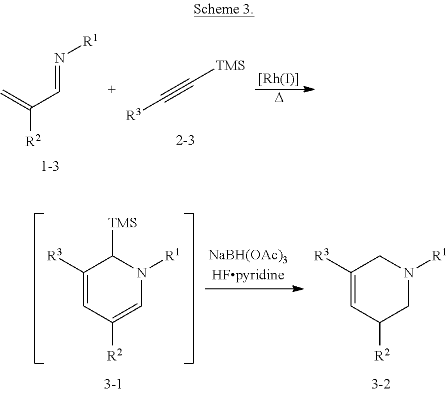

### Preparation of Rhodium Catalyst Stock Solution

[RhCl(coe)₂]₂ was purchased from Strem and stored inside a N₂-filled inert atmosphere glovebox at −25° C. Stock solutions of the rhodium catalyst were prepared in the glovebox. For the preparation of a 50 mM solution in toluene, a 4 mL glass vial was charged with [RhCl(coe)₂]₂ (100 mg, 0.139 mmol) and p-Me₂N—C₆H₄-PEt₂ (58 mg, 0.278 mmol) in anhydrous toluene (2.8 mL). For the preparation of a 100 mM solution in THF, a 4 mL glass vial was charged with [RhCl(coe)₂]₂ (100 mg, 0.139 mmol) and p-Me₂N—C₆H₄-PEt₂ (58 mg, 0.278 mmol) in anhydrous THE (1.4 mL). Stock solutions, when stored in a −25° C. freezer inside a N₂-filled glovebox, could be used for several months without loss of catalytic activity.

### General Procedure C (Synthesis of DHPs)

Inside a N₂-filled inert atmosphere glovebox, imine (1.0 equiv) was dissolved in toluene or THE (0.4 M total concentration), and the solution was transferred to a flame-dried schlenk tube. A stock solution of Rh catalyst (see General Information) at the catalyst loading indicated was added to the tube, followed by alkyne (1.25-2.0 equiv). A small portion (˜0.3 mL) of this reaction mixture was transferred to an oven-dried J. Young NMR tube that was equipped with a flame-sealed melting point capillary containing benzene-d₆ for reaction monitoring. The J. Young NMR tube and schlenk tube were then sealed, removed from the glovebox, and heated at the indicated temperature. The reaction progress was monitored by ¹H NMR spectroscopy based on the disappearance of the imine signals and the increasing resonances of the dihydropyridine. Upon reaction completion, the tubes were taken back inside the glovebox for the reduction of the DHP intermediates (General Procedure D).

### General Procedure D (Reduction of DHPs to THPs)

Inside a N₂-filled inert atmosphere glovebox, the crude DHP solution (1.0 equiv) from General Procedure C was transferred and rinsed with THE (0.4 M relative to DHP) to an oven-dried scintillation vial. Separately, to a flame-dried round-bottom flask was added sodium triacetoxyborohydride (3.0 equiv). In a fume hood, THE (0.1 M relative to DHP) was added to the round-bottom flask, which was then submerged in an −78° C. dry ice-acetone bath. HF-pyridine (85 equiv) was added dropwise followed by crude dihydropyridine solution transferred via syringe. The reaction mixture was stirred for 2 h at −78° C. and was then allowed to warm to rt for 2 h. Upon reaction completion, 6 M NaOH was added until pH ˜11 was reached. The mixture was then transferred to a separatory funnel and extracted three times with CH₂Cl₂. The combined organic layers were washed with brine, dried over MgSO₄, filtered, and concentrated in vacuo. Purified material was obtained by flash-column chromatography or preparative thin-layer chromatography.

### General Procedure E (Cleavage of Acid-Labile Protecting Groups)

To an oven-dried scintillation vial was added THP (1.0 equiv) in CH₂Cl₂ (0.12 M). The reaction mixture was cooled to 0° C., and trifluoroacetic acid (10.0 equiv) was added. The ice bath was removed and the reaction mixture was stirred at rt until the reaction was complete as monitored by thin-layer chromatography. The reaction mixture was then diluted with CH₂Cl₂ and saturated NaHCO₃ (aq) and extracted three times with CH₂Cl₂. The combined organic layers were washed with brine, dried with MgSO₄, filtered and concentrated in vacuo. The crude material was purified by preparative thin-layer chromatography.

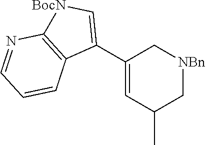 [SMILES: CC1C=C(c2cn(C(=O)OC(C)(C)C)c3ncccc23)CN(Cc2ccccc2)C1]

(±) tert-Butyl 3-(1-benzyl-5-methyl-1,2,5,6-tetrahydropyridin-3-yl)-1H-pyrrolo[2,3-b]pyridine-1-carboxylate: General procedure C was followed with minor modifications using [RhCl(coe)₂]₂ (135 mg, 0.188 mmol, 0.05 equiv), followed by ligand (78.5 mg, 0.375 mmol, 0.10 equiv), (E)-N-benzyl-2-methylprop-2-en-1-imine (597 mg, 3.75 mmol, 1.0 equiv) and tert-butyl 3-((trimethylsilyl)ethynyl)-1H-pyrrolo[2,3-b]pyridine-1-carboxylate (1.47 g, 4.69 mmol, 1.25 equiv) in toluene (9.4 mL). The reaction was run at 90° C. for 3 h to produce the DHP intermediate. Following general procedure D, DHP reduction was performed with Na(OAc)₃BH (2.38 g, 11.3 mmol, 3.0 equiv) in THE (38 mL), HF-pyridine (7.5 mL, 319 mmol, 85 equiv), and crude DHP solution (3.75 mmol, 1.0 equiv). The crude DHP solution was transferred and rinsed with THE (9.4 mL). The resulting crude THP product was purified by flash-column chromatography (15% EtOAc/hexanes+1% Et₃N) to afford the title compound (734 mg, 49% yield from imine) as a thick pale-yellow oil. ¹H NMR (500 MHz, CDCl₃) δ 8.50 (dd, J=4.7, 1.3 Hz, 1H) 8.09 (dd, J=7.9, 1.4 Hz, 1H), 7.46 (s, 1H), 7.41 (d, J=7.3 Hz, 2H), 7.35 (t, J=7.5 Hz, 2H), 7.29 (t, J=7.2 Hz, 1H), 7.20 (dd, J=7.9, 4.8 Hz, 1H), 6.12 (s, 1H), 3.75 (d, J=13.1 Hz, 1H), 3.66 (d, J=13.1 Hz, 1H), 3.45 (d, J=15.4 Hz, 1H), 3.22 (d, J=15.4 Hz, 1H), 2.87 (dd, J=11.0, 5.3 Hz, 1H), 2.69-2.58 (m, 1H), 2.09 (dd, J=11.0, 8.1 Hz, 1H), 1.66 (s, 9H), 1.08 (d, J=7.0 Hz, 3H).

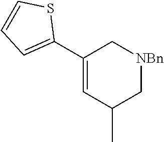 [SMILES: CC1C=C(c2cccs2)CN(Cc2ccccc2)C1]

(±) 1-Benzyl-3-methyl-5-(thiophen-2-yl)-1,2,3,6-tetrahydropyridine: General procedure C was followed using (E)-N-benzyl-2-methylprop-2-en-1-imine (271 mg, 1.70 mmol, 1.0 equiv) and trimethyl(thiophen-2-ylethynyl)silane (460 mg, 2.55 mmol, 1.5 equiv) in toluene (3.4 mL). Rh catalyst (2.5 mol %, 0.85 mL, 43 μmol, 50 mM in toluene) was added, and the reaction was run at 90° C. for 6 h to produce the desired intermediate. Following general procedure D, the reduction was performed with Na(OAc)₃BH (1.08 g, 5.10 mmol, 3.0 equiv) in THE (17 mL), HF-pyridine (3.4 mL, 150 mmol, 85 equiv), and crude solution (1.70 mmol, 1.0 equiv). The crude solution was transferred and rinsed with THE (4.3 mL). The resulting crude THP product was purified by flash-column chromatography (6% EtOAc/hexanes+1% Et₃N) followed by preparative thin-layer chromatography (10% EtOAc/hexanes+1% Et₃N) with only pure fractions isolated to afford 1-benzyl-3-methyl-5-(thiophen-2-yl)-1,2,3,6-tetrahydropyridine (163 mg, 36% yield from imine) as a pale-yellow oil. ¹H NMR (500 MHz, CDCl₃) δ 7.39 (d, J=7.3 Hz, 2H), 7.34 (t, J=7.4 Hz, 2H), 7.28 (t, J=7.3 Hz, 1H), 7.11 (d, J=5.6 Hz, 1H), 6.93 (dd, J=5.1, 3.6 Hz, 1H), 6.86 (d, J=3.3 Hz, 1H), 6.04 (s, 1H), 3.72 (d, J=13.1 Hz, 1H), 3.65 (d, J=13.2 Hz, 1H), 3.48 (d, J=15.3 Hz, 1H), 3.20 (d, J=15.5 Hz, 1H), 2.82 (dd, J=10.8, 5.0 Hz, 1H), 2.61-2.51 (m, 1H), 2.05 (t, J=11.8, 8.8 Hz, 1H), 1.03 (d, J=7.1 Hz, 3H).

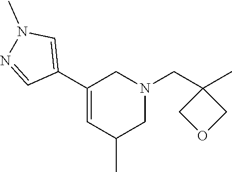 [SMILES: CC1C=C(c2cnn(C)c2)CN(CC2(C)COC2)C1]

(±) 3-Methyl-5-(1-methyl-1H-pyrazol-4-yl)-1-((3-methyloxetan-3-yl)methyl)-1,2,3,6-tetrahydropyridine (Compound 1): General procedure C was followed using (E)-2-methyl-N-((3-methyloxetan-3-yl)methyl)prop-2-en-1-imine (42.9 mg, 0.280 mmol, 1.0 equiv) and 1-methyl-4-((trimethylsilyl)ethynyl)-1H-pyrazole (74.9 mg, 0.420 mmol, 1.5 equiv) in THE (0.42 mL). Rh catalyst (10 mol %, 0.28 mL, 28 μmol, 100 mM in THF) was added, and the reaction was carried out at 65° C. for 86 h to produce the DHP intermediate. Following general procedure D, DHP reduction was carried out with Na(OAc)₃BH (82.3 mg, 0.390 mmol, 3.0 equiv) in THE (1.3 mL), HF-pyridine (0.26 mL, 0.011 mol, 85 equiv), and crude DHP solution (0.130 mmol based upon ¹H NMR, 1.0 equiv). The crude DHP solution was transferred and rinsed with THF (0.3 mL). The resulting crude THP product was purified by preparative thin-layer chromatography (5% MeOH/CH₂Cl₂+0.5% NH₄OH) with only pure fractions isolated to afford the title compound (5.8 mg, 8% yield from imine) as a pale-yellow oil. ¹H NMR (600 MHz, C₆D₆) δ 7.68 (s, 1H), 6.67 (s, 1H), 5.76 (s, 1H), 4.44 (d, J=5.6 Hz, 1H), 4.42 (d, J=5.6 Hz, 1H), 4.23 (d, J=5.6 Hz, 1H), 4.22 (d, J=5.4 Hz, 1H), 3.21 (s, 3H), 3.03 (d, J=15.2 Hz, 1H), 2.94 (d, J=15.3 Hz, 1H), 2.42-2.37 (m, 2H), 2.37 (s, 2H), 1.94-1.86 (m, 1H), 1.30 (s, 3H), 0.95 (d, J=6.8 Hz, 3H). ¹³C NMR (151 MHz, C₆D₆) δ 135.9, 127.3, 125.4, 124.6, 122.4, 82.0, 81.9, 65.5, 58.2, 55.7, 39.7, 38.4, 31.2, 22.6, 19.5. HRMS (ESI+, m/z) [M+H]⁺ calcd for C₁₅H₂₄N₃O+: 262.1919. found: 262.1944.

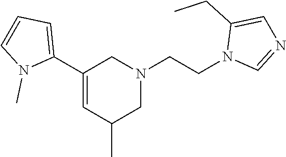 [SMILES: CCc1cncn1CCN1CC(c2cccn2C)=CC(C)C1]

(±) 1-(2-(5-Ethyl-1H-imidazol-1-yl)ethyl)-3-methyl-5-(1-methyl-1H-pyrrol-2-yl)-1,2,3,6-tetrahydropyridine (Compound 2): To access the benzyl intermediate, general procedure C was followed with minor modifications using [RhCl(coe)₂]₂ (113 mg, 0.157 mmol, 0.025 equiv), followed by ligand (65.7 mg, 0.341 mmol, 0.05 equiv), (E)-N-Benzyl-2-methylprop-2-en-1-imine (1.00 g, 6.26 mmol, 1.0 equiv) and 1-methyl-2-((trimethylsilyl)ethynyl)-1H-pyrrole (1.39 g, 7.85 mmol, 1.25 equiv) in toluene (16 mL). The reaction was carried out at 90° C. for 6 h to produce the DHP intermediate. Following general procedure D, DHP reduction was carried out with Na(OAc)₃BH (3.31 g, 15.6 mmol, 3.0 equiv) in THE (52 mL), HF-pyridine (10 mL, 0.44 mol, 85 equiv), and crude DHP solution (5.20 mmol based upon ¹H NMR, 1.0 equiv). The crude DHP solution was transferred and rinsed with THE (13 mL). The resulting crude THP product was purified by flash-column chromatography (10% EtOAc/hexanes+1% Et₃N) followed by preparative thin-layer chromatography (30% EtOAc/hexanes+1% Et₃N) with only pure fractions isolated to afford (±) 1-benzyl-3-methyl-5-(1-methyl-1H-pyrrol-2-yl)-1,2,3,6-tetrahydropyridine (324 mg, 19% yield from imine) as a pale-yellow oil. ¹H NMR (400 MHz, C₆D₆) δ 7.40 (d, J=7.3 Hz, 2H), 7.21 (t, J=7.4 Hz, 2H), 7.11 (t, J=7.3 Hz, 1H), 6.36-6.33 (m, 1H), 6.27-6.23 (m, 1H), 6.23-6.20 (m, 1H), 5.49 (s, 1H), 3.48 (d, J=13.9 Hz, 1H), 3.44 (d, J=13.3 Hz, 1H), 3.33 (d, J=15.8 Hz, 1H), 3.09 (d, J=15.8 Hz, 1H), 3.03 (s, 3H), 2.66 (dd, J=10.9, 5.1 Hz, 1H), 2.53-2.40 (m, 1H), 1.98 (dd, J=10.9, 7.4 Hz, 1H), 0.90 (d, J=7.0 Hz, 3H).

General procedure F was followed using this N-Bn THP (324 mg, 1.22 mmol, 1.0 equiv) and 1-chloroethyl chloroformate (0.16 mL, 1.46 mmol, 1.2 equiv) in DCE (6.1 mL). After 2 h, the reaction mixture was concentrated followed by flash chromatography (15% EtOAc/hexanes). The second step was carried out in MeOH (4.5 mL) and yielded the secondary amine salt (±) 3-methyl-5-(1-methyl-1H-pyrrol-2-yl)-1,2,3,6-tetrahydropyridin-1-ium chloride (135 mg, 52% yield over two steps) as a white solid. ¹H NMR (500 MHz, CD₃OD) δ 6.73-6.68 (m, 1H), 6.13-6.10 (m, 1H), 6.07-6.02 (m, 1H), 5.84 (s, 1H), 3.88 (d, J=16.3 Hz, 1H), 3.81 (d, J=16.3 Hz, 1H), 3.67 (s, 3H), 3.51 (dd, J=11.5, 5.0 Hz, 1H), 2.92-2.77 (m, 2H), 1.21 (d, J=6.8 Hz, 3H).

The carboxylic acid input, 1-(carboxymethyl)-5-ethyl-1H-imidazol-3-ium chloride, for N-alkylation was first prepared via an initial protection of 4-ethyl-1H-imidazole following a procedure adapted from the literature. To a flame-dried round-bottom flask was added 4-ethyl-1H-imidazole (1.50 g, 15.6 mmol, 1.0 equiv), Et₃N (4.4 mL, 31 mmol, 2.0 equiv), and trityl chloride (4.78 g, 17.16 mmol, 1.1 equiv) in DMF (45 mL). The reaction mixture stirred at rt for 4 h. Water (100 mL) was added, and the reaction mixture was extracted with CH₂Cl₂ (3×100 mL). The combined organic layers were washed with brine, dried over MgSO₄, filtered, and concentrated in vacuo. The crude material was purified by flash-column chromatography (50% EtOAc/hexanes) to yield 4.51 g (85% yield) of the protected product as a cream-colored solid. ¹H NMR (400 MHz, CDCl₃) δ 7.43-7.26 (m, 10H), 7.18-7.07 (m, 6H), 6.52 (s, 1H), 2.58 (q, J=7.5 Hz, 2H), 1.19 (t, J=7.5 Hz, 3H).

Next, 4-ethyl-1-trityl-imidazole (4.51 g, 13.3 mmol, 1.0 equiv) was dissolved in acetone (65 mL), transferred to a flame-dried round-bottom flask, and combined with methyl-2-bromoacetate (1.5 mL, 16 mmol, 1.2 equiv). The reaction mixture was heated to reflux and stirred for 6 h. The solvent was removed in vacuo, the resulting residue was dissolved in MeOH (13 mL), and the solution was again heated to reflux for 45 min. The reaction mixture was concentrated in vacuo and was triturated with Et₂O. The resulting precipitate was stirred at rt for 2 h with a mixture of NH₃ (4 mL, 7N in MeOH) and Et₂O (26 mL). The reaction mixture was filtered, and the filtrate was concentrated in vacuo. The residue was purified by flash-column chromatography (5% MeOH/CH₂Cl₂) to yield 780 mg (59% yield) of methyl 2-(5-ethyl-1H-imidazol-1-yl)acetate as a clear oil.

Methyl 2-(5-ethyl-1H-imidazol-1-yl)acetate (300 mg, 1.78 mmol, 1.0 equiv) was next refluxed in 4N HCl (aq) (9.0 mL, 36 mmol, 20 equiv) for 3.5 hours and concentrated to give 331 mg of 1-(carboxymethyl)-5-ethyl-1H-imidazol-3-ium chloride as a pale-yellow solid that was used directly in the next step without further purification. ¹H NMR (400 MHz, CD₃OD) δ 8.89 (s, 1H), 7.38 (s, 1H), 5.11 (s, 2H), 2.67 (q, J=7.5 Hz, 2H), 1.32 (t, J=7.5 Hz, 3H).

In an N₂ filled inert atmosphere, THP amine salt (50.0 mg, 0.235 mmol, 1.0 equiv), 1-(carboxymethyl)-5-ethyl-1H-imidazol-3-ium chloride (89.6 mg, 0.470 mmol, 2.0 equiv), Et₃N (0.16 mL, 1.2 mmol, 5.0 equiv), and BOP-Cl (120 mg, 0.470 mmol, 2.0 equiv) were combined in CH₂Cl₂ (0.8 mL). The crude material was purified by preparative thin-layer chromatography (10% MeOH/CH₂Cl₂+1% NH₄OH) to yield the desired product (46.6 mg, 64% yield) as a clear oil. ¹H NMR (400 MHz, CDCl₃; compound exists as a 1:1.25 mixture of rotamers; the major rotamer is denoted by *, minor rotamer denoted by ^{§}) δ 7.41 (s, 1H*, 1H^{§}), 6.82 (s, 1H*, 1H^{§}), 6.65 (s, 1H^{§}), 6.61 (s, 1H*), 6.18-6.12 (m, 1H*, 1H^{§}), 6.11-6.04 (m, 1H*, 1H^{§}), 5.79 (s, 1H^{§}), 5.68 (s, 1H*), 4.72 (s, 2H*), 4.69 (s, 2H^{§}), 4.46 (d, J=18.1 Hz, 1H*), 4.22-4.03 (m, 1H*, 3H^{§}), 3.75-3.68 (m, 1H*), 3.66 (s, 3H^{§}), 3.63 (s, 3H*), 3.15 (dd, J=13.3, 7.6 Hz, 1H*), 2.99 (dd, J=12.7, 8.1 Hz, 1H^{§}), 2.67-2.52 (m, 1H*, 1H^{§}), 2.47 (apparent p, J=7.2 Hz, 2H*, 2H^{§}), 1.34-1.20 (m, 3H*, 3H^{§}), 1.13 (d, J=7.0 Hz, 3H*), 1.08 (d, J=7.0 Hz, 3H^{§}).

The resulting amide (29.0 mg, 0.093 mmol, 1.0 equiv) was used with Zn(OAc)₂ (3.4 mg, 0.019 mmol, 0.20 equiv) and (EtO)₃SiH (0.10 mL, 0.56 mmol, 6.0 equiv) in THE (0.3 mL). Following purification by preparative thin-layer chromatography (10% MeOH/CH₂Cl₂+1% NH₄OH), the title compound was isolated as a clear oil (8.3 mg, 30% yield). ¹H NMR (600 MHz, CDCl₃) δ 7.51 (s, 1H), 6.78 (s, 1H), 6.59 (s, 1H), 6.10 (s, 1H), 6.03 (s, 1H), 5.62 (s, 1H), 4.00 (t, J=7.0 Hz, 2H), 3.62 (s, 3H), 3.30 (d, J=15.5 Hz, 1H), 3.07 (d, J=15.5 Hz, 1H), 2.84-2.71 (m, 3H), 2.65-2.48 (m, 3H), 2.16-2.08 (m, 1H), 1.28 (t, J=7.5 Hz, 3H), 1.05 (d, J=7.0 Hz, 3H). ¹³C NMR (151 MHz, CDCl₃) δ 137.2, 133.3, 132.5, 129.9, 127.2, 125.4, 123.7, 107.3, 107.2, 58.4, 58.0, 56.2, 42.7, 35.4, 31.2, 19.3, 17.6, 12.6. HRMS (ESI+, m/z) [M+H]⁺ calcd for C₁₈H₂₇N₄⁺: 299.2230. found: 299.2235.

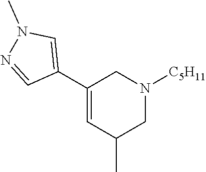

(±) 3-Methyl-5-(1-methyl-1H-pyrazol-4-yl)-1-pentyl-1,2,3,6-tetrahydropyridine (Compound 3): General procedure C was followed using (E)-2-Methyl-N-pentylprop-2-en-1-imine (49.0 mg, 0.352 mmol, 1.0 equiv) and 1-methyl-4-((trimethylsilyl)ethynyl)-1H-pyrazole (115 mg, 0.645 mmol, 1.8 equiv) in toluene (0.7 mL). Rh catalyst (2.5 mol %, 0.18 mL, 8.8 μmol, 50 mM in toluene) was added, and the reaction was carried out at 90° C. for 2.5 h to produce the DHP intermediate. Following general procedure D, DHP reduction was carried out with Na(OAc)₃BH (190 mg, 0.90 mmol, 3.0 equiv) in THE (3 mL), HF-pyridine (0.60 mL, 26 mmol, 85 equiv), and crude DHP solution (0.3 mmol based upon ¹H NMR, 1.0 equiv). The crude DHP solution was transferred and rinsed with THE (0.75 mL). The resulting crude THP product was purified by flash-column chromatography (50% EtOAc/hexanes+1% Et₃N) with only pure fractions isolated to afford the title compound as a pale-yellow oil (20.3 mg, 23% yield from imine). ¹H NMR (600 MHz, C₆D₆) δ 7.72 (s, 1H), 6.66 (s, 1H), 5.82 (s, 1H), 3.31 (d, J=15.2 Hz, 1H), 3.21 (s, 3H), 3.03 (d, J=15.3 Hz, 1H), 2.71 (dd, J=10.8, 5.0 Hz, 1H), 2.58-2.50 (m, 1H), 2.47-2.34 (m, 2H), 2.04 (dd, J=10.8, 7.4 Hz, 1H), 1.58 (p, J=7.3 Hz, 2H), 1.43-1.29 (m, 4H), 1.02 (d, J=7.0 Hz, 3H), 0.93 (t, J=7.1 Hz, 3H). ¹³C NMR (151 MHz, C₆D₆) δ 136.0, 127.7, 125.5, 124.9, 122.6, 58.7, 58.3, 55.4, 38.4, 31.5, 30.1, 27.4, 23.1, 19.6, 14.4. HRMS (ESI+, m/z) [M+H]⁺ calcd for C₁₅H₂₆N₃⁺: 248.2127. found: 248.2118.

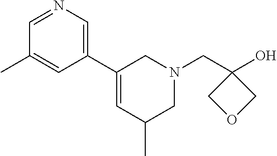 [SMILES: Cc1cncc(C2=CC(C)CN(CC3(O)COC3)C2)c1]

(±) 3-((5,5′-Dimethyl-5,6-dihydro-[3,3′-bipyridin]-1(2H)-yl)methyl)oxetan-3-ol (Compound 4) and (R) and (S) Enantiomers (Compounds 5 and 6): To access the benzyl intermediate, general procedure C was followed using (E)-N-benzyl-2-methylprop-2-en-1-imine (465 mg, 2.92 mmol, 1.0 equiv) and 3-methyl-5-((trimethylsilyl)ethynyl)pyridine (829 mg, 4.38 mmol, 1.5 equiv) in toluene (1.5 mL). Rh catalyst (10 mol %, 5.8 mL, 290 μmol, 50 mM in toluene) was added, and the reaction was carried out at 100° C. for 64 h to produce the DHP intermediate. Following general procedure D, DHP reduction was carried out with Na(OAc)₃BH (1.21 g, 5.71 mmol, 3.0 equiv) in THE (19 mL), HF-pyridine (3.8 mL, 162 mmol, 85 equiv), and crude DHP solution (1.90 mmol based upon ¹H NMR, 1.0 equiv). The crude DHP solution was transferred and rinsed with THE (4 mL). The resulting crude THP product was purified by flash-column chromatography (20% EtOAc/hexanes+1% Et₃N) followed by preparative thin-layer chromatography (60% EtOAc/hexanes+1% Et₃N) with only pure fractions isolated to afford (±) 1-benzyl-5,5′-dimethyl-1,2,5,6-tetrahydro-3,3′-bipyridine (254 mg, 31% yield from imine) as a pale-yellow oil. ¹H NMR (400 MHz, CDCl₃) δ 8.37 (s, 1H), 8.27 (s, 1H), 7.37 (d, J=8.8 Hz, 3H), 7.32 (t, J=7.4 Hz, 2H), 7.26 (t, J=7.1 Hz, 1H), 5.99 (s, 1H), 3.71 (d, J=13.1 Hz, 1H), 3.64 (d, J=13.1 Hz, 1H), 3.41 (d, J=16.0 Hz, 1H), 3.17 (d, J=16.0 Hz, 1H), 2.83 (dd, J=11.0, 5.3 Hz, 1H), 2.61-2.50 (m, 1H), 2.29 (s, 3H), 2.02 (dd, J=11.1, 8.1 Hz, 1H), 1.03 (d, J=7.1 Hz, 3H).

General procedure F was followed using this N-Bn THP (139 mg, 0.500 mmol, 1.0 equiv) and 1-chloroethyl chloroformate (60 μL, 0.60 mmol, 1.2 equiv) in DCE (2.5 mL). The reaction was carried out for 24 h before concentration. The crude secondary amine salt (±) 5,5′-dimethyl-1,2,5,6-tetrahydro-[3,3′-bipyridine]-1,1′-diium chloride was taken directly to the second step, which was carried out in MeOH (3.9 mL). This THP was used in the next step without purification.

The aldehyde input for N-alkylation was first prepared starting with the synthesis of 3-vinyloxetan-3-ol from oxetan-3-one (1.00 mL, 15.6 mmol, 1.0 equiv), which was subsequently protected. To a flame-dried round-bottom flask was added NaH (1.19 g, 31.2 mmol, 2.0 equiv, 60% dispersion in mineral oil), 4-methoxybenzylchloride (4.2 mL, 31 mmol, 1.0 equiv), and tetra-n-butylammonium iodide (576 mg, 1.56 mmol, 0.1 equiv) in THE (60 mL). The reaction mixture was stirred at rt for 16 h. The reaction was then quenched with saturated NH₄Cl (aq), and the resulting mixture was extracted with Et₂O (3×50 mL). The combined organic layers were washed with brine, dried with MgSO₄, filtered and concentrated in vacuo. The crude product was purified by flash-column chromatography (10% EtOAc/pentane) to yield 3-[(4-methoxyphenyl)methoxy]-3-vinyl-oxetane (2.07 g, 60% yield over two steps) as a clear oil. ¹H NMR (400 MHz, CDCl₃) δ 7.28 (d, J=8.6 Hz, 2H), 6.89 (d, J=8.6 Hz, 2H), 6.10 (dd, J=17.6, 10.9 Hz, 1H), 5.51 (d, J=17.6 Hz, 1H), 5.46 (d, J=10.9 Hz, 1H), 4.76 (d, J=6.9 Hz, 2H), 4.60 (d, J=7.0 Hz, 2H), 4.32 (s, 2H), 3.81 (s, 3H).

Next, 3-[(4-methoxyphenyl)methoxy]oxetane-3-carbaldehyde was synthesized. 3-[(4-Methoxyphenyl)methoxy]-3-vinyl-oxetane (1.24 g, 5.63 mmol, 1.0 equiv) was added to a flame-dried round-bottom flask and dissolved in CH₂Cl₂ (28 mL). The flask was cooled to −78° C., and the reaction mixture was purged with O₂ followed by a supply of O₃. The reaction was continued under 03 for 5 min. While still at −78° C., the reaction was quenched with Me-₂S (0.83 mL, 11 mmol, 2.0 equiv) and then the solution was allowed to warm to rt. The reaction mixture was concentrated in vacuo and purified by flash-column chromatography (30% EtOAc/Hexanes) with only pure fractions isolated to yield the desired product (239 mg, 19% yield) as a clear oil. ¹H NMR (400 MHz, CDCl₃) δ 9.78 (s, 1H), 7.28 (d, J=8.6 Hz, 2H), 6.90 (d, J=8.6 Hz, 2H), 4.74 (s, 4H), 4.50 (s, 2H), 3.80 (s, 3H).

General procedure G was then followed using the THP amine salt (192 mg, 0.854 mmol, 1.0 equiv), 3-[(4-methoxyphenyl)methoxy]oxetane-3-carbaldehyde (209 mg, 0.940 mmol, 1.1 equiv), Et₃N (0.24 mL, 1.7 mmol, 2.0 equiv), and Na(OAc)₃BH (272 mg, 1.28 mmol, 1.5 equiv) in CH₂Cl₂ (7.1 mL). The reaction was carried out for 16 h at rt. The crude material was purified by flash-column chromatography (20% acetone/CH₂Cl₂) with only pure fractions isolated to afford the desired product (130 mg, 39% yield) as a pale-yellow oil. ¹H NMR (500 MHz, CDCl₃) δ 8.39 (s, 1H), 8.30 (s, 1H), 7.34 (s, 1H), 7.29 (d, J=8.6 Hz, 2H), 6.87 (d, J=8.6 Hz, 2H), 6.02 (s, 1H), 4.80 (d, J=6.8, 2H), 4.56 (s, 2H), 4.54 (d, J=7.0, 2H), 3.80 (s, 3H), 3.52 (d, J=15.6 Hz, 1H), 3.34 (d, J=15.5 Hz, 1H), 3.09 (d, J=13.9 Hz, 1H), 3.06 (d, J=13.9 Hz, 1H), 2.91 (dd, J=10.9, 5.1 Hz, 1H), 2.62-2.53 (m, 1H), 2.32-2.25 (m, 1H), 2.29 (s, 3H), 1.07 (d, J=7.1 Hz, 3H).

General procedure E was followed using protected THP (130 mg, 0.33 mmol, 1.0 equiv) dissolved in CH₂Cl₂ (3.3 mL) and was reacted with trifluoroacetic acid (0.25 mL, 3.3 mmol, 10 equiv) at rt for 24 h. The crude product was purified by preparative thin-layer chromatography (10% MeOH/CH₂Cl₂+1% NH₄OH) followed by reverse-phase HPLC (1% MeCN/H₂O+0.1% formic acid) with only pure fractions isolated to yield the racemic title compound (4) (3.5 mg, 4% yield) as a clear oil. ¹H NMR (600 MHz, CDCl₃) δ 8.38 (s, 1H), 8.34 (s, 1H), 7.39 (s, 1H), 6.02 (s, 1H), 4.78 (t, J=6.5 Hz, 2H), 4.55 (d, J=6.5 Hz, 2H), 3.29 (s, 2H), 2.98 (s, 2H), 2.75 (dd, J=11.1, 5.1 Hz, 1H), 2.64-2.54 (m, 1H), 2.34 (s, 3H), 2.26 (dd, J=11.1, 7.7 Hz, 1H), 1.08 (d, J=7.0 Hz, 3H). ¹³C NMR (151 MHz, CDCl₃) δ 149.2, 143.9, 134.5, 133.0, 132.9, 131.4, 130.3, 84.0, 83.9, 71.2, 63.8, 57.3, 54.1, 31.1, 19.1, 18.6. HRMS (ESI+, m/z) [M+H]⁺ calcd for C₁₆H₂₃N₂O₂+: 275.1754. found: 275.1764.

Upon determining activity of racemate 4, a larger quantity was prepared (35 mg, 32% yield) for chiral separation according to the procedure described herein. A portion of this material was separated using semi-preparative chiral HPLC (Chiralpak AD-H column, 250×10 mm, 12% ethanol/hexanes+0.5% diethylamine, 3 mL/min) to provide the R- and S-enantiomers with tᵣ=66.0 min and 81.0 min respectively (Compounds 5 and 6).

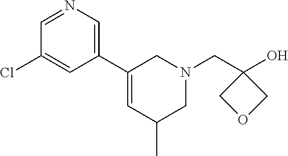 [SMILES: CC1C=C(c2cncc(Cl)c2)CN(CC2(O)COC2)C1]

(R) and (S) Enantiomers of 3-((5′-Chloro-5-methyl-5,6-dihydro-[3,3′-bipyridin]-1(2H)-yl)methyl)oxetan-3-ol (Compounds 7 and 8): To access the benzyl intermediate, general procedure C was followed using (E)-N-Benzyl-2-methylprop-2-en-1-imine (348 mg, 2.19 mmol, 1.0 equiv) and 3-chloro-5-((trimethylsilyl)ethynyl)pyridine (689 mg, 3.29 mmol, 1.5 equiv) in toluene (2.3 mL). Rh catalyst (7.5 mol %, 3.2 mL, 160 μmol, 50 mM in toluene) was added, and the reaction was carried out at 100° C. for 24 h to produce the DHP intermediate. Following general procedure D, DHP reduction was carried out with Na(OAc)₃BH (1.27 g, 6.00 mmol, 3.0 equiv) in THE (20 mL), HF-pyridine (4.0 mL, 170 mmol, 85 equiv), and crude DHP solution (2.00 mmol based upon ¹H NMR, 1.0 equiv). The crude DHP solution was transferred and rinsed with THE (4 mL). The resulting crude THP product was purified by preparative thin-layer chromatography (70% EtOAc/hexanes+1% Et₃N). This material was then converted into its salt form using trifluoroacetic acid to increase its water solubility for further purification by reverse-phase flash chromatography over C18 silica gel (30% MeCN/H₂O+0.1% TFA) with only pure fractions isolated to afford (1) 1-benzyl-5′-chloro-5-methyl-1,2,5,6-tetrahydro-3,3′-bipyridine (172 mg, 26% yield from imine) as a yellow oil. ¹H NMR (500 MHz, CDCl₃) δ 8.45 (s, 1H), 8.42 (s, 1H), 7.58 (s, 1H), 7.41-7.31 (m, 4H), 7.28 (t, J=7.5 Hz, 1H), 6.08 (s, 1H), 3.72 (d, J=13.1 Hz, 1H), 3.67 (d, J=13.1 Hz, 1H), 3.40 (d, J=15.6 Hz, 1H), 3.18 (d, J=15.7 Hz, 1H), 2.85 (dd, J=11.1, 5.3 Hz, 1H), 2.65-2.54 (m, 1H), 2.07 (dd, J=11.1, 8.0 Hz, 1H), 1.06 (d, J=7.1 Hz, 3H).

General procedure F was followed using this N-Bn THP (172 mg, 0.576 mmol, 1.0 equiv) and 1-chloroethyl chloroformate (0.075 mL, 0.69 mmol, 1.2 equiv) in DCE (2.9 mL). The reaction was carried out for 2.5 h before concentration and purification (20% EtOAc/hexanes). The second step was carried out in MeOH (1.9 mL) and yielded the secondary amine salt (±) 5′-chloro-5-methyl-1,2,5,6-tetrahydro-[3,3′-bipyridin]-1-ium chloride (63.7 mg, 45% yield over two steps) as a white solid. ¹H NMR (500 MHz, CD₃OD) δ 8.56 (d, J=1.9 Hz, 1H), 8.53 (d, J=2.1 Hz, 1H), 7.98 (t, J=2.1 Hz, 1H), 6.41 (d, J=1.9 Hz, 1H), 4.08 (s, 2H), 3.55 (dd, J=11.9, 5.4 Hz, 1H), 2.92 (dd, J=11.9, 9.6 Hz, 1H), 2.88-2.80 (m, 1H), 1.24 (d, J=7.0 Hz, 3H).

The aldehyde input for N-alkylation was prepared as described for compound 35. General procedure G was then followed using this 3-[(4-methoxyphenyl)methoxy]oxetane-3-carbaldehyde (25.2 mg, 0.114 mmol, 1.1 equiv), the THP amine salt (25.3 mg, 0.103 mmol, 1.0 equiv), Et₃N (29 μL, 0.21 mmol, 2.0 equiv), and Na(OAc)₃BH (32.8 mg, 0.155 mmol, 1.5 equiv) in CH₂Cl₂ (0.83 mL). The reaction was carried out for 2.5 h at rt. The crude material was purified by preparative thin-layer chromatography (80% EtOAc/hexanes+1% Et₃N) to afford the desired product (29.2 mg, 68% yield) as a clear oil. ¹H NMR (500 MHz, CDCl₃) δ 8.44 (s, 1H), 8.42 (s, 1H), 7.53 (s, 1H), 7.28 (d, J=8.3 Hz, 2H), 6.87 (d, J=8.3 Hz, 2H), 6.08 (s, 1H), 4.81 (d, J=5.3 Hz, 2H), 4.55 (s, 2H), 4.52 (d, J=4.6 Hz, 2H), 3.79 (s, 3H), 3.50 (d, J=15.6 Hz, 1H), 3.32 (d, J=15.6 Hz, 1H), 3.09 (d, J=14.0 Hz, 1H), 3.05 (d, J=14.0 Hz, 1H), 2.91 (dd, J=10.8, 4.8 Hz, 1H), 2.64-2.52 (m, 1H), 2.32-2.24 (m, 1H), 1.07 (d, J=7.0 Hz, 3H).

General procedure E was followed using protected THP (29.2 mg, 0.0704 mmol, 1.0 equiv) dissolved in CH₂Cl₂ (2.3 mL) and was reacted with trifluoroacetic acid (0.052 mL, 0.70 mmol, 10 equiv) at rt for 23 h. The crude product was purified by preparative thin-layer chromatography (10% MeOH/CH₂Cl₂+1% NH₄OH) followed by additional purification by preparative thin-layer chromatography (75% MTBE/hexanes+1% Et₃N) with only pure fractions isolated to yield 3-((5′-Chloro-5-methyl-5,6-dihydro-[3,3′-bipyridin]-1(2H)-yl)methyl)oxetan-3-ol (7.2 mg, 35% yield) as a clear oil. ¹H NMR (600 MHz, CDCl₃) δ 8.46 (s, 1H), 8.44 (s, 1H), 7.57 (s, 1H), 6.09 (s, 1H), 4.78 (m, 2H), 4.55 (d, J=6.7 Hz, 2H), 4.41 (br s, 1H), 3.28 (s, 2H), 2.98 (s, 2H), 2.75 (dd, J=11.2, 5.2 Hz, 1H), 2.67-2.53 (m, 1H), 2.26 (dd, J=11.2, 7.6 Hz, 1H), 1.09 (d, J=7.1 Hz, 3H). ¹³C NMR (151 MHz, CDCl₃) δ 147.4, 144.3, 136.1, 132.1, 132.0, 130.3, 83.89, 83.88, 71.3, 63.7, 57.1, 53.9, 31.2, 19.0. HRMS (ESI+, m/z) [M+H]⁺ calcd for C₁₅H₂₀ClN₂O₂⁺: 295.1213. found: 295.1194.

Upon confirmation of the activity of the racemate, a larger quantity of racemic material (23 mg, 33% yield) was prepared for chiral separation according the procedure described herein. A portion of this material was separated using semi-preparative chiral HPLC (Chiralpak AD-H column, 250×10 mm, 8% ethanol/hexanes+0.1% diethylamine, 2.5 mL/min) to provide the R- and S-enantiomers with tᵣ=87.5 min and 105.0 min respectively (Compounds 7 and 8).

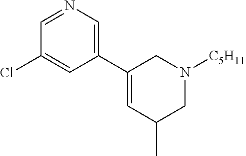

(R) and (S) enantiomers of 5′-chloro-5-methyl-1-pentyl-1,2,5,6-tetrahydro-3,3′-bipyridine (Compounds 9 and 10): General procedure C was followed using (E)-2-methyl-N-pentylprop-2-en-1-imine (89.3 mg, 0.641 mmol, 1.0 equiv) and 3-chloro-5-((trimethylsilyl)ethynyl)pyridine (202 mg, 0.962 mmol, 1.5 equiv) in THE (1.3 mL). Rh catalyst (5 mol %, 0.32 mL, 32 μmol, 100 mM in THF) was added, and the reaction was run at 65° C. for 24 h to produce the DHP intermediate. Following general procedure D, DHP reduction was performed with Na(OAc)₃BH (369 mg, 1.74 mmol, 3.0 equiv) in THE (5.8 mL), HF-pyridine (1.2 mL, 49 mmol, 85 equiv), and crude DHP solution (0.58 mmol based upon ¹H NMR, 1.0 equiv). The crude DHP solution was transferred and rinsed with THE (1.5 mL). The resulting crude THP product was purified by preparative thin-layer chromatography (30% EtOAc/hexanes) with only pure fractions isolated to afford 5′-chloro-5-methyl-1-pentyl-1,2,5,6-tetrahydro-3,3′-bipyridine (32.7 mg, 18% yield from imine) as a pale-yellow oil. ¹H NMR (600 MHz, CDCl₃) δ 8.48 (d, J=1.7 Hz, 1H), 8.43 (d, J=2.1 Hz, 1H), 7.60 (t, J=2.0 Hz, 1H), 6.05 (s, 1H), 3.43 (d, J=15.5 Hz, 1H), 3.08 (d, J=15.5 Hz, 1H), 2.89 (dd, J=11.1, 5.3 Hz, 1H), 2.71-2.58 (m, 1H), 2.57-2.36 (m, 2H), 1.99 (dd, J=11.0, 8.6 Hz, 1H), 1.58 (p, J=7.6 Hz, 2H), 1.43-1.25 (m, 4H), 1.07 (d, J=7.1 Hz, 3H), 0.91 (t, J=7.0 Hz, 3H). ¹³C NMR (151 MHz, CDCl₃) δ 146.9, 144.4, 136.7, 132.2, 132.0, 131.8, 131.0, 58.4, 57.3, 54.3, 31.5, 29.8, 26.8, 22.6, 18.8, 14.1. HRMS (ESI+, m/z) [M+H]⁺ calcd for C₁₆H₂₄ClN₂⁺: 279.1628. found: 279.1619. A portion of this material was separated using semi-preparative chiral HPLC (Chiralpak AD-H column, 250×10 mm, 2% isopropanol/hexanes, 2 mL/min) to provide the R and S enantiomers with tᵣ=15.0 min and 19.5 min respectively (Compounds 9 and 10).

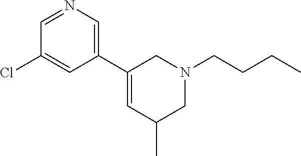 [SMILES: CCCCN1CC(c2cncc(Cl)c2)=CC(C)C1]

(R) and (S) enantiomers of 1-butyl-5′-chloro-5-methyl-1,2,5,6-tetrahydro-3,3′-bipyridine (Compounds 11 and 12): General procedure C was followed using (E)-N-butyl-2-methylprop-2-en-1-imine (150 mg, 1.20 mmol, 1.0 equiv) and 3-chloro-5-((trimethylsilyl)ethynyl)pyridine (378 mg, 1.80 mmol, 1.5 equiv) in THE (2.4 mL). Rh catalyst (5 mol %, 0.60 mL, 60 μmol, 100 mM in THF) was added, and the reaction was run at 65° C. for 24 h to produce the DHP intermediate. Following general procedure D, DHP reduction was performed with Na(OAc)₃BH (610 mg, 2.88 mmol, 3.0 equiv) in THE (9.5 mL), HF-pyridine (1.9 mL, 82 mmol, 85 equiv), and crude DHP solution (0.96 mmol based upon ¹H NMR, 1.0 equiv). The crude DHP solution was transferred and rinsed with THE (2.4 mL). The resulting crude THP product was purified by preparative thin-layer chromatography (50% EtOAc/hexanes) followed by a second preparative thin-layer chromatography purification (5% MeOH/CH₂Cl₂) with only pure fractions isolated to afford 1-butyl-5′-chloro-5-methyl-1,2,5,6-tetrahydro-3,3′-bipyridine (45.5 mg, 14% yield from imine) as a clear oil. ¹H NMR (600 MHz, CDCl₃) δ 8.48 (s, 1H), 8.43 (s, 1H), 7.60 (s, 1H), 6.06 (s, 1H), 3.43 (d, J=15.5 Hz, 1H), 3.08 (d, J=15.6 Hz, 1H), 2.88 (dd, J=11.1, 5.3 Hz, 1H), 2.66-2.57 (m, 1H), 2.57-2.43 (m, 2H), 1.99 (dd, J=10.9, 8.6 Hz, 1H), 1.57 (p, J=7.7 Hz, 2H), 1.37 (sx, J=7.4 Hz, 2H), 1.07 (d, J=7.0 Hz, 3H), 0.95 (t, J=7.4 Hz, 3H). ¹³C NMR (151 MHz, CDCl₃) δ 147.0, 144.6, 136.9, 132.3, 132.1, 132.0, 131.1, 58.2, 57.4, 54.5, 31.7, 29.4, 20.9, 19.0, 14.2. HRMS (ESI+, m/z) [M+H]⁺ calcd for C₁₅H₂₂ClN₂⁺: 265.1472. found: 265.1449. A portion of this material was separated using semi-preparative chiral HPLC (Chiralpak AD-H column, 250×10 mm, 2% isopropanol/hexanes, 2 mL/min) to provide the R and S enantiomers with tᵣ=15.4 min and 19.2 min respectively (Compounds 11 and 12).

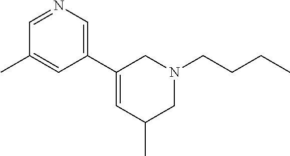 [SMILES: CCCCN1CC(c2cncc(C)c2)=CC(C)C1]

(R) and (S) Enantiomers of 1-Butyl-5,5′-dimethyl-1,2,5,6-tetrahydro-3,3′-bipyridine (Compounds 13 and 14): General procedure C was followed using (E)-N-Butyl-2-methylprop-2-en-1-imine (100 mg, 0.799 mmol, 1.0 equiv) and 3-methyl-5-((trimethylsilyl)ethynyl)pyridine (227 mg, 1.20 mmol, 1.5 equiv) in THE (1.6 mL). Rh catalyst (10 mol %, 0.80 mL, 80 μmol, 100 mM in THF) was added, and the reaction was carried out at 68° C. for 24 h to produce the DHP intermediate. Following general procedure D, DHP reduction was carried out with Na(OAc)₃BH (452 mg, 2.13 mmol, 3.0 equiv) in THE (7.1 mL), HF-pyridine (1.4 mL, 60 mmol, 85 equiv), and crude DHP solution (0.71 mmol based upon ¹H NMR, 1.0 equiv). The crude DHP solution was transferred and rinsed with THE (1.8 mL). The resulting crude THP product was purified by flash-column chromatography (50% EtOAc/hexanes) with only pure fractions isolated to afford the desired product 1-butyl-5,5′-dimethyl-1,2,5,6-tetrahydro-3,3′-bipyridine (130 mg, 67% yield from imine) as a red-brown oil. ¹H NMR (500 MHz, CDCl₃) δ 8.39 (s, 1H), 8.28 (s, 1H), 7.40 (s, 1H), 5.97 (s, 1H), 3.45 (d, J=15.6 Hz, 1H), 3.08 (d, J=16.0 Hz, 1H), 2.88 (dd, J=11.1, 5.4 Hz, 1H), 2.63-2.55 (m, 1H), 2.52-2.47 (m, 2H), 2.30 (s, 3H), 1.98 (t, J=11.1, 8.8 Hz, 1H), 1.60-1.51 (m, 2H), 1.35 (sx, J=7.3 Hz, 2H), 1.05 (d, J=7.0 Hz, 3H), 0.93 (t, J=7.3 Hz, 3H). ¹³C NMR (126 MHz, CDCl₃) δ 148.8, 144.0, 135.1, 133.0, 132.6, 132.1, 130.6, 58.2, 57.5, 54.6, 31.4, 29.3, 20.9, 19.1, 18.5, 14.2. HRMS (ESI+, m/z) [M+H]⁺ calcd for C₁₆H₂₅N₂⁺: 245.2012. found: 245.2012. A portion of this material was separated using semi-preparative chiral HPLC (Chiralpak AD-H column, 250×10 mm, 2% isopropanol/hexanes, 2 mL/min) to provide the R- and S-enantiomers with tᵣ=27.5 min and 34.0 min respectively (Compounds 13 and 14).

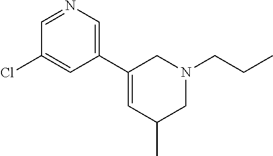 [SMILES: CCCN1CC(c2cncc(Cl)c2)=CC(C)C1]

(R) and (S) Enantiomers of 5′-Chloro-5-methyl-1-propyl-1,2,5,6-tetrahydro-3,3′-bipyridine (Compounds 15 and 16): General procedure C was followed using (E)-2-methyl-N-propylprop-2-en-1-imine (111 mg, 1.00 mmol, 1.0 equiv) and 3-chloro-5-((trimethylsilyl)ethynyl)pyridine (315 mg, 1.50 mmol, 1.5 equiv) in toluene (1.5 mL). Rh catalyst (5 mol %, 1.0 mL, 50 μmol, 50 mM in toluene) was added, and the reaction was carried out at 100° C. for 21 h to produce the DHP intermediate. Following general procedure D, DHP reduction was carried out with Na(OAc)₃BH (572 g, 2.70 mmol, 3.0 equiv) in THE (9 mL), HF-pyridine (1.8 mL, 76.5 mmol, 85 equiv), and crude DHP solution (0.90 mmol based upon ¹H NMR, 1.0 equiv). The crude DHP solution was transferred and rinsed with THE (1.8 mL). The resulting crude THP product was purified by preparative thin-layer chromatography (50% EtOAc/hexanes) followed by additional purification by preparative thin-layer chromatography (7% MeOH/CH₂Cl₂) with only pure fractions isolated to afford the desired product 5′-chloro-5-methyl-1-propyl-1,2,5,6-tetrahydro-3,3′-bipyridine (43.2 mg, 17% yield from imine) as a clear oil. ¹H NMR (400 MHz, C₆D₆) δ 8.48 (d, J=2.1 Hz, 2H), 7.27 (t, J=2.1 Hz, 1H), 5.66 (s, 1H), 3.01 (d, J=15.6 Hz, 1H), 2.85-2.71 (m, 1H), 2.51 (dd, J=10.9, 5.1 Hz, 1H), 2.38-2.27 (m, 2H), 2.27-2.06 (m, 2H), 1.85 (dd, J=10.9, 7.2 Hz, 1H), 1.42 (sx, J=7.3 Hz, 2H), 0.99-0.83 (m, 6H). ¹³C NMR (126 MHz, C₆D₆) δ 147.3, 144.9, 136.8, 132.0, 131.8, 131.7, 131.4, 60.2, 57.3, 54.2, 31.8, 20.5, 19.0, 12.1. HRMS (ESI+, m/z) [M+H]⁺ calcd for C₁₄H₂₀ClN₂⁺: 251.1315. found: 251.1308. A portion of this material was separated using semi-preparative chiral HPLC (Chiralpak AD-H column, 250×10 mm, 2% isopropanol/hexanes, 2.5 mL/min) to provide the R- and S-enantiomers with tᵣ=15.5 min and 18.0 min respectively (Compounds 15 and 16).

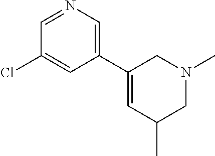

(R) and (S) enantiomers of 5′-chloro-1,5-dimethyl-1,2,5,6-tetrahydro-3,3′-bipyridine (Compounds 17 and 18): General procedure C was followed with minor modifications using [RhCl(coe)₂]₂ (71.8 mg, 0.200 mmol, 0.05 equiv), followed by ligand (41.9 mg, 0.100 mmol, 0.10 equiv), (E)-N,2-dimethylprop-2-en-1-imine solution (3.3 mL, 2.0 mmol, 1.0 equiv, 0.6 M solution in THF) and 3-chloro-5-((trimethylsilyl)ethynyl)pyridine (629 mg, 3.00 mmol, 1.5 equiv) in THE (mL). The reaction was run at 65° C. for 17 h to produce the DHP intermediate. Following general procedure D, DHP reduction was performed with Na(OAc)₃BH (1.02 g, 4.80 mmol, 3.0 equiv) in THE (16 mL), HF-pyridine (3.2 mL, 140 mmol, 85 equiv), and crude DHP solution (1.6 mmol based upon ¹H NMR, 1.0 equiv). The crude DHP solution was transferred and rinsed with THE (4 mL). The resulting crude THP product was purified by preparative thin-layer chromatography (50% EtOAc/hexanes+1% Et₃N) followed by additional purification by preparative thin-layer chromatography (5% MeOH/CH₂Cl₂+1% NH₄OH) with only pure fractions isolated to afford 5′-chloro-1,5-dimethyl-1,2,5,6-tetrahydro-3,3′-bipyridine (39.7 mg, 9% yield from imine) as a pale-yellow oil. ¹H NMR (600 MHz, C₆D₆) δ 8.48 (d, J=2.3 Hz, 1H), 8.45 (d, J=2.0 Hz, 1H), 7.23 (t, J=2.2 Hz, 1H), 5.66-5.59 (m, 1H), 2.86 (d, J=15.5 Hz, 1H), 2.67-2.61 (m, 1H), 2.41 (dd, J=10.9, 5.3 Hz, 1H), 2.37-2.29 (m, 1H), 2.13 (s, 3H), 1.77 (dd, J=10.9, 7.2 Hz, 1H), 0.86 (d, J=7.1 Hz, 3H). ¹³C NMR (151 MHz, C₆D₆) δ 146.9, 144.5, 136.2, 131.6, 131.2, 131.0, 130.9, 58.8, 55.3, 45.4, 31.4, 18.6. HRMS (ESI+, m/z) [M+H]⁺ calcd for C₁₂H₁₆ClN₂⁺: 223.1002. found: 223.0996. A portion of this material was separated using semi-preparative chiral HPLC (Chiralpak AD-H column, 250×10 mm, 2% ethanol/hexanes, 2.5 mL/min) to provide the S and R enantiomers with tᵣ=48.0 min and 60.0 min respectively (Compounds 17 and 18).

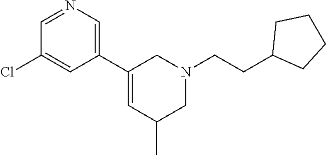 [SMILES: CC1C=C(c2cncc(Cl)c2)CN(CCC2CCCC2)C1]

(R) and (S) enantiomers of 5′-chloro-1-(2-cyclopentylethyl)-5-methyl-1,2,5,6-tetrahydro-3,3′-bipyridine (Compounds 19 and 20): General procedure C was followed using (E)-N-(2-cyclopentylethyl)-2-methylprop-2-en-1-imine (165 mg, 1.00 mmol, 1.0 equiv) and 3-chloro-5-((trimethylsilyl)ethynyl)pyridine (315 mg, 1.50 mmol, 1.5 equiv) in THF (2 mL). Rh catalyst (5 mol %, 0.50 mL, 50 μmol, 100 mM in THF) was added, and the reaction was run at 65° C. for 24 h to produce the DHP intermediate. Following general procedure D, DHP reduction was performed with Na(OAc)₃BH (572 mg, 2.70 mmol, 3.0 equiv) in THE (9 mL), HF-pyridine (1.8 mL, 76.5 mmol, 85 equiv), and crude DHP solution (0.90 mmol based upon ¹H NMR, 1.0 equiv). The crude DHP solution was transferred and rinsed with THE (2.3 mL). The resulting crude THP product was purified by flash-column chromatography (15% EtOAc/hexanes) followed by preparative thin-layer chromatography (30% EtOAc/hexanes) with only pure fractions isolated to afford 5′-chloro-1-(2-cyclopentylethyl)-5-methyl-1,2,5,6-tetrahydro-3,3′-bipyridine (11.2 mg, 4% yield from imine) as a pale-yellow oil. ¹H NMR (600 MHz, C₆D₆) δ 8.50 (d, J=1.9 Hz, 1H), 8.49 (d, J=2.3 Hz, 1H), 7.29 (s, 1H), 5.67 (s, 1H), 3.06 (d, J=15.5 Hz, 1H), 2.82 (d, J=15.5 Hz, 1H), 2.57 (dd, J=10.9, 5.1 Hz, 1H), 2.43-2.22 (m, 3H), 1.88 (dd, J=10.9, 7.3 Hz, 1H), 1.83-1.73 (m, 3H), 1.65-1.57 (m, 2H), 1.56-1.46 (m, 4H), 1.14-1.04 (m, 2H), 0.90 (d, J=7.1 Hz, 3H). ¹³C NMR (151 MHz, C₆D₆) δ 147.3, 144.9, 136.9, 132.0, 131.9, 131.7, 131.5, 57.8, 57.4, 54.4, 38.6, 33.9, 33.2, 33.1, 31.8, 25.54, 25.52, 19.0. HRMS (ESI+, m/z) [M+H]⁺ calcd for C₁₈H₂₆ClN₂⁺: 305.1785. found: 305.1775. A portion of this material was separated using semi-preparative chiral HPLC (Chiralpak AD-H column, 250×10 mm, 2% isopropanol/hexanes, 2 mL/min) to provide the R and S enantiomers with tᵣ=16.0 min and 20.5 min respectively (Compounds 19 and 20).

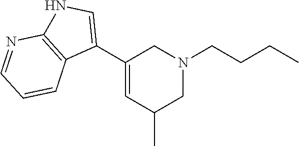 [SMILES: CCCCN1CC(c2c[nH]c3ncccc23)=CC(C)C1]

(R) and (S) enantiomers of 3-(1-butyl-5-methyl-1,2,5,6-tetrahydropyridin-3-yl)-1H-pyrrolo[2,3-b]pyridine (Compounds 21 and 22): General procedure C was followed using imine 8 g (87.6 mg, 0.700 mmol, 1.0 equiv) and tert-butyl 3-((trimethylsilyl)ethynyl)-1H-pyrrolo[2,3-b]pyridine-1-carboxylate (330 mg, 1.40 mmol, 1.5 equiv) in toluene (1 mL). Rh catalyst (5 mol %, 0.70 mL, 35 μmol, 50 mM in toluene) was added, and the reaction was run at 90° C. for 3.5 h to produce the DHP intermediate. Following general procedure D, DHP reduction was performed with Na(OAc)₃BH (445 mg, 2.10 mmol, 3.0 equiv) in THE (7.0 mL), HF-pyridine (1.4 mL, 60 mmol, 85 equiv), and crude DHP solution (0.7 mmol, 1.0 equiv). The crude DHP solution was transferred and rinsed with THE (1.8 mL). The resulting crude THP product was purified by preparative thin-layer chromatography (75% EtOAc/hexanes+1% Et₃N) followed by additional purification by preparative thin-layer chromatography (10% MeOH/CH₂Cl₂+1% NH₄OH) with only pure fractions isolated to afford the desired product (75.0 mg, 29% yield from imine) as a pale-yellow oil. ¹H NMR (500 MHz, CDCl₃) δ 8.50 (d, J=4.4 Hz, 1H), 8.10 (d, J=7.9 Hz, 1H), 7.50 (s, 1H), 7.20 (dd, J=7.9, 4.8 Hz, 1H), 6.10 (s, 1H), 3.47 (d, J=15.4 Hz, 1H), 3.11 (d, J=15.2 Hz, 1H), 2.93 (dd, J=10.8, 5.1 Hz, 1H), 2.73-2.61 (m, 1H), 2.52 (t, J=7.7 Hz, 2H), 2.04 (apparent t, J=9.7 Hz, 1H), 1.67 (s, 9H), 1.59 (p, J=7.6 Hz, 2H), 1.39 (sx, J=7.4 Hz, 2H), 1.10 (d, J=7.0 Hz, 3H), 0.95 (t, J=7.4 Hz, 3H). General procedure E was followed using this THP (75.0 mg, 0.203 mmol, 1.0 equiv) with trifluoroacetic acid (0.15 mL, 2.03 mmol, 10 equiv) in CH-₂Cl₂ (1.7 mL) at rt for 5 h. The crude product was purified by preparative thin-layer chromatography (10% MeOH/CH₂Cl₂+1% NH₄OH) followed by additional purification by preparative thin-layer chromatography (80% EtOAc/hexanes+1% Et₃N) to provide 3-(1-butyl-5-methyl-1,2,5,6-tetrahydropyridin-3-yl)-1H-pyrrolo[2,3-b]pyridine (31.6 mg, 58% yield) as a clear oil. ¹H NMR (600 MHz, CDCl₃) δ 11.31 (br s, 1H), 8.32 (d, J=4.6 Hz, 1H), 8.20 (d, J=7.9 Hz, 1H), 7.31 (s, 1H), 7.11 (dd, J=7.9, 4.7 Hz, 1H), 6.07 (s, 1H), 3.55 (d, J=15.2 Hz, 1H), 3.12 (d, J=15.2 Hz, 1H), 2.93 (dd, J=10.9, 5.3 Hz, 1H), 2.74-2.63 (m, 1H), 2.55-2.46 (m, 2H), 2.03 (dd, J=10.7, 9.0 Hz, 1H), 1.60 (p, J=7.6 Hz, 2H), 1.38 (sx, J=7.4 Hz, 2H), 1.10 (d, J=7.0 Hz, 3H), 0.95 (t, J=7.4 Hz, 3H). ¹³C NMR (151 MHz, CDCl₃) δ 149.4, 142.7, 129.4, 129.2, 126.9, 121.5, 118.5, 116.0, 115.0, 58.4, 58.1, 55.3, 31.3, 29.5, 21.0, 19.6, 14.3. HRMS (ESI+, m/z) [M+H]⁺ calcd for C₁₇H₂₄N₃⁺: 270.1970. found: 270.1979. A portion of this material was separated using semi-preparative chiral HPLC (Chiralpak AD-H column, 250×10 mm, 7% isopropanol/hexanes, 3 mL/min) to provide the R and S enantiomers with tᵣ=12.8 min and 19.2 min respectively (Compounds 21 and 22).

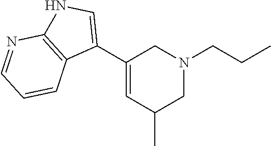 [SMILES: CCCN1CC(c2c[nH]c3ncccc23)=CC(C)C1]

(R) and (S) enantiomers of 3-(5-methyl-1-propyl-1,2,5,6-tetrahydropyridin-3-yl)-1H-pyrrolo[2,3-b]pyridine (Compounds 23 and 24): General procedure C was followed using (E)-2-methyl-N-propylprop-2-en-1-imine (77.8 mg, 0.700 mmol, 1.0 equiv) and tert-butyl 3-((trimethylsilyl)ethynyl)-1H-pyrrolo[2,3-b]pyridine-1-carboxylate (330 mg, 1.05 mmol, 1.5 equiv) in toluene (1 mL). Rh catalyst (5 mol %, 0.70 mL, 35 μmol, 50 mM in toluene) was added, and the reaction was run at 90° C. for 3.5 h to produce the DHP intermediate. Following general procedure D, DHP reduction was performed with Na(OAc)₃BH (445 mg, 2.10 mmol, 3.0 equiv) in THF (7 mL), HF-pyridine (1.4 mL, 60 mmol, 85 equiv), and crude DHP solution (0.7 mmol, 1.0 equiv). The crude DHP solution was transferred and rinsed with THE (1.8 mL). The resulting crude THP product was purified by preparative thin-layer chromatography (75% EtOAc/hexanes+1% Et₃N) with only pure fractions isolated to afford the desired product (82.2 mg, 33% yield from imine) as a pale-yellow oil. ¹H NMR (400 MHz, CDCl₃) δ 8.50 (d, J=3.9 Hz, 1H), 8.10 (d, J=7.3 Hz, 1H), 7.50 (s, 1H), 7.21 (dd, J=7.9, 4.8 Hz, 1H), 6.10 (s, 1H), 3.47 (d, J=15.4 Hz, 1H), 3.12 (d, J=15.6 Hz, 1H), 2.93 (dd, J=10.3, 4.8 Hz, 1H), 2.77-2.60 (m, 1H), 2.60-2.40 (m, 2H), 2.11-2.04 (m, 1H), 1.67 (s, 9H), 1.65-1.58 (m, 2H), 1.10 (d, J=7.0 Hz, 3H), 0.96 (t, J=7.4 Hz, 3H). General procedure E was followed using this THP (82.2 mg, 231 mmol, 1.0 equiv) with trifluoroacetic acid (0.17 mL, 2.3 mmol, 10 equiv) in CH₂Cl₂ (1.9 mL) at rt for 5 h. The crude product was purified by preparative thin-layer chromatography (10% MeOH/CH₂Cl₂+1% NH₄OH) followed by a second preparative thin-layer chromatography purification (80% EtOAc/hexanes+1% Et₃N) to yield 3-(5-methyl-1-propyl-1,2,5,6-tetrahydropyridin-3-yl)-1H-pyrrolo[2,3-b]pyridine (33.6 mg, 57% yield) as a clear oil. ¹H NMR (600 MHz, CDCl₃) δ 11.31 (br s, 1H), 8.32 (d, J=4.7 Hz, 1H), 8.20 (d, J=7.9 Hz, 1H), 7.31 (s, 1H), 7.11 (dd, J=7.9, 4.7 Hz, 1H), 6.07 (s, 1H), 3.55 (d, J=15.2 Hz, 1H), 3.12 (d, J=15.2 Hz, 1H), 2.93 (dd, J=10.9, 5.3 Hz, 1H), 2.78-2.61 (m, 1H), 2.59-2.43 (m, 2H), 2.13-1.99 (m, 1H), 1.64 (h, J=7.4 Hz, 2H), 1.10 (d, J=7.0 Hz, 3H), 0.96 (t, J=7.4 Hz, 3H). ¹³C NMR (151 MHz, CDCl₃) δ 149.5, 142.6, 129.4, 129.2, 126.9, 121.6, 118.5, 115.9, 114.9, 60.6, 58.0, 55.3, 31.3, 20.4, 19.6, 12.2. HRMS (ESI+, m/z) [M+H]⁺ calcd for C₁₆H₂₂N₃⁺: 256.1814. found: 256.1813. A portion of this material was separated using semi-preparative chiral HPLC (Chiralpak AD-H column, 250×10 mm, 7% isopropanol/hexanes, 3 mL/min) to provide the R and S enantiomers with tᵣ=21.0 min and 29.5 min respectively (Compounds 23 and 24).

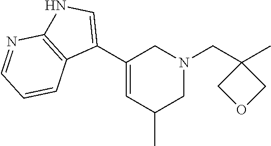 [SMILES: CC1C=C(c2c[nH]c3ncccc23)CN(CC2(C)COC2)C1]

(R) and (S) Enantiomers of 3-(5-Methyl-1-((3-methyloxetan-3-yl)methyl)-1,2,5,6-tetrahydropyridin-3-yl)-1H-pyrrolo[2,3-b]pyridine (Compounds 25 and 26): To access the benzyl THP intermediate, general procedure C was followed with minor modifications using [RhCl(coe)₂]₂ (135 mg, 0.188 mmol, 0.05 equiv), followed by ligand (78.5 mg, 0.375 mmol, 0.10 equiv), (E)-N-benzyl-2-methylprop-2-en-1-imine (597 mg, 3.75 mmol, 1.0 equiv) and tert-butyl 3-((trimethylsilyl)ethynyl)-1H-pyrrolo[2,3-b]pyridine-1-carboxylate (1.47 g, 4.69 mmol, 1.25 equiv) in toluene (9.4 mL). The reaction was carried out at 90° C. for 3 h to produce the DHP intermediate. Following general procedure D, DHP reduction was carried out with Na(OAc)₃BH (2.38 g, 11.3 mmol, 3.0 equiv) in THE (38 mL), HF-pyridine (7.5 mL, 319 mmol, 85 equiv), and crude DHP solution (3.75 mmol, 1.0 equiv). The crude DHP solution was transferred and rinsed with THF (9.4 mL). The resulting crude THP product was purified by flash-column chromatography (15% EtOAc/hexanes+1% Et₃N) to afford (±) tert-butyl 3-(1-benzyl-5-methyl-1,2,5,6-tetrahydropyridin-3-yl)-1H-pyrrolo[2,3-b]pyridine-1-carboxylate (734 mg, 49% yield from imine) as a thick pale-yellow oil. ¹H NMR (500 MHz, CDCl₃) δ 8.50 (dd, J=4.7, 1.3 Hz, 1H) 8.09 (dd, J=7.9, 1.4 Hz, 1H), 7.46 (s, 1H), 7.41 (d, J=7.3 Hz, 2H), 7.35 (t, J=7.5 Hz, 2H), 7.29 (t, J=7.2 Hz, 1H), 7.20 (dd, J=7.9, 4.8 Hz, 1H), 6.12 (s, 1H), 3.75 (d, J=13.1 Hz, 1H), 3.66 (d, J=13.1 Hz, 1H), 3.45 (d, J=15.4 Hz, 1H), 3.22 (d, J=15.4 Hz, 1H), 2.87 (dd, J=11.0, 5.3 Hz, 1H), 2.69-2.58 (m, 1H), 2.09 (dd, J=11.0, 8.1 Hz, 1H), 1.66 (s, 9H), 1.08 (d, J=7.0 Hz, 3H).

General procedure F was followed with minor modifications using this N-Bn THP (435 mg, 1.08 mmol, 1.0 equiv) and 1-chloroethyl chloroformate (0.17 mL, 1.62 mmol, 1.5 equiv) in DCE (5.4 mL). The reaction was carried out for 5 h before concentration and purification (20% EtOAc/hexanes). The second step was carried out in MeOH (6.7 mL) and yielded the secondary amine salt (±) 5-(1-(tert-butoxycarbonyl)-1H-pyrrolo[2,3-b]pyridin-3-yl)-3-methyl-1,2,3,6-tetrahydropyridin-1-ium (283 mg, 75% yield over two steps) as a white solid. ¹H NMR (500 MHz, CD₃OD) δ 8.43 (dd, J=4.8, 1.3 Hz, 1H), 8.31 (dd, J=8.0, 1.3 Hz, 1H), 7.82 (s, 1H), 7.38 (dd, J=8.0, 4.8 Hz, 1H), 6.42 (s, 1H), 4.12 (d, J=16.1 Hz, 1H), 4.06 (d, J=16.2 Hz, 1H), 3.57 (dd, J=11.7, 5.2 Hz, 1H), 3.03-2.84 (m, 2H), 1.69 (s, 9H), 1.28 (d, J=6.9 Hz, 3H).

General procedure G was followed using the THP amine salt (75.0 mg, 0.214 mmol, 1.0 equiv), 3-methyl-oxetane-3-carbaldehyde (23.6 mg, 0.236 mmol, 1.1 equiv), Et₃N (60 μL, 0.43 mmol, 2.0 equiv), and Na(OAc)₃BH (68.2 mg, 0.322 mmol, 1.5 equiv) in CH₂Cl₂ (1.7 mL). The reaction was carried out for 5 h at rt. The crude material was purified by preparative thin-layer chromatography (30% EtOAc/hexanes+1% Et₃N) to afford the desired product (76.0 mg, 89% yield) as a clear oil. ¹H NMR (500 MHz, CDCl₃) δ 8.51 (dd, J=4.7, 1.4 Hz, 1H), 8.09 (dd, J=8.2, 1.4 Hz, 1H), 7.46 (s, 1H), 7.21 (dd, J=7.9, 4.8 Hz, 1H), 6.12 (s, 1H), 4.56 (d, J=5.6 Hz, 2H), 4.39 (m, 2H), 3.28 (d, J=15.3 Hz, 1H), 3.20 (d, J=15.2 Hz, 1H), 2.75 (s, 2H), 2.66-2.58 (m, 2H), 2.14-2.06 (m, 1H), 1.67 (s, 9H), 1.46 (s, 3H), 1.10 (d, J=6.8 Hz, 3H). General procedure E was followed using protected THP (76.0 mg, 0.191 mmol, 1.0 equiv) dissolved in CH₂Cl₂ (1.6 mL) and was reacted with trifluoroacetic acid (0.14 mL, 1.91 mmol, 10 equiv) at rt for 8 h. The crude product was purified by preparative thin-layer chromatography (10% MeOH/CH₂Cl₂+1% NH₄OH) to yield 3-(5-methyl-1-((3-methyloxetan-3-yl)methyl)-1,2,5,6-tetrahydropyridin-3-yl)-1H-pyrrolo[2,3-b]pyridine (38.2 mg, 67% yield) as a clear oil. ¹H NMR (600 MHz, CDCl₃) δ 10.00 (s, 1H), 8.32 (d, J=5.1 Hz, 1H), 8.18 (d, J=7.9 Hz, 1H), 7.23 (s, 1H), 7.12 (dd, J=7.8, 4.8 Hz, 1H), 6.09 (s, 1H), 4.57 (m, 2H), 4.44-4.34 (m, 2H), 3.33 (d, J=15.0 Hz, 1H), 3.22 (d, J=15.0 Hz, 1H), 2.75 (s, 2H), 2.68-2.56 (m, 2H), 2.15-2.04 (m, 1H), 1.47 (s, 3H), 1.11 (d, J=6.7 Hz, 3H). ¹³C NMR (151 MHz, cdcl₃) δ 149.2, 143.3, 129.3, 128.9, 126.9, 121.0, 118.2, 116.2, 115.0, 82.8, 82.6, 65.6, 58.0, 55.9, 39.8, 31.2, 22.7, 19.6. HRMS (ESI+, m/z) [M+H]⁺ calcd for C₁₈H₂₄N₃O⁺: 298.1919. found: 298.1903. A portion of this material was separated using semi-preparative chiral HPLC (Chiralpak AD-H column, 250×10 mm, 5% ethanol/hexanes, 2.5 mL/min) to provide the R- and S-enantiomers with tᵣ=43.0 min and 50.0 min respectively (Compounds 25 and 26).

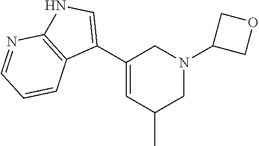 [SMILES: CC1C=C(c2c[nH]c3ncccc23)CN(C2COC2)C1]

(R) and (S) Enantiomers of 3-(5-Methyl-1-(oxetan-3-yl)-1,2,5,6-tetrahydropyridin-3-yl)-1H-pyrrolo[2,3-b]pyridine (Compounds 27 and 28): General procedure C was followed using (E)-2-methyl-N-(oxetan-3-yl)prop-2-en-1-imine (175 mg, 1.20 mmol, 1.0 equiv) and tert-butyl 3-((trimethylsilyl)ethynyl)-1H-pyrrolo[2,3-b]pyridine-1-carboxylate (566 mg, 1.80 mmol, 1.5 equiv) in toluene (1.3 mL). Rh catalyst (7.5 mol %, 1.8 mL, 90 μmol, 50 mM in toluene) was added, and the reaction was carried out at 90° C. for 6.5 h to produce the DHP intermediate. Following general procedure D, DHP reduction was carried out with Na(OAc)₃BH (763 g, 3.60 mmol, 3.0 equiv) in THE (12 mL), HF-pyridine (2.4 mL, 100 mmol, 85 equiv), and crude DHP solution (1.20 mmol, 1.0 equiv). The crude DHP solution was transferred and rinsed with THE (2.4 mL). The resulting crude THP product was purified by preparative thin-layer chromatography (50% EtOAc/hexanes+1% Et₃N) with only pure fractions isolated to afford the desired product (57.2 mg, 13% yield from imine) as a pale-yellow oil. ¹H NMR (500 MHz, CDCl₃) δ 8.51 (dd, J=4.7, 1.5 Hz, 1H), 8.08 (dd, J=8.0, 1.5 Hz, 1H), 7.47 (s, 1H), 7.22 (dd, J=7.9, 4.8 Hz, 1H), 6.13 (s, 1H), 4.75 (d, J=6.6 Hz, 4H), 3.73 (p, J=6.5 Hz, 1H), 3.32 (d, J=15.0 Hz, 1H), 3.05 (d, J=15.3 Hz, 1H), 2.77 (dd, J=10.9, 5.3 Hz, 1H), 2.73-2.62 (m, 1H), 2.00 (dd, J=10.9, 8.3 Hz, 1H), 1.67 (s, 9H), 1.12 (d, J=7.0 Hz, 3H). General procedure E was followed using this THP (57.0 mg, 0.154 mmol, 1.0 equiv) with trifluoroacetic acid (0.12 mL, 1.5 mmol, 10 equiv) in CH₂Cl₂ (1.2 mL) at rt for 5 h. The crude product was purified by preparative thin-layer chromatography (80% EtOAc/hexanes+1% Et₃N) to provide 3-(5-methyl-1-(oxetan-3-yl)-1,2,5,6-tetrahydropyridin-3-yl)-1H-pyrrolo[2,3-b]pyridine (26.7 mg, 64% yield) as a clear oil. ¹H NMR (600 MHz, CDCl₃) δ 9.91 (br s, 1H), 8.32 (d, J=4.5 Hz, 1H), 8.17 (d, J=8.0 Hz, 1H), 7.25 (s, 1H), 7.13 (dd, J=7.9, 4.7 Hz, 1H), 6.10 (s, 1H), 4.86-4.70 (m, 4H), 3.73 (p, J=6.5 Hz, 1H), 3.39 (d, J=15.0 Hz, 1H), 3.06 (d, J=14.8 Hz, 1H), 2.77 (dd, J=10.8, 5.4 Hz, 1H), 2.74-2.62 (m, 1H), 1.99 (dd, J=10.7, 8.7 Hz, 1H), 1.13 (d, J=7.0 Hz, 3H). ¹³C NMR (151 MHz, CDCl₃) δ 149.2, 143.4, 129.2, 128.3, 127.3, 121.1, 118.2, 116.3, 115.0, 76.1, 75.9, 59.0, 54.2, 51.5, 30.9, 19.5. HRMS (ESI+, m/z) [M+H]⁺ calcd for C₁₆H₂₀N₃O⁺: 270.1606. found: 270.1594. A portion of this material was separated using semi-preparative chiral HPLC (Chiralpak AD-H column, 250×10 mm, 11% ethanol/hexanes, 3 mL/min) to provide the R- and S-enantiomers with tᵣ=27.5 min and 36.5 min respectively (Compounds 27 and 28).

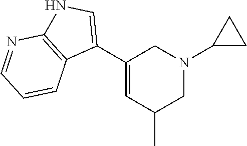 [SMILES: CC1C=C(c2c[nH]c3ncccc23)CN(C2CC2)C1]

(R) and (S) enantiomers of 3-(1-cyclopropyl-5-methyl-1,2,5,6-tetrahydropyridin-3-yl)-1H-pyrrolo[2,3-b]pyridine (Compounds 29 and 30): General procedure C was followed using (E)-N-cyclopropyl-2-methylprop-2-en-1-imine (131 mg, 1.20 mmol, 1.0 equiv) and 3-(5-methyl-1-propyl-1,2,5,6-tetrahydropyridin-3-yl)-1H-pyrrolo[2,3-b]pyridine (566 mg, 1.80 mmol, 1.5 equiv) in toluene (1.8 mL). Rh catalyst (5 mol %, 1.2 mL, 60 μmol, 50 mM in toluene) was added, and the reaction was run at 90° C. for 6.5 h to produce the DHP intermediate. Following general procedure D, DHP reduction was performed with Na(OAc)₃BH (668 mg, 3.15 mmol, 3.0 equiv) in THF (10 mL), HF-pyridine (2.1 mL, 89 mmol, 85 equiv), and crude DHP solution (1.05 mmol based upon ¹H NMR, 1.0 equiv). The crude DHP solution was transferred and rinsed with THE (2.6 mL). The resulting crude THP product was purified by flash-column chromatography (15% EtOAc/hexanes+1% Et₃N) followed by preparative thin-layer chromatography (15% EtOAc/hexanes+1% Et₃N) with only pure fractions isolated to afford the desired product (124 mg, 29% yield from imine) as a pale-yellow oil. ¹H NMR (400 MHz, CDCl₃) δ 8.50 (dd, J=4.7, 1.4 Hz, 1H), 8.10 (dd, J=8.0, 1.5 Hz, 1H), 7.52 (s, 1H), 7.21 (dd, J=7.9, 4.8 Hz, 1H), 6.08 (s, 1H), 3.58 (d, J=15.4 Hz, 1H), 3.33 (d, J=15.4 Hz, 1H), 3.11 (dd, J=10.9, 5.4 Hz, 1H), 2.70-2.56 (m, 1H), 2.32-2.20 (m, 1H), 1.88-1.77 (m, 1H), 1.68 (s, 9H), 1.09 (d, J=7.0 Hz, 3H), 0.60-0.50 (m, 4H). General procedure E was followed using this THP (124 mg, 0.351 mmol, 1.0 equiv) with trifluoroacetic acid (0.26 mL, 3.51 mmol, 10 equiv) in CH₂Cl₂ (2.9 mL) at rt for 4.5 h. The crude product was purified by preparative thin-layer chromatography (10% MeOH/CH₂Cl₂+1% NH₄OH) to yield 3-(1-cyclopropyl-5-methyl-1,2,5,6-tetrahydropyridin-3-yl)-1H-pyrrolo[2,3-b]pyridine (60 mg, 68% yield) as a pale-yellow oil. ¹H NMR (600 MHz, CDCl₃) δ 10.11 (br s, 1H), 8.32 (s, 1H), 8.26-8.15 (m, 1H), 7.31 (s, 1H), 7.12 (dd, J=7.9, 4.7 Hz, 1H), 6.06 (s, 1H), 3.66 (d, J=15.4 Hz, 1H), 3.36 (d, J=15.5 Hz, 1H), 3.12 (dd, J=11.0, 5.5 Hz, 1H), 2.73-2.57 (m, 1H), 2.27 (apparent t, J=9.9 Hz, 1H), 1.90-1.75 (m, 1H), 1.10 (d, J=7.0 Hz, 3H), 0.63-0.49 (m, 4H). ¹³C NMR (151 MHz, CDCl₃) δ 149.3, 143.2, 129.3, 129.1, 127.0, 121.2, 118.3, 116.2, 115.2, 58.4, 54.9, 38.3, 31.2, 19.5, 6.2, 6.1. HRMS (ESI+, m/z) [M+H]⁺ calcd for C₁₆H₂₀N₃⁺: 254.1657. found: 254.1635. A portion of this material was separated using semi-preparative chiral HPLC (Chiralpak AD-H column, 250×10 mm, 3% ethanol/hexanes, 2.5 mL/min) to provide the R and S enantiomers with tᵣ=22.6 min and 27.0 min respectively (Compounds 29 and 30).

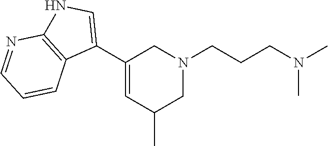 [SMILES: CC1C=C(c2c[nH]c3ncccc23)CN(CCCN(C)C)C1]

(R) and (S) Enantiomers of N,N-Dimethyl-3-(3-methyl-5-(1H-pyrrolo[2,3-b]pyridin-3-yl)-3,6-dihydropyridin-1(2H)-yl)propan-1-amine (Compounds 31 and 32): General procedure C was followed with minor modifications using [RhCl(coe)₂]₂ (45.0 mg, 0.0626 mmol, 0.05 equiv), followed by ligand (26.2 mg, 0.125 mmol, 0.10 equiv), (E)-N,N-dimethyl-3-((2-methylallylidene)amino)propan-1-amine (193 mg, 1.25 mmol, 1.0 equiv) and tert-butyl 3-((trimethylsilyl)ethynyl)-1H-pyrrolo[2,3-b]pyridine-1-carboxylate (590 mg, 1.88 mmol, 1.5 equiv) in toluene (3.1 mL). The reaction was carried out at 90° C. for 7 h to produce the DHP intermediate. Following general procedure D, DHP reduction was carried out with Na(OAc)₃BH (796 g, 3.75 mmol, 3.0 equiv) in THF (12.5 mL), HF-pyridine (2.5 mL, 110 mmol, 85 equiv), and crude DHP solution (1.25 mmol, 1.0 equiv). The crude DHP solution was transferred and rinsed with THF (2.5 mL). The resulting crude THP product was purified by flash-column chromatography (90% EtOAc/hexanes+1% Et₃N) with only pure fractions isolated to afford the desired product (160 mg, 32% yield from imine) as a pale-yellow oil. ¹H NMR (500 MHz, CDCl₃) δ 8.50 (dd, J=4.7, 1.4 Hz, 1H), 8.10 (dd, J=7.9, 1.4 Hz, 1H), 7.49 (s, 1H), 7.20 (dd, J=7.9, 4.8 Hz, 1H), 6.11 (s, 1H), 3.46 (d, J=15.3 Hz, 1H), 3.12 (d, J=15.4 Hz, 1H), 2.91 (dd, J=11.0, 5.3 Hz, 1H), 2.71-2.62 (m, 1H), 2.55 (t, J=7.5 Hz, 2H), 2.36 (t, J=8.3 Hz, 2H), 2.25 (s, 6H), 2.06 (dd, J=10.9, 8.4 Hz, 1H), 1.79 (p, J=7.5 Hz, 2H), 1.67 (s, 9H), 1.10 (d, J=7.0 Hz, 3H). General procedure E was followed using this THP (160 mg, 0.401 mmol, 1.0 equiv) with trifluoroacetic acid (0.30 mL, 4.0 mmol, 10 equiv) in CH₂Cl₂ (3.3 mL) at rt for 5 h. The crude product was purified by preparative thin-layer chromatography (15% MeOH/CH₂Cl₂+3% NH₄OH) to provide N,N-dimethyl-3-(3-methyl-5-(1H-pyrrolo[2,3-b]pyridin-3-yl)-3,6-dihydropyridin-1(2H)-yl)propan-1-amine (46.2 mg, 39% yield) as a yellow oil. ¹H NMR (600 MHz, CDCl₃) δ 10.74 (br s, 1H), 8.31 (dd, J=4.9, 0.8 Hz, 1H), 8.17 (dd, J=8.0, 1.3 Hz, 1H), 7.29 (s, 1H), 7.10 (dd, J=7.9, 4.7 Hz, 1H), 6.06 (s, 1H), 3.53 (d, J=15.2 Hz, 1H), 3.12 (d, J=15.1 Hz, 1H), 2.92 (dd, J=11.0, 5.3 Hz, 1H), 2.72-2.62 (m, 1H), 2.60-2.52 (m, 2H), 2.47 (t, J=7.5 Hz, 2H), 2.33 (s, 6H), 2.06 (dd, J=10.9, 8.8 Hz, 1H), 1.86 (p, J=7.5 Hz, 2H), 1.10 (d, J=7.0 Hz, 3H). ¹³C NMR (151 MHz, CDCl₃) δ 149.3, 143.0, 129.3, 128.9, 126.8, 121.5, 118.3, 116.1, 114.8, 58.0, 57.8, 56.2, 55.1, 45.3, 31.2, 25.0, 19.6. HRMS (ESI+, m/z) [M+H]⁺ calcd for C₁₈H₂₇N₄⁺: 299.2236. found: 299.2225. A portion of this material was separated using semi-preparative chiral HPLC (Chiralpak AD-H column, 250×10 mm, 5% ethanol/hexanes+0.1% diethylamine, 2 mL/min) to provide the R- and S-enantiomers with tᵣ=30.0 min and 36.0 min, respectively (Compounds 31 and 32).

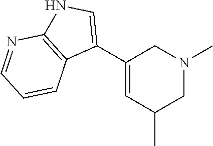 [SMILES: CC1C=C(c2c[nH]c3ncccc23)CN(C)C1]

(R) and (S) enantiomers of 3-(1,5-dimethyl-1,2,5,6-tetrahydropyridin-3-yl)-1H-pyrrolo[2,3-b]pyridine (Compounds 33 and 34): General procedure C was followed with minor modifications using [RhCl(coe)₂]₂ (108 mg, 0.150 mmol, 0.075 equiv), followed by ligand (62.8 mg, 0.300 mmol, 0.15 equiv), (E)-N,2-dimethylprop-2-en-1-imine solution (3.3 mL, 2.0 mmol, 1.0 equiv, 0.6 M solution in THF) and tert-butyl 3-((trimethylsilyl)ethynyl)-1H-pyrrolo[2,3-b]pyridine-1-carboxylate (629 mg, 3.00 mmol, 1.5 equiv) in THE (0.66 mL). The reaction was run at 65° C. for 3 h to produce the DHP intermediate.

Following general procedure D, DHP reduction was performed with Na(OAc)₃BH (1.27 g, 6.00 mmol, 3.0 equiv) in THF (20 mL), HF-pyridine (4.0 mL, 170 mmol, 85 equiv), and crude DHP solution (2.0 mmol, 1.0 equiv). The crude DHP solution was transferred and rinsed with THE (5 mL). The resulting crude THP product was purified by flash-column chromatography (60% EtOAc/hexanes+1% Et₃N) followed by preparative thin-layer chromatography (80% EtOAc/hexanes+1% Et₃N) with only pure fractions isolated to afford the desired product (173 mg, 26% yield from imine) as a pale-yellow oil. ¹H NMR (400 MHz, CDCl₃) δ 8.50 (dd, J=4.7, 1.3 Hz, 1H), 8.10 (dd, J=7.9, 1.4 Hz, 1H), 7.49 (s, 1H), 7.21 (dd, J=7.9, 4.8 Hz, 1H), 6.10 (s, 1H), 3.41 (d, J=15.4 Hz, 1H), 3.07 (d, J=15.4 Hz, 1H), 2.86 (dd, J=11.0, 5.4 Hz, 1H), 2.75-2.62 (m, 1H), 2.46 (s, 3H), 2.05 (dd, J=10.9, 8.6 Hz, 1H), 1.67 (s, 9H), 1.11 (d, J=7.0 Hz, 3H). General procedure E was followed using this THP (173 mg, 0.528 mmol, 1.0 equiv) with trifluoroacetic acid (0.39 mL, 5.28 mmol, 10 equiv) in CH₂Cl₂ (4.4 mL) at rt for 5.5 h. The crude product was purified by preparative thin-layer chromatography (80% EtOAc/hexanes+1% Et₃N) to yield 3-(1,5-dimethyl-1,2,5,6-tetrahydropyridin-3-yl)-1H-pyrrolo[2,3-b]pyridine (87.9 mg, 73% yield) as a pale-yellow solid. ¹H NMR (600 MHz, CDCl₃) δ 10.06 (br s, 1H), 8.32 (d, J=4.4 Hz, 1H), 8.19 (d, J=7.9 Hz, 1H), 7.27 (s, 1H), 7.12 (dd, J=7.9, 4.7 Hz, 1H), 6.07 (s, 1H), 3.47 (d, J=15.2 Hz, 1H), 3.09 (d, J=15.2 Hz, 1H), 2.86 (dd, J=10.9, 5.5 Hz, 1H), 2.76-2.64 (m, 1H), 2.46 (s, 3H), 2.05 (dd, J=10.8, 8.7 Hz, 1H), 1.11 (d, J=7.0 Hz, 3H). ¹³C NMR (151 MHz, CDCl₃) δ 149.3, 143.2, 129.3, 129.0, 126.7, 121.2, 118.3, 116.2, 115.1, 60.1, 56.8, 46.1, 31.5, 19.6. HRMS (ESI+, m/z) [M+H]⁺ calcd for C₁₄H₁₈N₃⁺: 228.1501. found: 228.1476. A portion of this material was separated using semi-preparative chiral HPLC (Chiralpak AD-H column, 250×10 mm, 5% ethanol/hexanes+0.1% diethylamine, 3 mL/min) to provide the R and S enantiomers with tᵣ=27.0 min and 34.8 min respectively (Compounds 33 and 34).

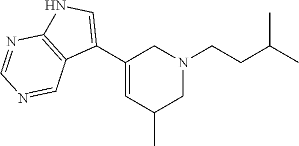 [SMILES: CC(C)CCN1CC(c2c[nH]c3ncncc23)=CC(C)C1]

(±) 5-(1-Isopentyl-5-methyl-1,2,5,6-tetrahydropyridin-3-yl)-7H-pyrrolo[2,3-d]pyrimidine (35): General procedure C was followed using (E)-N-isopentyl-2-methylprop-2-en-1-imine (55.7 mg, 0.400 mmol, 1.0 equiv) and tert-butyl 5-((trimethylsilyl)ethynyl)-7H-pyrrolo[2,3-d]pyrimidine-7-carboxylate (189 mg, 0.600 mmol, 1.5 equiv) in toluene (0.2 mL). Rh catalyst (10 mol %, 0.80 mL, 40 μmol, 50 mM in toluene) was added, and the reaction was run at 90° C. for 4 h to produce the DHP intermediate. Following general procedure D, DHP reduction was performed with Na(OAc)₃BH (216 mg, 1.02 mmol, 3.0 equiv) in THE (3.4 mL), HF-pyridine (0.68 mL, 29 mmol, 85 equiv), and crude DHP solution (0.34 mmol based upon ¹H NMR, 1.0 equiv). The crude DHP solution was transferred and rinsed with THE (0.85 mL). The resulting crude THP product was purified by preparative thin-layer chromatography (80% EtOAc/hexanes+1% Et₃N) with only pure fractions isolated to afford the desired product (53.5 mg, 35% yield from imine) as a pale-yellow oil. ¹H NMR (400 MHz, CDCl₃) δ 9.18 (s, 1H), 9.07 (s, 1H), 7.48 (s, 1H), 6.16 (s, 1H), 3.46 (d, J=15.3 Hz, 1H), 3.10 (d, J=15.3 Hz, 1H), 2.92 (dd, J=11.0, 5.3 Hz, 1H), 2.72-2.58 (m, 1H), 2.58-2.45 (m, 2H), 2.03 (apparent t, J=9.8 Hz, 1H), 1.68 (s, 9H), 1.65-1.58 (m, 1H), 1.48 (apparent q, J=7.2 Hz, 2H), 1.10 (d, J=7.0 Hz, 3H), 0.93 (d, J=6.6 Hz, 6H).

General procedure E was followed using this THP (53.5 mg, 0.139 mmol, 1.0 equiv) with trifluoroacetic acid (0.10 mL, 1.4 mmol, 10 equiv) in CH₂Cl₂ (1.2 mL) at rt for 24 h. The crude product was purified by preparative thin-layer chromatography (13% MeOH/CH₂Cl₂+1% NH₄OH) followed by a second preparative thin-layer chromatography purification (80% MTBE/hexanes+1% Et₃N), collecting only pure material to yield the title compound (7.5 mg, 19% yield) as a clear oil. ¹H NMR (600 MHz, CDCl₃) δ 10.59 (br s, 1H), 9.24 (s, 1H), 8.90 (s, 1H), 7.28 (s, 1H), 6.14 (s, 1H), 3.54 (d, J=15.0 Hz, 1H), 3.13 (d, J=15.3 Hz, 1H), 2.94 (dd, J=11.0, 5.3 Hz, 1H), 2.75-2.63 (m, 1H), 2.54 (apparent dd, J=9.5, 6.3 Hz, 2H), 2.05 (dd, J=10.7, 8.9 Hz, 1H), 1.70-1.59 (m, 1H), 1.58-1.45 (m, 2H), 1.11 (d, J=7.0 Hz, 3H), 0.94 (d, J=6.6 Hz, 6H). ¹³C NMR (151 MHz, CDCl₃) δ 152.2, 151.6, 150.1, 128.9, 128.2, 121.3, 116.9, 115.8, 58.0, 56.8, 55.1, 36.3, 31.4, 26.8, 22.9, 19.4. HRMS (ESI+, m/z) [M+H]⁺ calcd for C₁₇H₂₅N₄⁺: 285.2079. found: 285.2068.

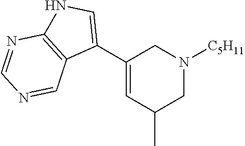

(±) 5-(5-Methyl-1-pentyl-1,2,5,6-tetrahydropyridin-3-yl)-7H-pyrrolo[2,3-d]pyrimidine (36): General procedure C was followed using (E)-2-methyl-N-pentylprop-2-en-1-imine (55.7 mg, 0.400 mmol, 1.0 equiv) and tert-butyl 5-((trimethylsilyl)ethynyl)-7H-pyrrolo[2,3-d]pyrimidine-7-carboxylate (189 mg, 0.600 mmol, 1.5 equiv) in toluene (0.2 mL). Rh catalyst (10 mol %, 0.80 mL, 40 μmol, 50 mM in toluene) was added, and the reaction was run at 90° C. for 4 h to produce the DHP intermediate. Following general procedure D, DHP reduction was performed with Na(OAc)₃BH (191 mg, 0.900 mmol, 3.0 equiv) in THE (3 mL), HF-pyridine (0.60 mL, 26 mmol, 85 equiv), and crude DHP solution (0.30 mmol based upon ¹H NMR, 1.0 equiv). The crude DHP solution was transferred and rinsed with THE (0.75 mL). The resulting crude THP product was purified by flash-column chromatography (80% EtOAc/hexanes+1% Et₃N) with only pure fractions isolated to afford the desired product (53.5 mg, 35% yield from imine) as a pale-yellow oil. ¹H NMR (400 MHz, CDCl₃) δ 9.18 (s, 1H), 9.06 (s, 1H), 7.47 (s, 1H), 6.15 (s, 1H), 3.45 (d, J=15.4 Hz, 1H), 3.09 (d, J=15.4 Hz, 1H), 2.91 (dd, J=11.1, 5.3 Hz, 1H), 2.73-2.61 (m, 1H), 2.56-2.45 (m, 2H), 2.07-1.99 (m, 1H), 1.67 (s, 9H), 1.58 (p, J=7.4 Hz, 2H), 1.39-1.29 (m, 4H), 1.09 (d, J=7.0 Hz, 3H), 0.90 (t, J=6.8 Hz, 3H). General procedure E was followed using this THP (53.5 mg, 0.139 mmol, 1.0 equiv) with trifluoroacetic acid (0.10 mL, 1.39 mmol, 10 equiv) in CH₂Cl₂ (1.2 mL) at rt for 24 h. The crude product was purified by preparative thin-layer chromatography (87% MeOH/CH₂Cl₂+1% NH₄OH) with only pure fractions isolated to yield the title compound (6.4 mg, 16% yield) as a white solid. ¹H NMR (600 MHz, CDCl₃) δ 10.98 (br s, 1H), 9.25 (s, 1H), 8.91 (s, 1H), 7.29 (s, 1H), 6.15 (s, 1H), 3.55 (d, J=15.1 Hz, 1H), 3.12 (d, J=15.0 Hz, 1H), 2.95 (dd, J=10.9, 5.2 Hz, 1H), 2.82-2.61 (m, 1H), 2.59-2.45 (m, 2H), 2.04 (t, J=9.8 Hz, 1H), 1.61 (p, J=7.3 Hz, 2H), 1.41-1.28 (m, 4H), 1.11 (d, J=7.0 Hz, 3H), 0.91 (t, J=6.8 Hz, 3H). ¹³C NMR (151 MHz, CDCl₃) δ 152.2, 151.5, 150.1, 128.8, 128.2, 121.5, 117.0, 115.7, 58.6, 57.8, 55.0, 31.4, 30.0, 27.0, 22.8, 19.4, 14.3. HRMS (ESI+, m/z) [M+H]⁺ calcd for C₁₇H₂₅N₄⁺: 285.2079. found: 285.2083.

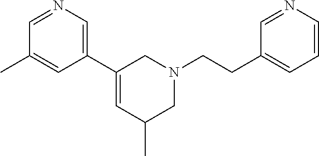 [SMILES: Cc1cncc(C2=CC(C)CN(CCc3cccnc3)C2)c1]

(±) 5,5′-Dimethyl-1-(2-(pyridin-3-yl)ethyl)-1,2,5,6-tetrahydro-3,3′-bipyridine (37): General procedure C was followed using (E)-2-methyl-N-(2-(pyridin-3-yl)ethyl)prop-2-en-1-imine (167 mg, 0.960 mmol, 1.0 equiv) and 3-methyl-5-((trimethylsilyl)ethynyl)pyridine (273 mg, 1.44 mmol, 1.5 equiv) in toluene (0.5 mL). Rh catalyst (10 mol %, 1.9 mL, 96 μmol, 50 mM in toluene) was added, and the reaction was run at 100° C. for 42 h to produce the DHP intermediate. Following general procedure D, DHP reduction was performed with Na(OAc)₃BH (488 mg, 2.30 mmol, 3.0 equiv) in THF (7.7 mL), HF-pyridine (1.5 mL, 65 mmol, 85 equiv), and crude DHP solution (0.77 mmol based upon ¹H NMR, 1.0 equiv). The crude DHP solution was transferred and rinsed with THE (2.3 mL). The resulting crude THP product was purified by preparative thin-layer chromatography (10% MeOH/CH₂Cl₂+1% NH₄OH). This material was then converted into its salt form using trifluoroacetic acid to increase its water solubility for further purification by reverse-phase flash chromatography over C18 silica gel (1-8% MeCN/H₂O+0.1% TFA) with only pure fractions isolated to afford the title compound (22.6 mg, 8% yield from imine) as a pale-yellow oil. ¹H NMR (600 MHz, CDCl₃) δ 8.50 (s, 1H), 8.47-8.44 (m, 1H), 8.41 (s, 1H), 8.31 (s, 1H), 7.56 (d, J=7.8 Hz, 1H), 7.41 (s, 1H), 7.24-7.17 (m, 1H), 6.00 (s, 1H), 3.50 (d, J=15.4 Hz, 1H), 3.20 (d, J=15.4 Hz, 1H), 2.93 (dd, J=10.9, 5.1 Hz, 1H), 2.89 (t, J=7.8 Hz, 2H), 2.80-2.74 (m, 2H), 2.66-2.58 (m, 1H), 2.32 (s, 3H), 2.15-2.08 (m, 1H), 1.07 (d, J=7.1 Hz, 3H). ¹³C NMR (151 MHz, CDCl₃) δ 150.3, 149.0, 147.8, 144.0, 136.3, 135.7, 134.9, 133.0, 132.7, 132.0, 130.6, 123.4, 59.5, 57.4, 54.5, 31.5, 31.1, 19.1, 18.6. HRMS (ESI+, m/z) [M+H]⁺ calcd for C₁₉H₂₄N₃⁺: 294.1970. found: 294.1970.

### Example 4: Synthesis of THPs (Debenzylation and Reductive Amination)

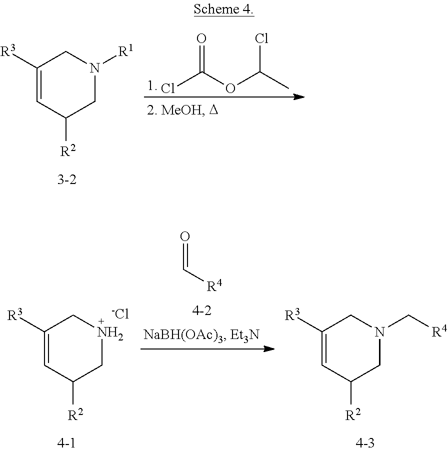

Compounds of the present disclosure may be prepared as shown in Scheme 4, wherein R¹, R², R³, and R⁴ are defined within the scope of the present disclosure. In certain embodiments, aldehyde starting materials were commercially available. In other embodiments, carboxylic acid and/or aldehyde starting materials were synthesized

### General Procedure F (Debenzylation of N-benzyl THPs)

To a flame-dried round-bottom flask was added THP (1.0 equiv) and DCE (0.2 M). The reaction mixture was cooled to 0° C., and 1-chloroethyl chloroformate (1.2 equiv) was added. The mixture warmed to room temperature over 1 h and was stirred under N₂ at rt until complete as monitored by thin-layer chromatography. The reaction mixture was then concentrated in vacuo, and the crude material was purified by silica gel chromatography with only pure fractions isolated to afford the carbamate intermediate. To a flame-dried round-bottom flask was added the purified intermediate followed by MeOH (0.13 M), and the reaction solution was stirred at 40° C. for 2 h. Upon reaction completion, the reaction mixture was concentrated in vacuo and was taken on to the next step without further purification.

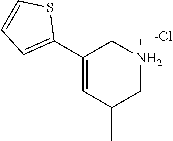

(±) 3-Methyl-5-(thiophen-2-yl)-1,2,3,6-tetrahydropyridin-1-ium chloride: General procedure F was followed using 1-benzyl-3-methyl-5-(thiophen-2-yl)-1,2,3,6-tetrahydropyridine (163 mg, 0.604 mmol, 1.0 equiv) and 1-chloroethyl chloroformate (0.08 mL, 0.73 mmol, 1.2 equiv) in DCE (3 mL). The reaction was performed for 3 h before concentration and purification (15% EtOAc/hexanes). The second step was run in MeOH (3 mL) and yielded 3-methyl-5-(thiophen-2-yl)-1,2,3,6-tetrahydropyridin-1-ium chloride (65 mg, 50% yield over two steps) as a white solid. ¹H NMR (500 MHz, CDCl₃) δ 10.07 (br s, 2H), 7.21-7.13 (m, 1H), 6.99-6.90 (m, 2H), 6.13-6.04 (m, 1H), 4.13 (d, J=16.2 Hz, 1H), 3.88 (d, J=15.9 Hz, 1H), 3.56-3.41 (m, 1H), 3.08-2.89 (m, 1H), 2.73-2.58 (m, 1H), 1.17-1.08 (m, 3H).

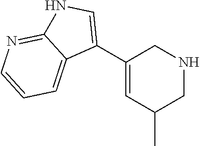 [SMILES: CC1C=C(c2c[nH]c3ncccc23)CNC1]

(R) and (S) enantiomers of 3-(5-methyl-1,2,5,6-tetrahydropyridin-3-yl)-1H-pyrrolo[2,3-b]pyridine (Compounds 38 and 39): General procedure F was followed with minor modifications using tert-butyl 3-(1-benzyl-5-methyl-1,2,5,6-tetrahydropyridin-3-yl)-1H-pyrrolo[2,3-b]pyridine-1-carboxylate (435 mg, 1.08 mmol, 1.0 equiv) and 1-chloroethyl chloroformate (0.17 mL, 1.62 mmol, 1.5 equiv) in DCE (5.4 mL). The reaction was performed for 5 h before concentration and purification (20% EtOAc/hexanes). The second step was run in MeOH (6.7 mL) and yielded the desired product as a HCl salt (283 mg, 75% yield over two steps) as a white solid. ¹H NMR (500 MHz, MeOD) δ 8.43 (dd, J=4.8, 1.3 Hz, 1H), 8.31 (dd, J=8.0, 1.3 Hz, 1H), 7.82 (s, 1H), 7.38 (dd, J=8.0, 4.8 Hz, 1H), 6.42 (s, 1H), 4.12 (d, J=16.1 Hz, 1H), 4.06 (d, J=16.2 Hz, 1H), 3.57 (dd, J=11.7, 5.2 Hz, 1H), 3.03-2.84 (m, 2H), 1.69 (s, 9H), 1.28 (d, J=6.9 Hz, 3H). The crude salt (˜60 mg) was dissolved in CH₂Cl₂ (5 mL), diluted with saturated NaHCO₃ (aq) (5 mL), and then extracted with CH₂Cl₂ (3×5 mL). The combined organic layers were washed with brine, dried with MgSO₄, filtered and concentrated to provide tert-butyl 3-(5-methyl-1,2,5,6-tetrahydropyridin-3-yl)-1H-pyrrolo[2,3-b]pyridine-1-carboxylate as a free base for chiral separation. Semi-preparative chiral HPLC (Chiralpak AD-H column, 250×10 mm, 7% ethanol/hexanes+0.1% diethylamine, 2.5 mL/min) provided the S and R enantiomers with tᵣ=24.0 min and 30.2 min respectively.

Each enantiomer was then deprotected in accordance with General procedure E. Free base (S)-tert-butyl 3-(5-methyl-1,2,5,6-tetrahydropyridin-3-yl)-1H-pyrrolo[2,3-b]pyridine-1-carboxylate (15.6 mg, 0.050 mmol, 1.0 equiv) was dissolved in CH₂Cl₂ (0.4 mL) and reacted with trifluoroacetic acid (0.04 mL, 0.5 mmol, 10 equiv). Likewise, (R)-tert-butyl 3-(5-methyl-1,2,5,6-tetrahydropyridin-3-yl)-1H-pyrrolo[2,3-b]pyridine-1-carboxylate (15.1 mg, 0.048 mmol, 1.0 equiv) was dissolved in CH₂Cl₂ (0.4 mL) and reacted with trifluoroacetic acid (0.04 mL, 0.5 mmol, 10 equiv). Each reaction was run for 5 h at rt. The crude products were purified by preparative thin-layer chromatography (15% MeOH/CH₂Cl₂+1% NH₄OH) to afford (S)-3-(5-methyl-1,2,5,6-tetrahydropyridin-3-yl)-1H-pyrrolo[2,3-b]pyridine (8.5 mg, 89% yield) and (R)-3-(5-methyl-1,2,5,6-tetrahydropyridin-3-yl)-1H-pyrrolo[2,3-b]pyridine (7.1 mg, 77% yield) as white solids. ¹H NMR (400 MHz, CD₃OD) δ 8.26 (d, J=8.8 Hz, 1H), 8.23 (d, J=4.6 Hz, 1H), 7.47 (s, 1H), 7.17 (dd, J=8.0, 4.8 Hz, 1H), 6.25 (s, 1H), 3.97 (d, J=16.5 Hz, 1H), 3.90 (d, J=16.1 Hz, 1H), 3.49-3.38 (m, 1H), 2.85-2.71 (m, 2H), 1.22 (d, J=6.1 Hz, 3H). ¹³C NMR (151 MHz, CD₃OD) δ 149.8, 143.7, 130.4, 128.8, 127.1, 123.8, 119.5, 117.1, 114.4, 49.7, 45.8, 30.1, 19.31. HRMS (ESI+, m/z) [M+H]⁺ calcd for C₁₃H₁₆N₃⁺: 214.1344. found: 214.1335.

### General Procedure G (Reductive Amination of THPs)

Inside a N₂-filled inert atmosphere glovebox, to a one dram vial was added THP salt (1.0 equiv), CH₂Cl₂ (0.12 M), triethylamine (2.0 equiv), aldehyde (1.1 equiv), and sodium triacetoxyborohydride (1.5 equiv). The vial was removed from the glovebox, and the reaction mixture stirred under N₂ at rt. Upon reaction completion, saturated NaHCO₃ (aq) was added. The resulting mixture was extracted three times with EtOAc, and the combined organic layers were washed with brine, dried over MgSO₄, filtered, and concentrated in vacuo. The crude material was purified by flash-column chromatography or preparative thin-layer chromatography.

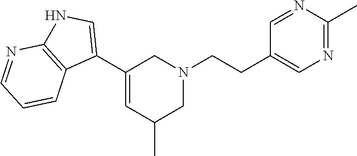 [SMILES: Cc1ncc(CCN2CC(c3c[nH]c4ncccc34)=CC(C)C2)cn1]

(R) and (S) enantiomers of 3-(5-Methyl-1-(2-(2-methylpyrimidin-5-yl)ethyl)-1,2,5,6-tetrahydropyridin-3-yl)-1H-pyrrolo[2,3-b]pyridine (Compounds 40 and 41): The aldehyde input was first prepared starting with the synthesis of 5-allyl-2-methylpyrimidine from 5-bromo-2-methylpyrimidine. Inside a glovebox, Pd₂(dba)₃ (397 mg, 0.434 mmol, 0.015 equiv), P(tBu₃) (4.3 mL, 0.87 mmol, 0.030 equiv, 0.20 M solution in toluene), 5-bromo-2-methylpyrimidine (5.00 g, 28.9 mmol, 1.0 equiv), allyltributylstannane (9.4 mL, 30 mmol, 1.05 equiv), and CsF (8.78 g, 57.8 mmol, 2.0 equiv) were added to a flame-dried round bottom flask. Toluene (24 mL) was added, and the reaction mixture was removed from the glovebox into a fume hood and was allowed to stir at rt for 5 h. Upon reaction completion, KF·2H₂O (15 g) was added to the reaction mixture, and the solution was allowed to stir for 30 min. The reaction mixture was filtered through a pad of silica gel with copious washings (EtOAc) and then concentrated in vacuo. The crude product was purified by flash-column chromatography (50% EtOAc/Hexanes) to yield 5-allyl-2-methylpyrimidine (853 mg, 22% yield) as a clear yellow oil. ¹H NMR (500 MHz, CDCl₃) δ 8.46 (s, 2H), 5.96-5.86 (m, 1H), 5.20-5.06 (m, 2H), 3.33 (d, J=6.5 Hz, 2H), 2.71 (s, 3H).

Next, 2-(2-methylpyrimidin-5-yl)acetaldehyde was synthesized. 5-Allyl-2 methylpyrimidine (853 mg, 6.36 mmol, 1.0 equiv) was added to a flame-dried round bottom flask and dissolved in MeOH (63 mL). The flask was cooled to −78° C., and the reaction mixture was purged with 02 followed by a supply of 03. The reaction ran under 03 for 5 min to generate the ozonide. While still at −78° C., the reaction was quenched with Me₂S (2.3 mL, 32 mmol, 5.0 equiv). The reaction mixture was allowed to come to rt and stirred for 8 h. The reaction was concentrated in vacuo and purified by flash-column chromatography (8% MeOH/CH₂Cl₂) to yield the desired product (424 mg, 49% yield) as a pale-yellow oil. ¹H NMR (500 MHz, CDCl₃) δ 9.77 (t, J=1.4 Hz, 1H), 8.45 (s, 2H), 3.69 (d, J=1.6 Hz, 2H), 2.66 (s, 3H).

General procedure G was followed using tert-butyl 3-(5-methyl-1,2,5,6-tetrahydropyridin-3-yl)-1H-pyrrolo[2,3-b]pyridine-1-carboxylate (120 mg, 0.342 mmol, 1.0 equiv), 2-(2-methylpyrimidin-5-yl)acetaldehyde (140 mg, 1.03 mmol, 3.0 equiv), Et₃N (95 L, 0.68 mmol, 2.0 equiv), and Na(OAc)₃BH (362 mg, 1.71 mmol, 5.0 equiv) in CH₂Cl₂ (1.7 mL). The reaction ran for 20 h at rt. The crude material was purified by preparative thin-layer chromatography (8% CH₂Cl₂/MeOH) to afford the desired product (115 mg, 77% yield) as a pale-yellow oil. ¹H NMR (500 MHz, CDCl₃) δ 8.53 (s, 2H), 8.48 (dd, J=4.7, 1.6 Hz, 1H), 8.06 (dd, J=8.1, 1.7 Hz, 1H), 7.47 (s, 1H), 7.18 (dd, J=7.9, 4.7 Hz, 1H), 6.08 (s, 1H), 3.43 (d, J=15.1 Hz, 1H), 3.20 (d, J=15.1, 1H), 2.91 (dd, J=11.0, 5.2 Hz, 1H), 2.82 (t, J=7.6 Hz, 2H), 2.74 (t, J=7.0 Hz, 2H), 2.68 (s, 3H), 2.63 (m, 1H), 2.16 (dd, J=11.0, 8.0 Hz, 1H), 1.64 (s, 9H), 1.08 (d, J=7.1 Hz, 3H).

Following reductive amination, the product was deprotected in accordance with general procedure E. Protected THP tert-butyl 3-(5-methyl-1-(2-(2-methylpyrimidin-5-yl)ethyl)-1,2,5,6-tetrahydropyridin-3-yl)-1H-pyrrolo[2,3-b]pyridine-1-carboxylate (115 mg, 0.27 mmol, 1.0 equiv) was dissolved in CH₂Cl₂ (2.2 mL) and was reacted with trifluoroacetic acid (207 μL, 2.7 mmol, 10 equiv) at rt for 5 h. The crude product was purified by preparative thin-layer chromatography (8% MeOH/CH₂Cl₂+1% NH₄OH) to yield 3-(5-methyl-1-(2-(2-methylpyrimidin-5-yl)ethyl)-1,2,5,6-tetrahydropyridin-3-yl)-1H-pyrrolo[2,3-b]pyridine (51 mg, 57% yield) as a pale-yellow oil. A portion of this material was separated using semi-preparative chiral HPLC (Chiralpak AD-H column, 250×10 mm, 50% ethanol/hexanes, 2.5 mL/min) to provide the R and S enantiomers with tᵣ=21.5 min and 38.5 min respectively (Compounds 40 and 41). ¹H NMR (500 MHz, CD₃OD) δ 8.64 (s, 2H), 8.23 (dd, J=8.0, 1.6 Hz, 1H), 8.19 (dd, J=4.8, 1.5 Hz, 1H), 7.42 (s, 1H), 7.14 (dd, J=7.9, 4.8 Hz, 1H), 6.11 (s, 1H), 3.65 (d, J=15.0 Hz, 1H), 3.27 (m, 1H), 3.07 (dd, J=11.2, 5.4 Hz, 1H), 2.96 (m, 2H), 2.85 (t, J=7.3 Hz, 2H), 2.73-2.66 (m, 1H), 2.65 (s, 3H), 2.22 (dd, J=11.1, 8.8 Hz, 1H), 1.13 (d, J=7.0 Hz, 3H). ¹³C NMR (126 MHz, CD₃OD) δ 165.3, 157.1, 148.4, 142.1, 130.7, 129.0, 128.3, 125.8, 122.2, 118.3, 115.5, 113.8, 58.0, 57.1, 54.0, 30.7, 26.5, 23.5, 18.2. HRMS (ESI+, m/z) [M+H]⁺ calcd for C₂₀H₂₄N₅⁺: 334.2032. found: 334.2016.

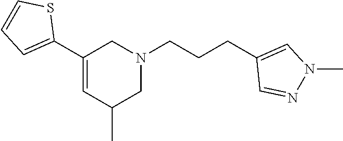 [SMILES: CC1C=C(c2cccs2)CN(CCCc2cnn(C)c2)C1]

(R) and (S) enantiomers of 3-methyl-1-(3-(1-methyl-1H-pyrazol-4-yl)propyl)-5-(thiophen-2-yl)-1,2,3,6-tetrahydropyridine (42): The aldehyde input, 3-(1-methyl-1H-pyrazol-4-yl)propanal, was prepared as follows. To a flame-dried round-bottom flask was added 3-(1-methyl-1H-pyrazol-4-yl)propan-1-ol (300 mg, 2.14 mmol, 1.0 equiv) dissolved in CH₂Cl₂ (2.7 mL). The reaction mixture was cooled to 0° C., and Dess-Martin periodinane (DMP) (1.09 g, 2.57 mmol, 1.2 equiv) was added. The reaction mixture was stirred at 0° C. for 3 h. The reaction mixture was then poured into water (4 mL) and extracted with EtOAc (3×4 mL). The combined organic layers were washed with saturated NaHCO₃ (aq), dried with MgSO₄ and concentrated. The crude material was purified by flash-column chromatography (90% Et₂O/pentane) to afford 3-(1-methyl-1H-pyrazol-4-yl)propanal (66.8 mg, 23% yield) as a clear oil. ¹H NMR (500 MHz, CDCl₃) δ 9.80 (apparent s, 1H), 7.30 (s, 1H), 7.16 (s, 1H), 3.84 (s, 3H), 2.80 (t, J=6.8 Hz, 2H), 2.70 (apparent t, J=7.1 Hz, 2H).

Next, general procedure G was followed using 3-methyl-5-(thiophen-2-yl)-1,2,3,6-tetrahydropyridine (32.4 mg, 0.150 mmol, 1.0 equiv), 3-(1-methyl-1H-pyrazol-4-yl)propanal (22.8 mg, 0.165 mmol, 1.1 equiv), Et₃N (40 μL, 0.30 mmol, 2.0 equiv), and Na(OAc)₃BH (47.7 mg, 0.225 mmol, 1.5 equiv) in CH₂Cl₂ (1.3 mL). The reaction was performed for 3 h at rt. The crude material was purified by preparative thin-layer chromatography (100% EtOAc+1% Et₃N) followed by a second preparative thin-layer chromatography purification (5% MeOH/CH₂Cl₂+0.5% NH₄OH) to afford 3-methyl-1-(3-(1-methyl-1H-pyrazol-4-yl)propyl)-5-(thiophen-2-yl)-1,2,3,6-tetrahydropyridine (34 mg, 75% yield) as a clear oil. ¹H NMR (600 MHz, CDCl₃) δ 7.32 (s, 1H), 7.15 (s, 1H), 7.11 (d, J=5.1 Hz, 1H), 6.95 (dd, J=5.0, 3.7 Hz, 1H), 6.90 (d, J=3.4 Hz, 1H), 6.02 (s, 1H), 3.85 (s, 3H), 3.48 (d, J=15.1 Hz, 1H), 3.10 (d, J=15.3 Hz, 1H), 2.83 (dd, J=11.0, 5.2 Hz, 1H), 2.63-2.56 (m, 1H), 2.52 (t, J=7.6 Hz, 4H), 2.01 (dd, J=11.0, 8.4 Hz, 1H), 1.83 (p, J=7.5 Hz, 2H), 1.05 (d, J=7.1 Hz, 3H). ¹³C NMR (151 MHz, CDCl₃) δ 144.1, 138.9, 129.9, 128.4, 128.2, 127.3, 123.2, 121.6, 121.5, 57.7, 57.6, 54.8, 38.9, 31.3, 28.6, 22.1, 19.1. HRMS (ESI+, m/z) [M+H]⁺ calcd for C₁₇H₂₄N₃S⁺: 302.1691. found: 302.1698.

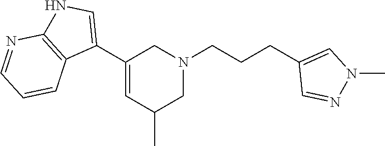 [SMILES: CC1C=C(c2c[nH]c3ncccc23)CN(CCCc2cnn(C)c2)C1]

(R) and (S) enantiomers of 3-(5-methyl-1-(3-(1-methyl-1H-pyrazol-4-yl)propyl)-1,2,5,6-tetrahydropyridin-3-yl)-1H-pyrrolo[2,3-b]pyridine (Compounds 43 and 44): The aldehyde input, 3-(1-methyl-1H-pyrazol-4-yl)propanal, was prepared as described for 3-methyl-1-(3-(1-methyl-1H-pyrazol-4-yl)propyl)-5-(thiophen-2-yl)-1,2,3,6-tetrahydropyridine.

General procedure G was then followed using the 3-(1-methyl-1H-pyrazol-4-yl)propanal (32.6 mg, 0.236 mmol, 1.1 equiv), THP tert-butyl 3-(5-methyl-1,2,5,6-tetrahydropyridin-3-yl)-1H-pyrrolo[2,3-b]pyridine-1-carboxylate (75.0 mg, 0.214 mmol, 1.0 equiv), Et₃N (60 μL, 0.43 mmol, 2.0 equiv), and Na(OAc)₃BH (68.2 mg, 0.322 mmol, 1.5 equiv) in CH₂Cl₂ (1.7 mL). The reaction was performed for 6 h at rt. The crude material was purified by preparative thin-layer chromatography (90% EtOAc/hexanes+1% Et₃N) to afford the desired product (76.8 mg, 82% yield) as a pale-orange oil. ¹H NMR (500 MHz, CDCl₃) δ 8.50 (dd, J=4.7, 1.5 Hz, 1H), 8.09 (dd, J=8.0, 1.6 Hz, 1H), 7.49 (s, 1H), 7.33 (s, 1H), 7.21 (dd, J=7.9, 4.8 Hz, 1H), 7.16 (s, 1H), 6.10 (s, 1H), 3.86 (s, 3H), 3.44 (d, J=15.4 Hz, 1H), 3.12 (d, J=15.5 Hz, 1H), 2.90 (dd, J=11.0, 5.3 Hz, 1H), 2.69-2.60 (m, 1H), 2.59-2.49 (m, 4H), 2.06 (dd, J=11.0, 8.4 Hz, 1H), 1.86 (p, J=7.6 Hz, 2H), 1.67 (s, 9H), 1.10 (d, J=7.0 Hz, 3H).

General procedure E was followed using protected THP tert-butyl 3-(5-methyl-1-(3-(1-methyl-1H-pyrazol-4-yl)propyl)-1,2,5,6-tetrahydropyridin-3-yl)-1H-pyrrolo[2,3-b]pyridine-1-carboxylate (76.0 mg, 0.174 mmol, 1.0 equiv) dissolved in CH₂Cl₂ (1.5 mL) and was reacted with trifluoroacetic acid (0.13 mL, 1.7 mmol, 10 equiv) at rt for 7.5 h. The crude product was purified by preparative thin-layer chromatography (10% MeOH/CH₂Cl₂+1% NH₄OH) to yield 3-(5-methyl-1-(3-(1-methyl-1H-pyrazol-4-yl)propyl)-1,2,5,6-tetrahydropyridin-3-yl)-1H-pyrrolo[2,3-b]pyridine (21.8 mg, 37% yield) as a pale-yellow oil. ¹H NMR (600 MHz, CDCl₃) δ 9.29 (br s, 1H), 8.31 (d, J=4.7 Hz, 1H), 8.17 (d, J=7.9 Hz, 1H), 7.33 (s, 1H), 7.24 (s, 1H), 7.16 (s, 1H), 7.11 (dd, J=8.4, 4.2 Hz, 1H), 6.07 (s, 1H), 3.86 (s, 3H), 3.50 (d, J=15.2 Hz, 1H), 3.13 (d, J=15.1 Hz, 1H), 2.91 (dd, J=10.9, 5.2 Hz, 1H), 2.71-2.61 (m, 1H), 2.54 (t, J=6.9 Hz, 4H), 2.06 (t, J=9.7 Hz, 1H), 1.87 (p, J=7.5 Hz, 2H), 1.11 (d, J=7.0 Hz, 3H). ¹³C NMR (151 MHz, CDCl₃) δ 149.3, 143.0, 138.9, 129.3, 129.0, 128.4, 127.0, 121.6, 121.3, 118.3, 116.1, 115.0, 58.0, 57.8, 55.3, 38.9, 31.3, 28.6, 22.2, 19.6. HRMS (ESI+, m/z) [M+H]⁺ calcd for C₂₀H₂₆N₅⁺: 336.2188. found: 336.2161. A portion of this material was separated using semi-preparative chiral HPLC (Chiralpak AD-H column, 250×10 mm, 12% ethanol/hexanes, 3 mL/min) to provide the S and R enantiomers with tᵣ=34.0 min and 47.0 min respectively (Compounds 43 and 44).

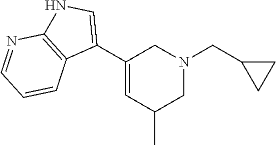 [SMILES: CC1C=C(c2c[nH]c3ncccc23)CN(CC2CC2)C1]

(R) and (S) enantiomers of 3-(1-(cyclopropylmethyl)-5-methyl-1,2,5,6-tetrahydropyridin-3-yl)-1H-pyrrolo[2,3-b]pyridine (Compounds 45 and 46): General procedure G was followed using tert-butyl 3-(5-methyl-1,2,5,6-tetrahydropyridin-3-yl)-1H-pyrrolo[2,3-b]pyridine-1-carboxylate (70.0 mg, 0.200 mmol, 1.0 equiv), cyclopropanecarbaldehyde (15.4 mg, 0.220 mmol, 1.1 equiv), Et₃N (60 μL, 0.40 mmol, 2.0 equiv), and Na(OAc)₃BH (63.6 mg, 0.300 mmol, 1.5 equiv) in CH₂Cl₂ (1.7 mL). The reaction ran for 4 h at rt. The crude material was purified by preparative thin-layer chromatography (80% EtOAc/hexanes+1% Et₃N) to afford the desired product (66.0 mg, 90% yield) as a pale-yellow oil. ¹H NMR (500 MHz CDCl₃) δ 8.50 (dd, J=4.7, 1.4 Hz, 1H), 8.10 (dd, J=7.9, 1.5 Hz, 1H), 7.51 (s, 1H), 7.21 (dd, J=7.9, 4.8 Hz, 1H), 6.10 (s, 1H), 3.59 (d, J=15.4 Hz, 1H), 3.14 (d, J=15.4 Hz, 1H), 3.07 (dd, J=11.1, 5.5 Hz, 1H), 2.76-2.65 (m, 1H), 2.51-2.38 (m, 2H), 2.09 (dd, J=11.0, 8.8 Hz, 1H), 1.67 (s, 9H), 1.11 (d, J=7.1 Hz, 3H), 0.99 (apparent p, J=6.1, 5.5 Hz, 1H), 0.64-0.49 (m, 2H), 0.19 (apparent q, J=4.8 Hz, 2H).

General procedure E was followed using protected THP tert-butyl 3-(1-(cyclopropylmethyl)-5-methyl-1,2,5,6-tetrahydropyridin-3-yl)-1H-pyrrolo[2,3-b]pyridine-1-carboxylate (66.0 mg, 0.180 mmol, 1.0 equiv) dissolved in CH₂Cl₂ (1.5 mL), which was reacted with trifluoroacetic acid (0.13 mL, 1.8 mmol, 10 equiv) at rt for 4 h. The crude product was purified by preparative thin-layer chromatography (10% MeOH/CH₂Cl₂+1% NH₄OH) to yield 3-(1-(cyclopropylmethyl)-5-methyl-1,2,5,6-tetrahydropyridin-3-yl)-1H-pyrrolo[2,3-b]pyridine (26.1 mg, 54% yield) as a pale-yellow oil. ¹H NMR (600 MHz, CDCl₃) δ 9.57 (br s, 1H), 8.32 (d, J=5.0 Hz, 1H), 8.18 (d, J=7.9 Hz, 1H), 7.27 (s, 1H), 7.12 (dd, J=7.9, 4.7 Hz, 1H), 6.07 (s, 1H), 3.68 (d, J=15.6 Hz, 1H), 3.15 (d, J=15.1 Hz, 1H), 3.08 (dd, J=11.0, 5.5 Hz, 1H), 2.76-2.66 (m, 1H), 2.48 (dd, J=12.5, 6.4 Hz, 1H), 2.40 (dd, J=12.5, 6.6 Hz, 1H), 2.08 (dd, J=11.1, 9.3 Hz, 1H), 1.11 (d, J=7.0 Hz, 3H), 1.00 (apparent sept, J=6.8 Hz, 1H), 0.62-0.53 (m, 2H), 0.19 (apparent q, J=4.3 Hz, 2H). ¹³C NMR (151 MHz, CDCl₃) δ 149.2, 143.4, 129.3, 129.0, 127.2, 121.0, 118.2, 116.4, 115.4, 63.6, 58.1, 55.1, 31.3, 19.6, 8.8, 4.18, 4.17. HRMS (ESI+, m/z) [M+H]⁺ calcd for C₁₇H₂₂N₃⁺: 268.1814. found: 268.1798. A portion of this material was separated using semi-preparative chiral HPLC (Chiralpak AD-H column, 250×10 mm, 3% ethanol/hexanes, 3 mL/min) to provide the R and S enantiomers with tᵣ=31.5 min and 40.0 min respectively (Compounds 45 and 46).

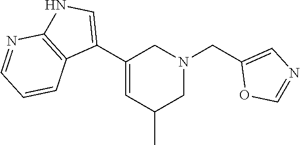 [SMILES: CC1C=C(c2c[nH]c3ncccc23)CN(Cc2cnco2)C1]

(R) and (S) Enantiomers of 5-((3-Methyl-5-(1H-pyrrolo[2,3-b]pyridin-3-yl)-3,6-dihydropyridin-1(2H)-yl)methyl)oxazole (Compounds 47 and 48): General procedure G was followed using the racemic THP amine salt intermediate described in the preparation of 3-(5-methyl-1-((3-methyloxetan-3-yl)methyl)-1,2,5,6-tetrahydropyridin-3-yl)-1H-pyrrolo[2,3-b]pyridine (75.0 mg, 0.214, 1.0 equiv), oxazole-5-carbaldehyde (22.9 mg, 0.236 mmol, 1.1 equiv), Et₃N (60 μL, 0.43 mmol, 2.0 equiv), and Na(OAc)₃BH (68.2 mg, 0.322 mmol, 1.5 equiv) in CH₂Cl₂ (1.7 mL). The reaction was carried out for 6 h at rt. The crude material was purified by preparative thin-layer chromatography (80% EtOAc/hexanes+1% Et₃N) to afford the desired product (46.0 mg, 54% yield) as a clear oil. ¹H NMR (500 MHz, CDCl₃) δ 8.50 (dd, J=4.7, 1.3 Hz, 1H), 8.07 (dd, J=7.9, 1.4 Hz, 1H), 7.88 (s, 1H), 7.48 (s, 1H), 7.21 (dd, J=7.9, 4.8 Hz, 1H), 7.04 (s, 1H), 6.10 (s, 1H), 3.85 (d, J=14.6 Hz, 1H), 3.80 (d, J=14.6 Hz, 1H), 3.47 (d, J=15.2 Hz, 1H), 3.25 (d, J=15.1 Hz, 1H), 2.93 (dd, J=11.0, 5.4 Hz, 1H), 2.74-2.61 (m, 1H), 2.15 (dd, J=11.0, 8.5 Hz, 1H), 1.67 (s, 9H), 1.09 (d, J=7.0 Hz, 3H). General procedure E was followed using protected THP (46.0 mg, 0.117 mmol, 1.0 equiv) dissolved in CH₂Cl₂ (1.0 mL) and was reacted with trifluoroacetic acid (0.087 mL, 1.2 mmol, 10 equiv) at rt for 7.5 h. The crude product was purified by preparative thin-layer chromatography (10% MeOH/CH₂Cl₂+1% NH₄OH) to provide 5-((3-methyl-5-(1H-pyrrolo[2,3-b]pyridin-3-yl)-3,6-dihydropyridin-1(2H)-yl)methyl)oxazole (24.9 mg, 73% yield) as a clear oil. ¹H NMR (600 MHz, CDCl₃) δ 10.29 (br s, 1H), 8.32 (d, J=4.7 Hz, 1H), 8.16 (d, J=7.9 Hz, 1H), 7.88 (s, 1H), 7.25 (s, 1H), 7.12 (dd, J=7.9, 4.7 Hz, 1H), 7.05 (s, 1H), 6.07 (s, 1H), 3.82 (s, 2H), 3.53 (d, J=15.0 Hz, 1H), 3.26 (d, J=15.0 Hz, 1H), 2.94 (dd, J=10.9, 5.4 Hz, 1H), 2.74-2.64 (m, 1H), 2.15 (dd, J=10.6, 8.9 Hz, 1H), 1.10 (d, J=7.0 Hz, 3H). ¹³C NMR (151 MHz, CDCl₃) δ 151.3, 149.3, 149.2, 143.2, 129.2, 128.6, 126.9, 125.4, 121.2, 118.2, 116.2, 114.8, 57.3, 54.4, 51.9, 31.2, 19.4. HRMS (ESI+, m/z) [M+H]⁺ calcd for C₁₇H₁₉N₄O⁺: 295.1559. found: 295.1544. A portion of this material was separated using semi-preparative chiral HPLC (Chiralpak AD-H column, 250×10 mm, 10% ethanol/hexanes, 3 mL/min) to provide the R- and S-enantiomers with tᵣ=40.0 min and 50.0 min, respectively (Compounds 47 and 48).

### Synthesis of N-Unsubstituted THPs

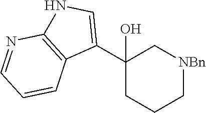 [SMILES: OC1(c2c[nH]c3ncccc23)CCCN(Cc2ccccc2)C1]

(±) 1-Benzyl-3-(1H-pyrrolo[2,3-b]pyridin-3-yl)piperidin-3-ol: A 60% dispersion of NaH in mineral oil (1.8 g, 34 mmol, 4.0 equiv) was slowly added to 28.3 mL of EtOH at 0° C. This solution was added to 7-azaindole (1.0 g, 8.5 mmol, 1.0 equiv) and 1-benzyl-piperidin-3-one (as HCl salt) (2.1 g, 8.5 mmol, 1.0 equiv). The resulting mixture was stirred for 72 h at rt. EtOAc was added to the mixture and the organic layer was washed three times with a saturated NaHCO₃ solution, dried (Na₂SO₄), filtered and concentrated in vacuo. The resulting residue was purified by flash-column chromatography (50% EtOAc/Hexanes) to afford the desired product (1.8 g, 71% yield) as a yellow oil. ¹H NMR (400 MHz, CDCl₃) δ 11.93 (s, 1H), 8.24 (dd, J=4.8, 1.5 Hz, 1H), 8.14 (dd, J=7.9, 1.6 Hz, 1H), 7.36-7.16 (m, 6H), 6.98 (dd, J=7.9, 4.8 Hz, 1H), 4.53 (s, 1H), 3.55 (m, 2H), 3.01 (d, J=11.1 Hz, 1H), 2.84 (d, J=10.2 Hz, 1H), 2.35 (d, J=11.1 Hz, 1H), 2.17-1.89 (m, 3H), 1.83 (m, 1H), 1.67-1.53 (m, 1H).

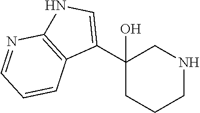 [SMILES: OC1(c2c[nH]c3ncccc23)CCCNC1]

(±) 3-(1H-Pyrrolo[2,3-b]pyridin-3-yl)-piperidin-3-ol: 1-Benzyl-3-(1H-pyrrolo[2,3-b]pyridin-3-yl)piperidin-3-ol (500 mg, 1.6 mmol, 1.0 equiv), ammonium formate (922 mg, 14.6 mmol, 9.1 equiv) and 20% Pd(OH)₂/C (148 mg) were combined in MeOH (30 mL) and warmed to reflux for 2 h. The mixture was cooled, filtered, and concentrated in vacuo. The resulting residue was purified by flash-column chromatography (20% MeOH/CH₂Cl₂+4% NH₄OH) to afford the desired product (265 mg, 75% yield) as a pale-yellow oil. ¹H NMR (400 MHz, CD₃OD) δ 8.28 (dd, J=8.0, 1.6 Hz, 1H), 8.15 (dd, J=4.8, 1.5 Hz, 1H), 7.35 (s, 1H), 7.07 (dd, J=8.0, 4.8 Hz, 1H), 3.35 (m, 1H), 3.24 (m, 1H), 3.16 (m, 1H), 2.96-2.81 (m, 1H), 2.29-2.09 (m, 3H), 1.80-1.69 (m, 1H).

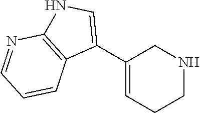 [SMILES: C1=C(c2c[nH]c3ncccc23)CNCC1]

3-(1,2,5,6-Tetrahydro-pyridin-3-yl)-1H-pyrrolo[2,3-b]pyridine (Compound 49): Acetyl chloride (1.4 mL) was slowly added with stirring to EtOH (28 mL) at −10° C. After 15 minutes, this solution was added to 3-(1H-pyrrolo[2,3-b]pyridin-3-yl)-piperidin-3-ol (500 mg, 2.3 mmol, 1.0 equiv) and heated to reflux for 1 h. The mixture was cooled and concentrated in vacuo. The resulting residue was purified by flash-column chromatography (20% MeOH/CH₂Cl₂+4% NH₄OH) to afford the desired product 3-(1,2,5,6-tetrahydro-pyridin-3-yl)-1H-pyrrolo[2,3-b]pyridine (156 mg, 34% yield) as a yellow oil. ¹H NMR (600 MHz, CD₃OD) δ 8.22 (d, J=8.0 Hz, 1H), 8.16 (d, J=4.8 Hz, 1H), 7.34 (s, 1H), 7.10 (dd, J=7.9, 4.8 Hz, 1H), 6.27 (s, 1H), 3.65 (s, 2H), 2.98 (t, J=5.9 Hz, 2H), 2.33 (m, 2H). ¹³C NMR (151 MHz, CD₃OD) δ 148.3, 142.0, 130.1, 129.06, 121.7, 119.7, 118.3, 115.4, 114.2, 45.7, 41.9, 24.7. HRMS (ESI+, m/z) [M+H]⁺ calcd for C₁₂H₁₄N₃⁺: 200.1182. found: 200.1186.

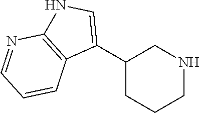 [SMILES: c1cnc2[nH]cc(C3CCCNC3)c2c1]

(R) and (S) enantiomers of 3-piperidin-3-yl-1H-pyrrolo[2,3-b]pyridine (Compounds 50 and 51): Acetyl chloride (0.35 mL) was slowly added to MeOH (7 mL) with stirring at −10° C. After 15 minutes, this solution was added to 3-(1,2,5,6-tetrahydro-pyridin-3-yl)-1H-pyrrolo[2,3-b]pyridine (50 mg, 0.25 mmol, 1.0 equiv) and 20% Pd(OH)₂/C (4.5 mg). The mixture was hydrogenated at 50 psi for 1 h. The mixture was filtered and concentrated in vacuo, and the crude product was purified by preparative thin-layer chromatography (20% MeOH/CH₂Cl₂+4% NH₄OH) to yield the racemic product (27.8 mg, 55% yield) as a pale-yellow oil. A portion of this material was separated using semi-preparative chiral HPLC (Chiralpak AD-H column, 250×10 mm, 5% ethanol/hexanes+1% DEA, 2.5 mL/min) to provide the two enantiomers of 3-(piperidin-3-yl)-1H-pyrrolo[2,3-b]pyridine with tᵣ=64.0 min (Enantiomer I, compound 50) and 91.0 min (Enantiomer II, compound 51). ¹H NMR (600 MHz, CD₃OD) δ 8.15 (s, 1H), 8.05 (d, J=7.9 Hz, 1H), 7.20 (s, 1H), 7.12-7.03 (m, 1H), 3.33 (m, 1H), 3.20-3.14 (d, J=8.6 Hz, 1H), 3.06 (m, 1H), 2.75 (m, 2H), 2.13 (m, 1H), 1.87 (m, 1H), 1.76 (m, 2H). ¹³C NMR (151 MHz, CD₃OD) δ 148.0, 141.8, 127.5, 121.4, 119.4, 116.4, 114.7, 51.5, 45.1, 33.7, 30.6, 25.1. HRMS (ESI+, m/z) [M+H]⁺ calcd for C₁₂H₁₆N₃⁺: 202.1339. found: 202.1344.

### Example 5: X-Ray Crystallographic Data for Selected Compounds of the Present Disclosure

### Compound 19

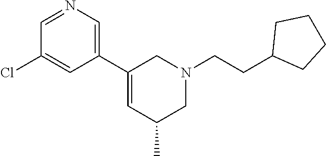 [SMILES: C[C@@H]1C=C(c2cncc(Cl)c2)CN(CCC2CCCC2)C1]

As described herein, 3 mg of compound 19 was obtained in enantiomerically pure form by semi-preparative chiral HPLC. This material was crystallized for X-Ray characterization as described below.

### Crystal Growth and X-Ray Data Collection

Compound 19 (3 mg) and picrylsulfonic acid dihydrate (0.9 equiv) were dissolved in a small amount of MeOH (dropwise addition until complete dissolution occurred) combined with CH₂Cl₂ (˜0.5 mL). The solution was transferred to an NMR tube and layered with hexanes (˜1.0 mL). Single crystals suitable for X-ray diffraction grew at 4° C. overnight.

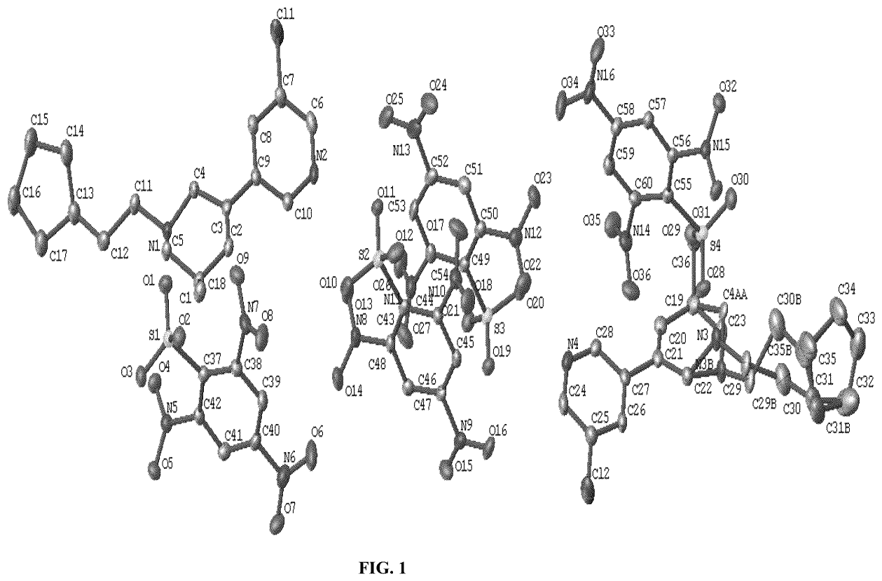

Low-temperature diffraction data (ω-scans) were collected on a Rigaku MicroMax-007HF diffractometer coupled to a Saturn994+ CCD detector with Cu Kα (λ=1.54178 Å) for the structure of 19. The diffraction images were processed and scaled using Rigaku Oxford Diffraction software (CrysAlisPro; Rigaku OD: The Woodlands, TX, 2015). The structure was solved (FIG. 1) with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups). One of the models in this CIF has significant disorder. The site occupancies were freely refined and fixed near their converged values of 0.70/0.30. All chemically similar, 1,2 and 1,3 distances that are chemically identical and disordered were restrained to be similar. Due to the small amount of electron density, the minor model thermal parameters were constrained to be identical to those of their major counterpart.

TABLE 1
Crystal data and structure refinement for compound 19

| Identification code | 19 | |
|---|---|---|
| Empirical formula | C30 H31 Cl N8 O18 S2 | |
| Formula weight | 891.20 | |
| Temperature | 93(2) K | |
| Wavelength | 1.54184 Å | |
| Crystal system | Triclinic | |
| Space group | P1 | |
| Unit cell dimensions | a = 13.0919(5) Å | α = 62.466(4)° |
| | b = 13.3018(5) Å | β = 85.549(3)° |
| | c = 13.4713(5) Å | γ = 65.661(4)° |

| | |
|---|---|
| Volume | 1876.77(15) Å³ |
| Z | 2 |
| Density (calculated) | 1.577 Mg/m³ |
| Absorption coefficient | 2.744 mm⁻¹ |
| F(000) | 920 |
| Crystal size | 0.200 × 0.200 × 0.020 mm³ |
| Crystal color and habit | Colorless Plate |
| Diffractometer | Rigaku Saturn 944+ CCD |
| Theta range for data collection | 3.737 to 66.595°. |
| Index ranges | −15 <= h <= 15, −15 <= k <= |
| | 15, −15 <= l <= 15 |
| Reflections collected | 67395 |
| Independent reflections | 12390 [R(int) = 0.0401] |
| Observed reflections (I > | 12133 |
| 2sigma(I)) | |
| Completeness to theta = 66.595° | 98.6% |
| Absorption correction | Semi-empirical from equivalents |
| Max. and min. transmission | 1.00000 and 0.78302 |
| Solution method | SHELXT-2014/5 |
| Refinement method | SHELXL-2014/7 |
| Data/restraints/parameters | 12390/18/1083 |
| Goodness-of-fit on F² | 1.024 |
| Final R indices [I > 2sigma(D)] | R1 = 0.0344, wR2 = 0.0904 |
| R indices (all data) | R1 = 0.0351, wR2 = 0.0909 |
| Absolute structure parameter | 0.020(4) |
| Largest diff. peak and hole | 1.409 and −0.725 e.Å⁻³ |

TABLE 2
Atomic coordinates (×10⁴) and equivalent isotropic displacement
parameters (Å² × 10³) for compound 19

| Atom | X | Y | Z | U(eq) |
|---|---|---|---|---|
| Cl(1) | 7236(1) | −1295(1) | 11701(1) | 42(1) |
| N(1) | 12262(3) | −4200(3) | 10284(3) | 19(1) |
| N(2) | 8158(3) | 763(3) | 8963(3) | 25(1) |
| C(1) | 12875(3) | −2491(3) | 9304(3) | 20(1) |
| C(2) | 11702(3) | −1625(4) | 9337(3) | 21(1) |
| C(3) | 10948(3) | −2028(4) | 9880(3) | 20(1) |
| C(4) | 11218(3) | −3397(4) | 10551(4) | 21(1) |
| C(5) | 13212(3) | −3824(4) | 10229(4) | 20(1) |
| C(6) | 7531(3) | 397(4) | 9765(4) | 26(1) |
| C(7) | 8019(3) | −805(4) | 10657(4) | 26(1) |
| C(8) | 9134(3) | −1596(4) | 10701(4) | 24(1) |
| C(9) | 9772(3) | −1188(3) | 9863(3) | 19(1) |
| C(10) | 9240(3) | 26(4) | 8971(4) | 23(1) |
| C(11) | 12552(3) | −5552(4) | 11141(4) | 24(1) |
| C(12) | 13592(3) | −6521(4) | 10990(4) | 29(1) |
| C(13) | 13676(4) | −7815(4) | 11773(4) | 33(1) |
| C(14) | 13670(4) | −8159(4) | 13019(5) | 38(1) |
| C(15) | 14088(6) | −9595(5) | 13618(5) | 52(2) |
| C(16) | 14860(5) | −10034(4) | 12849(5) | 47(1) |
| C(17) | 14769(4) | −8847(4) | 11784(5) | 43(1) |
| C(18) | 13769(3) | −2059(4) | 9384(4) | 26(1) |
| Cl(2) | 3580(1) | 11270(1) | −833(1) | 45(1) |
| N(3) | −1846(4) | 14838(5) | −624(5) | 25(1) |
| N(3B) | −1926(8) | 15031(10) | −1320(12) | 25(1) |
| N(4) | 1852(3) | 9615(3) | 1260(3) | 27(1) |
| C(4AA) | −2698(11) | 14808(13) | −420(13) | 19(3) |
| C(19) | −2739(3) | 13415(4) | −104(4) | 29(1) |
| C(20) | −1548(4) | 12430(4) | 127(4) | 25(1) |
| C(21) | −648(3) | 12677(4) | −184(4) | 22(1) |
| C(22) | −747(3) | 13983(4) | −776(4) | 23(1) |
| C(23) | −2827(5) | 14649(6) | −872(8) | 29(2) |
| C(24) | 2729(4) | 9817(4) | 749(4) | 28(1) |
| C(25) | 2496(4) | 10983(4) | −151(4) | 29(1) |
| C(26) | 1391(3) | 11921(4) | −491(4) | 24(1) |
| C(27) | 513(3) | 11689(4) | 71(3) | 20(1) |
| C(28) | 799(4) | 10470(4) | 962(4) | 24(1) |
| C(29) | −1924(10) | 16157(8) | −1344(7) | 29(2) |
| C(29B) | −1990(30) | 16310(20) | −1730(20) | 29(2) |
| C(30) | −3061(7) | 17367(8) | −2309(9) | 55(2) |
| C(30B) | −2959(17) | 17148(15) | −1150(20) | 55(2) |
| C(31) | −3054(14) | 18741(13) | −2919(11) | 64(3) |
| C(31B) | −2900(40) | 19010(40) | −3110(30) | 64(3) |
| C(32) | −4111(5) | 19859(9) | −3576(6) | 81(2) |
| C(33) | −4822(5) | 20040(6) | −2739(6) | 63(2) |
| C(34) | −4003(5) | 19503(7) | −1678(6) | 66(2) |
| C(35) | −2935(10) | 18578(12) | −1775(11) | 66(2) |
| C(35B) | −3000(30) | 18350(30) | −1930(30) | 66(2) |
| C(36) | −3272(4) | 13232(4) | 980(4) | 35(1) |
| S(1) | 11610(1) | −4424(1) | 7889(1) | 20(1) |
| O(1) | 10953(3) | −4830(3) | 8767(3) | 28(1) |
| O(2) | 12131(3) | −3711(3) | 8041(3) | 25(1) |
| O(3) | 12369(2) | −5361(3) | 7603(3) | 25(1) |
| O(4) | 10576(3) | −5506(3) | 6743(3) | 27(1) |
| O(5) | 11554(3) | −5120(3) | 5344(3) | 26(1) |
| O(6) | 7978(3) | 536(3) | 3312(3) | 52(1) |
| O(7) | 9002(3) | −604(3) | 2547(3) | 35(1) |
| O(8) | 10386(3) | −685(3) | 6846(3) | 37(1) |
| O(9) | 9802(3) | −2055(3) | 8048(3) | 33(1) |
| N(5) | 10916(3) | −4822(3) | 5975(3) | 21(1) |
| N(6) | 8741(3) | −413(4) | 3356(4) | 32(1) |
| N(7) | 10116(3) | −1552(3) | 7139(3) | 26(1) |
| C(37) | 10626(3) | −3242(4) | 6601(4) | 20(1) |
| C(38) | 10089(3) | −1969(4) | 6310(4) | 22(1) |
| C(39) | 9474(4) | −1042(4) | 5269(4) | 25(1) |
| C(40) | 9403(4) | −1397(4) | 4477(4) | 24(1) |
| C(41) | 9908(4) | −2608(4) | 4672(4) | 24(1) |
| C(42) | 10481(3) | −3511(4) | 5753(4) | 21(1) |
| S(2) | 7965(1) | 3620(1) | 6755(1) | 25(1) |
| O(10) | 8930(2) | 2567(3) | 6770(3) | 34(1) |
| O(11) | 7177(2) | 3242(3) | 7507(3) | 25(1) |
| O(12) | 8222(3) | 4525(3) | 6852(3) | 35(1) |
| O(13) | 7320(3) | 1934(3) | 6086(3) | 38(1) |
| O(14) | 8461(3) | 1959(3) | 4827(3) | 39(1) |
| O(15) | 5948(3) | 5828(3) | 1383(3) | 34(1) |
| O(16) | 5532(3) | 7594(3) | 1381(3) | 31(1) |
| O(17) | 5945(3) | 6401(4) | 6088(3) | 44(1) |
| O(18) | 6956(3) | 7261(3) | 4947(3) | 38(1) |
| N(8) | 7720(3) | 2465(3) | 5267(3) | 28(1) |
| N(9) | 5893(3) | 6441(3) | 1848(3) | 25(1) |
| N(10) | 6520(3) | 6528(3) | 5320(3) | 29(1) |
| C(43) | 7197(3) | 4459(4) | 5344(4) | 21(1) |
| C(44) | 6655(3) | 5759(4) | 4756(4) | 20(1) |
| C(45) | 6204(3) | 6425(4) | 3622(4) | 21(1) |
| C(46) | 6298(3) | 5756(4) | 3067(4) | 20(1) |
| C(47) | 6809(3) | 4464(4) | 3590(4) | 21(1) |
| C(48) | 7230(3) | 3851(4) | 4726(4) | 21(1) |
| S(3) | 2904(1) | 6409(1) | 3081(1) | 21(1) |
| O(19) | 3684(3) | 6801(3) | 2349(3) | 26(1) |
| O(20) | 1876(2) | 7477(3) | 3000(3) | 26(1) |
| O(21) | 2700(3) | 5459(3) | 3025(3) | 27(1) |
| O(22) | 3346(3) | 8273(3) | 3632(3) | 35(1) |
| O(23) | 2173(3) | 8268(3) | 4873(3) | 37(1) |
| O(24) | 4823(3) | 4619(4) | 8385(3) | 42(1) |
| O(25) | 5186(3) | 2796(3) | 8537(3) | 41(1) |
| O(26) | 3822(3) | 2860(3) | 5046(3) | 42(1) |
| O(27) | 4980(3) | 3539(4) | 3985(4) | 42(1) |
| N(11) | 4321(3) | 3537(3) | 4678(3) | 28(1) |
| N(12) | 2956(3) | 7745(3) | 4469(3) | 26(1) |
| N(13) | 4853(3) | 3940(4) | 7994(4) | 34(1) |
| C(49) | 3589(3) | 5683(4) | 4504(4) | 20(1) |
| C(50) | 3498(3) | 6365(4) | 5067(4) | 22(1) |
| C(51) | 3906(3) | 5831(4) | 6188(4) | 26(1) |
| C(52) | 4450(4) | 4539(4) | 6778(4) | 28(1) |
| C(53) | 4585(4) | 3800(4) | 6289(4) | 28(1) |
| C(54) | 4154(3) | 4381(4) | 5166(4) | 25(1) |
| S(4) | −852(1) | 14634(1) | 1926(1) | 21(1) |
| O(28) | −149(3) | 14969(3) | 1070(3) | 29(1) |
| O(29) | −1400(3) | 13946(3) | 1784(3) | 29(1) |
| O(30) | −1602(2) | 15634(3) | 2150(3) | 28(1) |
| O(31) | 217(2) | 15742(3) | 3066(3) | 25(1) |
| O(32) | −840(3) | 15438(3) | 4405(3) | 28(1) |
| O(33) | 1877(3) | 11036(3) | 7249(3) | 41(1) |
| O(34) | 2559(3) | 9668(3) | 6647(3) | 45(1) |
| O(35) | 140(3) | 10981(3) | 3061(3) | 41(1) |
| O(36) | 947(3) | 12128(3) | 1918(3) | 39(1) |
| N(14) | 540(3) | 11741(4) | 2786(3) | 29(1) |
| N(15) | −177(3) | 15094(3) | 3812(3) | 20(1) |
| N(16) | 1966(3) | 10711(3) | 6515(3) | 32(1) |
| C(55) | 91(3) | 13473(4) | 3251(4) | 19(1) |
| C(56) | 249(3) | 13777(4) | 4066(4) | 19(1) |
| C(57) | 820(4) | 12911(4) | 5140(4) | 22(1) |
| C(58) | 1295(4) | 11664(4) | 5396(4) | 25(1) |
| C(59) | 1196(4) | 11270(4) | 4644(4) | 27(1) |
| C(60) | 585(4) | 12199(4) | 3588(4) | 22(1) |

### Compound 34

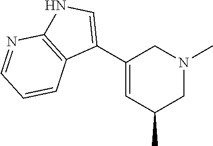 [SMILES: C[C@H]1C=C(c2c[nH]c3ncccc23)CN(C)C1]

As described previously, compound 34, was obtained in enantiomerically pure form by semi-preparative chiral HPLC. This material was crystallized for X-ray characterization as described below.

### Crystal Growth and X-Ray Data Collection

Compound 34 (7 mg) was dissolved in a small amount of MeOH (dropwise addition until complete dissolution occurred) combined with toluene (˜0.5 mL). The solution was transferred to an NMR tube and layered with hexanes (1.0 mL). Single crystals suitable for X-ray diffraction grew at room temperature over two days.

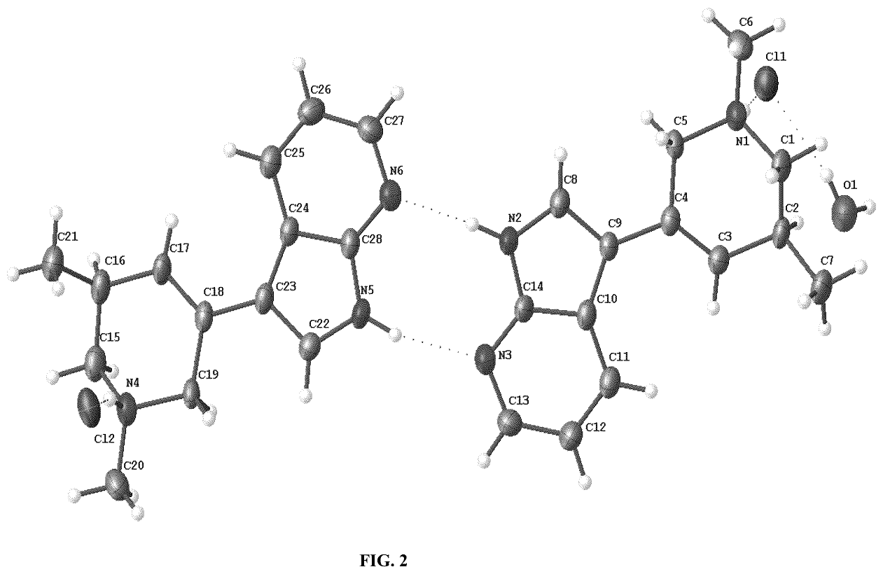

Low-temperature diffraction data (ω-scans) were collected on a Rigaku MicroMax-007HF diffractometer coupled to a Saturn994+ CCD detector with Cu Kα (λ=1.54178 Å) for the structure of 34. The diffraction images were processed and scaled using Rigaku Oxford Diffraction software (CrysAlisPro; Rigaku GD: The Woodlands, TX, 2015). The structure (FIG. 2) was solved with SELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl and water groups).

TABLE 3
Crystal data and structure refinement for compound 34

| Identification code | 34 | |
|---|---|---|
| Empirical formula | C14 H19 Cl N3 O0.50 | |
| Formula weight | 272.77 | |
| Temperature | 93(2) K | |
| Wavelength | 1.54184 Å | |
| Crystal system | Monoclinic | |
| Space group | P2_{I} | |
| Unit cell dimensions | a = 7.6898(2) Å | α = 90° |
| | b = 16.4131(3) Å | β = 96.355(2)° |
| | c = 11.0059(2) Å | γ = 90° |

| | |
|---|---|
| Volume | 1380.56(5) Å³ |
| Z | 4 |
| Density (calculated) | 1.312 Mg/m³ |
| Absorption coefficient | 2.371 mm⁻¹ |
| F(000) | 580 |
| Crystal size | 0.200 × 0.080 × 0.050 mm³ |
| Crystal color and habit | Colorless Plate |
| Diffractometer | Rigaku Saturn 944+ CCD |
| Theta range for data collection | 4.042 to 66.572°. |
| Index ranges | −9 <= h <= 9, −19 <= k <= |
| | 19, −13 <= l <= 13 |
| Reflections collected | 34922 |
| Independent reflections | 4808 [R(int) = 0.1476] |
| Observed reflections (I > | 4578 |
| 2sigma(I)) | |
| Completeness to theta = 66.572° | 99.4% |
| Absorption correction | None |
| Solution method | SHELXT-2014/5 (Sheldrick, 2014) |
| Refinement method | SHELXL-2014/7 (Sheldrick, 2014) |
| Data/restraints/parameters | 4808/1/341 |
| Goodness-of-fit on F² | 1.168 |
| Final R indices [I > 2sigma(I)] | R1 = 0.0658, wR2 = 0.1800 |
| R indices (all data) | R1 = 0.0678, wR2 = 0.1827 |
| Absolute structure parameter | −0.060(19) |
| Largest diff. peak and hole | 0.277 and −0.460 e.Å⁻³ |
| Volume | 1380.56(5) Å³ |

TABLE 4
Atomic coordinates (×10⁴) and equivalent isotropic displacement
parameters (Å² × 10³) for compound 34

| Atom | X | Y | Z | U(eq) |
|---|---|---|---|---|
| Cl(1) | 4890(2) | 1517(1) | −366(1) | 44(1) |
| Cl(2) | 10170(2) | 9004(1) | 9681(1) | 47(1) |
| O(1) | 3039(7) | 192(3) | 1082(4) | 51(1) |
| N(1) | 8561(6) | 1493(3) | 942(4) | 33(1) |
| N(2) | 8321(6) | 4124(3) | 4257(4) | 35(1) |
| N(3) | 7218(7) | 4130(3) | 6198(4) | 38(1) |
| C(1) | 8877(8) | 691(3) | 1584(6) | 38(1) |
| C(2) | 7569(8) | 577(3) | 2500(5) | 35(1) |
| C(3) | 7505(8) | 1324(3) | 3289(5) | 33(1) |
| C(4) | 8107(7) | 2051(3) | 2995(5) | 31(1) |
| C(5) | 8885(8) | 2187(3) | 1815(5) | 32(1) |
| C(6) | 9611(9) | 1589(4) | −96(6) | 46(2) |
| C(7) | 8023(11) | −186(4) | 3264(6) | 48(2) |
| C(8) | 8572(8) | 3551(3) | 3382(5) | 35(1) |
| C(9) | 8064(7) | 2793(3) | 3754(5) | 30(1) |
| C(10) | 7484(7) | 2902(3) | 4940(5) | 31(1) |
| C(11) | 6828(8) | 2421(4) | 5835(5) | 38(1) |
| C(12) | 6389(9) | 2797(4) | 6876(6) | 42(1) |
| C(13) | 6590(9) | 3642(4) | 7022(6) | 41(1) |
| C(14) | 7636(8) | 3747(3) | 5201(5) | 32(1) |
| N(4) | 6264(6) | 8831(3) | 8855(4) | 37(1) |
| N(5) | 7132(6) | 5953(3) | 6104(4) | 34(1) |
| N(6) | 8085(7) | 5881(3) | 4109(4) | 38(1) |
| C(15) | 5316(9) | 9488(4) | 8104(6) | 42(1) |
| C(16) | 6087(9) | 9612(4) | 6914(6) | 41(1) |
| C(17) | 6532(8) | 8813(3) | 6349(5) | 36(1) |
| C(18) | 6575(7) | 8088(3) | 6924(5) | 31(1) |
| C(19) | 6139(8) | 8031(3) | 8217(5) | 32(1) |
| C(20) | 5633(10) | 8756(4) | 10076(6) | 49(2) |
| C(21) | 7653(10) | 10194(4) | 7040(6) | 48(2) |
| C(22) | 6740(7) | 6559(3) | 6871(5) | 32(1) |
| C(23) | 6956(8) | 7310(3) | 6353(5) | 33(1) |
| C(24) | 7539(7) | 7154(3) | 5181(5) | 30(1) |
| C(25) | 8041(8) | 7600(4) | 4197(6) | 39(1) |
| C(26) | 8502(9) | 7186(4) | 3200(6) | 45(2) |
| C(27) | 8477(9) | 6343(4) | 3176(5) | 42(2) |
| C(28) | 7619(7) | 6295(3) | 5063(5) | 33(1) |

### Compound 38

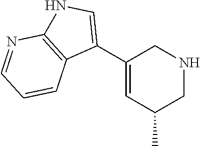 [SMILES: C[C@@H]1C=C(c2c[nH]c3ncccc23)CNC1]

As described herein, compound 38 was obtained in enantiomerically pure form by semi-preparative chiral HPLC. This material was crystallized for X-ray characterization as described below.

### Crystal Growth and X-Ray Data Collection

Compound 38 (3 mg) was dissolved in MeOH, and HCl (3 equiv, 4 N in dioxane) was added. The mixture was concentrated in vacuo. The resulting salt was then dissolved in a small amount of MeOH (dropwise addition until complete dissolution occurred) combined with EtOAc (˜0.5 mL). The solution was transferred to an NMR tube and layered with hexanes (1.0 mL). Single crystals suitable for X-ray diffraction grew at room temperature over three days.

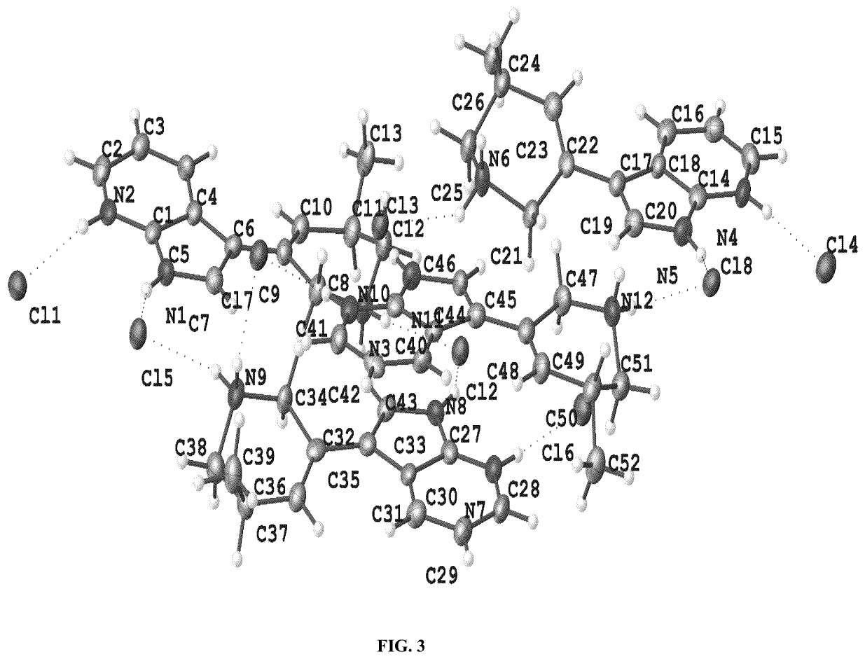

Low-temperature diffraction data (ω-scans) were collected on a Rigaku MicroMax-007HF diffractometer coupled to a Saturn994+ CCD detector with Cu Kα (λ=1.54178 Å) for the structure of 38. The diffraction images were processed and scaled using Rigaku Oxford Diffraction software (CrysAlisPro; Rigaku OD: The Woodlands, TX, 2015). The structure (FIG. 3) was solved with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups).

TABLE 5
Crystal data and structure refinement for compound 38

| Identification code | 38 | |
|---|---|---|
| Empirical formula | C13 H17 Cl2 N3 | |
| Formula weight | 286.19 | |
| Temperature | 93(2) K | |
| Wavelength | 1.54184 Å | |
| Crystal system | Triclinic | |
| Space group | P1 | |
| Unit cell dimensions | a = 9.8964(6) Å | α = 106.476(5)° |
| | b = 11.6241(6) Å | β = 97.436(5)° |
| | c = 12.5240(9) Å | γ = 92.881(5)° |

| | |
|---|---|
| Volume | 1364.20(15) Å³ |
| Z | 4 |
| Density (calculated) | 1.393 Mg/m³ |
| Absorption coefficient | 4.157 mm⁻¹ |
| F(000) | 600 |
| Crystal size | 0.200 × 0.200 × 0.200 mm³ |
| Crystal color and habit | Colorless Plate |
| Diffractometer | Rigaku Saturn 944+ CCD |
| Theta range for data collection | 3.723 to 66.600°. |
| Index ranges | −11 <= h <= 11, −13 <= k <= |
| | 13, −14 <= l <= 14 |
| Reflections collected | 42289 |
| Independent reflections | 8827 [R(int) = 0.0967] |
| Observed reflections (I > | 7846 |
| 2sigma(I)) | |
| Completeness to theta = 66.600° | 98.3% |
| Absorption correction | Semi-empirical from equivalents |
| Max. and min. transmission | 1.00000 and 0.61042 |
| Solution method | SHELXT-2014/5 (Sheldrick, 2014) |
| Refinement method | SHELXL-2014/7 (Sheldrick, 2014) |
| Data/restraints/parameters | 8827/3/653 |
| Goodness-of-fit on F² | 1.054 |
| Final R indices [I > 2sigma(D)] | R1 = 0.0710, wR2 = 0.1930 |
| R indices (all data) | R1 = 0.0795, wR2 = 0.2062 |
| Absolute structure parameter | −0.02(2) |
| Extinction coefficient | n/a |
| Largest diff. peak and hole | 0.989 and -0.677 e.Å⁻³ |

TABLE 6
Atomic coordinates (×10⁴) and equivalent isotropic displacement
parameters (Å² × 10³) for compound 38

| Atom | X | Y | Z | U(eq) |
|---|---|---|---|---|
| Cl(1) | 13469(2) | 3843(2) | 10722(2) | 30(1) |
| Cl(2) | 3926(2) | 7559(2) | 5529(2) | 26(1) |
| Cl(3) | 5660(2) | 4531(2) | 4373(2) | 27(1) |
| Cl(4) | −3174(2) | 9168(2) | −725(2) | 32(1) |
| Cl(5) | 9119(2) | 2378(2) | 8594(2) | 28(1) |
| Cl(6) | −401(2) | 6431(2) | 3606(2) | 32(1) |
| Cl(7) | 6214(2) | 871(2) | 5183(2) | 30(1) |
| Cl(8) | −2589(2) | 5544(2) | −162(2) | 27(1) |
| N(1) | 10531(8) | 4808(6) | 8521(6) | 24(2) |
| N(2) | 12958(8) | 5339(6) | 9110(6) | 25(2) |
| N(3) | 6851(8) | 7667(6) | 6758(6) | 27(2) |
| C(1) | 11704(8) | 5481(7) | 8621(6) | 22(2) |
| C(2) | 14025(10) | 6081(8) | 9117(8) | 30(2) |
| C(3) | 13878(9) | 7006(7) | 8617(7) | 27(2) |
| C(4) | 12610(9) | 7190(7) | 8137(7) | 25(2) |
| C(5) | 11458(9) | 6434(7) | 8140(7) | 23(2) |
| C(6) | 10020(9) | 6298(7) | 7759(7) | 23(2) |
| C(7) | 9516(10) | 5290(7) | 7994(7) | 24(2) |
| C(8) | 7662(10) | 6676(7) | 6945(8) | 28(2) |
| C(9) | 9157(9) | 7105(7) | 7295(7) | 24(2) |
| C(10) | 9648(9) | 8187(7) | 7246(7) | 27(2) |
| C(11) | 8821(9) | 9016(7) | 6754(7) | 29(2) |
| C(12) | 7511(9) | 8360(8) | 6087(8) | 30(2) |
| C(13) | 9628(10) | 9540(8) | 6019(9) | 35(2) |
| N(4) | −144(8) | 9356(7) | 1(6) | 29(2) |
| N(5) | −126(8) | 7586(6) | 649(6) | 28(2) |
| N(6) | 4591(8) | 6117(6) | 2871(6) | 29(2) |
| C(14) | 463(9) | 8563(7) | 469(7) | 25(2) |
| C(15) | 624(11) | 10339(8) | −59(8) | 32(2) |
| C(16) | 1977(10) | 10533(8) | 340(8) | 29(2) |
| C(17) | 2648(10) | 9724(7) | 825(7) | 27(2) |
| C(18) | 1878(9) | 8699(7) | 876(7) | 22(2) |
| C(19) | 2119(9) | 7713(7) | 1327(7) | 24(2) |
| C(20) | 879(10) | 7072(7) | 1171(7) | 28(2) |
| C(21) | 3274(9) | 6630(7) | 2618(7) | 24(2) |
| C(22) | 3429(9) | 7425(7) | 1866(7) | 22(2) |
| C(23) | 4641(9) | 7784(7) | 1663(7) | 28(2) |
| C(24) | 5995(9) | 7466(7) | 2156(7) | 28(2) |
| C(25) | 5815(9) | 7006(8) | 3143(8) | 30(2) |
| C(26) | 7097(10) | 8508(8) | 2480(8) | 31(2) |
| N(7) | 85(8) | 4674(6) | 4952(6) | 25(2) |
| N(8) | 2486(8) | 5226(6) | 5588(6) | 24(2) |
| N(9) | 6036(8) | 2451(6) | 7582(6) | 28(2) |
| C(27) | 1295(10) | 4549(7) | 5469(7) | 25(2) |
| C(28) | −1010(10) | 3907(8) | 4894(7) | 30(2) |
| C(29) | −854(10) | 2975(8) | 5368(7) | 30(2) |
| C(30) | 406(10) | 2816(7) | 5912(8) | 29(2) |
| C(31) | 1516(9) | 3612(7) | 5981(7) | 23(2) |
| C(32) | 2967(9) | 3774(7) | 6433(7) | 24(2) |
| C(33) | 3478(9) | 4771(7) | 6175(7) | 25(2) |
| C(34) | 5308(9) | 3392(7) | 7214(7) | 24(2) |
| C(35) | 3775(9) | 3095(7) | 7068(7) | 24(2) |
| C(36) | 3236(9) | 2304(7) | 7519(7) | 26(2) |
| C(37) | 4033(9) | 1601(7) | 8164(7) | 28(2) |
| C(38) | 5499(10) | 2140(8) | 8524(8) | 32(2) |
| C(39) | 3928(10) | 257(8) | 7519(8) | 34(2) |
| N(10) | 3310(8) | 815(6) | 4188(6) | 27(2) |
| N(11) | 3182(8) | 2585(6) | 3549(6) | 26(2) |
| N(12) | −1532(8) | 3699(6) | 1023(6) | 29(2) |
| C(40) | 2644(9) | 1525(7) | 3677(7) | 25(2) |
| C(41) | 2640(10) | −217(8) | 4230(8) | 29(2) |
| C(42) | 1302(11) | −517(7) | 3762(7) | 29(2) |
| C(43) | 590(9) | 219(7) | 3237(7) | 26(2) |
| C(44) | 1260(9) | 1303(7) | 3201(7) | 21(2) |
| C(45) | 953(9) | 2278(7) | 2735(7) | 25(2) |
| C(46) | 2151(9) | 3014(7) | 2984(7) | 24(2) |
| C(47) | −242(10) | 3590(8) | 1696(8) | 30(2) |
| C(48) | −334(9) | 2499(7) | 2140(7) | 24(2) |
| C(49) | −1514(9) | 1833(8) | 1989(8) | 30(2) |
| C(50) | −2849(9) | 2098(7) | 1452(7) | 29(2) |
| C(51) | −2771(10) | 3423(8) | 1495(8) | 30(2) |
| C(52) | −4025(10) | 1768(9) | 2016(9) | 36(2) |

### Example 6: Calcium Flux Assay

Stable cell lines for 5-HT_{2A}R, 5-HT_{2B}R and 5-HT_{2C}R generated using the Flp-In 293 T-Rex Tetracycline inducible system (Invitrogen). Tetracycline-induced cells were seeded in 384-well poly-L-lysine plates at a density of 10,000 cells/well in DMEM containing 1% dialyzed FBS at least 16-24 hr before the calcium flux assay. On the day of the assay, the cells were incubated (20 ul/well) for 1 hr at 37° C. with Fluo-4 Direct dye (Invitrogen) reconstituted in FLIPR buffer (1×HBSS, 2.5 mM probenecid, and 20 mM HEPES, pH 7.4). After dye loading, cells were placed in a FLIPR^{TETRA} fluorescence imaging plate reader (Molecular Dynamics). Drug dilutions were prepared at 3× final concentration in drug buffer (1×HBSS, 20 mM HEPES, 0.1% BSA, 0.01% ascorbic acid, pH 7.4) and aliquoted into 384-well plates and placed in the FLIPR^{TETRA} for drug stimulation. The fluidics module and plate reader of the FLIPR^{TETRA} were programmed to read baseline fluorescence for 10s (1 read/s), then 10 ul of drug/well was added and read for 5 min (1 read/s). Fluorescence in each well was normalized to the average of the first 10 reads (i.e., baseline fluorescence). Then, the maximum-fold increase, which occurred within the first 60 s after drug addition, was determined and fold over baseline was plotted as a function of drug concentration. Data were normalized to % 5-HT stimulation and analyzed using “log(agonist) vs. response” in GraphPad Prism 9.0.

TABLE 7
Compounds of the present disclosure

| Cmpd | Structure | Name |
|---|---|---|
| 1 | 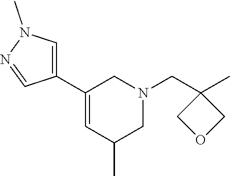 [SMILES: CC1C=C(c2cnn(C)c2)CN(CC2(C)COC2)C1] | 3-methyl-5-(1-methyl-1H-pyrazol-4-yl)-1-((3- methyloxetan-3-yl)methyl)-1,2,3,6- tetrahydropyridisne |
| 2 | 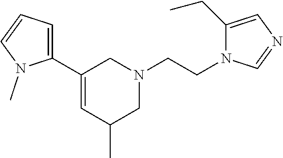 [SMILES: CCc1cncn1CCN1CC(c2cccn2C)=CC(C)C1] | 1-(2-(5-ethyl-1H-imidazol-1-yl)ethyl)-3- methyl-5-(1-methyl-1H-pyrrol-2-yl)-1,2,3,6- tetrahydropyridine |
| 3 | 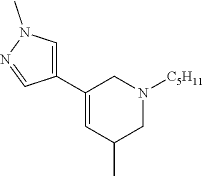 | 3-methyl-5-(1-methyl-1H-pyrazol-4-yl)-1- pentyl-1,2,3,6-tetrahydropyridine |
| 4 | 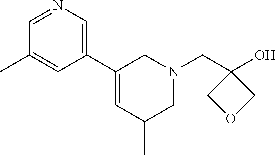 [SMILES: Cc1cncc(C2=CC(C)CN(CC3(O)COC3)C2)c1] | 3-((5,5′-dimethyl-5,6-dihydro-[3,3′-bipyridin]- 1(2H)-yl)methyl)oxetan-3-ol |
| 5 | 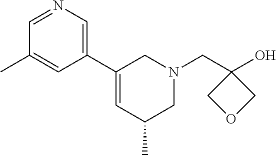 [SMILES: Cc1cncc(C2=C[C@@H](C)CN(CC3(O)COC3)C2)c1] | (R)-3-((5,5′-dimethyl-5,6-dihydro-[3,3′- bipyridin]-1(2H)-yl)methyl)oxetan-3-ol |
| 6 | 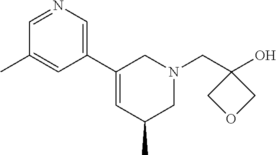 [SMILES: Cc1cncc(C2=C[C@H](C)CN(CC3(O)COC3)C2)c1] | (R)-3-((5,5′-dimethyl-5,6-dihydro-[3,3′- bipyridin]-1(2H)-yl)methyl)oxetan-3-ol |
| 7 | 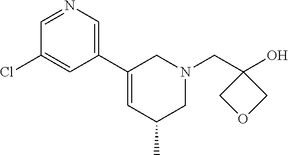 [SMILES: C[C@@H]1C=C(c2cncc(Cl)c2)CN(CC2(O)COC2)C1] | (R)-3-((5′-chloro-5-methyl-5,6-dihydro-[3,3′- bipyridin]-1(2H)-yl)methyl)oxetan-3-ol |
| 8 | 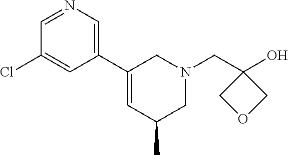 [SMILES: C[C@H]1C=C(c2cncc(Cl)c2)CN(CC2(O)COC2)C1] | (S)-3-((5′-chloro-5-methyl-5,6-dihydro-[3,3′- bipyridin]-1(2H)-yl)methyl)oxetan-3-ol |
| 9 | 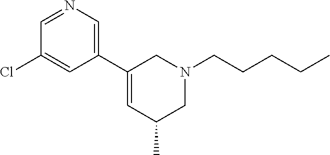 [SMILES: CCCCCN1CC(c2cncc(Cl)c2)=C[C@@H](C)C1] | (R)-5′-chloro-5-methyl-1-pentyl-1,2,5,6- tetrahydro-3,3′-bipyridine |
| 10 | 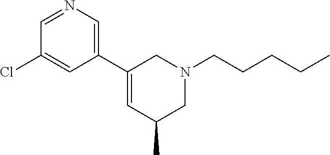 [SMILES: CCCCCN1CC(c2cncc(Cl)c2)=C[C@H](C)C1] | (S)-5′-chloro-5-methyl-1-pentyl-1,2,5,6- tetrahydro-3,3′-bipyridine |
| 11 | 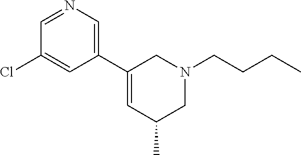 [SMILES: CCCCN1CC(c2cncc(Cl)c2)=C[C@@H](C)C1] | (R)-1-butyl-5′-chloro-5-methyl-1,2,5,6- tetrahydro-3,3′-bipyridine |
| 12 | 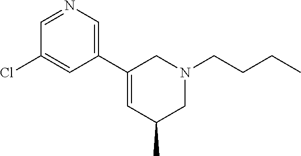 [SMILES: CCCCN1CC(c2cncc(Cl)c2)=C[C@H](C)C1] | (S)-1-butyl-5′-chloro-5-methyl-1,2,5,6- tetrahydro-3,3′-bipyridine |
| 13 | 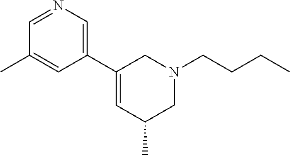 [SMILES: CCCCN1CC(c2cncc(C)c2)=C[C@@H](C)C1] | (R)-1-butyl-5,5′-dimethyl-1,2,5,6-tetrahydro- 3,3′-bipyridine |
| 14 | 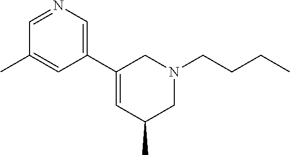 [SMILES: CCCCN1CC(c2cncc(C)c2)=C[C@H](C)C1] | (R)-1-butyl-5,5′-dimethyl-1,2,5,6-tetrahydro- 3,3′-bipyridine |
| 15 | 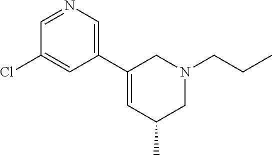 [SMILES: CCCN1CC(c2cncc(Cl)c2)=C[C@@H](C)C1] | (R)-5′-chloro-5-methyl-1-propyl-1,2,5,6- tetrahydro-3,3′-bipyridine |
| 16 | 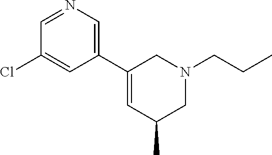 [SMILES: CCCN1CC(c2cncc(Cl)c2)=C[C@H](C)C1] | (S)-5′-chloro-5-methyl-1-propyl-1,2,5,6- tetrahydro-3,3′-bipyridine |
| 17 | 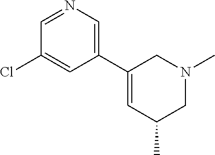 | (R)-5′-chloro-1,5-dimethyl-1,2,5,6-tetrahydro- 3,3′-bipyridine |
| 18 | 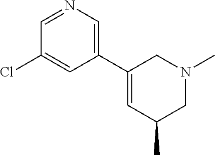 | (S)-5′-chloro-1,5-dimethyl-1,2,5,6-tetrahydro- 3,3′-bipyridine |
| 19 | 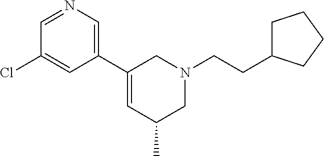 [SMILES: C[C@@H]1C=C(c2cncc(Cl)c2)CN(CCC2CCCC2)C1] | (R)-5′-chloro-1-(2-cyclopentylethyl)-5-methyl- 1,2,5,6-tetrahydro-3,3′-bipyridine |
| 20 | 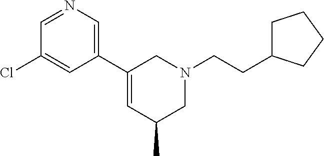 [SMILES: C[C@H]1C=C(c2cncc(Cl)c2)CN(CCC2CCCC2)C1] | (S)-5′-chloro-1-(2-cyclopentylethyl)-5-methyl- 1,2,5,6-tetrahydro-3,3′-bipyridine |
| 21 | 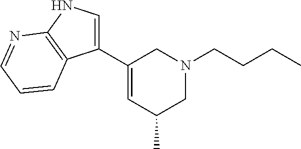 [SMILES: CCCCN1CC(c2c[nH]c3ncccc23)=C[C@@H](C)C1] | (R)-3-(1-butyl-5-methyl-1,2,5,6- tetrahydropyridin-3-yl)-1H-pyrrolo[2,3- b]pyridine |
| 22 | 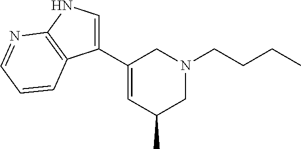 [SMILES: CCCCN1CC(c2c[nH]c3ncccc23)=C[C@H](C)C1] | (S)-3-(1-butyl-5-methyl-1,2,5,6- tetrahydropyridin-3-yl)-1H-pyrrolo[2,3- b]pyridine |
| 23 | 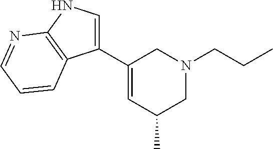 [SMILES: CCCN1CC(c2c[nH]c3ncccc23)=C[C@@H](C)C1] | (R)-3-(5-methyl-1-propyl-1,2,5,6- tetrahydropyridin-3-yl)-1H-pyrrolo[2,3- b]pyridine |
| 24 | 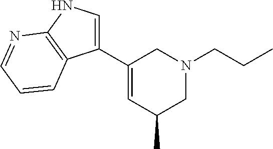 [SMILES: CCCN1CC(c2c[nH]c3ncccc23)=C[C@H](C)C1] | (S)-3-(5-methyl-1-propyl-1,2,5,6- tetrahydropyridin-3-yl)-1H-pyrrolo[2,3- b]pyridine |
| 25 | 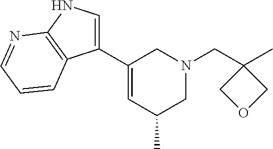 [SMILES: C[C@@H]1C=C(c2c[nH]c3ncccc23)CN(CC2(C)COC2)C1] | (R)-3-(5-methyl-1-((3-methyloxetan-3- yl)methyl)-1,2,5,6-tetrahydropyridin-3-yl)-1H- pyrrolo[2,3-b]pyridine |
| 26 | 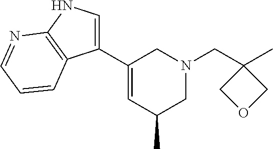 [SMILES: C[C@H]1C=C(c2c[nH]c3ncccc23)CN(CC2(C)COC2)C1] | (S)-3-(5-methyl-1-((3-methyloxetan-3- yl)methyl)-1,2,5,6-tetrahydropyridin-3-yl)-1H- pyrrolo[2,3-b]pyridine |
| 27 | 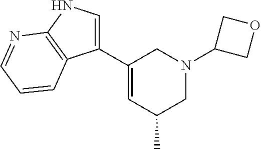 [SMILES: C[C@@H]1C=C(c2c[nH]c3ncccc23)CN(C2COC2)C1] | (R)-3-(5-methyl-1-(oxetan-3-yl)-1,2,5,6- tetrahydropyridin-3-yl)-1H-pyrrolo[2,3- b]pyridine |
| 28 | 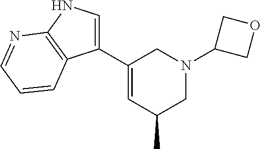 [SMILES: C[C@H]1C=C(c2c[nH]c3ncccc23)CN(C2COC2)C1] | (S)-3-(5-methyl-1-(oxetan-3-yl)-1,2,5,6- tetrahydropyridin-3-yl)-1H-pyrrolo[2,3- b]pyridine |
| 29 | 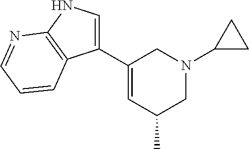 [SMILES: C[C@@H]1C=C(c2c[nH]c3ncccc23)CN(C2CC2)C1] | (R)-3-(1-cyclopropyl-5-methyl-1,2,5,6- tetrahydropyridin-3-yl)-1H-pyrrolo[2,3- b]pyridine |
| 30 | 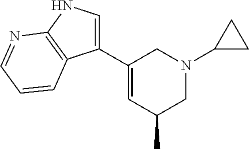 [SMILES: C[C@H]1C=C(c2c[nH]c3ncccc23)CN(C2CC2)C1] | (S)-3-(1-cyclopropyl-5-methyl-1,2,5,6- tetrahydropyridin-3-yl)-1H-pyrrolo[2,3- b]pyridine |
| 31 | 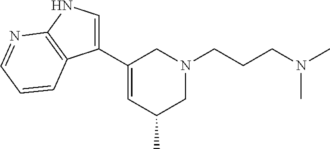 [SMILES: C[C@@H]1C=C(c2c[nH]c3ncccc23)CN(CCCN(C)C)C1] | (R)-N,N-dimethyl-3-(3-methyl-5-(1H- pyrrolo[2,3-b]pyridin-3-yl)-3,6- dihydropyrridin-1(2H)-yl)propan-1-amine |
| 32 | 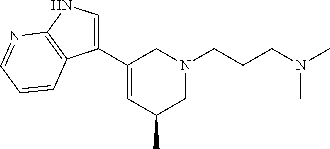 [SMILES: C[C@H]1C=C(c2c[nH]c3ncccc23)CN(CCCN(C)C)C1] | (S)-N,N-dimethyl-3-(3-methyl-5-(1H- pyrrolo[2,3-b]pyridin-3-yl)-3,6- dihydropyridin-1(2H)-yl)propan-1-amine |
| 33 | 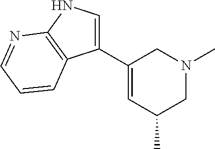 [SMILES: C[C@@H]1C=C(c2c[nH]c3ncccc23)CN(C)C1] | (R)-3-(1,5-dimethyl-1,2,5,6-tetrahydropyridin- 3-yl)-1H-pyrrolo[2,3-b]pyridine |
| 34 | 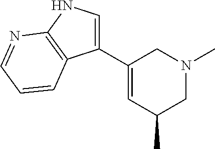 [SMILES: C[C@H]1C=C(c2c[nH]c3ncccc23)CN(C)C1] | (S)-3-(1,5-dimethyl-1,2,5,6-tetrahydropyridin- 3-yl)-1H-pyrrolo[2,3-b]pyridine |
| 35 | 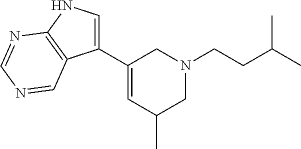 [SMILES: CC(C)CCN1CC(c2c[nH]c3ncncc23)=CC(C)C1] | 5-(1-Isopentyl-5-methyl-1,2,5,6- tetrahydropyridin-3-yl)-7H-pyrrolo[2,3- d]pyrimidine |
| 36 | 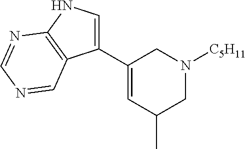 | 5-(5-Methyl-1-pentyl-1,2,5,6- tetrahydropyridin-3-yl)-7H-pyrrolo[2,3- d]pyrimidine |
| 37 | 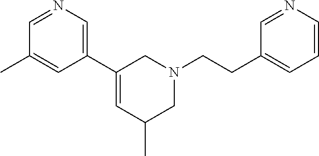 [SMILES: Cc1cncc(C2=CC(C)CN(CCc3cccnc3)C2)c1] | 5,5′-dimethyl-1-(2-(pyridin-3-yl)ethyl)-1,2,5,6- tetrahydro-3,3′-bipyridine |
| 38 | 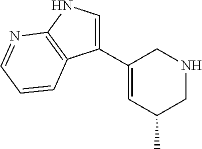 [SMILES: C[C@@H]1C=C(c2c[nH]c3ncccc23)CNC1] | (R)-3-(5-methyl-1,2,5,6-tetrahydropyridin-3- yl)-1H-pyrrolo[2,3-b]pyridine |
| 39 | 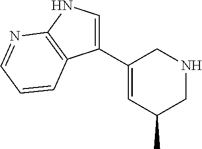 [SMILES: C[C@H]1C=C(c2c[nH]c3ncccc23)CNC1] | (S)-3-(5-methyl-1,2,5,6-tetrahydropyridin-3- yl)-1H-pyrrolo[2,3-b]pyridine |
| 40 | 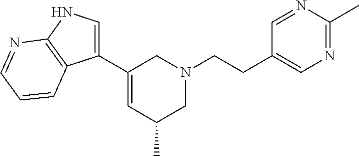 [SMILES: Cc1ncc(CCN2CC(c3c[nH]c4ncccc34)=C[C@@H](C)C2)cn1] | (R)-3-(5-methyl-1-(2-(2-methylpyrimidin-5- yl)ethyl)-1,2,5,6-tetrahydropyridin-3-yl)-1H- pyrrolo[2,3-b]pyridine |
| 41 | 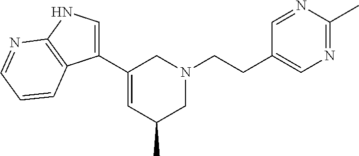 [SMILES: Cc1ncc(CCN2CC(c3c[nH]c4ncccc34)=C[C@H](C)C2)cn1] | (S)-3-(5-methyl-1-(2-(2-methylpyrimidin-5- yl)ethyl)-1,2,5,6-tetrahydropyridin-3-yl)-1H- pyrrolo[2,3-b]pyridine |
| 42 | 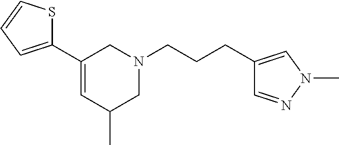 [SMILES: CC1C=C(c2cccs2)CN(CCCc2cnn(C)c2)C1] | 3-methyl-1-(3-(1-methyl-1H-pyrazol-4- yl)propyl)-5-(thiophen-2-yl)-1,2,3,6- tetrahydropyridine |
| 43 | 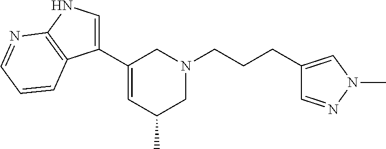 [SMILES: C[C@@H]1C=C(c2c[nH]c3ncccc23)CN(CCCc2cnn(C)c2)C1] | (R)-3-(5-methyl-1-(3-(1-methyl-1H-pyrazol-4- yl)propyl)-1,2,5,6-tetrahydropyridin-3-yl)-1H- pyrrolo[2,3-b]pyridine |
| 44 | 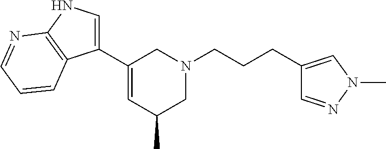 [SMILES: C[C@H]1C=C(c2c[nH]c3ncccc23)CN(CCCc2cnn(C)c2)C1] | (S)-3-(5-methyl-1-(3-(1-methyl-1H-pyrazol-4- yl)propyl)-1,2,5,6-tetrahydropyridin-3-yl)-1H- pyrrolo[2,3-b]pyridine |
| 45 | 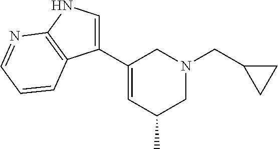 [SMILES: C[C@@H]1C=C(c2c[nH]c3ncccc23)CN(CC2CC2)C1] | (R)-3-(1-(cyclopropylmethyl)-5-methyl- 1,2,5,6-tetrahydropyridin-3-yl)-1H- pyrrolo[2,3-b]pyridine |
| 46 | 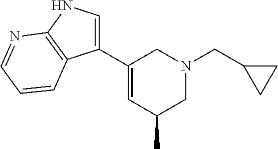 [SMILES: C[C@H]1C=C(c2c[nH]c3ncccc23)CN(CC2CC2)C1] | (S)-3-(1-(cyclopropylmethyl)-5-methyl- 1,2,5,6-tetrahydropyridin-3-yl)-1H- pyrrolo[2,3-b]pyridine |
| 47 | 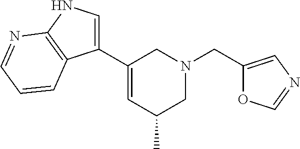 [SMILES: C[C@@H]1C=C(c2c[nH]c3ncccc23)CN(Cc2cnco2)C1] | (R)-5-((3-methyl-5-(1H-pyrrolo[2,3-b]pyridin- 3-yl)-3,6-dihydropyridin-1(2H)- yl)methyl)oxazole |
| 48 | 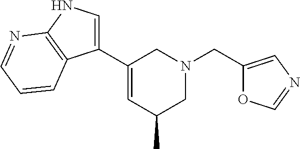 [SMILES: C[C@H]1C=C(c2c[nH]c3ncccc23)CN(Cc2cnco2)C1] | (S)-5-((3-methyl-5-(1H-pyrrolo[2,3-b]pyridin- 3-yl)-3,6-dihydropyridin-1(2H)- yl)methyl)oxazole |
| 49 | 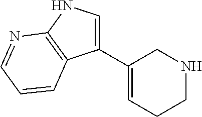 [SMILES: C1=C(c2c[nH]c3ncccc23)CNCC1] | 3-(1,2,5,6-tetrahydropyridin-3-yl)-1H- pyrrolo[2,3-b]pyridine |
| 50 | 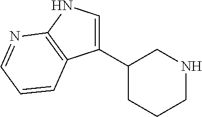 [SMILES: c1cnc2[nH]cc(C3CCCNC3)c2c1] | 3-(piperidin-3-yl)-1H-pyrrolo[2,3-b]pyridine Enantiomer I |
| 51 | 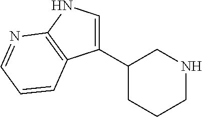 [SMILES: c1cnc2[nH]cc(C3CCCNC3)c2c1] | 3-(piperidin-3-yl)-1H-pyrrolo[2,3-b]pyridine Enantiomer II |

TABLE 8
5-HT₂R activity of selected compounds of the present disclosure

| | 5-HT_{2A}R | | | | 5-HT_{2B}R | | | | 5-HT_{2C}R | | | |
|---|---|---|---|---|---|---|---|---|---|---|---|---|
| | | | EC₅₀ | | | | EC₅₀ | | | | EC₅₀ | |
| Cmpd | Kᵢ (μM)^{a} | | (μM)^{b} | | Kᵢ (μM)^{a} | | (μM)^{b} | | Kᵢ (μM)^{a} | | (μM)^{b} | |
| 1 | — | | — | | 3.88 | | 1.94 | | — | | — | |
| | | | | | [5.41 | ± 0.09] | [5.71 | ± 0.12] | | | | |
| | | | | | | | 48.9% | ± 2.6 | | | | |
| 2 | 6.23 | | antagonist | | 2.67 | | 2.98 | | — | | — | |
| | [5.21 | ± 0.08] | | | [5.57 | ± 0.12] | [5.53 | ± 014] | | | | |
| | | | | | | | 56.3% | ± 4.1 | | | | |
| 3 | — | | >10 | | 1.48 | | antagonist | | — | | — | |
| | | | | | [5.83 | ± 0.14] | | | | | | |
| 4 | — | | — | | 0.67 | | antagonist | | — | | — | |
| | | | | | [6.17 | ± 0.08] | | | | | | |
| 5 | >10 | | — | | 3.2 | | antagonist | | >10 | | — | |
| | | | | | [5.49 | ± 0.11] | | | | | | |
| 6 | >10 | | — | | 0.25 | | 0.12 | | >10 | | — | |
| | | | | | [6.61 | ± 0.10] | [6.94 | ± 0.26] | | | | |
| | | | | | | | (16.0% | ± 1.8) | | | | |
| 7 | >10 | | >10 | | 0.96 | | antagonist | | >10 | | — | |
| | | | | | [6.02 | ± 0.12] | | | | | | |
| 8 | >10 | | — | | 0.08 | | 0.13 | | >10 | | — | |
| | | | | | [7.10 | ± 0.06] | [6.90 | ± 0.18] | | | | |
| | | | | | | | (20.4% | ± 1.5) | | | | |
| 9 | >10 | | 7.63 | | 0.56 | | antagonist | | >10 | | — | |
| | | | [5.11 | ± 0.21] | [6.26 | ± 0.09] | | | | | | |
| | | | (40.2% | ± 5.2) | | | | | | | | |
| 10 | 7.01 | | 0.33 | | 0.005 | | 0.025 | | 1.66 | | >10 | |
| | [5.15 | ± 0.38] | [6.48 | ± 0.11] | [8.30 | ± 0.10] | [7.61 | ± 0.04] | [5.78 | ± 0.27] | | |
| | | | (68.6% | ± 3.4) | | | (88.9% | ± 1.4) | | | | |
| 11 | >10 | | 4.24 | | 0.37 | | antagonist | | >10 | | — | |
| | | | [5.37 | ± 0.35] | [6.43 | ± 0.10] | | | | | | |
| | | | (25.0% | ± 4.8) | | | | | | | | |
| 12 | 4.54 | | 0.43 | | 0.007 | | 0.023 | | >10 | | — | |
| | [5.34 | ± 0.10] | [6.37 | ± 0.21] | [8.15 | ± 0.06] | [7.64 | ± 0.56] | | | | |
| | | | (53.0% | ± 5.0) | | | (69.6% | ± 5.5) | | | | |
| 13 | >10 | | >10 | | 0.58 | | antagonist | | >10 | | — | |
| | | | | | [6.24 | ± 0.19] | | | | | | |
| 14 | >10 | | 1.83 | | 0.03 | | 0.028 | | >10 | | — | |
| | | | [5.74 | ± 0.15] | [7.51 | ± 0.08] | [7.55 | ± 0.12] | | | | |
| | | | (57.3% | ± 4.3) | | | (71.7% | ± 3.4) | | | | |
| 15 | >10 | | 5.00 | | 0.15 | | 0.13 | | >10 | | — | |
| | | | [5.30 | ± 0.18] | [6.83 | ± 0.07] | [6.91 | ± 0.12] | | | | |
| | | | (42.1% | ± 4.1) | | | (50.9% | ± 2.5) | | | | |
| 16 | 7.32 | | 1.06 | | 0.013 | | 0.020 | | 6.52 | | 6.76 | |
| | [5.14 | ± 0.14] | [5.98 | ± 0.12] | [7.88 | ± 0.05] | [7.70 | ± 0.09] | [5.20 | ± 0.6] | [5.17 | ± 0.15] |
| | | | (48.7% | ± 2.8) | | | (74.9% | ± 3.7) | | | (34.4% | ± 3.0) |
| 17 | 2.10 | | 0.35 | | 0.030 | | 0.027 | | >10 | | — | |
| | [5.68 | ± 0.08] | [6.46 | ± 0.40] | [7.52 | ± 0.05] | [7.57 | ± 0.20] | | | | |
| | | | (14.9% | ± 2.6) | | | (42.1% | ± 3.3) | | | | |
| 18 | 3.21 | | 0.87 | | 0.14 | | antagonist | | >10 | | — | |
| | [5.50 | ± 0.08] | [6.06 | ± 0.36] | [6.86 | ± 0.09] | | | | | | |
| | | | (13.7% | ± 2.3) | | | | | | | | |
| 19 | >10 | | 4.31 | | 0.57 | | antagonist | | >10 | | — | |
| | | | [5.37 | ± 0.28] | [6.24 | ± 0.11] | | | | | | |
| | | | (34.9% | ± 5.3) | | | | | | | | |
| 20 | 4.36 | | 0.54 | | 0.011 | | 0.085 | | >10 | | — | |
| | [5.36 | ± 0.09] | [6.27 | ± 0.13] | [7.97 | ± 0.04] | [7.07 | ± 0.06] | | | | |
| | | | (49.1% | ± 2.9) | | | (66.2% | ± 1.5) | | | | |
| 21 | 2.19 | | 2.16 | | 2.92 | | antagonist | | >10 | | — | |
| | [5.66 | ± 0.15] | [5.67 | ± 0.21] | [5.34 | ± 0.14] | | | | | | |
| | | | (12.8% | ± 1.4) | | | | | | | | |
| 22 | 5.27 | | 2.26 | | 1.97 | | antagonist | | 7.03 | | antagonist | |
| | [5.28 | ± 0.16] | [5.65 | ± 0.14] | [5.71 | ± 0.08] | | | [5.13 | ± 1.33] | | |
| | | | (23.8% | ± 1.7) | | | | | | | | |
| 23 | 1.22 | | 0.25 | | 1.64 | | antagonist | | >10 | | — | |
| | [5.92 | ± 0.14] | [6.61 | ± 0.06] | [5.78 | ± 0.11] | | | | | | |
| | | | (65.6% | ± 1.6) | | | | | | | | |
| 24 | >10 | | 0.86 | | 1.67 | | antagonist | | >10 | | — | |
| | | | [6.06 | ± 0.25] | [5.78 | ± 0.10] | | | | | | |
| | | | (14.8% | ± 1.7) | | | | | | | | |
| 25 | >10 | | 4.00 | | 3.29 | | 6.38 | | >10 | | — | |
| | | | [5.40 | ± 0.18] | [5.48 | ± 0.10] | [5.20 | ± 0.26] | | | | |
| | | | (29.5% | ± 2.9) | | | (44.6% | ± 6.8) | | | | |
| 26 | >10 | | >10 | | 5.83 | | 9.23 | | >10 | | — | |
| | | | | | [5.24 | ± 0.12] | [5.04 | ± 0.19] | | | | |
| | | | | | | | (71.0% | ± 8.3) | | | | |
| 27 | >10 | | >10 | | >10 | | antagonist | | >10 | | — | |
| 28 | >10 | | >10 | | 9.89 | | 5.24 | | >10 | | — | |
| | | | | | [5.01 | ± 0.12] | [5.28 | ± 0.23] | | | | |
| | | | | | | | (48.1% | ± 6.2) | | | | |
| 29 | 6.14 | | 1.36 | | 1.66 | | 5.15 | | >10 | | — | |
| | [5.21 | ± 0.12] | [5.87 | ± 0.09] | [5.78 | ± 0.11] | [5.29 | ± 0.15] | | | | |
| | | | (63.0% | ± 2.7) | | | (74.7% | ± 6.2) | | | | |
| 30 | >10 | | 3.54 | | 2.54 | | 4.36 | | >10 | | — | |
| | | | [5.45 | ± 0.16] | [5.60 | ± 0.15] | [5.36 | ± 0.19] | | | | |
| | | | (29.1% | ± 2.5) | | | (58.3% | ± 6.1) | | | | |
| 31 | >10 | | antagonist | | >10 | | antagonist | | >10 | | — | |
| 32 | >10 | | antagonist | | 9.72 | | antagonist | | >10 | | — | |
| | | | | | [5.01 | ± 0.16] | | | | | | |
| 33 | 0.88 | | 0.11 | | 0.44 | | 0.70 | | 0.48 | | 3.25 | |
| | [6.06 | ± 0.12] | [6.96 | ± 0.04] | [6.36 | ± 0.13] | [6.15 | ± 0.08] | [6.32 | ± 0.11] | [5.49 | ± 0.08] |
| | | | (73.3% | ± 1.3) | | | (50.5% | ± 1.8) | | | (51.7% | ± 2.2) |
| 34 | 1.52 | | 1.14 | | 0.64 | | antagonist | | 0.75 | | antagonist | |
| | [5.82 | ± 0.11] | [5.94 | ± 0.19] | [6.19 | ± 0.13] | | | [6.13 | ± 0.36] | | |
| | | | (7.1% | ± 0.6) | | | | | | | | |
| 38 | 0.68 | | 0.041 | | 0.30 | | 0.19 | | 0.46 | | 2.06 | |
| | [6.17 | ± 0.06] | [7.38 | ± 0.04] | [6.53 | ± 0.14] | [6.73 | ± 0.06] | [6.35 | ± 0.09] | [5.69 | ± 0.06] |
| | | | (90.1% | ± 1.6) | | | (82.9% | ± 1.9) | | | (75.0% | ± 2.2) |
| 39 | 1.86 | | 0.52 | | 1.81 | | 1.23 | | 1.01 | | antagonist | |
| | [5.73 | ± 0.11] | [6.29 | ± 0.21] | [5.74 | ± 0.17] | [5.91 | ± 0.17] | [6.00 | ± 0.31] | | |
| | | | (14.0% | ± 1.1) | | | (26.8% | ± 2.1) | | | | |
| 40 | 0.40 | | antagonist | | 4.09 | | 3.82 | | 0.84 | | antagonist | |
| | [6.40 | ± 0.07] | | | [5.40 | ± 0.18] | [5.42 | ± 0.23] | [6.08 | ± 0.42] | | |
| | | | | | | | (54.5% | ± 6.7) | | | | |
| 41 | 0.025 | | antagonist | | 0.82 | | antagonist | | 0.03 | | antagonist | |
| | [7.59 | ± 0.07] | | | [6.09 | ± 0.10] | | | [7.54 | ± 0.05] | | |
| 43 | 0.67 | | antagonist | | 0.70 | | 0.79 | | >10 | | — | |
| | [6.18 | ± 0.15] | | | [6.16 | ± 0.11] | [6.10 | ± 0.27] | | | | |
| | | | | | | | (39.5% | ± 4.9) | | | | |
| 44 | 2.37 | | 0.27 | | 0.96 | | 0.36 | | >10 | | — | |
| | [5.63 | ± 0.14] | [6.57 | ± 0.22] | [6.02 | ± 0.09] | [6.45 | ± 0.16] | | | | |
| | | | (40.2% | ± 3.8) | | | (56.2% | ± 3.8) | | | | |
| 45 | 2.92 | | 0.45 | | >10 | | antagonist | | 4.41 | | antagonist | |
| | [5.54 | ± 0.08] | [6.34 | ± 0.26] | | | | | [5.36 | ± 0.62] | | |
| | | | (25.0% | ± 2.8) | | | | | | | | |
| 46 | 2.44 | | antagonist | | 2.14 | | 1.17 | | 0.78 | | antagonist | |
| | [5.61 | ± 0.09] | | | [5.67 | ± 0.14] | [5.93 | ± 0.07] | [6.11 | ± 0.30] | | |
| | | | | | | | (43.8% | ± 1.3) | | | | |
| 47 | 7.19 | | 0.99 | | >10 | | 0.94 | | >10 | | >10 | |
| | [5.14 | ± 0.16] | [6.01 | ± 0.08] | | | [6.03 | ± 0.16] | | | | |
| | | | (68.3% | ± 0.1) | | | (75.8% | ± 5.6) | | | | |
| 48 | >10 | | antagonist | | 8.01 | | 8.86 | | >10 | | — | |
| | | | | | [5.10 | ± 0.12] | [5.05 | ± 0.12] | | | | |
| | | | | | | | (48.8% | ± 3.7) | | | | |
| 49 | 0.40 | | 0.15 | | 0.26 | | 0.63 | | 0.29 | | 0.82 | |
| | [6.40 | ± 0.10] | [6.82 | ± 0.10] | [6.59 | ± 0.19] | [6.20 | ± 0.11] | [6.54 | ± 0.16] | [6.08 | ± 0.09] |
| | | | (43.9% | ± 1.9) | | | (34.8% | ± 1.6) | | | (45.9% | ± 1.8) |

| 50 | 5.25 | | 1.17 | | 0.62 | | 0.68 | | 2.73 | | >10 |
|---|---|---|---|---|---|---|---|---|---|---|---|
| | [5.28 | ± 0.18] | [5.93 | ± 0.07] | [6.21 | ± 0.20] | [6.17 | ± 0.20] | [5.56 | ± 0.54] | |
| | | | (65.1% | ± 2.2) | | | (47.0% | ± 4.2) | | | |

| 51 | 2.73 | | 4.21 | | 0.50 | | antagonist | 0.48 | | antagonist |
|---|---|---|---|---|---|---|---|---|---|---|
| | [5.56 | ± 0.14] | [5.38 | ± 0.15] | [6.30 | ± 0.14] | | [6.32 | ± 0.28] | |
| | | | (18.2% | ± 1.5) | | | | | | |

^{a}pKᵢ ± S.E.M;
^{b}[pEC₅₀ ± S.E.M](Rₘₐₓ ± S.E.M)

### Example 7: Behavioral Pharmacology

### Behavioral Methods and Statistics

Open field: this apparatus has been described in the literature (Acta Crystallographica Section D, 2010, 66:486-501). Mice were placed into the open field for 30 min, injected with vehicle, (+)-LSD-(+)-tartrate (NIDA Drug Supply Program, Bethesda, MID), 38, or 33 (i.p.), and returned immediately to the open field for 30 min. Locomotion was monitored using Fusion Integra software (Omnitech, Columbus, OH).

Head twitch response: HTRs were filmed in the open field and were scored following drug administration for 30 min as described in the literature (Acta Crystallographica Section D, 2019, 75:861-877).

Tail suspension: This test was conducted as described in the literature (Acta Crystallographica Section D, 2010, 66:486-501). Mice were administered vehicle, fluoxetine (Sigma-Aldrich, St. Louis, MO), 38, or 33 (i.p.). Immobility times were assessed over 6 min using MedAssociates software.

Statistics: Statistical analyses were performed with IBM SPSS Statistics 27 programs (IBM, Chicago, IL). The data are presented as means and standard errors of the mean. No sex effects were detected. One- or two-way ANOVA or repeated measures ANOVA (RMANOVA) were used, followed by Bonferroni corrected post-hoc analyses. A p<0.05 was considered significant. All behavioral results were plotted using GraphPad Prism.

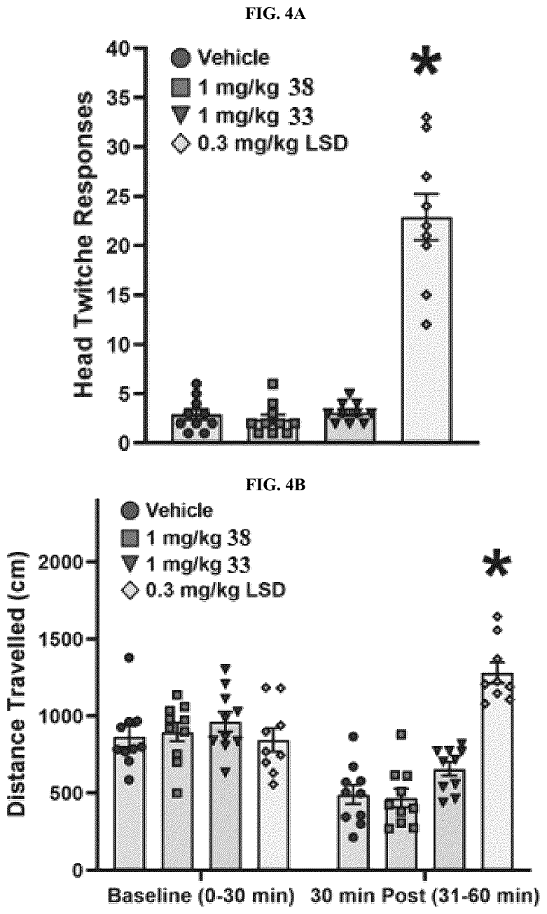

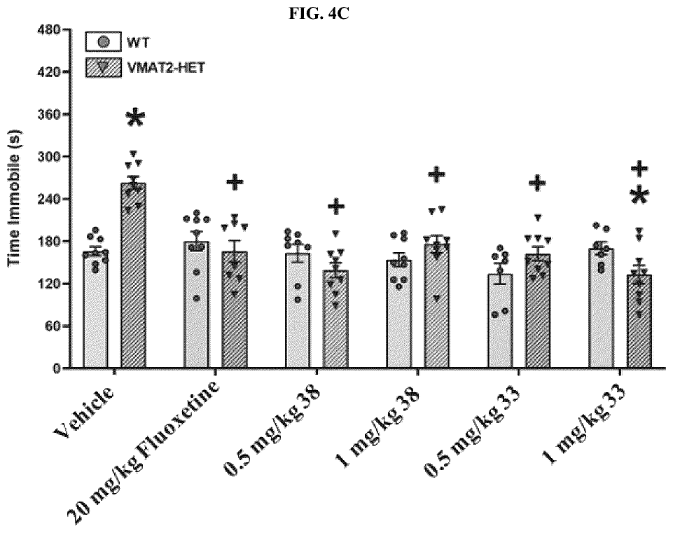

Psychedelic 5-HT_{2A}R agonists like psilocybin are reported to exert anxiolytic, anti-depressive, and anti-drug abuse actions, albeit with hallucinogenic actions. Behavioral studies were conducted in mice which are thought to reflect psychedelic drug-like and antidepressant drug-like activities. In the head twitch response (HTR) assay, which is thought to predict psychedelic drug-like actions, neither 38 nor 33 induced substantial HTRs, unlike the well-known psychedelic 5-HT_{2A}R agonist LSD (FIG. 4A). Nor did either compound show substantial stimulation of open field locomotion (FIG. 4B). The absence of psychedelic drug-like actions in these tests was in stark contrast to results obtained with LSD and psilocybin. At the doses administered, 38 and 33 are devoid of psychedelic drug like actions and do not enhance locomotion in the open field.

Conversely, both molecules were highly active in a mouse immobility assay that is thought to reflect antidepressant drug-like activities. Using VMAT2-heterozygous mice, which are predisposed to immobility (depressed-like behavior), 0.5 mg/kg of 38 and 1 mg/kg of 33 restored wild-type levels of mobility to mice. This compares favorably to the well-known antidepressant fluoxetine (Prozac), which only restores this level of activity at doses 40 to 20-fold higher (FIG. 4C). Thus, the new agonists appear to confer anti-depressant like activities without incurring psychedelic drug-like actions in these mouse behavioral assays.

### Enumerated Embodiments

The following exemplary embodiments are provided, the numbering of which is not to be construed as designating levels of importance:

Embodiment 1 provides a compound of formula (I), or a salt, solvate, tautomer, N-oxide, geometric isomer, and/or stereoisomer thereof:

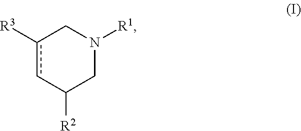

wherein:
[symbol] represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)-C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution independently comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂;
each occurrence of R is independently H, C₁-C₁₂ alkyl, C₃-C₁₂ cycloalkyl, or —(C₁-C₁₂ alkyl)C₃-C₁₂ cycloalkyl.

Embodiment 2 provides the compound of Embodiment 1, having a structure of formula (I-A) or (I-B):

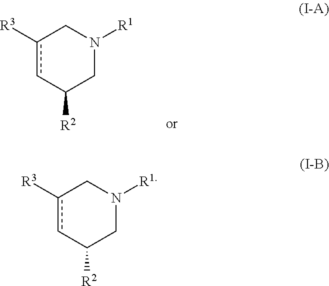

Embodiment 3 provides the compound of Embodiment 1 or 2, having a structure of formula (II-A) or (II-B);

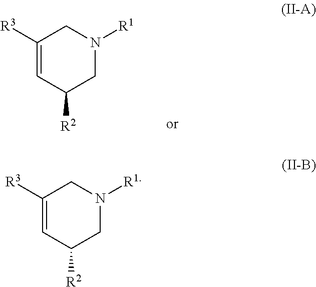

Embodiment 4 provides the compound of any of Embodiments 1-3, wherein [symbol] is a double bond.

Embodiment 5 provides the compound of any of Embodiments 1-2, having a structure of formula (III-A), (III-B), (III-C), or (III-D):

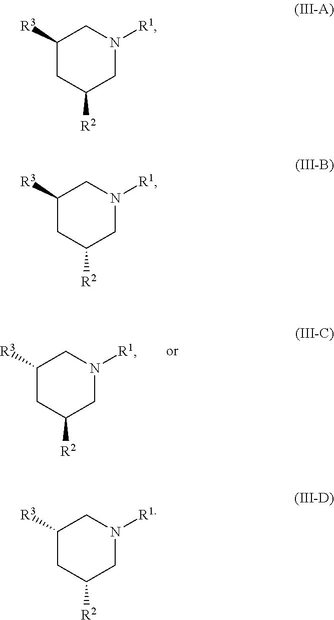

Embodiment 6 provides the compound of any of Embodiments 1-2 or 5, wherein [symbol] is a single bond.

Embodiment 7 provides the compound of any of Embodiments 1-6, wherein R¹ is selected from the group consisting of H, C₁-C₁₂ alkyl, —(C₁-C₁₂ alkyl)-C₃-C₁₂ cycloalkyl, and optionally substituted —(C₁-C₁₂ alkyl)-C₂-C₁₈ heterocyclyl.

Embodiment 8 provides the compound of any of Embodiments 1-7, wherein R¹ is selected from the group consisting of H, methyl, ethyl, n-propyl, n-butyl, i-pentyl, n-pentyl, —(CH₂)ₙ-cyclopropyl, —(CH₂)ₙ-cyclobutyl,

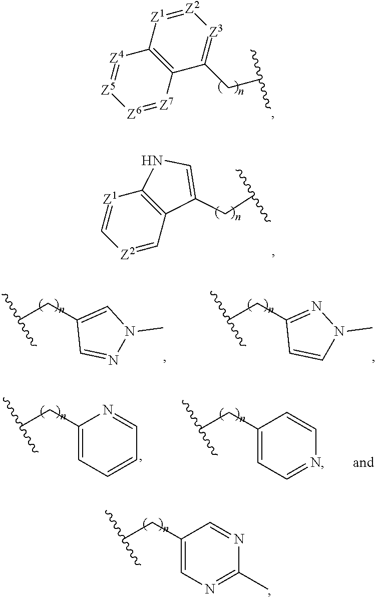

wherein
each of Z¹, Z², Z³, Z⁴, Z⁵, Z⁶, and Z⁷ is independently CH or N, and
n is independently an integer from 0 to 6.

Embodiment 9 provides the compound of any of Embodiments 1-8, wherein R¹ is selected from the group consisting of:

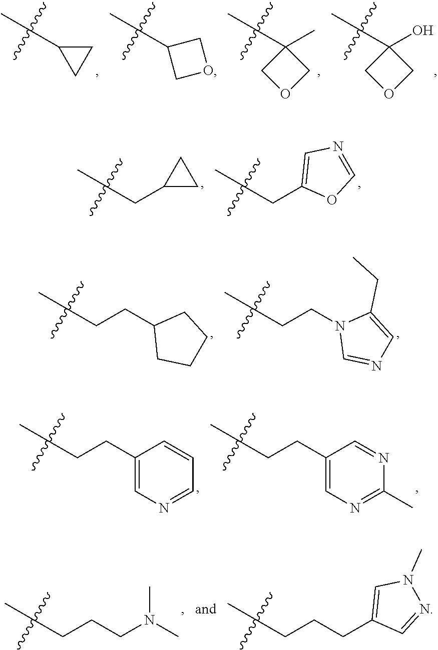

Embodiment 10 provides the compound of any of Embodiments 1-9, wherein R² is selected from the group consisting of H and C₁-C₁₂ alkyl.

Embodiment 11 provides the compound of any of Embodiments 1-10, wherein R² is methyl.

Embodiment 12 provides the compound of any of Embodiments 1-11, wherein R³ is optionally substituted C₂-C₁₀ heterocyclyl.

Embodiment 13 provides the compound of any of Embodiments 1-12, wherein R³ is selected from the group consisting of:

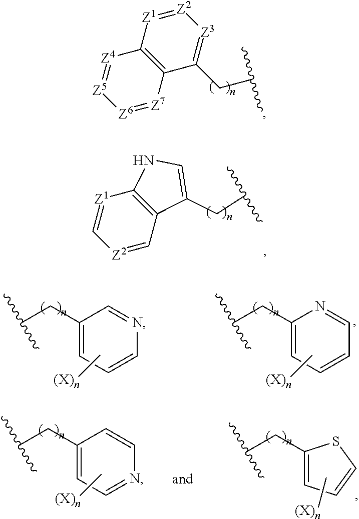

wherein
   m is independently an integer from 0 to 4,
   n is independently an integer from 0 to 6,
   each of Z¹, Z², Z³, Z⁴, Z⁵, Z⁶, and Z⁷ is independently CH or N, and
   each occurrence of X is independently selected from the group consisting of H, F, Cl, Br, I, OR, CN, NO₂, CF₃, OCF₃, R, N(R)₂, SOR, SO₂R, SO₂N(R)₂, C(O)R, and C(O)N(R)₂.

Embodiment 14 provides the compound of Embodiment 13, wherein n is 0 and m is 1.

Embodiment 15 provides the compound of any of Embodiments 13-14, wherein X is selected from the group consisting of C₁-C₃ alkyl, F, Cl, Br, OH, and C₁-C₃ alkoxy.

Embodiment 16 provides the compound of any of Embodiments 1-15, wherein R³ is selected from the group consisting of

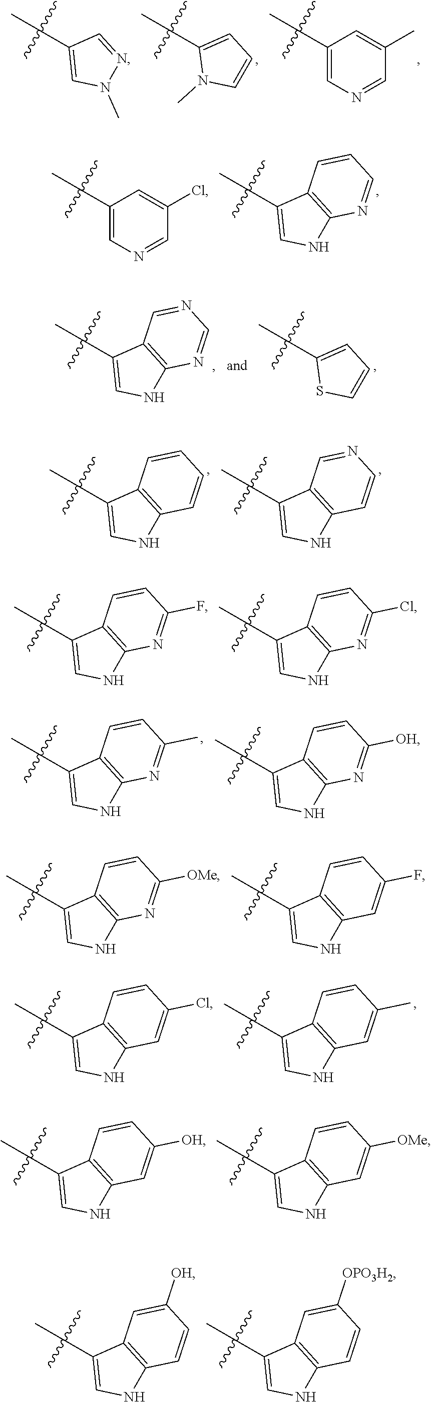

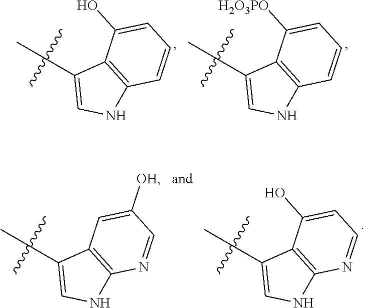

Embodiment 17 provides the compound of any of Embodiments 1-16, which is selected from the group consisting of:

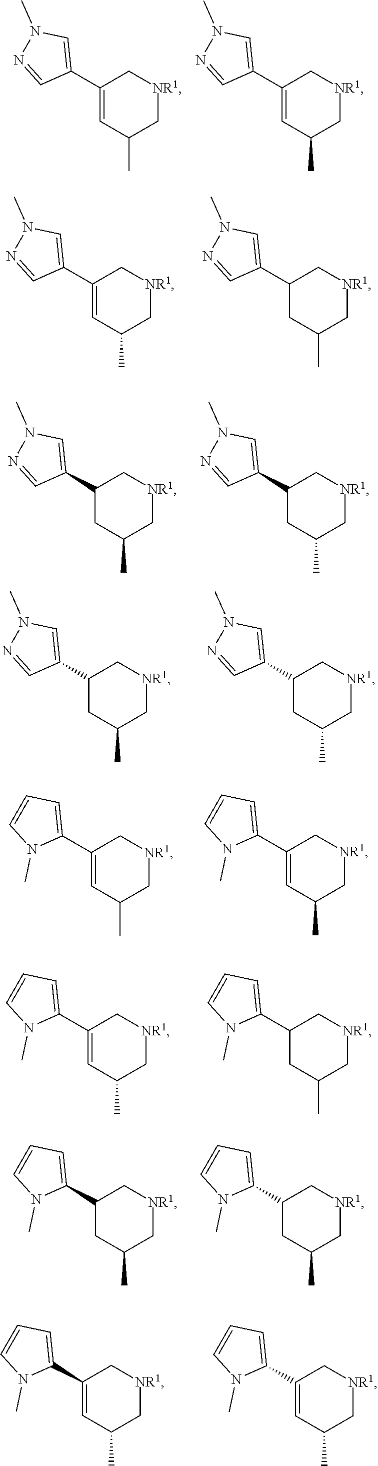

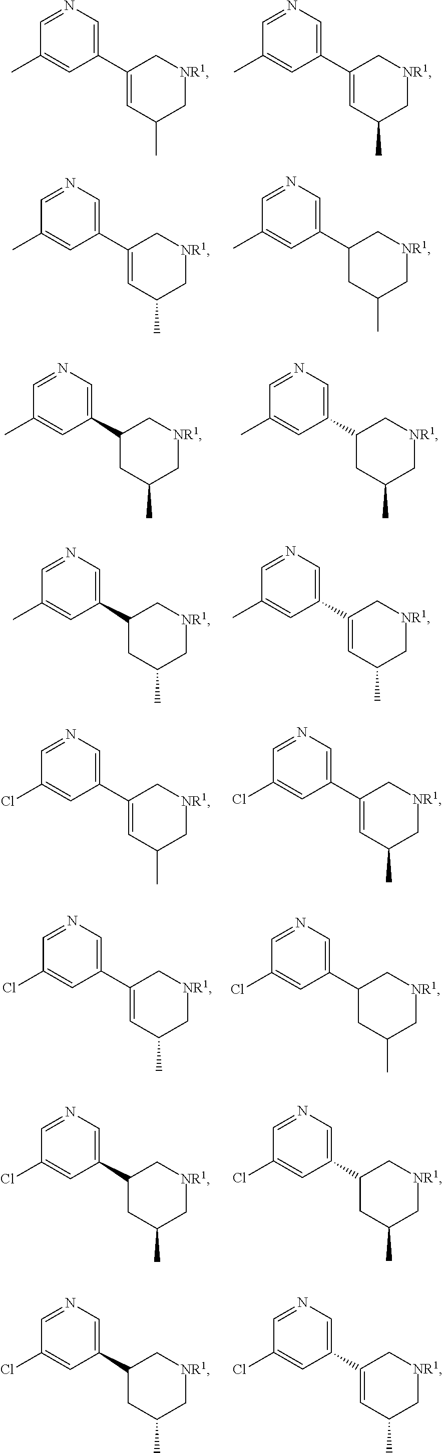

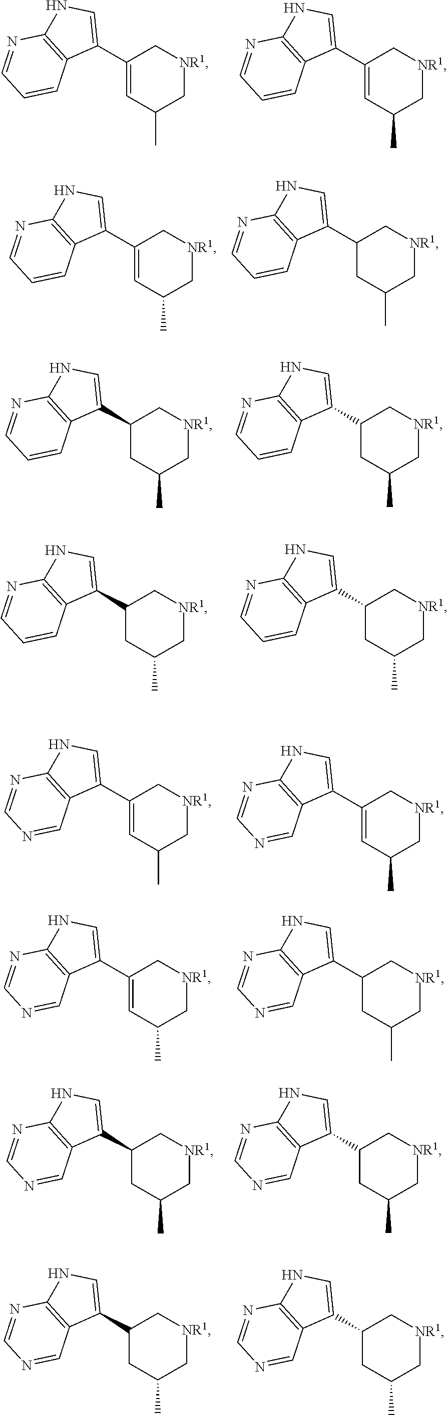

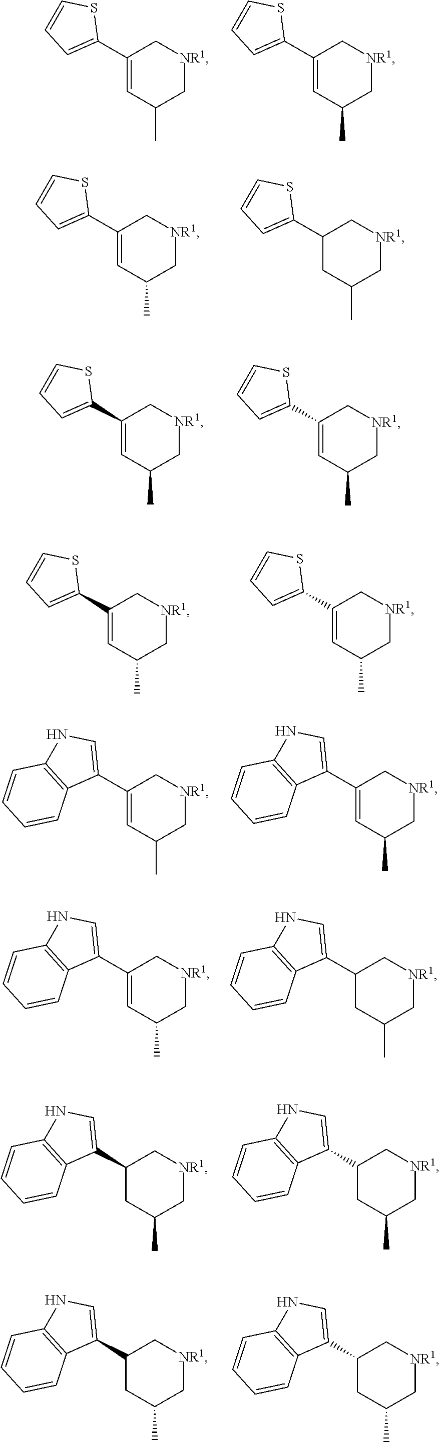

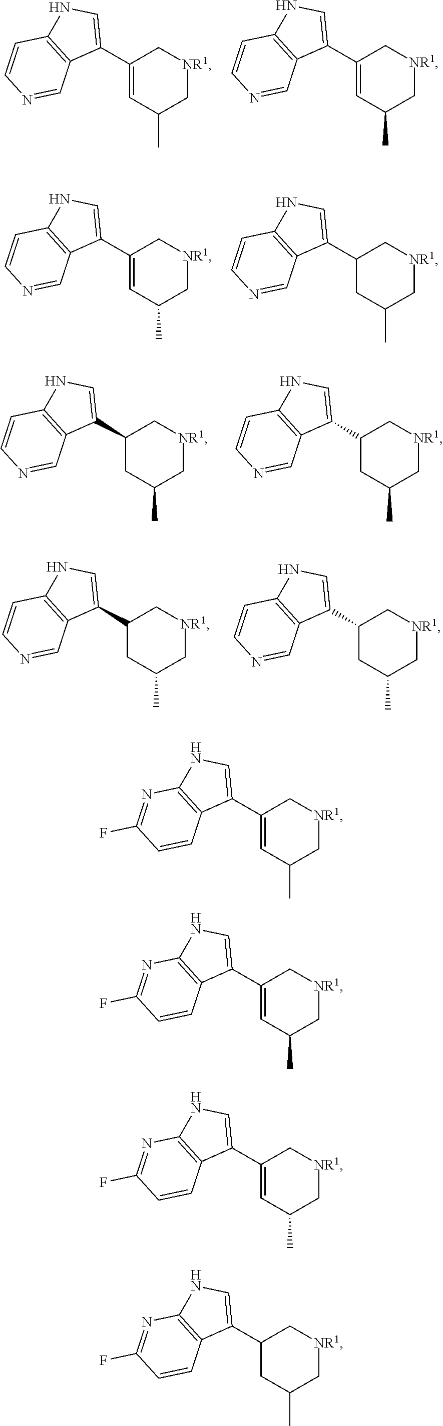

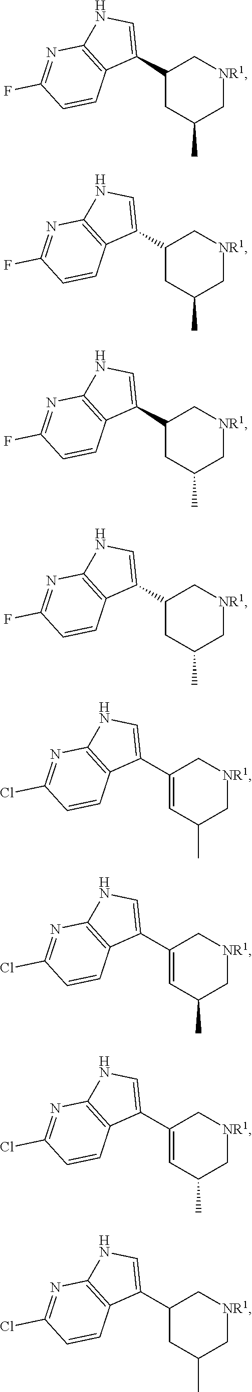

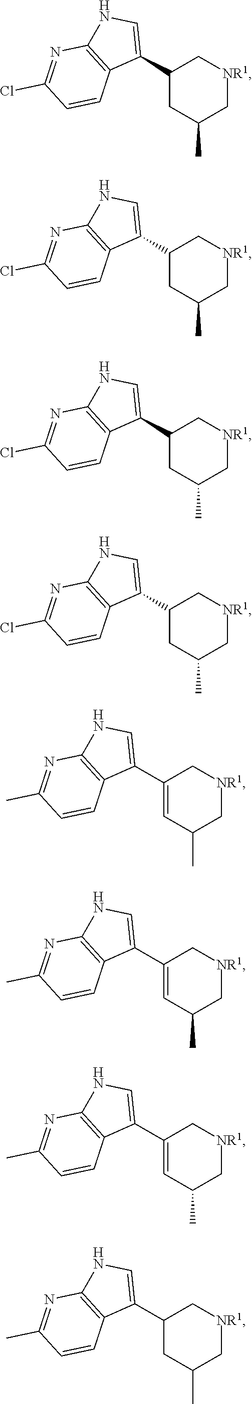

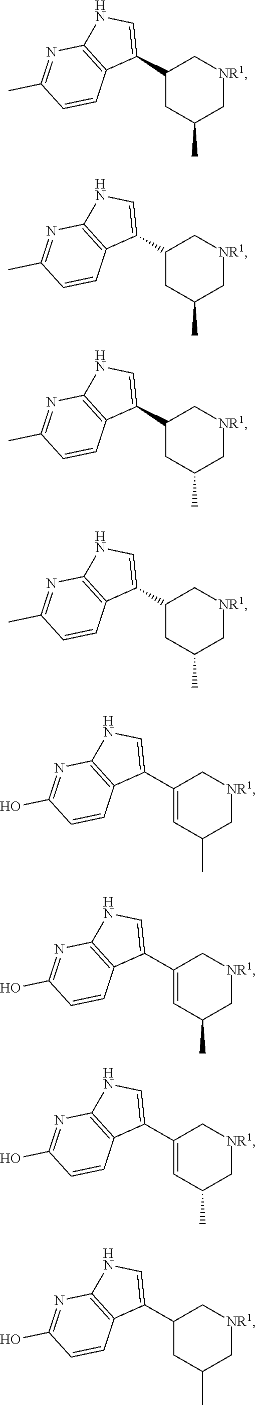

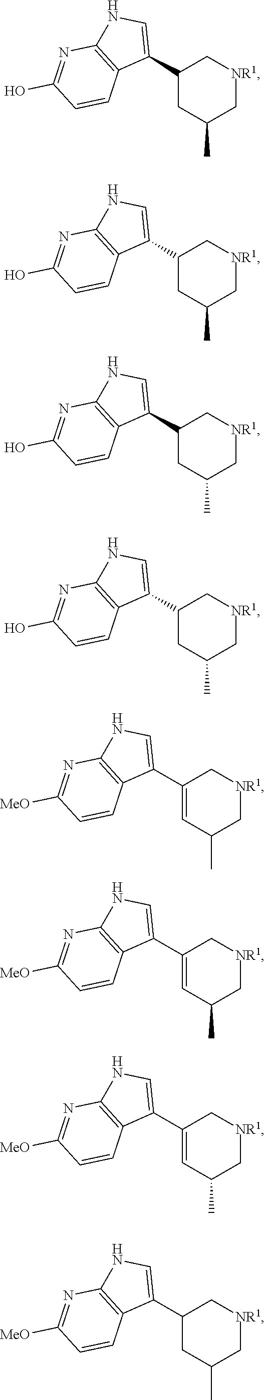

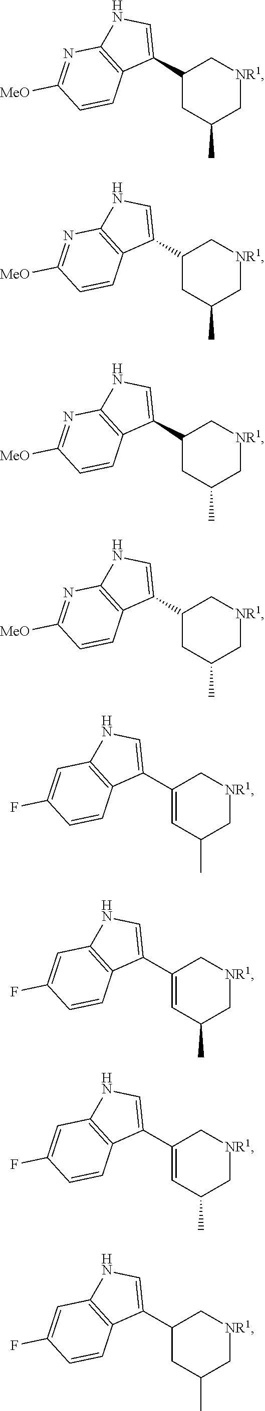

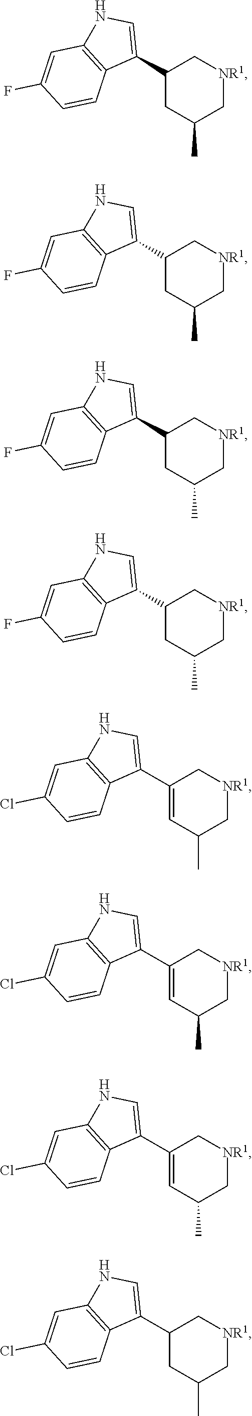

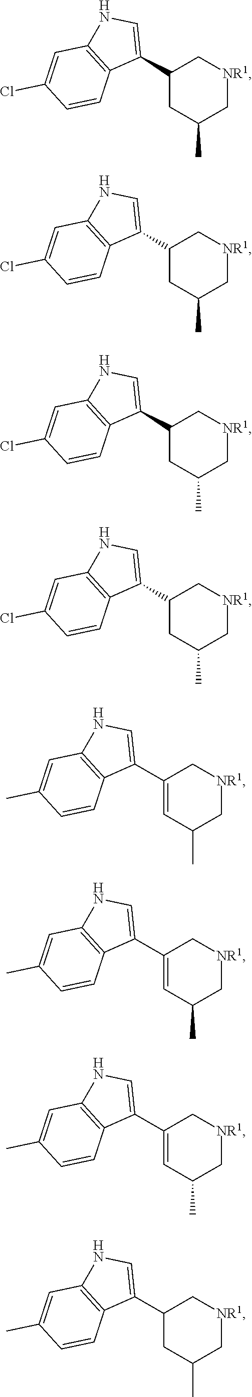

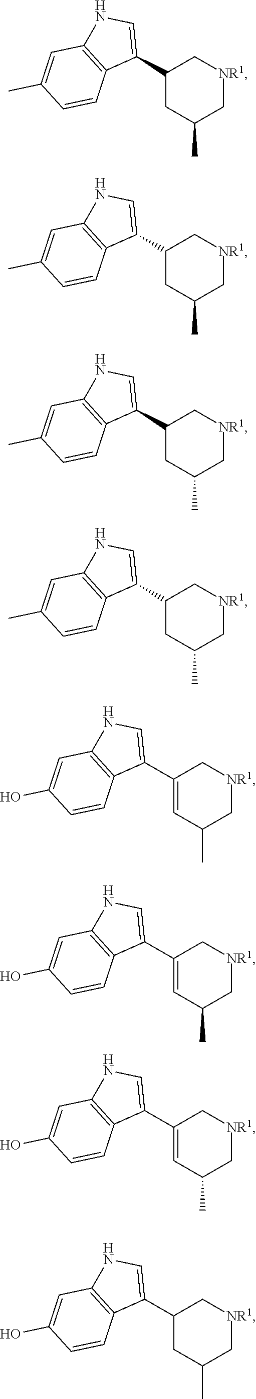

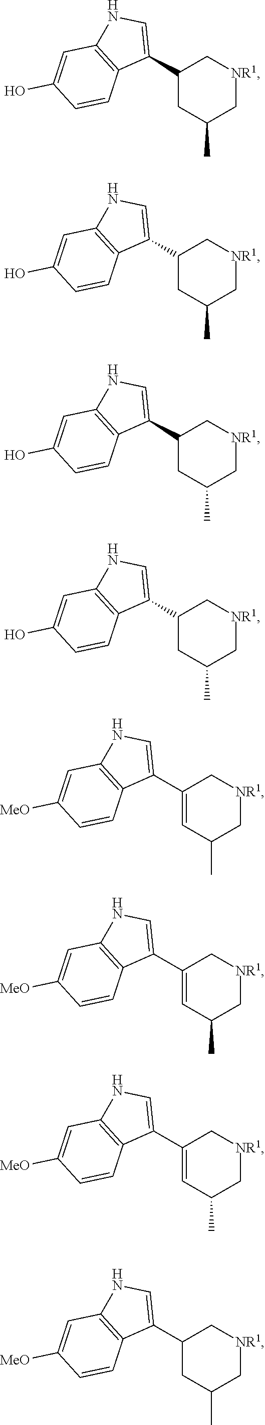

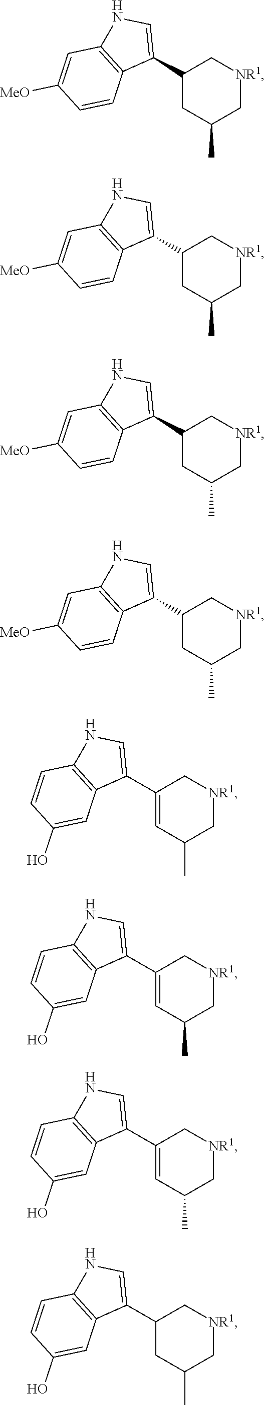

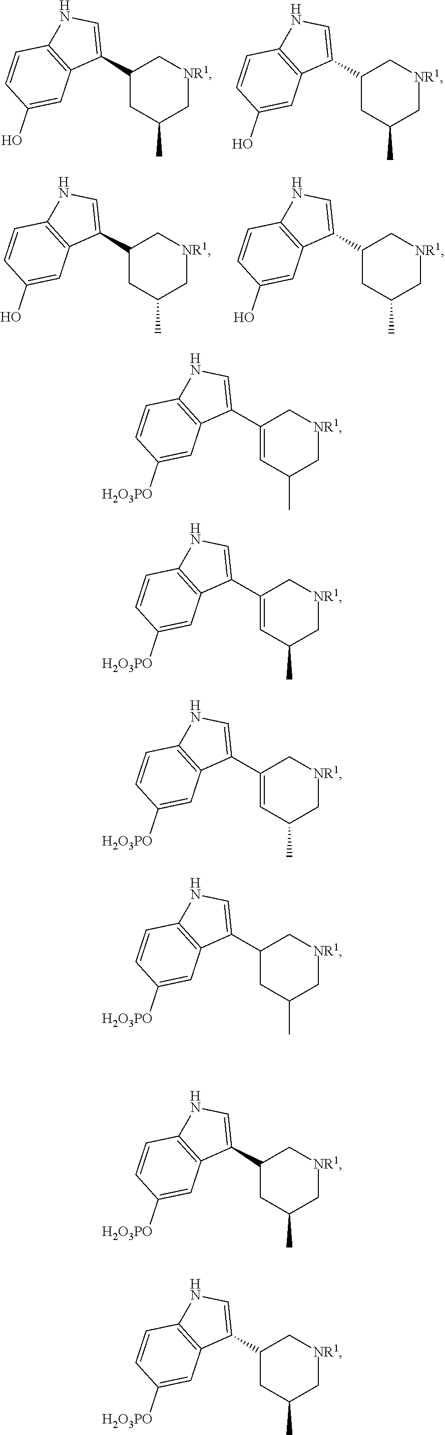

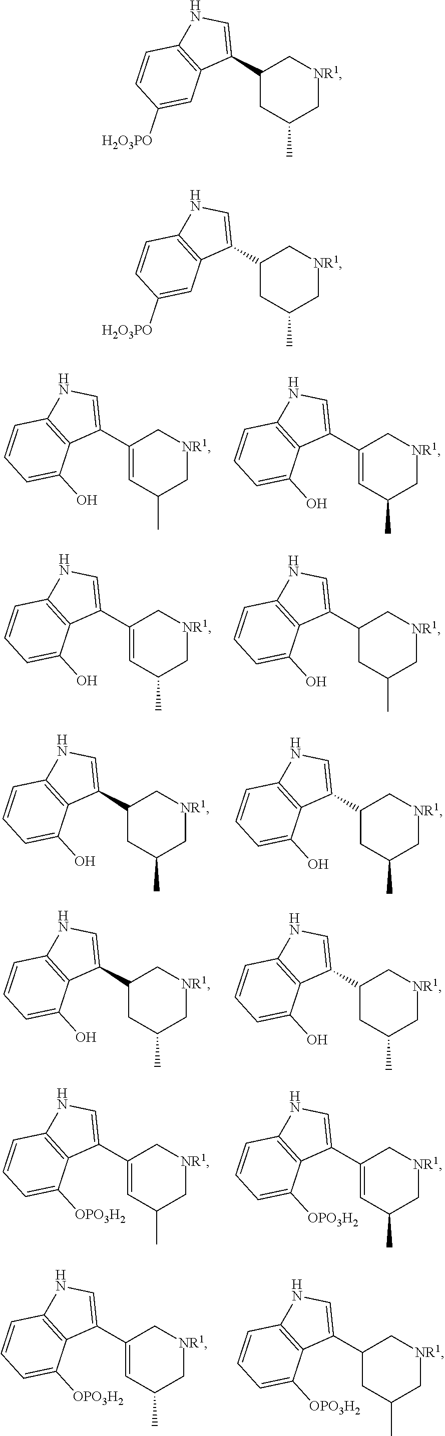

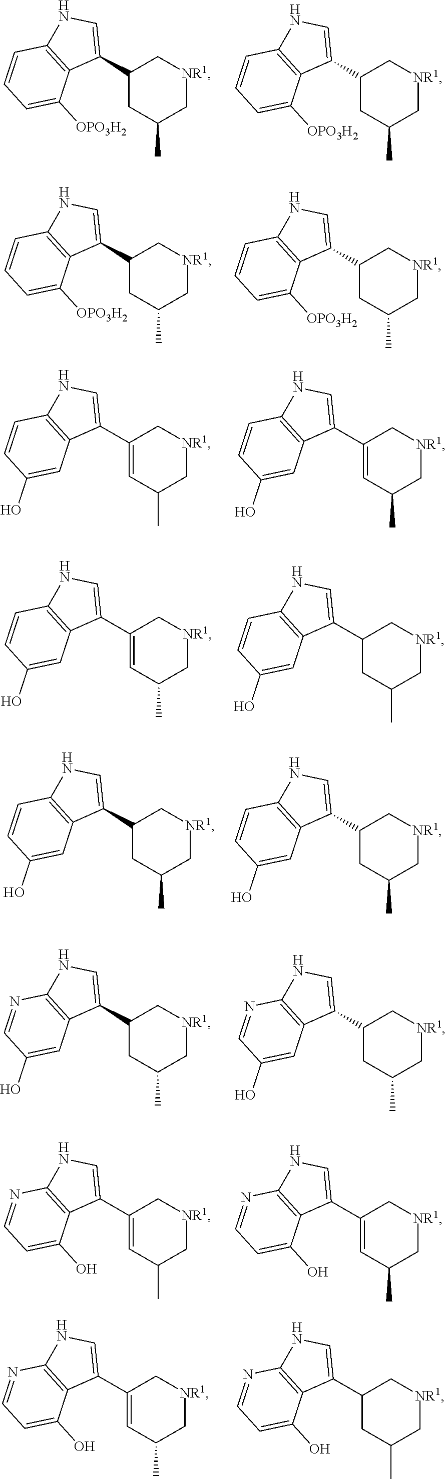

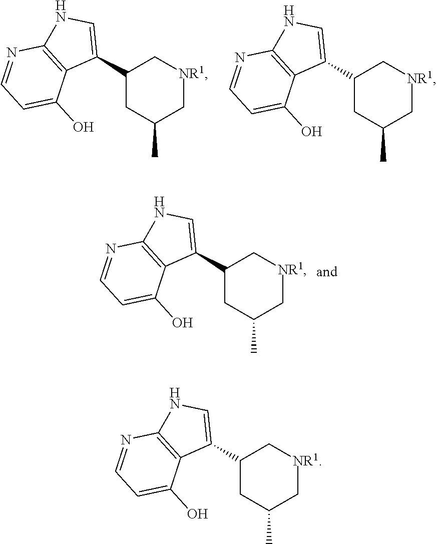

Embodiment 18 provides the compound of any of Embodiments 1-17, which is selected from the group consisting of:

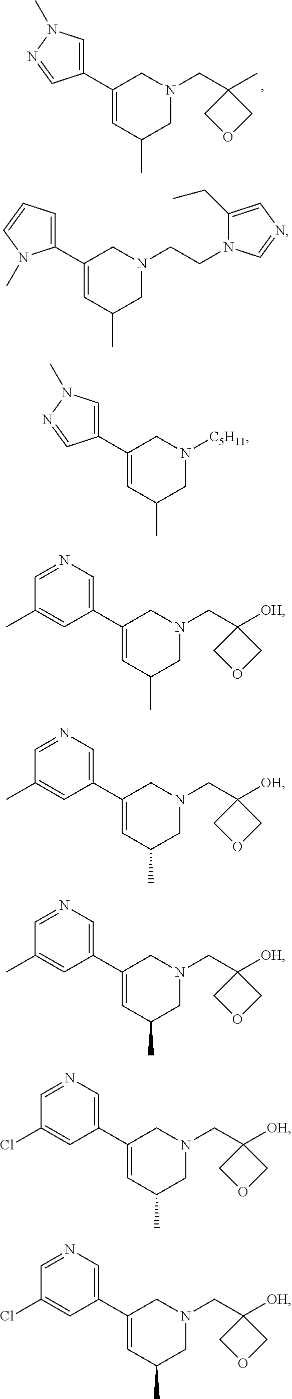

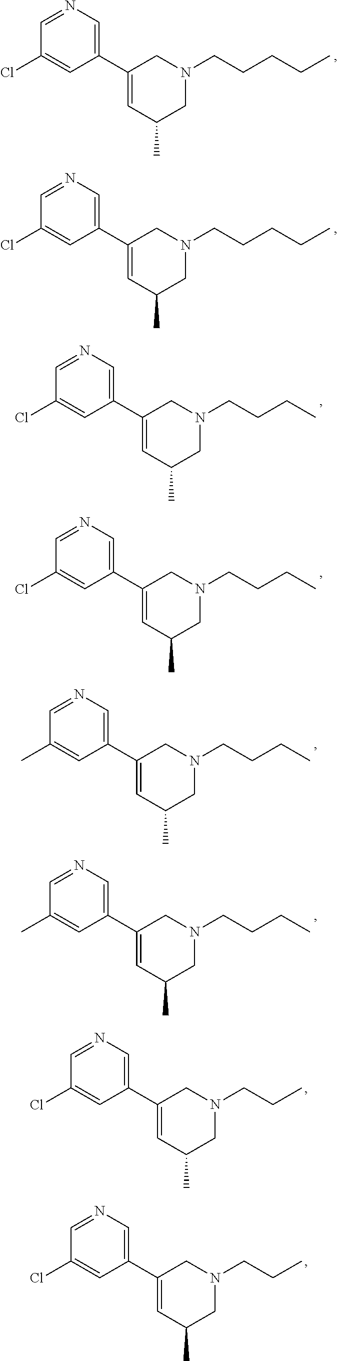

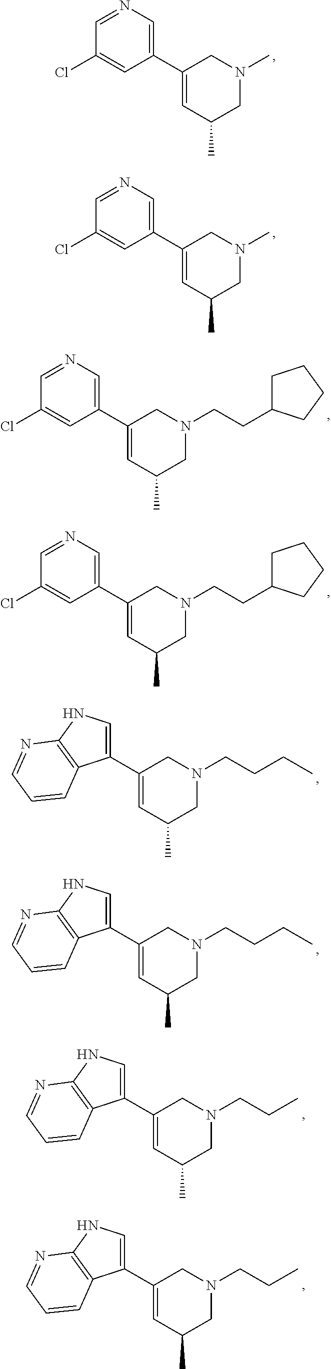

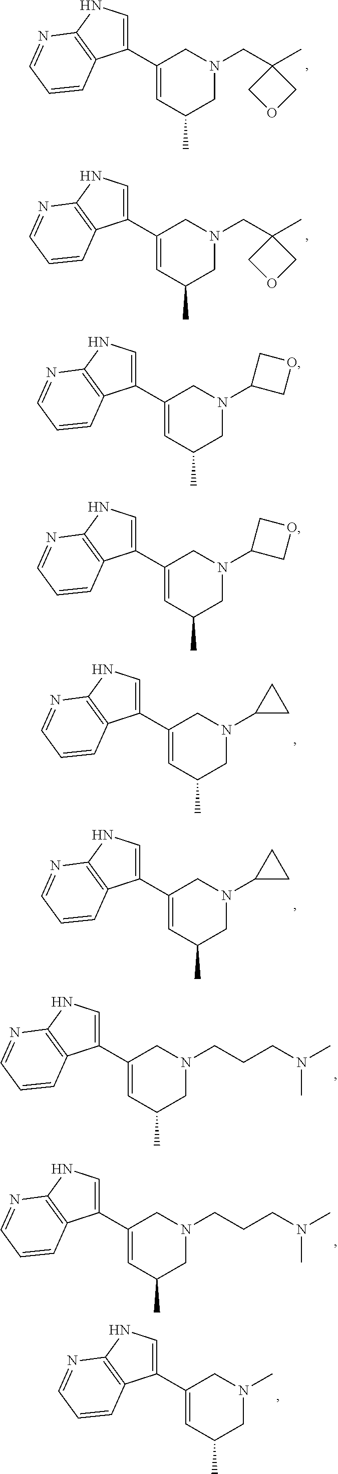

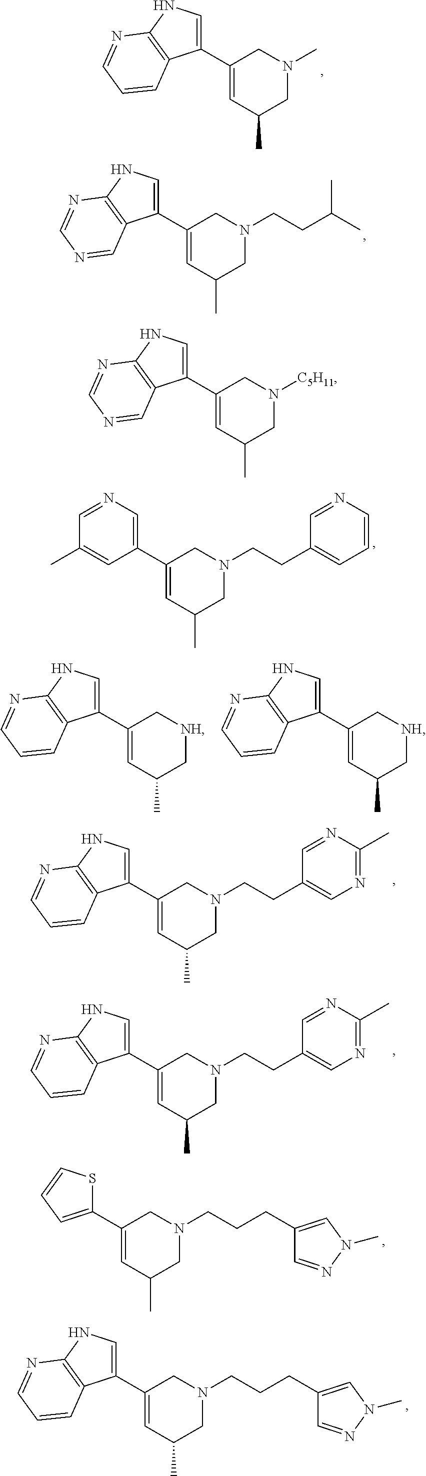

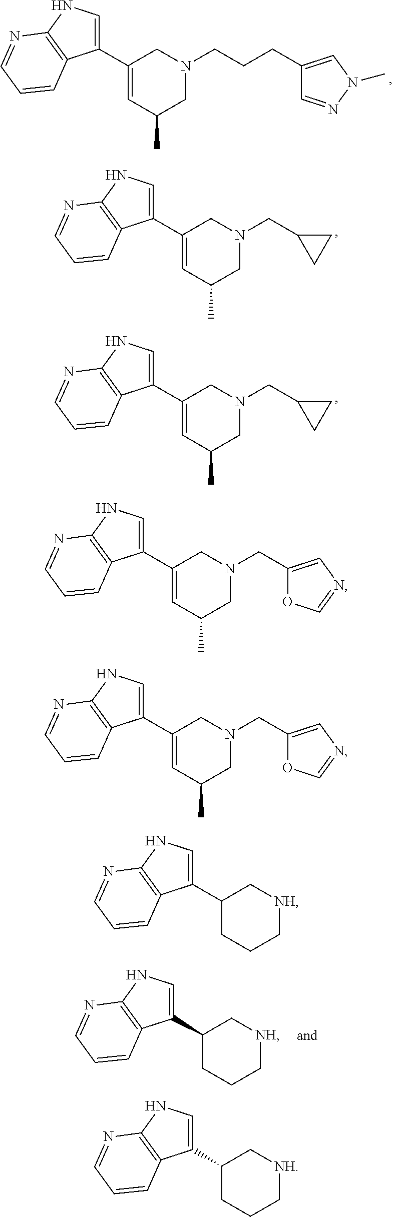

Embodiment 19 provides a pharmaceutical composition comprising the compound of any one of Embodiments 1-18 and at least one pharmaceutically acceptable excipient.

Embodiment 20 provides the pharmaceutical composition of Embodiment 19, further comprising an additional therapeutic agent that treats, ameliorates, and/or prevents a neurological disease and/or disorder.

Embodiment 21 provides a method of treating, ameliorating, and/or preventing a neurological disease and/or disorder, the method comprising administering to a subject in need thereof a therapeutically effective amount of a compound of formula (II):

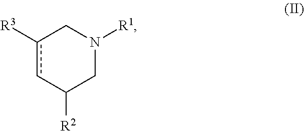

wherein:
[symbol] represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, and optionally substituted C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂; and
each occurrence of R is independently H, C₁-C₁₂ alkyl, C₃-C₁₂ cycloalkyl, or —(C₁-C₁₂ alkyl)C₃-C₁₂ cycloalkyl;
   or a salt, solvate, tautomer, N-oxide, geometric isomer, and/or stereoisomer thereof.

Embodiment 22 provides the method of Embodiment 21, wherein the neurological disease and/or disorder is selected from the group consisting of depression, anxiety, substance abuse, and headaches.

Embodiment 23 provides the method of any of Embodiments 21-22, wherein the compound is formulated as a pharmaceutical composition further comprising at least one pharmaceutically acceptable excipient.

Embodiment 24 provides the method of any of Embodiments 21-23, wherein the compound is administered by a route selected from the group consisting of oral, transdermal, intravesical, intrapulmonary, intraduodenal, intragastrical, intrathecal, subcutaneous, intramuscular, intradermal, intra-arterial, intravenous, intrabronchial, inhalation, and topical.

Embodiment 25 provides the method of any of Embodiments 21-24, wherein the subject is a mammal.

Embodiment 26 provides the method of Embodiment 25, wherein the mammal is a human.

Embodiment 27 provides a method of selectively agonizing the 5-hydroxytryptamine 2A (5-HT_{2A}) receptor in a subject in need thereof, the method comprising administering to a subject in need thereof a therapeutically effective amount of a compound of formula (II):

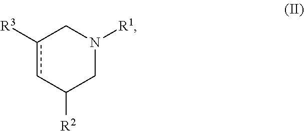

wherein:
[symbol] represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, and optionally substituted C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂; and
each occurrence of R is independently H, C₁-C₁₂ alkyl, C₃-C₁₂ cycloalkyl, or —(C₁-C₁₂ alkyl)C₃-C₁₂ cycloalkyl; or a salt, solvate, tautomer, N-oxide, geometric isomer, and/or stereoisomer thereof.

Embodiment 28 provides the method of Embodiment 27, wherein the compound is formulated as a pharmaceutical composition further comprising at least one pharmaceutically acceptable excipient.

Embodiment 29 provides the method of any of Embodiments 27-28, wherein the compositions is administered by a route selected from the group consisting of oral, transdermal, intravesical, intrapulmonary, intraduodenal, intragastrical, intrathecal, subcutaneous, intramuscular, intradermal, intra-arterial, intravenous, intrabronchial, inhalation, and topical.

Embodiment 30 provides the method of any of Embodiments 27-29, wherein the subject is a mammal.

Embodiment 31 provides the method of Embodiment 30, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of formula (I), or a salt, solvate, tautomer, N-oxide, geometric isomer, and/or stereoisomer thereof: 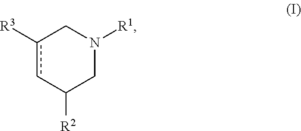
wherein: [symbol]
represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)-C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution independently comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂;
each occurrence of R is independently H, C₁-C₁₂ alkyl, C₃-C₁₂ cycloalkyl, or —(C₁-C₁₂ alkyl)C₃-C₁₂ cycloalkyl.

2. The compound of claim 1, having a structure selected from the group consisting of:

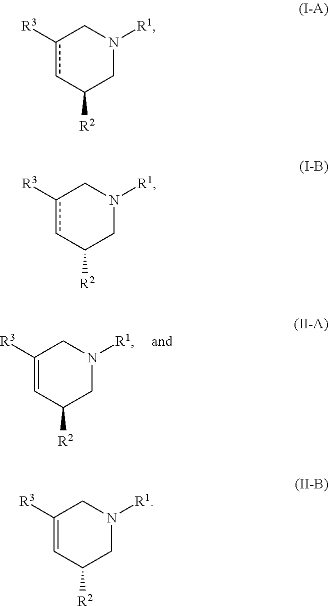

3-4. (canceled)

5. The compound of claim 1, having a structure of formula (III-A), (III-B), (III-C), or (III-D):

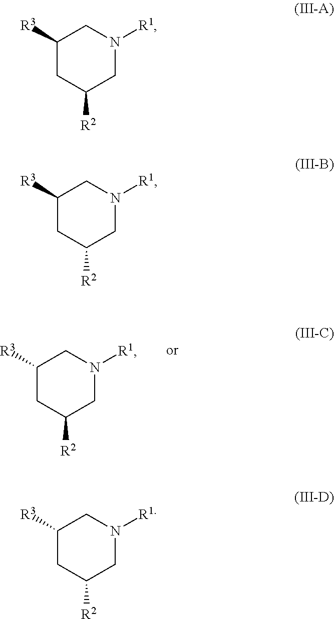

6. (canceled)

7. The compound of claim 1, wherein R¹ is selected from the group consisting of H, C₁-C₁₂ alkyl, —(C₁-C₁₂ alkyl)-C₃-C₁₂ cycloalkyl, and optionally substituted —(C₁-C₁₂ alkyl)-C₂-C₁₈ heterocyclyl.

8. The compound of claim 1, wherein R¹ is selected from the group consisting of
H, methyl, ethyl, n-propyl, n-butyl, i-pentyl, n-pentyl, —(CH₂)ₙ-cyclopropyl, —(CH₂)ₙ-cyclobutyl, —(CH₂)ₙ-cyclopentyl, 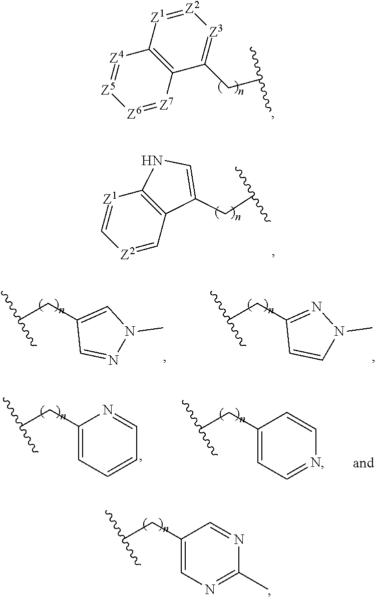
wherein:
each of Z¹, Z², Z³, Z⁴, Z⁵, Z⁶, and Z⁷ is independently CH or N, and n is independently an integer from 0 to 6.

9. The compound of claim 1, wherein R¹ is selected from the group consisting of:

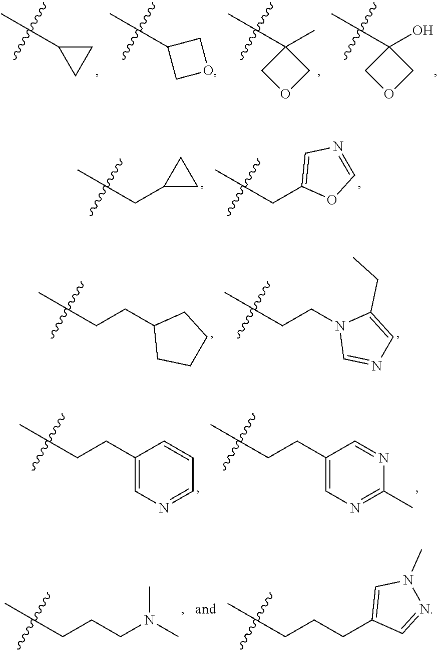

10. The compound of claim 1, wherein R² is selected from the group consisting of H and C₁-C₁₂ alkyl.

11. The compound of claim 1, wherein R² is methyl.

12. The compound of claim 1, wherein R³ is optionally substituted C₂-C₁₀ heterocyclyl.

13. The compound of claim 1, wherein R³ is selected from the group consisting of: 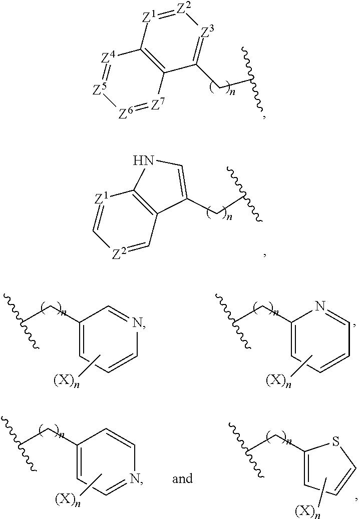
wherein;
m is independently an integer from 0 to 4,
n is independently an integer from 0 to 6,
each of Z¹, Z², Z³, Z⁴, Z⁵, Z⁶, and Z⁷ is independently CH or N, and
each occurrence of X is independently selected from the group consisting of H, F, Cl, Br, I, OR, CN, NO₂, CF₃, OCF₃, R, N(R)₂, SOR, SO₂R, SO₂N(R)₂, C(O)R, and C(O)N(R)₂.

14. The compound of claim 13, wherein n is 0 and m is 1.

15. The compound of claim 13, wherein X is selected from the group consisting of C₁-C₃ alkyl, F, Cl, Br, OH, and C₁-C₃ alkoxy.

16. The compound of claim 1, wherein R³ is selected from the group consisting of

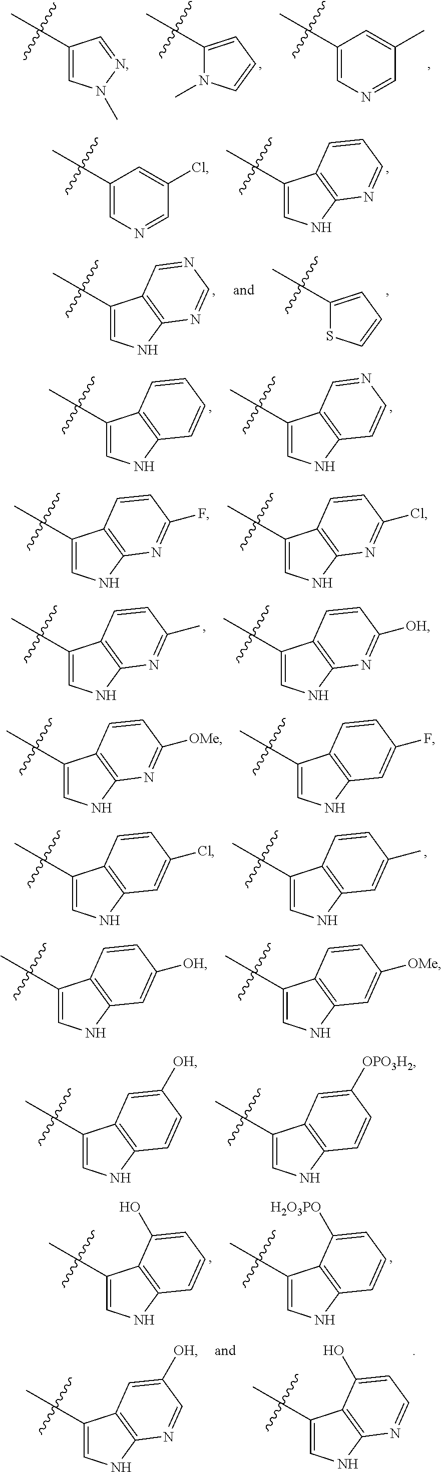

17. The compound of claim 1, which is selected from the group consisting of:

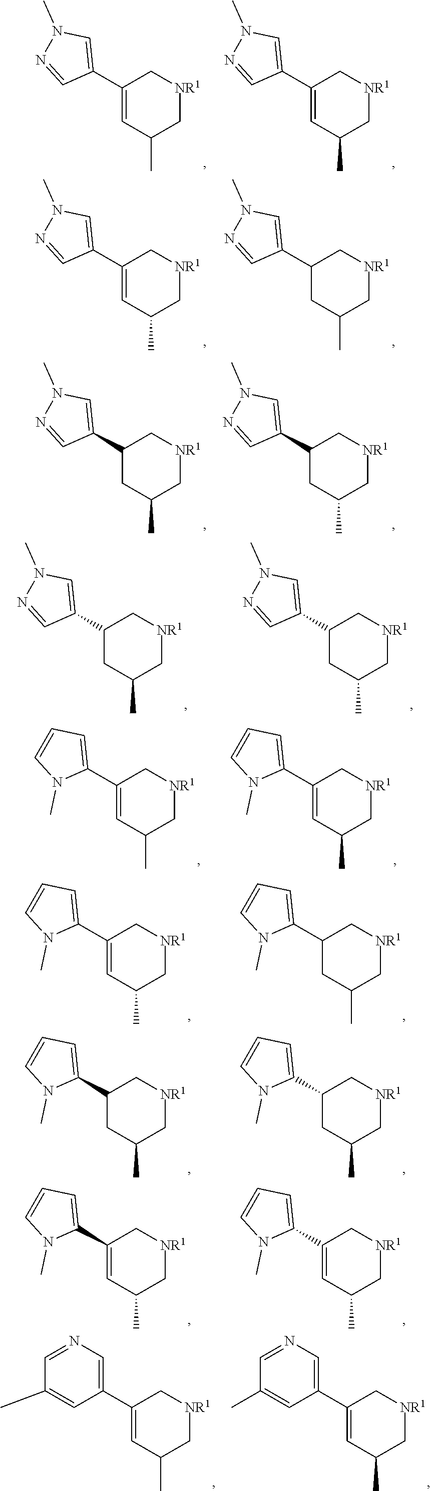

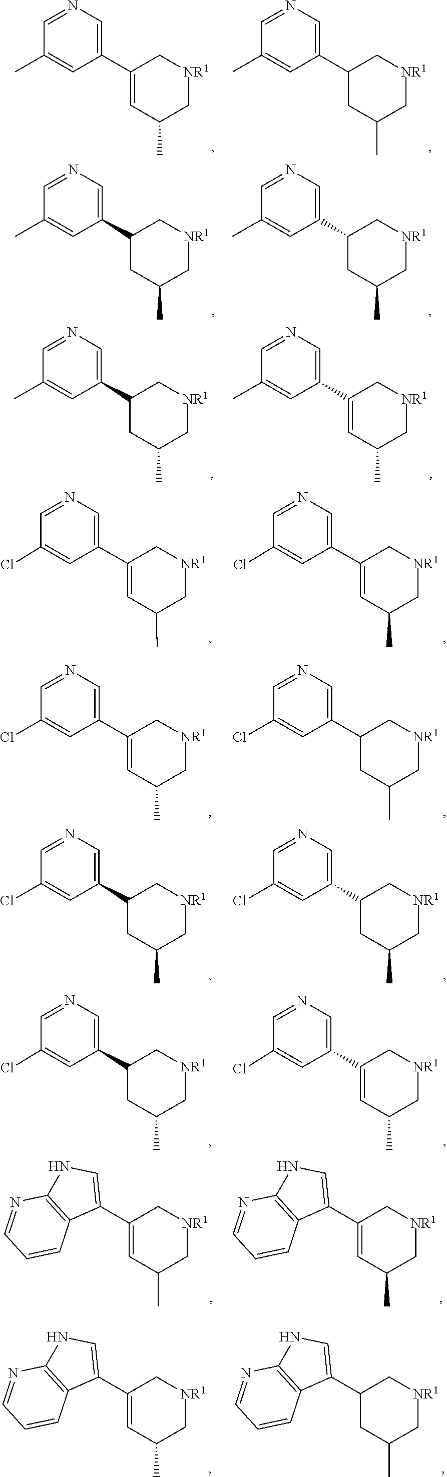

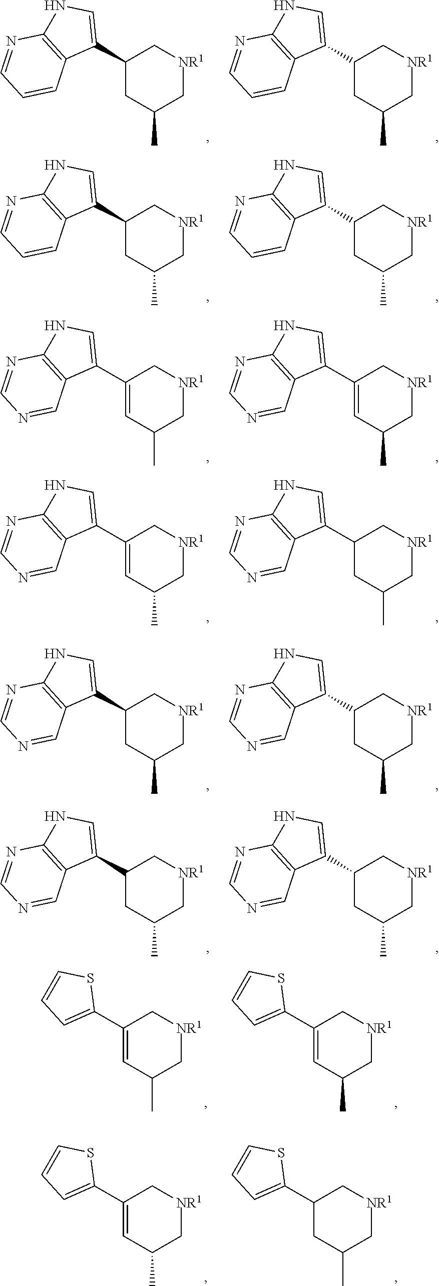

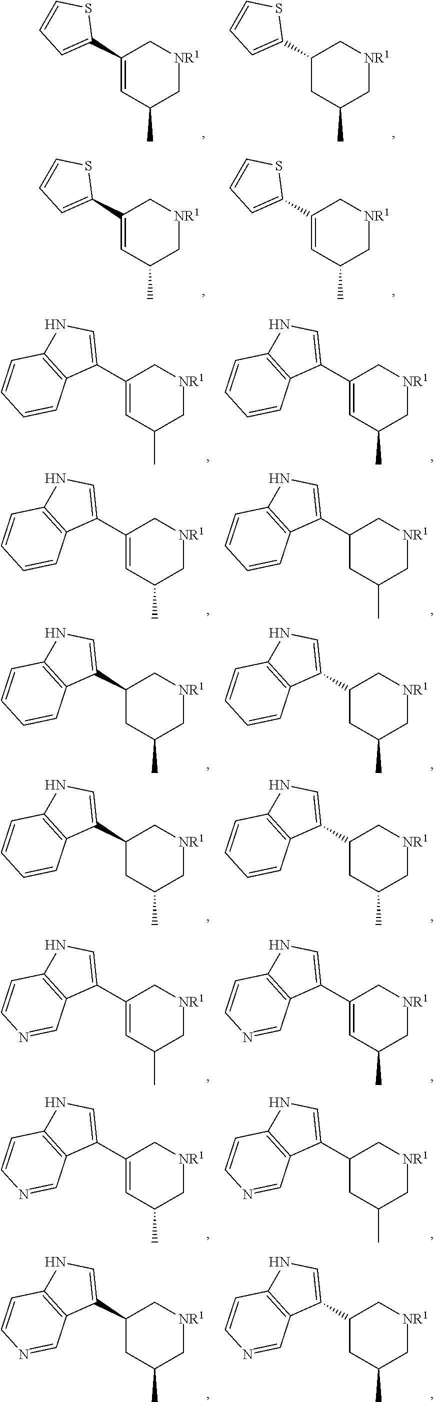

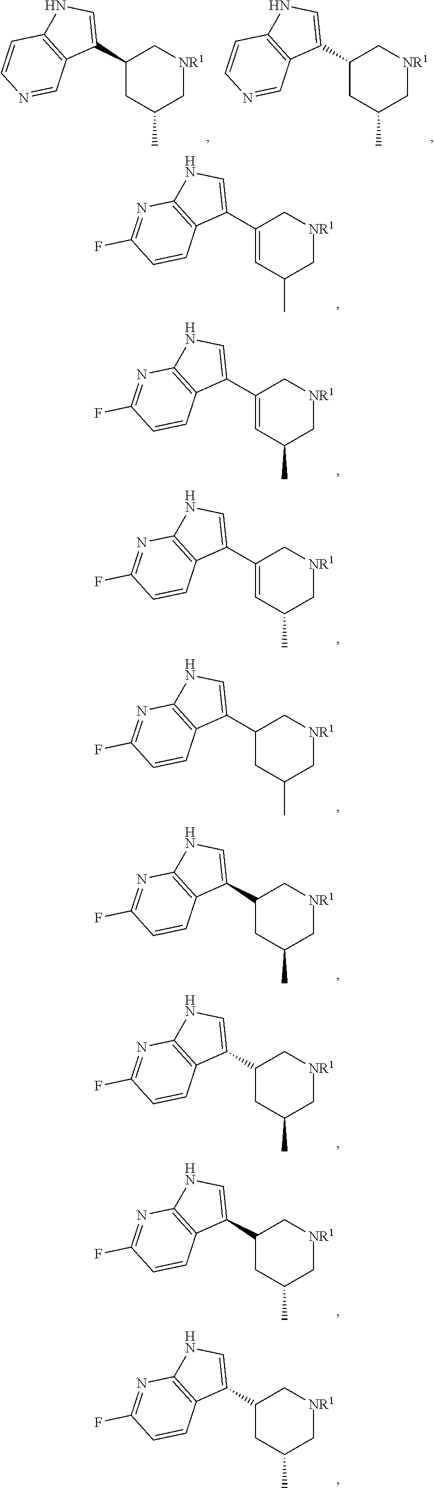

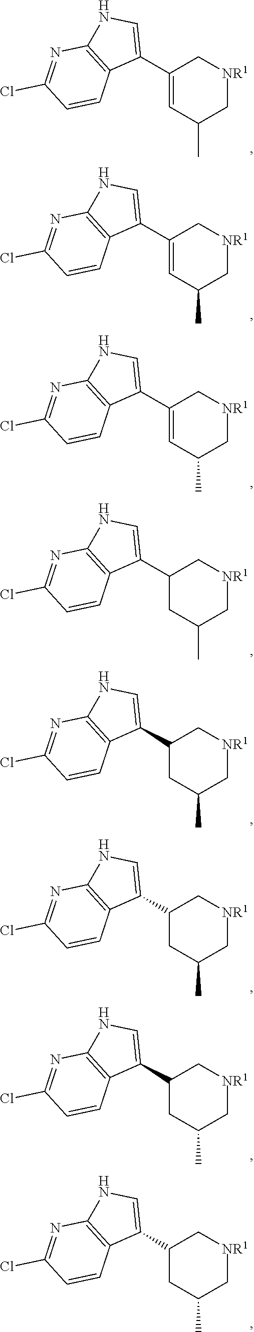

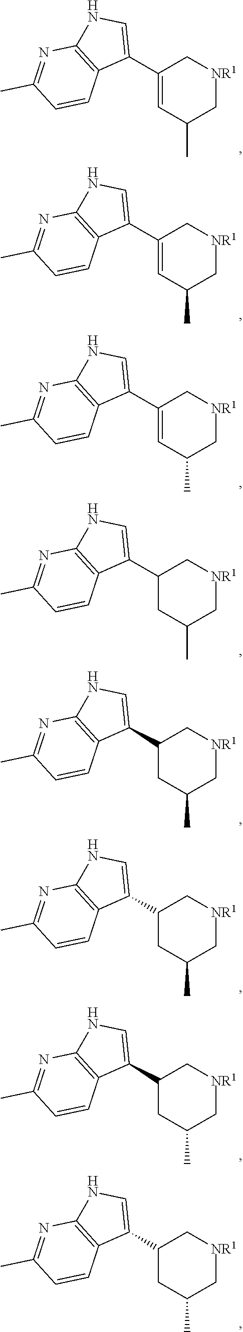

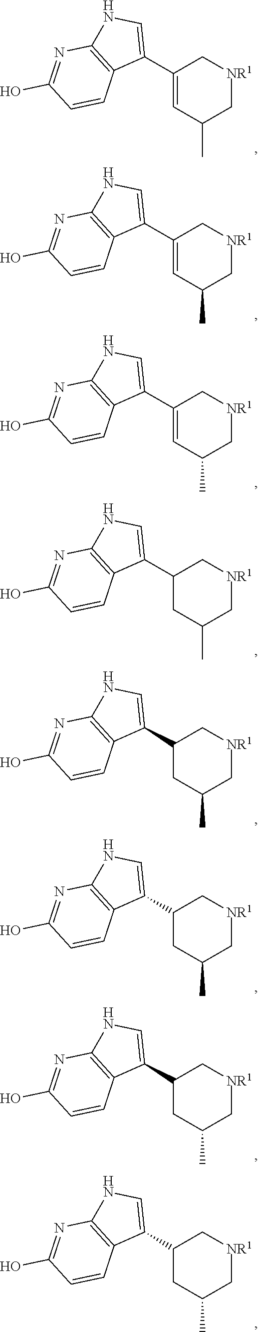

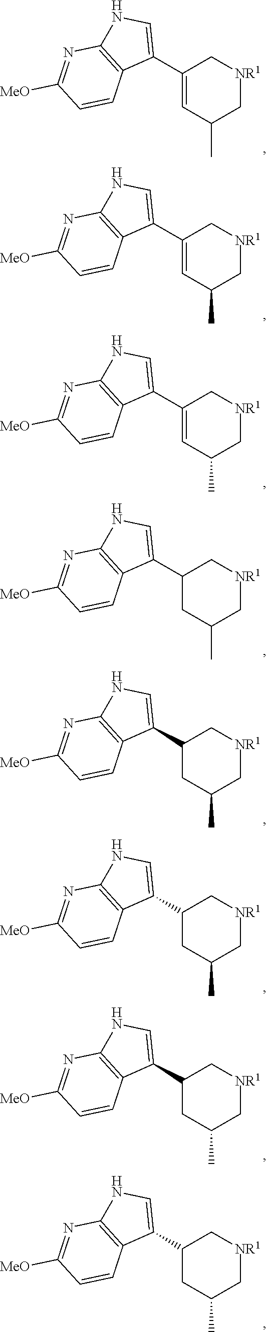

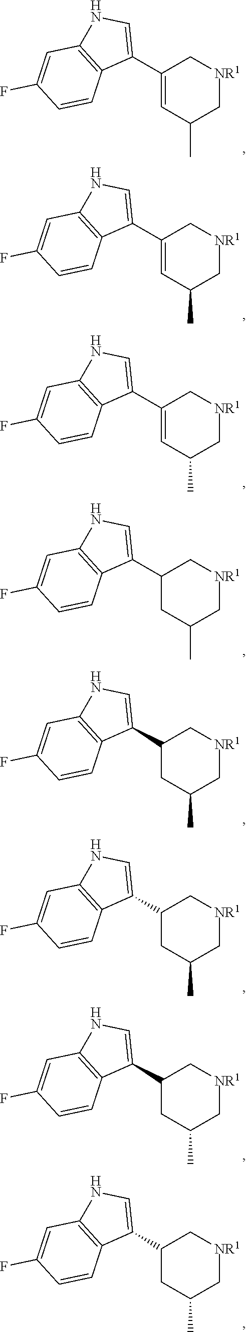

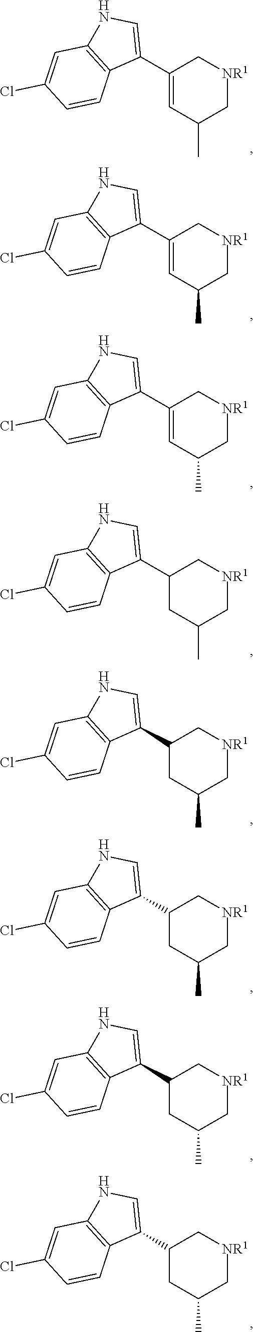

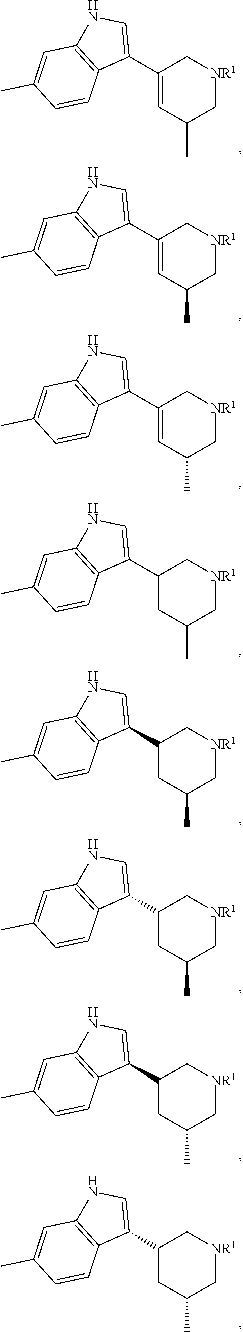

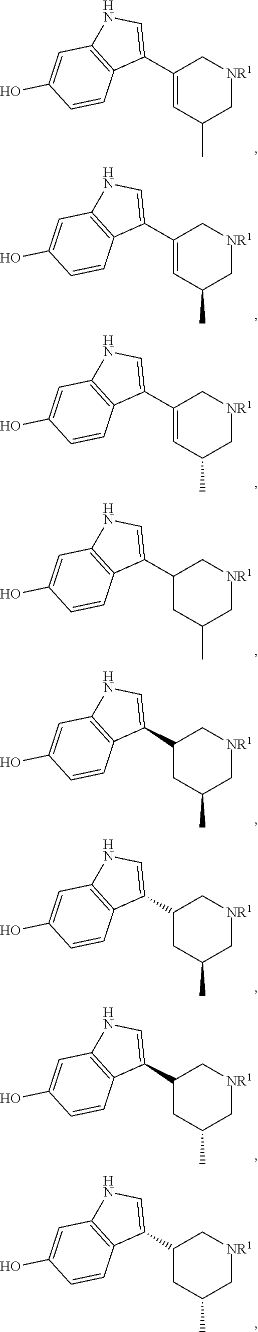

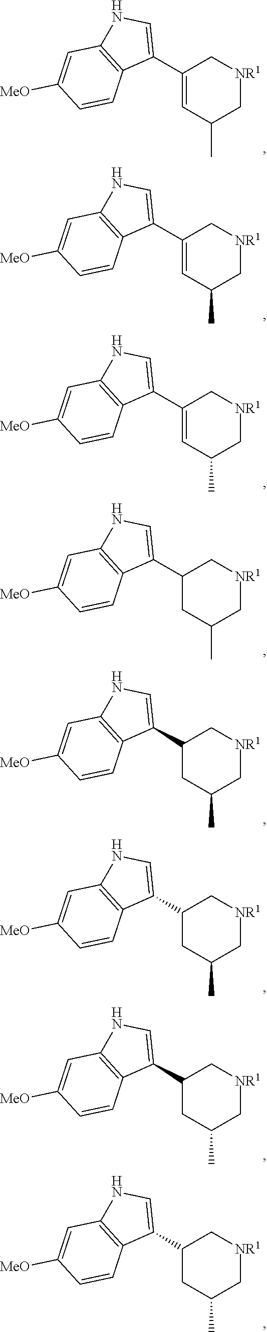

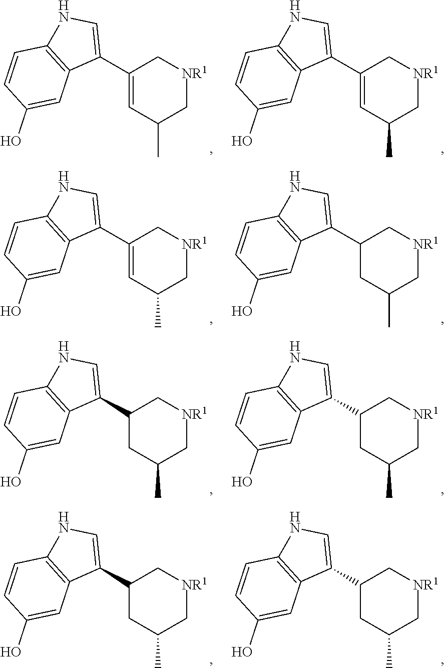

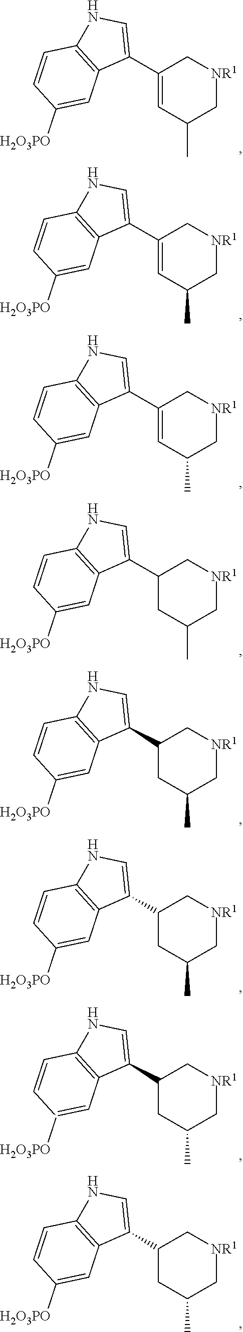

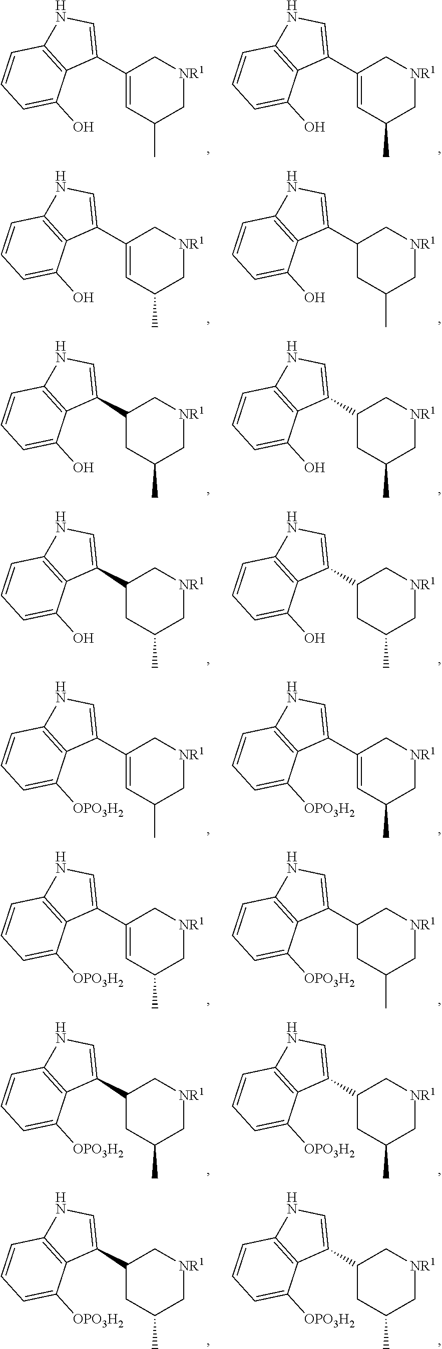

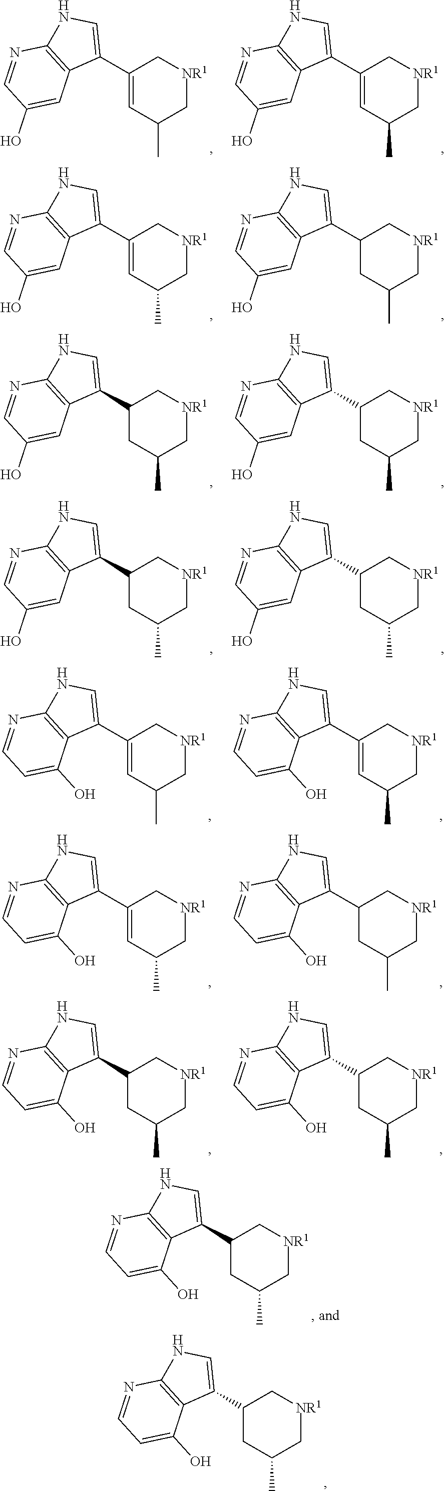

18. The compound of claim 1, which is selected from the group consisting of:

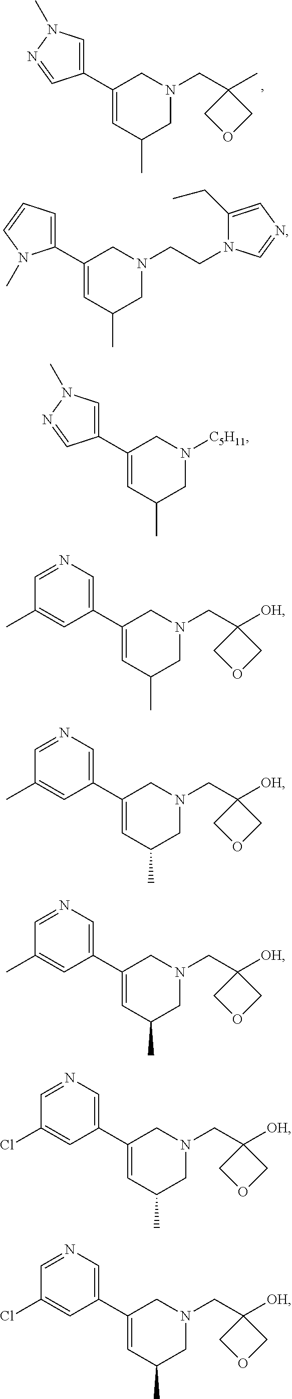

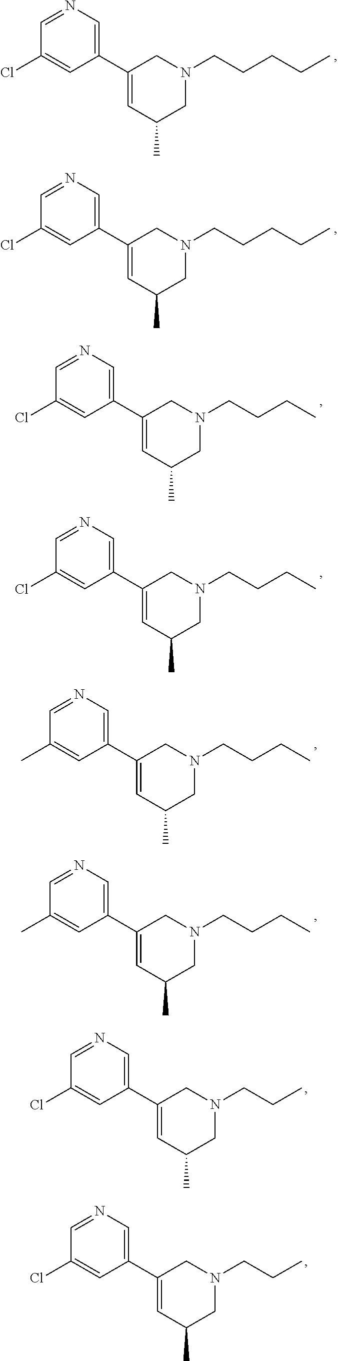

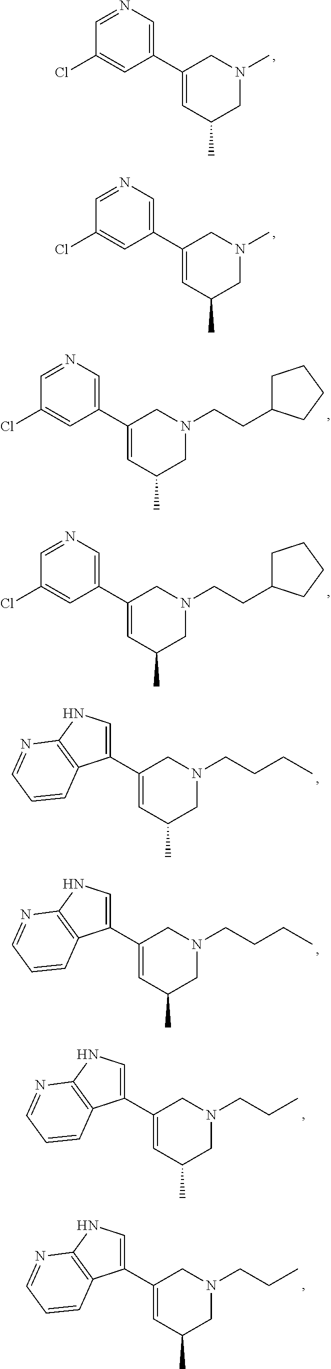

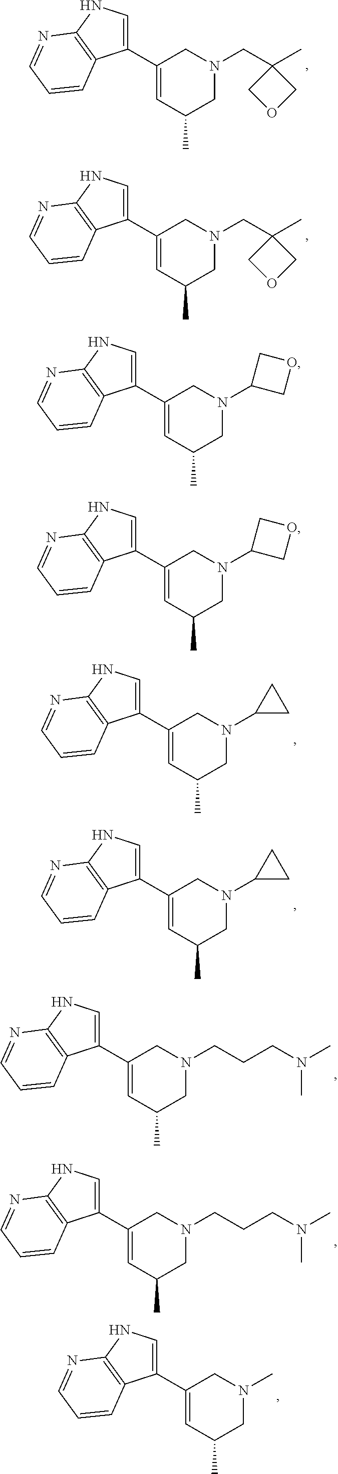

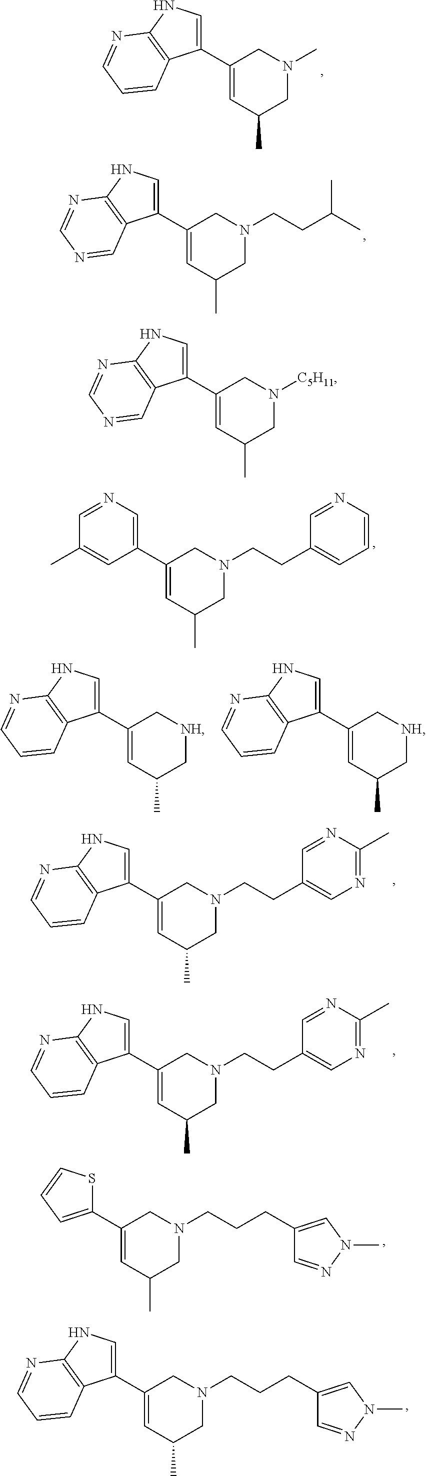

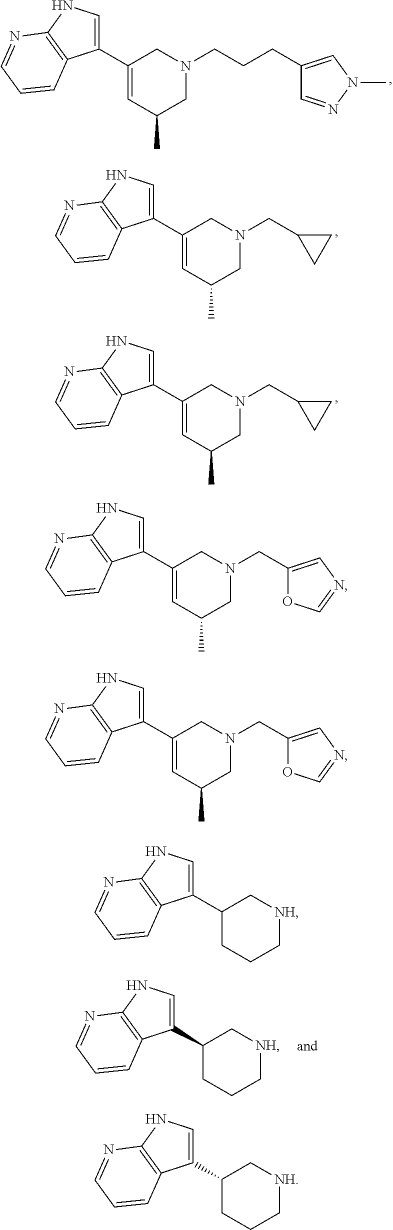

19. A pharmaceutical composition comprising the compound of claim 1 and at least one pharmaceutically acceptable excipient, optionally further comprising an additional therapeutic agent that treats, ameliorates, or prevents a neurological disease or disorder.

20. (canceled)

21. A method of treating, ameliorating, or preventing a neurological disease or disorder, the method comprising administering to a subject in need thereof a therapeutically effective amount of a compound of formula (II): 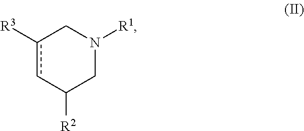
wherein: [symbol]
represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, and optionally substituted C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂; and
each occurrence of R is independently H, C₁-C₁₂ alkyl, C₃-C₁₂ cycloalkyl, or —(C₁-C₁₂ alkyl)C₃-C₁₂ cycloalkyl;
or a salt, solvate, tautomer, N-oxide, geometric isomer, or stereoisomer thereof.

22. The method of claim 21, wherein at least one of the following applies:
(a) the neurological disease or disorder is selected from the group consisting of depression, anxiety, substance abuse, and headaches;
(b) the compound is formulated as a pharmaceutical composition further comprising at least one pharmaceutically acceptable excipient;
(c) the compound is administered by a route selected from the group consisting of oral, transdermal, intravesical, intrapulmonary, intraduodenal, intragastrical, intrathecal, subcutaneous, intramuscular, intradermal, intra-arterial, intravenous, intrabronchial, inhalation, and topical; and
(d) the subject is a mammal, optionally wherein the mammal is a human.

23-26. (canceled)

27. A method of selectively agonizing the 5-hydroxytryptamine 2A (5-HT_{2A}) receptor in a subject in need thereof, the method comprising administering to a subject in need thereof a therapeutically effective amount of a compound of formula (II): 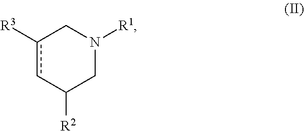
wherein: [symbol]
represents a single or double bond;
R¹ is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, and optionally substituted C₂-C₁₈ heterocyclyl;
R² is selected from the group consisting of H, optionally substituted C₁-C₁₂ alkyl, optionally substituted C₁-C₁₂ heteroalkyl, optionally substituted C₃-C₁₂ cycloalkyl, optionally substituted —(C₁-C₁₂ alkyl)C₃-C₁₂ cycloalkyl, optionally substituted C₂-C₁₈ heterocyclyl, and optionally substituted —(C₁-C₁₂ alkyl)C₂-C₁₈ heterocyclyl;
R³ is selected from the group consisting of optionally substituted C₂-C₁₈ heterocyclyl and optionally substituted —(C₁-C₁₂ alkyl)C₂-C₁₈ heterocyclyl;
each occurrence of optional substitution comprises 1 to 6 substituents independently selected from the group consisting of F, Cl, Br, I, OR, CN, NO₂, CF₃, OCF₃, R, N(R)₂, SOR, SO₂R, SO₂N(R)₂, C(O)R, and C(O)N(R)₂; and
each occurrence of R is independently H, C₁-C₁₂ alkyl, C₃-C₁₂ cycloalkyl, or —(C₁-C₁₂ alkyl)C₃-C₁₂ cycloalkyl;
or a salt, solvate, tautomer, N-oxide, geometric isomer, or stereoisomer thereof.

28. The method of claim 27, wherein at least one of the following applies:
(a) the compound is formulated as a pharmaceutical composition further comprising at least one pharmaceutically acceptable excipient;
(b) the composition is administered by a route selected from the group consisting of oral, transdermal, intravesical, intrapulmonary, intraduodenal, intragastrical, intrathecal, subcutaneous, intramuscular, intradermal, intra-arterial, intravenous, intrabronchial, inhalation, and topical;
(c) the subject is a mammal, optionally wherein the mammal is a human.

29-31. (canceled)
